(19) United States
(12) Patent Application Publication
(10) Pub. No.: US 2020/0150123 A1
(43) Pub. Date: May 14, 2020

(54) TUMOR MARKERS FUS, SMAD4, DERL1, YBX1, PS6, PDSS2, CUL2, AND HSPA9 FOR ANALYZING PROSTATE TUMOR SAMPLES

(71) Applicant: METAMARK GENETICS, INC., Cambridge, MA (US)

(72) Inventors: Michail V. Shipitsin, Brookline, MA (US); Eldar Y. Giladi, Arlington, MA (US); Clayton G. Small, III, Newbury, MA (US); Thomas P. Nifong, Winchester, MA (US); James Patrick Dunyak, Lexington, MA (US); Peter Blume-Jensen, Newton, MA (US)

(21) Appl. No.: 16/432,199

(22) Filed: Jun. 5, 2019

Related U.S. Application Data
(63) Continuation of application No. 14/776,448, filed on Sep. 14, 2015, now abandoned.
(60) Provisional application No. 61/792,003, filed on Mar. 15, 2013.

(51) Int. Cl. G01N 33/574; G06T 7/136; G06T 7/11; G06T 7/00; C12Q 1/6886

(52) U.S. Cl. CPC G01N 33/57434; G06T 7/136; G06T 7/11; G16H 50/30; C12Q 1/6886; Y02A 90/26; G06T 7/0012

(57) ABSTRACT

Provided herein are methods, e.g., computer-implemented methods or automated methods, of evaluating a cancer sample, e.g., a prostate tumor sample, from a patient. Also provided are biomarker panels for prognosticating prostate cancer. Also provided are methods of treating prostate cancer by identifying aggressive prostate cancer or prostate cancer that may have lethal outcome.

### RELATED APPLICATIONS

The present application is a Continuation of U.S. application Ser. No. 14/776,448, filed Sep. 14, 2015, which is a U.S. National Phase Application under 35 U.S.C. § 371 of International Application No. PCT/US2014/029158, filed Mar. 14, 2014, which claims the benefit of U.S. Provisional Application No. 61/792,003, filed Mar. 15, 2013, the entire content of which are hereby incorporated by reference in their entireties.

### SEQUENCE LISTING

The instant application contains a Sequence Listing which has been submitted electronically in ASCII format and is hereby incorporated by reference in its entirety. Said ASCII copy, created on Nov. 12, 2015, is named M2129-7003US_SL.txt and is 157,365 bytes in size.

### FIELD OF THE INVENTION

This invention relates to using biomarker panels to predict prognosis in cancer patients.

### BACKGROUND OF THE INVENTION

Prostate cancer (PCA) is the most common cancer in men. Most elderly men harbor prostatic neoplasia, with the vast majority of cases remaining localized and indolent without a need for therapeutic intervention. But there are a subset of early stage PCAs that are “hardwired” for aggressive malignancy and, if left untreated, will spread beyond the prostate and progress relentlessly to metastatic disease and ultimately death. The current inability to accurately distinguish indolent and aggressive diseases has subjected many men with potentially indolent disease to unnecessary radical therapeutic interventions, such as prostatectomy and beam radiation, with high morbidity. In the U.S. alone, costs associated with over-treatment of prostate cancer is estimated to be in excess of 2 billion dollars annually. And this does not include the quality-of-life impact from treatment procedures. In the meantime, some patients with potentially aggressive PCA are undertreated, and die due to disease progression.

Current methods of stratifying prostate cancer to predict outcome are based on clinical factors including Gleason grade, prostate-specific antigen (PSA) level, and tumor stage. However, these factors do not fully predict outcome and are not reliably linked to the most meaningful clinical endpoints of metastatic risk and PCA-specific death. This unmet medical need has fueled efforts to define the genetic and biological bases of PCA progression with the goals of identifying biomarkers capable of assigning progression risk and providing opportunities for targeted interventional therapies. Genetic studies of human PCA have identified a number of signature events, including PTEN tumor suppressor inactivation and ETS family translocation and disregulation, as well as other genetic or epigenetic alterations such as Nkx3.1, c-Myc, and SPINK. Global molecular analyses have also identified an array of potential recurrence/metastasis biomarkers, such as ECAD, AIPC, Pim-1 Kinase, hepsin, AMACR, and EZH2. However, the intense heterogeneity of human PCA has limited the utility of single biomarkers in clinical settings, thus prompting more comprehensive transcriptional profiling studies to define prognostic multi-gene biomarker panels or signatures. These panels or signatures may seem more robust, but their clinical utility remains uncertain due to the inherent noise and context-specific nature of transcriptional networks and the extreme instability of cancer genomes with myriad bystander genetic and epigenetic events that produce significant disease heterogeneity. Accordingly, a need exists for more accurate prognostic tests in early stage tumors that can be used to predict the occurrence and behavior of cancer, particularly at an early stage, and therefore are useful in guiding appropriate treatment for prostate cancer patients.

### SUMMARY OF THE INVENTION

In one aspect provided herein is a method, e.g., a computer-implemented method or automated method, of evaluating a cancer sample, e.g., a prostate tumor sample, from a patient. The method comprises identifying, the level, e.g., the amount of, or the level of expression for, 1, 2, 3, 4, 5, 6, 7, or 8 tumor markers of FUS, SMAD4, DERL1, YBX1, pS6, PDSS2, CUL2, and HSPA9 (the tumor marker set), or of a DNA or mRNA for said tumor marker(s), thereby evaluating said tumor sample.

In embodiments, the method comprises acquiring, e.g., directly or indirectly, a signal for a tumor marker. In embodiments, the method comprises directly acquiring the signal.

In embodiments, the method comprises directly or indirectly acquiring the cancer sample.

Also provided herein is a reaction mixture comprising (a) a cancer sample; and (b) a detection reagent for 1, 2, 3, 4, 5, 6, 7, or 8 tumor markers of FUS, SMAD4, DERL1, YBX1, pS6, PDSS2, CUL2, and HSPA9 (the tumor marker set), or of a DNA or mRNA for said tumor marker. In some embodiments of the reaction mixture, the cancer sample comprises a plurality of portions, e.g., slices or aliquots. In some embodiments of the reaction mixture, a first portion of the cancer sample comprises a detection reagent for a first, but not all of said markers, and a second portion of the cancer sample comprises a detection reagent for a detection marker for one of the markers but does not comprise a detection reagent for the first marker.

Also provided herein is a method, e.g., a computer-implemented method or automated method, of evaluating a sample, e.g., a tissue sample, e.g., a cancer sample, e.g., a prostate tumor sample, from a patient. The method comprises: (a) identifying, in a region of interest (ROI), from said sample, a level of a first region phenotype marker, e.g., a first tumor marker, thereby evaluating said sample.

In embodiments, the sample is a cancer sample. In embodiments, the sample comprises cells from a solid tumor. In embodiments, the sample comprises cells from a liquid tumor. In embodiments, the ROI is defined by or selected by a morphological characteristic.

In embodiments, the ROI is defined by or selected by manual or automated means and physical separation of the ROI from other cells or material, e.g., by dissection of a ROI, e.g., a cancerous region, from other tissue, e.g., noncancerous cells. In embodiments, the ROI is defined by or selected by a non-morphological characteristics, e.g., a ROI marker. In embodiments, the ROI is identified or selected by virtue of inclusion of a ROI marker by way of cell sorting. In embodiments, the ROI is identified or selected by a combination of a morphological and a non-morphological selection.

In embodiments, the level of a first region phenotype marker, e.g., a first tumor marker, is identified in a first ROI, e.g., a first cancerous region, and the level of a second region phenotype marker, e.g., a second tumor marker in a second ROI, e.g., a second cancerous region.

In embodiments, the level of a first and the level of a second region phenotype marker, e.g., a tumor marker, are identified in the same ROI, e.g., the same cancerous region.

In embodiments, the method further comprises: (b) identifying a ROI, e.g., a ROI that corresponds to a cancerous region;

In some embodiments, (a) is performed prior to (b).

In other embodiments, (b) is performed prior to (a).

In embodiments, identifying a level of a first region phenotype marker, e.g., a first tumor marker, comprises acquiring, e.g., directly or indirectly, a signal related to, e.g., proportional to, the binding of a detection reagent to said first region phenotype marker, e.g., a first tumor marker.

In embodiments, the method comprises contacting the sample with a detection reagent for a first region phenotype marker, e.g., a first tumor marker.

In embodiments, the method comprises contacting the sample with a detection reagent for a ROI marker, e.g., an epithelial marker,

In embodiments, the method further comprises acquiring an image of the sample, and analyzing the image. In some such embodiments, the method of comprises calculating from said image, a risk score for said patient.

In embodiments, the method comprises contacting the sample with a detection reagent for the first region phenotype marker, e.g., tumor marker, and acquiring a value for binding of the detection reagent. In some such embodiments, the method comprises calculating from the value a risk score for said patient.

In embodiments, the method further comprises (b) contacting the sample with a detection reagent for a ROI marker. In embodiments, the method further comprises (c) defining a ROI. In embodiments, the method further comprises (d) identifying the level of a region-phenotype marker, e.g., a tumor marker, in said ROI. In embodiments, the method further comprises (e) analyzing said level to provide a risk score. In embodiments, the method further comprises repeating steps (a)-(d).

In embodiments, the method further comprises (i) subjecting said sample to a sample to one or more physical preparation steps, e.g., dissociating, e.g., trypsinizing, said sample, dissecting said sample, or contacting said sample with a detection reagent for a ROI marker; (ii) contacting said ROI with a detection reagent; and/or (iii) detecting a signal from said ROI.

Also featured herein is a method, e.g., a computer-implemented method or automated method, of evaluating a tumor sample, e.g., a prostate tumor sample, from a patient, comprising:

(a) identifying, in a ROI, e.g., a cancerous ROI, a level of, e.g., the amount of, a first region-phenotype marker, e.g., a first tumor marker, e.g., wherein said first tumor marker is selected from FUS, SMAD4, DERL1, YBX1, pS6, PDSS2, CUL2, and HSPA9 (the tumor marker set), or of a DNA or mRNA for said first tumor marker, thereby evaluating said tumor sample.

In embodiments, the level of a first region-phenotype marker, e.g., a first tumor marker, from said tumor marker set is identified in a first ROI, e.g., cancerous ROI, and the level of a second region-phenotype marker, e.g., a second tumor marker from said tumor marker set is identified in a second ROI, e.g., a second cancerous ROI. In embodiments, said first ROI, e.g., cancerous ROI, and said ROI, e.g., a second cancerous ROI, are identified or selected by the same method or criteria. In embodiments, the level of a first and the level of a second region-phenotype marker, e.g., a first and second tumor marker, both from said tumor marker set, are identified in the same ROI, e.g., the same cancerous ROI.

In embodiments, the method further comprises: (b) identifying a ROI, e.g., a ROI of said tumor sample that corresponds to tumor epithelium. In some embodiments of the method, (a) is performed prior to (b). In some embodiments of the method, (b) is performed prior to (a).

In embodiments, identifying a level of a first region-phenotype marker, e.g., a first tumor marker, comprises acquiring, e.g., directly or indirectly, a signal related to, e.g., proportional to, the binding of a detection reagent to said first region-phenotype marker, e.g., a first tumor marker. In embodiments, the tumor marker is DNA that encodes FUS, SMAD4, DERL1, YBX1, pS6, PDSS2, CUL2, or HSPA9HSPA9. In embodiments, the tumor marker is mRNA that encodes FUS, SMAD4, DERL1, YBX1, pS6, PDSS2, CUL2, or HSPA9. In embodiments, the tumor marker is a protein selected from FUS, SMAD4, DERL1, YBX1, pS6, PDSS2, CUL2, or HSPA9.

In embodiments, the method comprises contacting the sample with a detection reagent for a marker of the tumor marker set, acquiring, directly or indirectly, an image of the sample, and analyzing the image. In embodiments, the method comprises calculating from the image a risk score for the patient.

In embodiments, the method comprises contacting the sample with a detection reagent for the first marker of the tumor marker set, acquiring, directly or indirectly, a value for binding of the detection reagent. In embodiments, the method comprises calculating from said value a risk score for said patient.

In embodiments of any of any one of the foregoing methods, the method further comprises identifying, in an ROI (e.g., the same or a different ROI), e.g., an ROI that corresponds to tumor epithelium, a level of a second tumor marker selected from said tumor marker set, or a DNA or mRNA for said second tumor marker.

In embodiments, said second tumor marker is a protein from said tumor market set.

In embodiments, the method further comprises identifying, in an ROI (e.g., the same or a different ROI), e.g., a ROI that corresponds to tumor epithelium, a level of a third tumor marker selected from said tumor marker set, or a DNA or mRNA for said third tumor marker.

In embodiments, the method further comprises identifying, in an ROI (e.g., the same or a different ROI), e.g., a ROI that corresponds to tumor epithelium, a level of a fourth tumor marker selected from said tumor marker set, or a DNA or mRNA for said fourth tumor marker.

In embodiments, the method further comprises identifying, in an ROI (e.g., the same or a different ROI), e.g., a ROI that corresponds to tumor epithelium, a level of a fifth tumor marker selected from said tumor marker set, or a DNA or mRNA for said fifth tumor marker.

In embodiments, the method further comprises identifying, in an ROI (e.g., the same or a different ROI), e.g., a ROI that corresponds to tumor epithelium, a level of a sixth tumor marker selected from said tumor marker set, or a DNA or mRNA for said sixth tumor marker.

In embodiments, the method further comprises identifying, in an ROI (e.g., the same or a different ROI), e.g., a ROI that corresponds to tumor epithelium, a level of a seventh tumor marker selected from said tumor marker set, or a DNA or mRNA for said seventh tumor marker.

In embodiments, the method further comprises identifying, in an ROI (e.g., the same or a different ROI), e.g., a ROI that corresponds to tumor epithelium, a level of a eighth tumor marker selected from said tumor marker set, or a DNA or mRNA for said eighth tumor marker.

In embodiments, the method further comprises identifying the level of an additional marker disclosed herein, other than a marker or said tumor marker set.

In embodiments, the level of said additional marker is identified in a cancerous ROI.

In embodiments, the level of said additional marker is identified in a benign ROI.

In embodiments of any of any one of the foregoing methods, wherein the method further comprises providing said tumor sample or said cancer sample. (As used herein, unless the context indicates otherwise, the terms “cancer sample” and “tumor sample” are interchangeable.)

In embodiments of any of any one of the foregoing methods, the method further comprises said tumor sample from another entity, e.g., a hospital, laboratory, or clinic.

In embodiments of any of any one of the foregoing methods, said cancer sample or said tumor sample comprises a prostate tissue section or slice.

In embodiments of any of any one of the foregoing methods, said cancer sample or said tumor sample comprises a plurality of portions, e.g., a plurality of prostate tissue sections or slices.

In embodiments of any of any one of the foregoing methods, said cancer sample or said tumor sample is fixed, e.g., formalin fixed.

In embodiments of any of any one of the foregoing methods, said cancer sample or said tumor sample is embedded in a matrix.

In embodiments of any of any one of the foregoing methods, said cancer sample or said tumor sample is paraffin embedded.

In embodiments of any of any one of the foregoing methods, said cancer sample or said tumor sample is de-paraffinated.

In embodiments of any of any one of the foregoing methods, said cancer sample or said tumor sample is a formalin-fixed, paraffin-embedded, sample, or its equivalent.

In embodiments, the cancer sample or tumor sample preparation (e.g., de-paraffination) is automated.

In embodiments of any of any one of the foregoing methods, the contact of detection reagents with said cancer sample or tumor sample is automated.

In embodiments of any of any one of the foregoing methods, the cancer sample or tumor sample is placed in an automated scanner.

In embodiments of any of any one of the foregoing methods, the cancer sample or tumor sample, e.g., a portion, e.g., a section or slice, of prostate tissue, is disposed on a substrate, e.g., a solid or rigid substrate, e.g., a glass or plastic substrate, e.g., a glass slide. In some such embodiments, a first portion, e.g., a section or slice, of said tumor sample, is disposed on a first substrate, e.g., a solid or rigid substrate, e.g., a glass or plastic substrate, e.g., a glass slide. In embodiments, a second portion, e.g., a section or slice, of said tumor sample, is disposed on a second substrate, e.g., a solid or rigid substrate, e.g., a glass or plastic substrate, e.g., a glass slide. In embodiments, a third portion, e.g., a section or slice, of said tumor sample, is disposed on a third substrate, e.g., a solid or rigid substrate, e.g., a glass or plastic substrate, e.g., a glass slide. In embodiments, a fourth portion, e.g., a section or slice, of said tumor sample, is disposed on a fourth substrate, e.g., a solid or rigid substrate, e.g., a glass or plastic substrate, e.g., a glass slide.

In embodiments, said first and second portions are analyzed simultaneously. In embodiments, said first and second portions are analyzed sequentially.

In embodiments of any of any one of the foregoing methods, said detection reagent comprises a tumor marker antibody, e.g., a tumor marker monoclonal antibody, e.g., a tumor marker antibody for FUS, SMAD4, DERL1, YBX1, pS6, PDSS2, CUL2, or HSPA9. In embodiments, said tumor marker antibody is conjugated to a label, e.g., a fluorescent moiety, e.g., a fluorescent dye.

In embodiments, said detection reagent comprises a second antibody, antibody, e.g., a monoclonal antibody, to said tumor marker antibody.

In embodiments, said detection reagent comprises a third antibody, antibody, e.g., a monoclonal antibody, to said second antibody.

In embodiments, said second antibody is conjugated to a label, e.g., a fluorescent moiety, e.g., a fluorescent dye.

In embodiments, said third antibody is conjugated to a label, e.g., a fluorescent moiety, e.g., a fluorescent dye.

In embodiments of any of any one of the foregoing methods, the cancer or tumor sample is contacted with:

a first ROI marker detection reagent, e.g., a total epithelial detection reagent, e.g., as described herein, having a first emission profile, e.g., a first peak emission, or which is measured in a first channel;

a second ROI marker detection reagent, e.g., a basal epithelial detection reagent, e.g., as described herein, having a second emission profile, e.g., a second peak emission, or which is measured in a second channel;

a region-phenotype marker, e.g., a tumor marker detection reagent, e.g., as described herein, having a third emission profile, e.g., a third peak emission, or which is measured in a third channel.

In embodiments, the cancer or tumor sample is further contacted with a nuclear detection reagent, having a fourth emission profile, e.g., a fourth peak emission, or which is measured in a fourth channel.

In embodiments, the cancer or tumor sample is further contacted is with a second region-phenotype marker, e.g., a second tumor marker detection reagent, e.g., as described herein, having a fifth emission profile, e.g., a fifth peak emission, or which is measured in a fifth channel.

In embodiments, the cancer or tumor sample is further contacted with a third region-phenotype marker, e.g., a third tumor marker detection reagent, e.g., as described herein, having a sixth emission profile, e.g., a sixth peak emission, or which is measured in a sixth channel.

In embodiments of any of any one of the foregoing methods, identifying a ROI, e.g., a cancerous ROI, comprises identifying a region having epithelial structure which lacks an outer layer of basal cells.

In embodiments, epithelial structure is detected with a first ROI-specific detection reagent, e.g., first total epithelial-specific detection reagent, e.g., an antibody, e.g., a monoclonal antibody, e.g., an anti-CK8 or anti-CK18 antibody, e.g., a monoclonal antibody.

In embodiments, epithelial structure is detected with said first ROI-specific detection reagent, e.g., said first total epithelial-specific detection reagent and a second ROI-specific detection reagent, e.g., a second total epithelial-specific detection reagent. In embodiments, one of said first ROI-specific detection reagent, e.g., said first total epithelial-specific detection reagent and said second ROI-specific detection reagent, e.g., said second total epithelial-specific detection reagent is a CK8 detection reagent, e.g., an anti-CK8 antibody, e.g., a monoclonal antibody, and the other is a CK18 biding reagent, e.g., an anti-CK18 antibody, e.g., a monoclonal antibody.

In embodiments, a signal for the binding of said first ROI-specific detection reagent, e.g., said first total epithelial detection reagent is detected through a first channel, e.g., at a first wavelength.

In embodiments, a signal for the binding of said first ROI-specific detection reagent, e.g., said first total epithelial detection reagent, and a signal for said second ROI-specific detection reagent, e.g., said second total epithelial detection reagent, are detected through said first channel, e.g., at a first wavelength.

In embodiments, said first (and if present, optionally, said second) ROI-specific detection reagent, e.g., said total epithelial detection reagent, comprises a marker antibody, e.g., a marker monoclonal antibody.

In embodiments, said first (and if present, optionally, said second) ROI-specific detection reagent, e.g., said total epithelial detection reagent, is conjugated to a label, e.g., a fluorescent moiety, e.g., a fluorescent dye.

In embodiments, said first (and if present, optionally, said second) ROI-specific detection reagent, e.g., said total epithelial binding agent, comprises a second antibody, antibody, e.g., a monoclonal antibody, to said marker antibody.

In embodiments, said first (and if present, optionally, said second) ROI-specific detection reagent, e.g., said total epithelial binding agent, comprises a third antibody, antibody, e.g., a monoclonal antibody, to said second antibody.

In embodiments, said second antibody is conjugated to a label, e.g., a fluorescent moiety, e.g., a fluorescent dye.

In embodiments, said third antibody is conjugated to a label, e.g., a fluorescent moiety, e.g., a fluorescent dye.

In embodiments, the presence or absence of basal cells is detected with a ROI-specific detection reagent, e.g., a basal epithelial detection reagent, e.g., a basal epithelial detection reagent described herein.

In embodiments, the methods further comprising indentifying an ROI, e.g., a second ROI, corresponding to a benign ROI of said tumor sample.

In embodiments, identifying a benign ROI comprises identifying a region having epithelial structure bounded by an outer layer of basal cells.

In embodiments, a basal cell is detected with an ROI-specific detection reagent for basal epithelium, e.g., an antibody, e.g., a monoclonal antibody, e.g., an anti-CK5 antibody, e.g., a monoclonal antibody or anti-TRIM29 antibody, e.g., a monoclonal antibody.

In embodiments, a basal cell is detected with said ROI-specific detection reagent for basal epithelium, and a second ROI-specific detection reagent for basal epithelium, e.g., an antibody, e.g., a monoclonal antibody, e.g., an anti-CK5 antibody, e.g., a monoclonal antibody or anti-TRIM29 antibody, e.g., a monoclonal antibody.

In embodiments, one of said first ROI-specific detection reagent for basal epithelium, and said ROI-specific detection reagent for basal epithelium, is a CK5 detection reagent, e.g., an anti-CK5 antibody, e.g., a monoclonal antibody, and the other is a TRIM29 detection reagent, e.g., an anti-TRIM29 antibody, e.g., a monoclonal antibody.

In embodiments, a signal for the binding of said first ROI-specific detection reagent for basal epithelium, is detected through a first channel, e.g., at a first wavelength.

In embodiments, a signal for the binding of said first ROI-specific detection reagent for basal epithelium, and a signal for said second ROI-specific detection reagent for basal epithelium, are detected through said first channel, e.g., at a first wavelength.

In embodiments, said first (and if present, optionally, said second) ROI-specific detection reagent for basal epithelium, comprises a marker antibody, e.g., a marker monoclonal antibody.

In embodiments, said first (and if present, optionally, said second) ROI-specific detection reagent for basal epithelium is conjugated to a label, e.g., a fluorescent moiety, e.g., a fluorescent dye.

In embodiments, said first (and if present, optionally, said second) ROI-specific detection reagent for basal epithelium, comprises a second antibody, e.g., a monoclonal antibody, to said marker antibody.

In embodiments, said first (and if present, optionally, said second) ROI-specific detection reagent for basal epithelium comprises a third antibody, antibody, e.g., a monoclonal antibody, to said second antibody.

In embodiments, said second antibody is conjugated to a label, e.g., a fluorescent moiety, e.g., a fluorescent dye.

In embodiments, said third antibody is conjugated to a label, e.g., a fluorescent moiety, e.g., a fluorescent dye.

In embodiments, the method further comprises identifying a ROI of said tumor sample as stromal.

In embodiments of any one of the foregoing methods, the method comprises (i.a) acquiring, directly or indirectly, a signal for a total epithelium specific marker, e.g., CK8; (ii.a) acquiring, directly or indirectly, a signal for a basal epithelium specific marker, e.g., CK5.

In embodiments of any one of the foregoing methods, the method further comprises: (i.b) acquiring, directly or indirectly, a signal for a second total epithelium specific marker, e.g., CK18; (ii.b) acquiring, directly or indirectly, a signal for a second basal epithelium specific marker, e.g., TRIM29. In embodiments, the method further comprises (iii) acquiring, directly or indirectly, a signal for a nuclear marker. In embodiments, the method further comprises (iv) acquiring, directly or indirectly, a signal for a second tumor marker of said tumor marker set. In embodiments, the method further comprises (v) acquiring, directly or indirectly, a signal for a third tumor marker of said tumor marker set. In embodiments, the method further comprises

(vi) acquiring, directly or indirectly, a signal for a fourth tumor marker of said tumor marker set. In embodiments, the method further comprises (vii) acquiring, directly or indirectly, a signal for a fifth tumor marker of said tumor marker set. In embodiments, the method further comprises (viii) acquiring, directly or indirectly, a signal for a sixth tumor marker of said tumor marker set. In embodiments, the method further comprises (ix) acquiring, directly or indirectly, a signal for a seventh tumor marker of said tumor marker set. In embodiments, the method further comprises (x) acquiring, directly or indirectly, a signal for an eighth tumor marker of said tumor marker set.

In embodiments, said signal for (i.a) and (i.b) have the same peak emission, or are collected in the same channel.

In embodiments, said signal for (ii.a) and (ii.b) have the same peak emission, or are collected in the same channel.

In embodiments of any one of the foregoing methods, the method comprises: (i.a) acquiring, directly or indirectly, a signal for a total epithelium specific marker, e.g., CK8; (i.b) acquiring, directly or indirectly, a signal for a second total epithelium specific marker, e.g., CK18; (ii.a) acquiring, directly or indirectly, a signal for a basal epithelium specific marker, e.g., CK5; (ii.b) acquiring, directly or indirectly, a signal for a second basal epithelium specific marker, e.g., TRIM29; (iii) acquiring, directly or indirectly, a signal for a nuclear marker; (iv) acquiring, directly or indirectly, a signal for a first tumor marker; (v) acquiring, directly or indirectly, a signal for a second tumor marker; or (vi) acquiring, directly or indirectly, a signal for a third tumor marker. In embodiments, the method comprises (i.a), (ii.a), (iii), and (iv). In embodiments, the method comprises (i.a), (i.b), (ii.a), (ii.b), (iii), and (iv). In embodiments, the method comprises all of (i.a)-(v). In embodiments, the method comprises all of (i.a)-(vi).

In embodiments of any one of the foregoing methods, the method further comprises identifying the level of a quality control marker, e.g., in a second ROI, e.g., a benign ROI. In embodiments, said quality control marker is selected from the tumor marker set, e.g., DERL1.

In embodiments, the method further comprises contacting said sample with a detection reagent for said quality control marker.

In embodiments, the method further comprises acquiring, e.g., directly or indirectly, a signal related to, e.g., proportional to, the binding of said detection reagent to said first quality control marker, e.g., in a second ROI, e.g., a benign ROI.

In embodiments, the method further comprises identifying the level of a second quality control marker, e.g., in a second ROI, e.g., a benign ROI. In embodiments, said second quality control marker is other than a marker from said tumor marker set. In embodiments, said second quality control marker is associated with lethality or aggressiveness of a tumor. In embodiments, said second quality control marker is a marker described herein, e.g., a tumor marker other than a marker from said tumor marker set. In embodiments, said second quality control marker is selected from ACTN and VDAC1.

In embodiments, the method further comprises identifying the level of a third quality control marker, e.g., in a second ROI, e.g., a benign ROI. In embodiments, said third quality control marker is other than a marker from said tumor marker set. In embodiments, said third quality control marker is a marker described herein, e.g., a tumor marker other than a marker from said tumor marker set. In embodiments, said third quality control marker is selected from ACTN and VDAC1.

In embodiments of any one of the foregoing methods, the method further comprises identifying, the level of, e.g., the amount of, a first quality control marker, e.g., DERL1, in a second ROI, e.g., a benign ROI; and identifying the level of a second quality control marker, e.g., one of ACTN and VDAC, in a second ROI, e.g., a benign ROI.

In embodiments, the method further comprises identifying the level of a third quality control marker, e.g., one of ACTN and VDAC, in a second ROI, e.g., a benign ROI. In embodiments, the level of the first, second and third quality controls markers are identified in the same second ROI, e.g., a benign ROI. In embodiments, the level of the first, second and third quality controls markers are identified in the different second ROIs, e.g., different benign ROIs.

In embodiments, the method further comprises identifying, the level of a first quality control marker, e.g., DERL1, in a second ROI, e.g., a benign ROI; identifying the level of a second quality control marker, e.g., one of ACTN and VDAC, in a second ROI, e.g., a benign ROI; and identifying the level of a third quality control marker, e.g., one of ACTN and VDAC, in a second ROI, e.g., a benign ROI, wherein, responsive to said levels, classifying the sample, e.g., as acceptable or not acceptable.

In embodiments, the method comprises detecting a signal for the level of one of said quality control markers. In embodiments, a first value for the detected signal indicates a first quality level, e.g., acceptable quality, and a second value for the detected signal indicates a second quality level, e.g., unacceptable quality. In embodiments, responsive to said value, the sample is processed or not processed, e.g., discarded, or a parameter for analysis is altered.

In embodiments of any one of the foregoing methods, the method comprises acquiring a multispectral image from said sample and unmixing said multi-spectral image into the following channels: a channel for a first ROI-specific detection reagent, e.g., an epithelial specific marker;

a channel for a second ROI-specific detection reagent, e.g., a basal epithelial specific marker; a channel for a nuclear specific signal, e.g., a DAPI signal; and a channel for a first population phenotype marker, e.g., a first tumor marker. In embodiments, the method comprises: use of a first channel to collect signal for a first ROI-specific detection reagent, e.g., a total epithelial marker; use of a second channel to collect signal for a second ROI-specific detection reagent, e.g., a basal epithelial marker; use of a third channel to collect signal for nuclear area; use of a fourth channel to collect signal for a first population phenotype marker, e.g., a first tumor marker selected from FUS, SMAD4, DERL1, YBX1, pS6, PDSS2, CUL2, and HSPA9. In embodiments, the method further comprises: use of a fifth channel to collect signal for a second population phenotype marker, e.g., a second tumor marker selected from FUS, SMAD4, DERL1, YBX1, pS6, PDSS2, CUL2, and HSPA9. In embodiments, the method further comprises: use of a sixth channel to collect signal for a third population phenotype marker, e.g., a third tumor marker selected from FUS, SMAD4, DERL1, YBX1, pS6, PDSS2, CUL2, and HSPA9.

In embodiments of any one of the foregoing methods, the method comprises acquiring an image of the area of the sample to be analyzed, e.g., as a DAPI filter image.

In embodiments of any one of the foregoing methods, the method comprises locating tissue, e.g., by application of a tissue-finding algorithm to an image collected from said sample.

In embodiments of any one of the foregoing methods, the method comprises re-acquisition of images with DAPI and FITC monochrome filters.

In embodiments of any one of the foregoing methods, the method comprises application of a tissue finding algorithm, e.g., to insure that images of a preselected number of fields containing sufficient tissue are acquired.

In embodiments of any one of the foregoing methods, the method comprises acquiring, directly or indirectly, consecutive exposures of DAPI, FITC, TRITC, and Cy5 filters.

In embodiments of any one of the foregoing methods, the method comprises acquiring a multispectral image of the area of the sample to be analyzed.

In embodiments of any one of the foregoing methods, the method comprises segmenting an area of said sample into epithelial cells, basal cells, and stroma.

In embodiments of any one of the foregoing methods, the method further comprises identifying areas of said sample into cytoplasmic and nuclear areas.

The method of any one of claims 1-166, comprising acquiring, e.g., directly or indirectly, a value for a population phenotype marker, e.g., a tumor marker, in the cytoplasm, nucleus, and/or whole cell of a cancerous ROI.

In embodiments of any one of the foregoing methods, the method comprises acquiring, e.g., directly or indirectly, a value for a population phenotype marker, e.g., a tumor marker in the cytoplasm, nucleus, and/or whole cell of benign ROI.

In embodiments of any one of the foregoing methods, said cancer or tumor sample comprises a plurality of portions, e.g., a plurality of section or slices.

In embodiments, the method comprises performing a step described herein, e.g., collecting or acquiring signal, or forming an image, e.g., identifying the level of a first population phenotype marker, e.g., a first tumor marker, from a first portion, e.g., section or slice; and performing a step described herein, e.g., collecting or acquiring signal, or forming an image, e.g., identifying the level of a second population phenotype marker, e.g., a second tumor marker, from a second portion, e.g., a second section or slice. In embodiments, said second tumor marker is selected from FUS, SMAD4, DERL1, YBX1, pS6, PDSS2, CUL2, and HSPA9. In embodiments, the method further comprises: identifying, in a second portion, e.g., a second section or slice, of said tumor sample, a ROI that corresponds to tumor epithelium; acquiring, e.g., directly or indirectly, from said ROI that corresponds to tumor epithelium, a signal for a second tumor marker selected from FUS, SMAD4, DERL1, YBX1, pS6, PDSS2, CUL2, and HSPA9.

In embodiments, the method comprises, for said second portion, e.g., a second section or slice, of said tumor sample, (i.a) acquiring a signal for a epithelium specific marker, e.g., CK8;

(ii.a) acquiring a signal for a basal epithelium specific marker, e.g., CK5.

In embodiments, the method further comprises, for said second portion, e.g., a second section or slice, of said tumor sample: (i.b) acquiring a signal for a second epithelium specific marker, e.g., CK18; (ii.b) acquiring a signal for a second basal epithelium specific marker, e.g., TRIM29.

In embodiments, the method further comprises, for said second portion, e.g., a second section or slice, of said tumor sample: (iii) acquiring a signal for a nuclear marker.

In embodiments, the method further comprises, for said second portion, e.g., a second section or slice, of said tumor sample; (iv) acquiring a signal for a second tumor marker of claim 1. In embodiments, the method further comprises, for said second portion, e.g., a second section or slice, of said tumor sample; (v) acquiring, directly or indirectly, a signal for a second tumor marker of said tumor marker set. In embodiments, the method further comprises, for said second portion, e.g., a second section or slice, of said tumor sample (vi) acquiring, directly or indirectly, a signal for a third tumor marker of said tumor marker set. In embodiments, the method further comprises, for said second portion, e.g., a second section or slice, of said tumor sample; (vii) acquiring, directly or indirectly, a signal for a fourth tumor marker of said tumor marker set. In embodiments, the method further comprises, for said second portion, e.g., a second section or slice, of said tumor sample; (viii) acquiring, directly or indirectly, a signal for a fifth tumor marker of said tumor marker set. In embodiments, the method further comprises, for said second portion, e.g., a second section or slice, of said tumor sample; (ix) acquiring, directly or indirectly, a signal for a sixth tumor marker of said tumor marker set. In embodiments, the method further comprises, for said second portion, e.g., a second section or slice, of said tumor sample; (x) acquiring, directly or indirectly, a signal for a seventh tumor marker of said tumor marker set. In embodiments, the method further comprises, for said second portion, e.g., a second section or slice, of said tumor sample; (xi) acquiring, directly or indirectly, a signal for an eighth tumor marker of said tumor marker set.

In embodiments, said signal for (i.a) and (i.b) have the same peak emission, or are collected in the same channel.

In embodiments, said signal for (ii.a) and (ii.b) have the same peak emission, or are collected in the same channel.

In embodiments, the method further comprises identifying, in a third portion, e.g., a third section or slice, of said tumor sample, a ROI that corresponds to tumor epithelium; acquiring, e.g., directly or indirectly, from said ROI that corresponds to tumor epithelium, a signal for a third tumor marker selected from FUS, SMAD4, DERL1, YBX1, pS6, PDSS2, CUL2, and HSPA9.

In embodiments, the method comprises, for a third portion, e.g., a third section or slice, of said tumor sample: (i.a) acquiring a signal for a epithelium specific marker, e.g., CK8; (ii.a) acquiring a signal for a basal epithelium specific marker, e.g., CK5. In embodiments, the method further comprises, for said third portion, e.g., a third section or slice, of said tumor sample: (i.b) acquiring a signal for a second epithelium specific marker, e.g., CK18; (ii.b) acquiring a signal for a second basal epithelium specific marker, e.g., TRIM29. In embodiments, the method further comprises, for said third portion, e.g., a third section or slice, of said tumor sample: (iii) acquiring a signal for a nuclear marker. In embodiments, the method further comprises, for said third portion, e.g., a third section or slice, of said tumor sample: (iv) acquiring a signal for a second tumor marker of claim 1. In embodiments, said signal for (i.a) and (i.b) have the same peak emission, or are collected in the same channel. In embodiments, said signal for (ii.a) and (ii.b) have the same peak emission, or are collected in the same channel.

In embodiments of any one of the foregoing methods, a first tumor sample portion, e.g., a first section or slice, is disposed on a first substrate. In embodiments, a second tumor sample portion, e.g., a second section or slice, is disposed on a second substrate. In embodiments, a third tumor sample portion, e.g., a third section or slice, is disposed on a third substrate.

In embodiments, a forth tumor sample portion, e.g., a fourth section or slice, is disposed on a fourth substrate.

In embodiments, a first tumor sample portion, e.g., a first section or slice, and a second tumor sample portion, e.g., a second section or slice, are disposed on the same substrate.

In embodiments of any one of the foregoing methods, the method further comprises saving or storing a value corresponding to a signal, value, or an image acquired from said sample, from any step in a method described herein, in digital or electronic media, e.g., in a computer database.

In embodiments of any one of the foregoing methods, the method comprises exporting a value or an image obtained from capture of signals from said tumor sample into software, e.g., pattern or object recognition software, to identify nuclear areas.

In embodiments of any one of the foregoing methods, the method comprises exporting a value or image obtained from capture of signals from said tumor sample into software, e.g., pattern or object recognition software, to identify cytoplasmic areas.

In embodiments of any one of the foregoing methods, the method comprises exporting a value or image obtained from capture of signals from said tumor sample into software, e.g., pattern or object recognition software, to identify cancerous ROIs.

In embodiments of any one of the foregoing methods, the method comprises exporting a value or image obtained from capture of signals from said tumor sample into software, e.g., pattern or object recognition software, to identify benign ROIs.

In embodiments of any one of the foregoing methods, the method comprises exporting a value or image obtained from capture of signals from said tumor sample into software, e.g., pattern or object recognition software, to provide a value for the level of said tumor marker in a cancerous ROI.

In embodiments of any one of the foregoing methods, the method comprises exporting a value or image obtained from capture of signals from said tumor sample into software, e.g., pattern or object recognition software, to provide a value for the level of said tumor marker in a benign ROI.

In embodiments of any one of the foregoing methods, the method comprises responsive to a signal for a region phenotype marker, e.g., a tumor marker, a signal for a first ROI marker, e.g., a total epithelium specific marker, and a signal for a second ROI marker, e.g., a basal epithelium specific marker, providing a value for the level of a region phenotype marker, e.g., a tumor marker, in a cancerous ROI. In embodiments, the method comprises calculating a risk score for said patient. In embodiments, the method comprises, responsive to said value, calculating a risk score for said patient.

In embodiments of any one of the foregoing methods, the method comprises responsive to a signal for a region phenotype marker, e.g., a tumor marker, a signal for a first ROI marker, e.g., a total epithelium specific marker, and a signal for a second ROI marker, e.g., a basal epithelium specific marker, providing a value for the level of a tumor marker in a benign ROI.

In embodiments of any one of the foregoing methods, the method comprises responsive to a signal for a region phenotype marker, e.g., a tumor marker, a signal for a first ROI marker, e.g., a total epithelium specific marker, and a signal for a second ROI marker, e.g., a basal epithelium specific marker, and a signal for a third ROI marker, e.g., a nucleus specific marker, providing a value for the cytoplasmic level of a tumor marker in a cancerous ROI.

In embodiments of any one of the foregoing methods, the method comprises responsive to a signal for a region phenotype marker, e.g., a tumor marker, a signal for a first ROI marker, e.g., a total epithelium specific marker, and a signal for a second ROI marker, e.g., a basal epithelium specific marker, and a signal for a third ROI marker, e.g., a nucleus specific marker, providing a value for the nuclear level of a tumor marker in a benign ROI.

In embodiments of any one of the foregoing methods, the method comprises, responsive to one or more of said values, calculating a risk score for said patient. In embodiments, the method comprises calculating a risk score for said patient, wherein said risk score is correlated to potential for extra-prostatic extension or metastasis.

In embodiments, the method comprises responsive to said risk score, prognosing said patient, classifying the patient, selecting a course of treatment for said patient, or administering a selected course of treatment to said patient.

In embodiments, said risk score corresponds to a ‘favorable’ case (e.g., surgical Gleason 3+3 or 3 with minimal 4, organ-confined (≤T2) tumors).

In embodiments, said risk score corresponds to a ‘non-favorable’ cases (e.g., capsular penetration (T3a), seminal vesicle invasion (T3b), lymph node metastases or dominant Gleason 4 pattern or higher).

In embodiments, said risk score allows discrimination between ‘favorable’ cases (e.g., surgical Gleason 3+3 or 3 with minimal 4, organ-confined (≤T2) tumors) and ‘non-favorable’ cases (e.g., capsular penetration (T3a), seminal vesicle invasion (T3b), lymph node metastases or dominant Gleason 4 pattern or higher).

In embodiments, said risk score corresponds to, or predicts: a surgical Gleason of 3+3 or localized disease (≤T3a) (defined as ‘low risk’); a surgical Gleason ≥3+4 or non-localized disease (T3b, N, or M) (defined as ‘intermediate-high risk’); a surgical Gleason ≤3+4 and organ confined disease (≤T2) (defined as ‘favorable’); or a surgical Gleason ≥4+3 or non-organ-confined disease (T3a, T3b, N, or M) (‘non-favorable’).

In embodiments wherein a risk score is calculated, the method further comprises, responsive to said risk score, identifying said patient as having aggressive cancer or having heightened risk or cancer related lethal outcome.

In embodiments wherein a risk score is calculated, the method further comprises (e.g., responsive to said risk score) selecting said patient for, or administering to said patient, adjuvant therapy.

Also provided herein is a kit comprising a detection reagent for 1, 2, 3, 4, 5, 6, 7, or all of the tumor markers FUS, SMAD4, DERL1, YBX1, pS6, PDSS2, CUL2, or HSPA9. In embodiments, the kit further comprises a detection reagent for a total epithelial marker and a basal epithelial marker.

Also provided herein is a cancer sample, e.g., a prostate tumor sample, having disposed thereon: a detection reagent for a total epithelial marker; a detection reagent for a basal epithelial marker; a detection reagent for a tumor marker selected from a FUS, SMAD4, DERL1, YBX1, pS6, PDSS2, CUL2, or HSPA9. In embodiments, the cancer sample, e.g., the prostate tumor sample, comprises a plurality of portions, e.g., slices. In embodiments, the cancer sample, e.g., the prostate tumor sample, has further disposed thereon, a detection reagent for a second tumor marker selected from a FUS, SMAD4, DERL1, YBX1, pS6, PDSS2, CUL2, or HSPA9.

Also featured herein is a computer-implemented method of evaluating a prostate tumor sample, from a patient, the method comprising: (i) identifying a ROI of said tumor sample that corresponds to tumor epithelium (a cancerous ROI); (ii) identifying, the level of, e.g., the amount of, each of the following tumor markers, FUS, SMAD4, DERL1, YBX1, pS6, PDSS2, CUL2, and HSPA9 (the tumor marker set), in a cancerous ROI, wherein identifying a level of tumor marker comprises acquiring, e.g., directly or indirectly, a signal related to, e.g., proportional to, the binding of an antibody for said tumor marker; (iii) providing a value for the level of each of the tumor markers in a cancerous ROI; and (iv) responsive to said values, evaluating said tumor sample, comprising, e.g., assigning a risk score to said patient by algorithmically combining said levels, thereby evaluating a prostate tumor sample.

In embodiments, the method comprises: use of a first channel to collect signal for a total epithelial marker; use of a second channel to collect signal for a basal epithelial marker; use of a third channel to collect signal for nuclear area; use of a fourth channel to collect signal for a tumor marker selected from FUS, SMAD4, DERL1, YBX1, pS6, PDSS2, CUL2, and HSPA9.

In embodiments, the level of a first tumor marker from said tumor marker set is identified in a first cancerous ROI and the level of a second tumor marker from said tumor marker set is identified in a second cancerous ROI.

In embodiments, the level of a first and the level of a second tumor marker, both from said tumor marker set, are identified in the same cancerous ROI.

In embodiments, the method further comprises: identifying, the level of a first quality control marker, e.g., DERL1, in a second ROI, e.g., a benign ROI; identifying the level of a second quality control marker, e.g., one of ACTN and VDAC, in a second ROI, e.g., a benign ROI; and identifying the level of a third quality control marker, e.g., one of ACTN and VDAC, in a second ROI, e.g., a benign ROI, wherein, responsive to said levels, classifying the sample, e.g., as acceptable or not acceptable.

This invention provides methods for predicting prognosis of cancer (e.g., prostate cancer) in a patient (e.g., a human patient). These methods provide reliable prediction on whether the patient has, or is at risk of having, an aggressive form of cancer, and/or on whether the patient is at risk of having a cancer-related lethal outcome.

In some embodiments, the prognostic methods of the invention comprise measuring, in a sample obtained from the patient, the levels of two or more Prognosis Determinants (PDs) selected from the group consisting of ACTN1, CUL2, DCC, DERL1, FUS, PDSS2, PLAG1, RpS6, SMAD2, SMAD4, VDAC1, and YBX1, wherein the measured levels are indicative of the prognosis of the cancer patient.

In some embodiments, the prognostic methods of the invention comprise measuring, in a sample obtained from a patient, the levels of two or more PDs selected from:

(1) at least one cytoskeletal gene or protein (e.g., an alpha-actinin such as alpha-actinin 1, 2, 3, and 4);

(2) at least one ubiquitination gene or protein (e.g., CUL1, CUL2, CUL3, CUL4A, CUL4B, CUL5, CULT, DERL1, DERL2, and DERL3);

(3) at least one dependence receptor gene or protein (e.g., DCC, neogenin, p75NTR, RET, TrkC, Ptc, EphA4, ALK, and MET);

(4) at least one DNA repair gene or protein (e.g., FUS, EWS, TAF15, SARF, and TLS);

(5) at least one terpenoid backbone biosynthesis gene or protein (e.g., PDSS1 and PDSS2);

(6) at least one PI3K pathway gene or protein (e.g., RpS6 and PLAG1);

(7) at least one TFG-beta pathway gene or protein (e.g., SMAD1, SMAD2, SMAD3, SMAD4, SMAD5, and SMAD9);

(8) at least one voltage-dependent anion channel gene or protein (e.g., VDAC1, VDAC2, VDAC3, TOMM40 and TOMM40L); and/or

(9) at least one RNA splicing gene or protein (e.g., U2AF or YBX1);

wherein the measured levels are indicative of the prognosis of the cancer patient.

The methods may comprise an additional step of obtaining a sample (e.g., a cancerous tissue sample) from the patient. The sample can be a solid tissue sample such as a tumor sample. A solid tissue sample may be a formalin-fixed paraffin-embedded (FFPE) tissue sample, a snap-frozen tissue sample, an ethanol-fixed tissue sample, a tissue sample fixed with an organic solvent, a tissue sample fixed with plastic or epoxy, a cross-linked tissue sample, a surgically removed tumor tissue, or a biopsy sample such as a core biopsy, an excisional tissue biopsy, or an incisional tissue biopsy. In other embodiments, the sample can be a liquid sample, including a blood sample and a circulating tumor cell (CTC) sample. In a further embodiment, the tissue sample is a prostate tissue sample such as an FFPE prostate tumor sample.

In some embodiments, the prognostic methods of the invention measure the RNA or protein levels of the two or more PDs comprise: at least ACTN1, YBX1, SMAD2, and FUS; at least ACTN1, YBX1, and SMAD2; at least ACTN1, YBX1, and FUS; at least ACTN1, SMAD2, and FUS; or at least YBX1, SMAD2, and FUS.

In some embodiments, the methods of the invention measure at least three, four, five, six, seven, eight, nine, ten, eleven, or twelve PDs. In further embodiments, the methods measure three PDs (i.e., PDs 1-3), four PDs (i.e., PDs 1-4), five PDs (i.e., PDs 1-5), six PDs (i.e., PDs 1-6), seven PDs (i.e., PDs 1-7), eight PDs (i.e., PDs 1-8), nine PDs (i.e., PDs 1-9), ten PDs (i.e., PDs 1-10), eleven PDs (i.e., PDs 1-11), or twelve PDs (i.e., PDs 1-12), wherein the PDs are all different from each other and are independently selected from the group consisting of ACTN1, CUL2, DCC, DERL1, FUS, PDSS2, PLAG1, RpS6, SMAD2, SMAD4, VDAC1, and YBX1.

In some embodiments, the prognostic methods of the invention measure one or more PDs whose levels are up-regulated, relative to a reference value, in an aggressive form of cancer or cancer with a high risk of lethal outcome. Such PDs may be, e.g., CUL2, DCC, DERL1, FUS, PDSS2, PLAG1, SMAD2, and VDAC1. The methods may measure one or more PDs whose levels are down-regulated, relative to a reference value, in an aggressive form of cancer or cancer with a high risk of lethal outcome. Such PDs may be, e.g., ACTN1, RpS6, SMAD4, and YBX1.

In further embodiments, the methods of the invention measure, in addition to PDs selected from the aforementioned twelve biomarker group, one or more of the PDs selected from the group consisting of HOXB13, FAK1, COX6C, FKBP5, PXN, AKAP8, DIABLO, CD75, LATS2, DEC1, LMO7, EIF3H, CDKN1B, MTDH2, MAOA, CCND1, HSD17B4, MAP3K5, and pPRAS40.

The prognostic methods of the invention may measure the expression levels of the selected PDs, by, e.g., antibodies or antigen-binding fragments thereof. The expression or protein levels may be measured by immunohistochemistry or immunofluorescence. For example, the antibodies or antigen-binding fragments directed to the PDs may each be labeled or bound by a different fluorophore and signals from the fluorophores may be detected separately or concurrently (multiplex) by an automated imaging machine. In some embodiments, the tissue sample may be stained with DAPI. In some embodiments, the methods may measure protein levels of selected PDs in subcellular compartments such as the nucleus, the cytoplasm, or the cell membrane. Alternatively, the measurement can be done in the whole cell.

The measurement can be done in a tissue sample in a defined region of interest, such as a tumor region where noncancerous cells are excluded. For example, noncancerous cells can be identified by their binding to (e.g., staining by) an anti-cytokeratin 5 antibody and/or an anti-TRIM29 antibody, and/or by their lack of specific binding (not significantly higher than background noise level) to an anti-cytokeratin 8 antibody or an anti-cytokeratin 18 antibody. Cancerous cells, on the other hand, can be identified by their binding to (e.g., staining by) an anti-cytokeratin 8 antibody and/or an anti-cytokeratin 18 antibody, and/or by their lack of specific binding to an anti-cytokeratin 5 antibody and an anti-TRIM29 antibody. In a specific embodiment, the methods comprise contacting a cross-section of the FFPE prostate tumor sample with an anti-cytokeratin 8 antibody, an anti-cytokeratin 18 antibody, an anti-cytokeratin 5 antibody, and an anti-TRIM29 antibody, wherein the measuring step is conducted in an area in the cross section that is bound by the anti-cytokeratin 8 and anti-cytokeratin 18 antibodies and is not bound by the anti-cytokeratin 5 and anti-TRIM29 antibodies.

In some embodiments, in addition to measruing the biomarkers of this invention, it may be desired that at least one standard parameter associated with the cancer of interest is assessed, e.g., Gleason score, tumor stage, tumor grade, tumor size, tumor visual characteristics, tumor location, tumor growth, lymph node status, tumor thickness (Breslow score), ulceration, age of onset, PSA level, and PSA kinetics.

The prognostic methods of this invention are useful clinically to improve the efficacy of cancer treatment and to avoid unnecessary treatment. For example, the biomarkers and the diagnostic methods of this invention can be used to identify a cancer patient in need of adjuvant therapy, comprising obtaining a tissue sample from the patient; measuring, in the sample, the levels of the biomarkers described herein, and patients with a prognosis of aggressive cancer or having a heightened risk of cancer-related lethal outcome can then be treated with adjuvant therapy. Accordingly, the present invention also provides methods of treating a cancer patient by identifying or selecting a patient with an unfavorable prognosis as determined by the present prognostic methods, and treating only those who have an unfavorable prognosis with adjuvant therapy. Adjuvant therapy may be administered to a patient who has received a standard-of-care therapy, such as surgery, radiation, chemotherapy, or androgen ablation. Examples of adjuvant therapy include, without limitation, radiation therapy, chemotherapy, immunotherapy, hormone therapy, and targeted therapy. The targeted therapy may targets a component of a signaling pathway in which one or more of the selected PD is a component and wherein the targeted component is or the same or different from the selected PD.

The present invention also provides diagnostic kits for measuring the levels of two or more PDs selected from the group consisting of ACTN1, CUL2, DCC, DERL1, FUS, PDSS2, PLAG1, RpS6, SMAD2, SMAD4, VDAC1, and YBX1, comprising reagents for specifically measuring the levels of the selected PDs. The reagents may comprise one or more antibodies or antigen-binding fragments thereof, oligonucleotides, or apatmers. The reagents may measure, e.g., the RNA transcript levels or the protein levels of the selected PDs.

The present invention also provides methods of identifying a compound capable of reducing the risk of cancer progression, or delaying or slowing the cancer progression, comprising: (a) providing a cell expressing a PD selected from the group consisting of ACTN1, CUL2, DCC, DERL1, FUS, PDSS2, PLAG1, RpS6, SMAD2, SMAD4, VDAC1, and YBX1; (b) contacting the cell with a candidate compound; and

(c) determining whether the candidate compound alters the expression or activity of the selected PD; whereby the alteration observed in the presence of the compound indicates that the compound is capable of reducing the risk of cancer progression, or delaying or slowing the cancer progression. The compounds so identified can be used in the present cancer treatment methods.

Also described herein are the following embodiments:

### Embodiment 1

A method for predicting prognosis of a cancer patient, comprising:

measuring, in a sample obtained from a patient, the levels of two or more Prognosis Determinants (PDs) selected from the group consisting of ACTN1, CUL2, DCC, DERL1, FUS, PDSS2, PLAG1, RpS6, SMAD2, SMAD4, VDAC1, and YBX1,

wherein the measured levels are indicative of the prognosis of the cancer patient.

### Embodiment 2

A method for predicting prognosis of a cancer patient, comprising:

measuring, in a sample obtained from a patient, the levels of two or more PDs selected from at least one cytoskeletal gene or protein; at least one ubiquitination gene or protein; at least one dependence receptor gene or protein; at least one DNA repair gene or protein; at least one terpenoid backbone biosynthesis gene or protein; at least one PI3K pathway gene or protein; at least one TFG-beta pathway gene or protein; at least one voltage-dependent anion channel gene or protein; or at least one RNA splicing gene or protein;

wherein the measured levels are indicative of the prognosis of the cancer patient.

### Embodiment 3

The method of embodiment 2, wherein the at least one cytoskeletal gene or protein is alpha-actinin 1, alpha-actinin 2, alpha-actinin 3, or alpha-actinin 4; the at least one ubiquitination gene or protein is CUL1, CUL2, CUL3, CUL4A, CUL4B, CUL5, CULT, DERL1, DERL2, or DERL3; the at least one dependence receptor gene or protein is DCC, neogenin, p75^{NTR}, RET, TrkC, Ptc, EphA4, ALK, or MET; the at least one DNA repair gene or protein is FUS, EWS, TAF15, SARF, or TLS; the at least one terpenoid backbone biosynthesis gene or protein is PDSS1, or PDSS2; the at least one PI3K pathway gene or protein is RpS6 or PLAG1; the at least one TFG-beta pathway gene or protein is SMAD1, SMAD2, SMAD3, SMAD4, SMAD5, or SMAD9; the at least one voltage-dependent anion channel gene or protein is VDAC1, VDAC2, VDAC3, TOMM40 or TOMM40L; or the at least one RNA splicing gene or protein is U2AF or YBX1.

### Embodiment 4

The method of any one of embodiments 1-3, further comprising the step of obtaining the sample from a patient.

### Embodiment 5

The method of any one of embodiments 1-4, wherein the prognosis is that the cancer is an aggressive form of cancer.

### Embodiment 6

The method of any one of embodiments 1-4, wherein the prognosis is that the patient is at risk of having an aggressive form of cancer.

### Embodiment 7

The method of any one of embodiments 1-4, wherein the prognosis is that the patient is at risk of having a cancer-related lethal outcome.

### Embodiment 8

A method for identifying a cancer patient in need of adjuvant therapy, comprising:

obtaining a tissue sample from the patient; and

measuring, in the sample, the levels of two or more PDs selected from the group consisting of ACTN1, CUL2, DCC, DERL1, FUS, PDSS2, PLAG1, RpS6, SMAD2, SMAD4, VDAC1, and YBX1, wherein the measured levels indicate that the patient is in need of adjuvant therapy.

### Embodiment 9

A method for identifying a cancer patient in need of adjuvant therapy, comprising:

obtaining a tissue sample from the patient; and

measuring, in the sample, the levels of two or more PDs selected from at least one cytoskeletal gene or protein; at least one ubiquitination gene or protein; at least one dependence receptor gene or protein; at least one DNA repair gene or protein; at least one terpenoid backbone biosynthesis gene or protein; at least one PI3K pathway gene or protein; at least one TFG-beta pathway gene or protein; at least one voltage-dependent anion channel gene or protein; or at least one RNA splicing gene or protein;

wherein the measured levels indicate that the patient is in need of adjuvant therapy.

### Embodiment 10

A method for treating a cancer patient, comprising:

measuring the levels of two or more PDs selected from the group consisting of ACTN1, CUL2, DCC, DERL1, FUS, PDSS2, PLAG1, RpS6, SMAD2, SMAD4, VDAC1, and YBX1; and

treating the patient with an adjuvant therapy if the measured levels indicate that the patient has an aggressive form of cancer, or is at risk of having a cancer-related lethal outcome.

### Embodiment 11

A method for treating a cancer patient, comprising:

identifying a patient with level changes in at least two PDs, wherein the level changes are selected from the group consisting of up-regulation of one or more of CUL2, DCC, DERL1, FUS, PDSS2, PLAG1, SMAD2, and VDAC land down-regulation of one or more of ACTN1, RpS6, SMAD4, and YBX1; and

treating the patient with an adjuvant therapy.

### Embodiment 12

A method for treating a cancer patient, comprising:

measuring the levels of two or more PDs selected from the group consisting of at least one cytoskeletal gene or protein; at least one ubiquitination gene or protein; at least one dependence receptor gene or protein; at least one DNA repair gene or protein; at least one terpenoid backbone biosynthesis gene or protein; at least one PI3K pathway gene or protein; at least one TFG-beta pathway gene or protein; at least one voltage-dependent anion channel gene or protein; or at least one RNA splicing gene or protein; and

treating the patient with an adjuvant therapy if the measured levels indicate that the patient has an aggressive form of cancer, or is at risk of having a cancer-related lethal outcome.

### Embodiment 13

The method of any one of embodiments 8-12, wherein the adjuvant therapy is selected from the group consisting of radiation therapy, chemotherapy, immunotherapy, hormone therapy, and targeted therapy.

### Embodiment 14

The method of embodiment 13, wherein the targeted therapy targets a component of a signaling pathway in which one or more of the selected PD is a component and wherein the targeted component is different from the selected PD.

### Embodiment 15

The method of embodiment 13, wherein the targeted therapy targets one or more of the selected PD.

### Embodiment 16

The method of any one of embodiments 8-12, wherein the patient has been subjected to a standard-of-care therapy.

### Embodiment 17

The method of embodiment 16, wherein the standard-of-care therapy is surgery, radiation, chemotherapy, or androgen ablation.

### Embodiment 18

The method of any one of embodiments 1-17, wherein the patient has prostate cancer.

### Embodiment 19

The method of any one of embodiments 1-18, wherein the two or more PDs comprise:

A) at least ACTN1, YBX1, SMAD2, and FUS;

B) at least ACTN1, YBX1, and SMAD2;

C) at least ACTN1, YBX1, and FUS;

D) at least ACTN1, SMAD2, and FUS; or

E) at least YBX1, SMAD2, and FUS.

### Embodiment 20

The method of any one of embodiments 1-19, wherein at least three, four, five, six, seven, eight, nine, ten, eleven, or twelve PDs are selected.

### Embodiment 21

The method of any one of embodiments 1, 4-8, 9, 10, and 12-19, wherein six PDs consisting of PD1, PD2, PD3, PD4, PD5, and PD6 are selected, and wherein PD1, PD2, PD3, PD4, PD5, and PD6 are different and are independently selected from the group consisting of ACTN1, CUL2, DCC, DERL1, FUS, PDSS2, PLAG1, RpS6, SMAD2, SMAD4, VDAC1, and YBX1.

### Embodiment 22

The method of any one of embodiments 1, 4-8, 9, 10, and 12-19, wherein seven PDs consisting of PD1, PD2, PD3, PD4, PD5, PD6, and PD7 are selected, and wherein PD1, PD2, PD3, PD4, PD5, PD6, and PD7 are different and are independently selected from the group consisting of ACTN1, CUL2, DCC, DERL1, FUS, PDSS2, PLAG1, RpS6, SMAD2, SMAD4, VDAC1, and YBX1.

### Embodiment 23

The method of any one of embodiments 1, 5-8, 10, and 13-22, further comprising measuring the levels of one or more PDs selected from the group consisting of HOXB13, FAK1, COX6C, FKBP5, PXN, AKAP8, DIABLO, CD75, LATS2, DEC1, LMO7, EIF3H, CDKN1B, MTDH2, MAOA, CCND1, HSD17B4, MAP3K5, and pPRAS40.

### Embodiment 24

The method of any one of embodiments 1-23, wherein the measured levels of at least one of the two or more selected PDs are up-regulated relative to a reference value.

### Embodiment 25

The method of any one of embodiments 1-24, wherein the measured levels of at least one of the two or more selected PDs are down-regulated relative to a reference value.

### Embodiment 26

The method of any one of embodiments 1-25, wherein the measured levels of at least one of the two or more selected PDs are up-regulated relative to a reference value and at least one of the two or more selected PDs are down-regulated relative to a reference value.

### Embodiment 27

The method of embodiment 24, wherein the selected PDs comprise one or more PDs selected from the group consisting of CUL2, DCC, DERL1, FUS, PDSS2, PLAG1, SMAD2, and VDAC1.

### Embodiment 28

The method of embodiment 25, wherein the selected PDs comprise one or more PDs selected from the group consisting of ACTN1, RpS6, SMAD4, and YBX1.

### Embodiment 29

The method of any one of embodiments 1-28, wherein the measuring step comprises measuring the protein levels of the selected PDs.

### Embodiment 30

The method of embodiment 29, wherein the protein levels are measured by antibodies or fragments thereof.

### Embodiment 31

The method of embodiment 30, wherein the protein levels are measured by immunohistochemistry or immunofluorescence.

### Embodiment 32

The method of embodiment 30, wherein the antibodies or fragments thereof are each labeled or bound by a different fluorophore and signals from the fluorophores are detected concurrently by an automated imaging machine.

### Embodiment 33

The method of embodiment 32, wherein the tissue sample is stained with DAPI.

### Embodiment 34

The method of embodiment 29, wherein the measuring step comprises measuring the protein level of a selected PD in subcellular compartments.

### Embodiment 35

The method of embodiment 29, wherein the measuring step comprises measuring the protein level of a selected PD in the nucleus, in the cytoplasm, or on the cell membrane.

### Embodiment 36

The method of any one of the above embodiments, wherein levels of the PDs are measured from a defined region of interest.

### Embodiment 37

The method of embodiment 36, wherein the noncancerous cells are excluded from the region of interest.

### Embodiment 38

The method of embodiment 37, wherein the noncancerous cells are bound by an anti-cytokeratin 5 antibody and an anti-TRIM29 antibody.

### Embodiment 39

The method of embodiment 38, wherein the noncancerous cells are not bound by an anti-cytokeratin 8 antibody and an anti-cytokeratin 18 antibody.

### Embodiment 40

The method of any one of embodiments 36 to 39, wherein cancerous cells are included in the region of interest.

### Embodiment 41

The method of embodiment 43, wherein the cancerous cells are bound by an anti-cytokeratin 8 antibody and an anti-cytokeratin 18 antibody.

### Embodiment 42

The method of embodiment 41, wherein the cancerous cells are not bound by an anti-cytokeratin 5 antibody and an anti-TRIM29 antibody.

### Embodiment 43

The method of any one of the above embodiments, wherein the measuring step comprises separately measuring the levels of the selected PDs.

### Embodiment 44

The method of any one of the above embodiments, wherein the measuring step comprises measuring the levels of the selected PDs in a multiplex reaction.

### Embodiment 45

The method of any one of the above embodiments, wherein the sample is a solid tissue sample.

### Embodiment 46

The method of embodiment 45, wherein the solid tissue sample is a formalin-fixed paraffin-embedded tissue sample, a snap-frozen tissue sample, an ethanol-fixed tissue sample, a tissue sample fixed with an organic solvent, a tissue sample fixed with plastic or epoxy, a cross-linked tissue sample, a surgically removed tumor tissue, or a biopsy sample.

### Embodiment 47

The method of embodiment 46, wherein said biopsy sample is a core biopsy, an excisional tissue biopsy, or an incisional tissue biopsy.

### Embodiment 48

The method of any one of the above embodiments, wherein the tissue sample is a cancerous tissue sample.

### Embodiment 49

The method of any one of the above embodiments, wherein the tissue sample is a prostate tissue sample.

### Embodiment 50

The method of embodiment 49, wherein the prostate tissue sample is a formalin-fixed paraffin-embedded (FFPE) prostate tumor sample.

### Embodiment 51

The method of embodiment 50, further comprising contacting a cross-section of the FFPE prostate tumor sample with an anti-cytokeratin 8 antibody, an anti-cytokeratin 18 antibody, an anti-cytokeratin 5 antibody, and an anti-TRIM29 antibody, wherein the measuring step is conducted in an area in the cross section that is bound by the anti-cytokeratin 8 and anti-cytokeratin 18 antibodies and is not bound by the anti-cytokeratin 5 and anti-TRIM29 antibodies.

### Embodiment 52

The method of any one of the above embodiments, further comprising measuring at least one standard parameter associated with said cancer.

### Embodiment 53

The method of embodiment 52, wherein the at least one standard parameter is selected from the group consisting of Gleason score, tumor stage, tumor grade, tumor size, tumor visual characteristics, tumor location, tumor growth, lymph node status, tumor thickness (Breslow score), ulceration, age of onset, PSA level, and PSA kinetics.

### Embodiment 54

A kit for measuring the levels of two or more PDs selected from the group consisting of ACTN1, CUL2, DCC, DERL1, FUS, PDSS2, PLAG1, RpS6, SMAD2, SMAD4, VDAC1, and YBX1, comprising reagents for specifically measuring the levels of the selected PDs.

### Embodiment 55

The kit of embodiment 54, wherein the reagents comprise one or more antibodies or fragments thereof, oligonucleotides, or apatmers.

### Embodiment 56

The kit of embodiment 54, wherein the reagents measure the RNA transcript levels or the protein levels of the selected PDs.

### Embodiment 57

A method of identifying a compound capable of reducing the risk of cancer progression, or delaying or slowing the cancer progression, comprising:

(a) providing a cell expressing a PD selected from the group consisting of ACTN1, CUL2, DCC, DERL1, FUS, PDSS2, PLAG1, RpS6, SMAD2, SMAD4, VDAC1, and YBX1;

(b) contacting the cell with a candidate compound; and
(c) determining whether the candidate compound alters the expression or activity of the selected PD;

whereby the alteration observed in the presence of the compound indicates that the compound is capable of reducing the risk of cancer progression, or delaying or slowing the cancer progression.

### Embodiment 58

A method for treating a cancer patient, comprising:

measuring the level of a PD selected from the group consisting of ACTN1, CUL2, DCC, DERL1, FUS, PDSS2, PLAG1, RpS6, SMAD2, SMAD4, VDAC1, and YBX1; and

administering an agent that modulates the level of the selected PD.

### Embodiment 59

A method for treating a cancer patient, comprising:

measuring the levels of two or more PDs selected from the group consisting of at least one cytoskeletal gene or protein; at least one ubiquitination gene or protein; at least one dependence receptor gene or protein; at least one DNA repair gene or protein; at least one terpenoid backbone biosynthesis gene or protein; at least one PI3K pathway gene or protein; at least one TFG-beta pathway gene or protein; at least one voltage-dependent anion channel gene or protein; or at least one RNA splicing gene or protein; and

administering an agent that modulates the level of the selected PD.

### Embodiment 60

A method for treating a cancer patient, comprising:

identifying patient with level changes in at least two PDs, wherein the level changes are selected from the group consisting of up-regulation of one or more of CUL2, DCC, DERL1, FUS, PDSS2, PLAG1, SMAD2, and VDACland down-regulation of one or more of ACTN1, RpS6, SMAD4, and YBX1; and

administering an agent that modulates the level of at least one of the PDs.

### Embodiment 61

A method for defining a region of interest in a tissue sample comprising contacting the tissue sample with one or more first reagents for specifically for identifying the region of interest.

### Embodiment 62

The method of embodiment 61, wherein the region of interest comprises cancerous cells.

### Embodiment 63

The method of embodiment 62, wherein the one or more first reagents comprise an anti-cytokeratin 8 antibody and an anti-cytokeratin 18 antibody.

### Embodiment 64

The method of any one of embodiments 61 to 63, further comprising defining a region of the tissue sample to be excluded from the region of interest by contacting the tissue sample with one or more second reagents for specifically for identifying the region to be excluded.

### Embodiment 65

The method of embodiment 64, wherein the region to be excluded comprises noncancerous cells.

### Embodiment 66

The method of embodiment 65, wherein the one or more second reagents comprise an anti-cytokeratin 5 antibody, and an anti-TRIM29 antibody.

Aspects and embodiments are also directed to a computer-implemented or automated method of evaluating a tumor sample, e.g., to assign a risk score to the patient.

Aspects and embodiments are also directed to a system including a memory and a processing unit operative to evaluate a tumor sample, e.g., to assign a risk score to the patient.

Aspects and embodiments are also directed to a system including a memory and a processing unit operative to evaluate a tumor sample, e.g., to analyze signals from the integral tumor sample or to assign a risk score to the patient.

Aspects and embodiments are also directed to a computer-readable medium comprising computer-executable instructions that, when executed on a processor of a computer, perform a method for evaluating a tumor sample, e.g., to analyze signals from the integral tumor sample or to assign a risk score to the patient.

### BRIEF DESCRIPTION OF THE DRAWINGS

U.S. Provisional Application No. 61/792,003, to which this application claims priority, contains at least one drawing executed in color. Copies of U.S. Provisional Application No. 61/792,003 with color drawing(s) will be provided by the United States Patent and Trademark Office upon request and payment of the necessary fee.

FIG. 1 depicts a hematoxylin and eosin stained section of surgically removed prostate tumor. American Board of Pathology certified anatomical pathologists annotated the section to identify the four areas of highest observed Gleason score pattern and the two areas of lowest observed Gleason score pattern. One high-observed core was extracted from the tumor sample for inclusion in a high-observed tissue microarray (TMA), and one low-observed core was extracted from the tumor sample for inclusion on a low-observed TMA.

FIG. 2 depicts a biomarker selection and validation engine that can be used to identify biomarkers for any disease or condition. The engine has three phases: a biological phase, a technical phase, and a performance phase. MoAb-monoclonal antibody; DAB-3,3′-Diaminobenzidine; IF-immunofluorescence; and TMA-tis sue microarray.

FIG. 3 depicts a prostate cancer-specific biomarker selection and validation engine. The engine has three phases: a biological phase, a technical phase, and a performance phase. Initially 160 potential biomarkers were identified. Using the biomarker selection and validation engine, 12 markers were identified as correlating with tumor aggression. MoAb-monoclonal antibody; DAB-3,3′-Diaminobenzidine; IF-immunofluorescence; and TMA-tissue microarray.

FIG. 4 demonstrates intersection reproducibility using quantitative multiplex immunofluorescence on a control cell line TMA (CTMA). Sections 27 and 41 of the CTMA were stained with immunofluorescent antibodies for FUS-N and DERL1. The fluorescent intensities for each cell line in the CTMA were compared between sections 27 and 41, and the results graphed, as shown. The linear relationship of the amount of immunofluorescence in the two cell lines and the high R² values demonstrate the reproducibility of the quantitative immunofluorescence assay between experiments.

FIG. 5 depicts the breakdown of the cohort of samples included on the low-observed TMA in terms of tumor aggression and lethal outcome. Of the 297 patients included in the tumor aggression study, 110 patients had indolent tumors, 122 patients had intermediate tumors, and 67 patients had aggressive tumors based on surgical Gleason scores. Of the 317 patients included in the lethal outcome study, 275 patients had indolent tumors (did not die of prostate cancer) and 42 patients had aggressive tumors (died of prostate cancer or a remote metastases). The first five columns provide clinical data, while the last four columns provide an estimate for the number of samples that were useful when training models with 3, 6, 9 or 12 markers.

FIG. 6 demonstrates the inter-system reproducibility of two Vectra Intelligent Slide Analysis Systems. A CTMA was evaluated in duplicate on two different systems for Alexa-568, Alexa-633 and Alexa-647 detection. The two systems differed in Alexa-568 detection by about 7%, Alexa-633 detection by about 20% and Alexa-647 detection by about 2%. Anti-VDAC1, FUS, and SMAD4 antibodies were used for the Alexa-568, 633, and 647 channels, respectively.

FIG. 7 depicts the automated image acquisition and processing by the Vectra Intelligent Slide Analysis System, and the automated image analysis by Definiens Developer XD™. The biomarker intensity score obtained by the automated analysis can then be used to determine biomarker correlation by bioinformatics or to evaluate a clinical sample using Harvest Laboratory Information System (LIS).

FIG. 8 depicts a quality control feature incorporated into the automated image analysis, wherein each image is analyze through each fluorescent layer to detect oversaturation, aberrant texture, or lack of tissue. The region marked as “artifact” indicates detection of oversaturation, such that the oversaturated region is excluded from the analysis of the image.

FIG. 9A to FIG. 9F depict the automated identification of a region of interest (ROI) using the Definiens Developer XD™. FIG. 9A shows a raw image imported into the system containing multiple channels of fluorescence. FIG. 9B shows that tumor epithelial structures are identified based on anti-cytokeratin 8 and anti-cytokeratin 18 staining. FIG. 9C shows that nuclei are overlaid to identify where cells are located within the tumor epithelial region. FIG. 9D shows that cells are defined as benign or malignant based on the presence of basal cell markers cytokeratin 5 and TRIM29. FIG. 9E shows that regions of benign and malignant tumor are defined. FIG. 9F shows that a region of interest is defined based on the location of benign and malignant tumor.

FIG. 10 depicts the quantitation of biomarker (PD) immunofluorescence within the region of interest. Note that two biomarkers (DERL1 (PD1) and FUS (PD2)) are expressed at lower levels in malignant tumor regions than benign tumor regions.

FIG. 11 depicts seventeen biomarkers that demonstrated univariate performance for prediction of tumor aggression and lethal outcome in the HLTMA study, using the low cores. The core Gleason scores are observed Gleason scores. The most reliable results were obtained when cores from intermediate tumors (based on surgical Gleason score) were excluded. By, defining cores from intermediate tumors as indolent or aggressive the correlations between the biomarker and tumor aggression could be skewed towards indolent or aggressive associations.

FIG. 12 depicts the bioinformatics analysis of the data from the HLTMA studies.

FIG. 13 depicts the frequency with which biomarkers appear in the top 1% of combinations sorted by AIC for correlation with tumor aggression. Frequencies are presented for combinations with a maximum of 3 biomarkers, a maximum of 4 biomarkers, a maximum of 5 biomarkers, a maximum of 6 biomarkers, a maximum of 8 biomarkers, and a maximum of 10 biomarkers. The biomarkers tested were selected from a pool of 17 biomarkers that had been pre-selected for univariate performance in mini TMA assays and in the HLTMA.

FIG. 14 depicts the frequency with which biomarkers appear in the top 5% of combinations sorted by AIC for correlation with tumor aggression. Frequencies are presented for combinations with a maximum of 3 biomarkers, a maximum of 4 biomarkers, a maximum of 5 biomarkers, a maximum of 6 biomarkers, a maximum of 8 biomarkers, and a maximum of 10 biomarkers. The biomarkers tested were selected from a pool of 17 biomarkers that had been pre-selected for univariate performance in mini TMA assays and the HLTMA.

FIG. 15 depicts the frequency with which biomarkers appear in the top 1% and top 5% of seven-member maximum combinations sorted by AIC and test data for correlation with tumor aggression. The biomarkers tested were selected from a pool of 17 biomarkers that had been pre-selected for univariate performance in mini TMA assays and in the HLTMA using low cores.

FIG. 16 depicts the frequency with which biomarkers appear in the top 1% of five-member maximum combinations sorted by AIC and test data for correlation with tumor aggression. The biomarkers tested were selected from a pool of 31 biomarkers that had not been pre-selected for univariate performance on the HLTMA.

FIG. 17 depicts the frequency with which biomarkers appear in the top 5% of five-member maximum combinations sorted by AIC and test data for correlation with tumor aggression. The biomarkers tested were selected from a pool of 31 biomarkers that had not been pre-selected for univariate performance in the HLTMA.

FIG. 18 depicts the top-12 markers for each type of analysis and the concordance between top markers for the various analyses. A core of 7 biomarkers was identified as appearing in top-12 marker lists for 75% or 100% of the analyses. A secondary set of 7 biomarkers was also identified as appearing in top-12 marker lists for 50% of the analyses.

FIG. 19 depicts the frequency with which biomarkers appear in the top 1% of combinations sorted by AIC for correlation with lethal outcome. Frequencies are presented for combinations with a maximum of 3 biomarkers, a maximum of 4 biomarkers, a maximum of 5 biomarkers, a maximum of 6 biomarkers, a maximum of 8 biomarkers, and a maximum of 10 biomarkers. The biomarkers tested were selected from a pool of 17 biomarkers that had been pre-selected for univariate performance in the HLTMA.

FIG. 20 depicts the frequency with which biomarkers appear in the top 5% of combinations sorted by AIC for correlation with lethal outcome. Frequencies are presented for combinations with a maximum of 3 biomarkers, a maximum of 4 biomarkers, a maximum of 5 biomarkers, a maximum of 6 biomarkers, a maximum of 8 biomarkers, and a maximum of 10 biomarkers. The biomarkers tested were selected from a pool of 17 biomarkers that had been pre-selected for univariate performance in the HLTMA.

FIG. 21 depicts the frequency with which biomarkers appear in the top 1% and top 5% of seven-member maximum combinations sorted by AIC and test data for correlation with lethal outcome. The biomarkers tested were selected from a pool of 17 biomarkers that had been pre-selected for univariate performance in the HLTMA.

FIG. 22 demonstrates that markers that partially overlap in their correlation with tumor aggression and lethal outcome could potentially be used to evaluate both endpoints in a single assay. For example, as shown in FIGS. 11, 13, and 19, ACTN1 and YBX1 show a high degree of correlation with both tumor aggression and lethal outcome.

FIG. 23 depicts a Triplex analysis, which can be used to evaluate three biomarkers (PDs) in addition to tumor mask markers and nuclear staining on a single slide. A first biomarker, PD1, can be detected with a FITC-conjugated primary antibody and an anti-FITC-Alexa 568 secondary antibody. A second biomarker, PD2, can be detected with a rabbit primary antibody, a biotin conjugated anti-rabbit secondary antibody and streptavidin conjugated to Alexa 633. A third biomarker, PD3, can be detected with a mouse primary antibody, a horseradish peroxidase (HRP) conjugated anti-mouse secondary antibody and an anti-HRP-Alexa 647 tertiary antibody. For the tumor mask, anti-CK8-Alexa 488 and anti-CK18-Alexa 488 can be used to identify tumor epithelial structures and anti-CK5-Alexa 555 and anti-TRIM29-Alexa 555 can be used to identify basal cell markers. The quality of the tumor section can be evaluated by general autofluorescence (AFL) and autofluorescence from erythrocytes and bright granules (BAFL). While any three biomarkers (PDs) can be used in a Triplex staining (provided the correct antibody combinations are available), in this figure, PD1 is HSD17B4, PD2 is FUS, and PD3 is LATS2.

FIG. 24 depicts combinations of biomarker antibodies that can be combined for a Triplex analysis. Using these combinations, 12 biomarkers can be evaluated on four sections from a tumor sample.

FIG. 25 demonstrates that minimal interference is observed when antibodies for multiple biomarkers (SMAD (PD1) and RpS6 (PD2)) are used in the same assay. The linear relationship of the amount of immunofluorescence in the two assays and the high R² values demonstrate the minimal interference by the second antibody on the first.

FIG. 26 illustrates an exemplary computer system upon which various aspects of the present embodiments may be implemented.

FIG. 27A-F provides an outline of the experimental approach for automated, quantitative multiplex immunofluorescence and biomarker measurements in defined regions of interest of prostatectomy tissue.

FIG. 27A shows spectral profiles of each fluorophore in the spectral library used in the assay and profiles for tissue autofluorescence signals (AFL) and bright autofluorescence (B AFL) signals, respectively.

FIG. 27B shows a general outline of the staining procedure for quantitative multiplex immunofluorescent biomarker measurements in tissue region of interest. SPP1 and SMAD4 were used as an example. Region of interest marker antibodies (CK8 and CK18 for total epithelium and CK5 and TRIM29 for basal epithelium) were directly conjugated to Alexa488 and Alexa 555, respectively. Biomarker antibodies were detected with a sequence of secondary and tertiary antibodies, as described. Colors in the table illustrate unique spectral positions of emission peaks for the indicated Alexa fluophore dyes.

FIG. 27C illustrates that a composite multispectral image (i) is unmixed into separate channels corresponding to autofluorescence (AFL) and bright autofluorescence (BAFL), region of interest markers, and biomarkers, as indicated (ii).

FIG. 27D shows Definiens script-based tissue segmentation and biomarker quantitation. Moving through parts 1-6, from the composite image (1), first total epithelial regions were identified (2), followed by nuclear areas (3). The epithelial regions were further segmented into tumor (which was visualized in red), benign (which was visualized in green), and undetermined (which was visualized in yellow) (4). Gray color denoted non-epithelial regions, e.g. stroma and vessels (4). Finally, biomarkers were quantified from tumor epithelium areas only, which were outlined in red (5 and 6).

FIG. 27E shows tissue annotation and quality control procedures. Left: A representative hematoxillin and eosin (H&E)-stained section of a human prostatectomy sample showing four (blue) and two (green) 1 mm diameter circles placed over the regions with the highest and lowest Gleason pattern, respectively, as annotated by expert pathologist. Two cores (1 mm diameter each) were taken from two of the four blue regions to generate TMA blocks. Right: A consecutive section of the same prostatectomy sample was stained with DAPI and CK8/CK18-Alexa488. Areas with bright staining of prostate epithelium by CK8/18 cytokeratin antibodies were considered good quality regions, while areas with little or no staining (as indicated within the yellow punctate area) were considered of low quality and not deemed suitable for TMA construction.

FIG. 27F shows intra-experimental reproducibility. Two consecutive sections from a prostate tumor test TMA were stained in the same experiment. Images were acquired using the Vectra system and processed with a Definiens script. Scatter plots compare mean values of CK8/18, PTEN, and SMAD4 staining intensities from the same cores of the consecutive TMA sections. Linear regression curves, equations, and R² values were generated using Excel software.

FIG. 28A-C shows the cohort description and univariate analysis of lethal outcome. FIG. 28A shows the composition of the lethal outcome-annotated prostatectomy cohort used in current study and comparison with the PHS cohort from Ding et al, Nature 2011, 470:269-273. FIG. 28B shows Kaplan-Meier curves for survival as a function of single biomarker protein expression in the study cohort. The population with the top one-third of risk score values was separated from the population with the bottom two-thirds of risk scores. P values (P) and Hazard ratios (HR) are annotated.

FIG. 29A-C shows multivariate model development and Kaplan-Meier survival plots. FIG. 29A shows multivariate Cox regression and logistic regression analyses of survival prediction for the present study cohort. The marker combinations were used to develop models based on training and testing on the whole cohort. Four markers: PTEN, SMAD4, CCND1, SPP1. Three markers: SMAD4, CCND1, SPP1. FIG. 29B shows Kaplan-Meier curves for survival as a function of risk scores generated by a Cox model trained on the whole cohort using the four markers or [three markers+pS6+pPRAS40]. The lowest two-thirds of risk scores was used as threshold for population separation. FIG. 29C shows comparison of the lethal outcome-predictive performance of the four markers (PTEN, SMAD4, CCND1, SPP1) between this study and that of Ding et al.

FIG. 30A-E show validation of PTEN, CCND1, SMAD4, SPP1, P-S6 and P-PRAS40 antibodies specificity. Doxycycline-inducible shRNA knockdown cell lines were established for PTEN (FIG. 30A), CCND1 (FIG. 30B) and SMAD4 (FIG. 30C). Doxycycline treatment reduced the abundance of the target protein in all cases as assessed by Western Blotting (WB). Cell lines with high or low/negative levels of expression of PTEN (FIG. 30A), CCND1 (FIG. 30B) and SMAD4 (FIG. 30C) were also examined by WB and immunohistochemistry (IHC) to further validate the specificity of the antibodies. SPP1 (FIG. 30D) antibody detected an SPP1-specific band and an additional band at a lower molecular weight as assessed by WB in PC3 cells, while the SPP1-specific upper band was significantly decreased in low SPP1-expressing BxPC3 cells. The staining intensity of the SPP1 antibody in PC3 and BxPC3 cells by IHC correlated well with the relative intensity of the SPP1-specific band detected by WB. The specificity of P-S6 and P-PRAS40 antibodies (FIG. 30E) was validated in DU145 cells. LY294002 treatment significantly reduced phosphorylation of S6 and PRAS40, as shown by WB and IHC, respectively.

FIG. 31 shows an outline of statistical analysis flow. For each patient, two tissue cores from the area with the highest Gleason score were placed into TMA blocks. Mean values of biomarker expression in the tumor epithelium region of each TMA core were used for analysis, resulting in two biomarker values per patient. For PTEN, SMAD4 and pS6, the lowest value from the two cores was used for analysis. For CCND1, SPP1, p90RSK, pPRAS40 and Foxo3a, the highest value from the two cores was used. Using these values, 10,000 bootstrap training samples were generated and both multivariate Cox and Logistic Regression models were trained on each training sample. Testing was performed on the complement set. Given the cohort included censored data, we used both Concordance Index (CI) and ‘Area Under the Curve’ (AUC) to estimate the model performance. The marker combinations that were tested in the models were as follows: four markers (PTEN, SMAD4, CCND1, SPP1), three markers (SMAD4, CCND1, SPP1), and three markers with each of the following combinations of phospho markers: pS6, pPRAS40, and [pS6+pPRAS40].

FIG. 32 illustrates creation of biopsy simulation tissue microarrays (TMAs). A tissue block from a prostatectomy sample was annotated with all visible Gleason patterns (top). The example shown is from a patient with an overall Gleason score (GS) of 4+3=7. As shown in a higher-magnification view (middle), patterns within the same block can be highly diverse. Two 1 mm cores were taken from each tissue block. One was taken from an area with the highest GS (4+4=8) and embedded into agarose/paraffin along with high-scoring cores from other blocks to create the H TMA (bottom left). The other was taken from an area with the lowest GS (3+3=6) and embedded into agarose/paraffin along with low-scoring cores from other blocks to create the L TMA (bottom right).

FIG. 33 shows biomarker selection strategy. Three types of criteria (biological, technical, and performance-based) were used to select 12 final biomarkers. (DAB: Ab specificity assessed based on chromogenic tissue staining with diamino benzidine (DAB); IF: Ab specificity and performance based on immunofluorescent tissue staining).

FIG. 34A and FIG. 34B show univariate performance of 39 biomarkers measured in both low (L TMA; black bars) and high (H TMA; brown bars) Gleason areas for disease aggressiveness and disease-specific mortality. FIG. 34A shows the odds ratio (OR) for predicting severe disease pathology (aggressiveness) calculated for each marker. Markers with an OR to the left of the vertical line are negatively correlated with the severity of the disease as assessed by pathology. Those to the right of the line are positively correlated. The markers were ranked based on OR when measured in L TMA. FIG. 34B shows the hazard ratio for death from disease (lethality) calculated for each marker and plotted as described for FIG. 34A. In FIG. 34A and FIG. 34B, biomarkers with two asterisks (**) indicate statistical significance at the 0.1 level in both L and H TMA. Biomarkers with one asterisk (*) indicate statistical significance in only H TMA, but not L TMA. Note the large overlap of biomarkers with statistically significant univariate performance for both aggressive disease and death from disease.

FIG. 35A and FIG. 35B show performance-based biomarker selection process for disease aggressiveness. FIG. 35A shows that the bioinformatics workflow selected the most frequently utilized biomarkers from all combinations of up to five markers from a set of 31. FIG. 35B shows an example of performance of top-ranked 5-marker models, including comparison with training on L TMA and then testing on independent samples from L TMA and H TMA. Note that the test performances on L TMA and H TMA are consistent, with substantial overlap in confidence intervals. FIG. 35C shows that combinations were generated allowing a maximum of three, four or five biomarkers. The figure shows the proteins most frequently included when five-biomarker models were used to predict aggressive disease, ranked by test.

FIG. 36A and FIG. 36B show the final biomarker set and selection criteria. FIG. 36A shows twelve biomarkers that were selected based on univariate performance for aggressiveness (shown as OR on left) and lethality as well as frequency of appearance in multivariate models for disease aggressiveness or lethal outcome (table on right). FIG. 36B summarizes the names and biological significance of the biomarkers. The biomarker set comprises proteins known to function in the regulation of cell proliferation, cell survival, and metabolism. FIG. 36C shows that a multivariate 12-marker model for disease aggressiveness was developed based on logistic regression. The resulting AUC and OR are shown. Subsequently, the risk scores generated by the aggressiveness model for all patients were correlated with lethal outcome. The resulting AUC and HR are shown.

FIG. 37A-L shows antibody specificity. The specificity of ACTN1 (FIG. 37A), CUL2 (FIG. 37B), Derlin1 (FIG. 37C), FUS (FIG. 37D), PDSS2 (FIG. 37E), SMAD2 (FIG. 37F), VDAC1 (FIG. 37G), and YBX1 (FIG. 37H) antibodies were validated by Western blotting (WB) and immunohistochemistry (IHC) of siRNA-treated cells and control cells. Marker-specific siRNA treatment significantly reduced the intensity of the band on WB, and the specific IHC staining in cells confirmed the specificity of the antibodies. The specificity of the SMAD4 antibody (FIG. 37I) was validated by WB and IHC of the SMAD4-positive control cell line PC3 and SMAD4-negative control cell line BxPC3. The specificity of the pS6 antibody (FIG. 37J) was validated by WB and IHC of naive DU145 cells and DU145 cells treated with PI3K inhibitor LY294002. LY294002 treatment significantly reduced phosphorylation of S6, as shown by WB and IHC. The Leica anti-DCC antibody (FIG. 37K) detected a band on WB that did not match the expected size for the DCC protein (marked “X” in K); IHC staining was also not reduced in DCC siRNA-treated cells (left panel in FIG. 37K). The Leica anti-DCC antibody appeared to recognize the HSPA9 protein, as shown by WB and IHC of HSPA9 siRNA-treated cells and control cells (right panel in FIG. 37K). β-Actin was used as a WB loading control.

FIG. 38A-G shows identification of HSPA9 instead of DCC as a prostate cancer prognosis biomarker. The Leica anti-DCC antibody was not validated by DCC siRNA knockdown cells by WB and IHC (FIG. 38A), because the size of the band detected by the antibody on WB was much smaller than what was expected for DCC protein (75 kDa vs 158 kDa) and the IHC staining intensity was not reduced in DCC siRNA-treated cells. Mass spectrometry identified the protein recognized by the Leica anti-DCC antibody on WB to be HSPA9. To confirm that the Leica anti-DCC antibody was indeed an anti-HSPA9 antibody, it was tested by WB and IHC on HSPA9 siRNA-treated cells and control cells; both the WB band and the IHC staining detected by the Leica anti-DCC antibody were significantly reduced in HSPA9 siRNA-treated cells (FIG. 38B). The WB and IHC patterns of the Leica anti-DCC antibody on HSPA9 siRNA-treated cells were similar to those detected by a Santa Cruz anti-HSPA9 antibody (FIG. 38C). Silencing HSPA9 by siRNA appeared to decrease the proliferation of HeLa cells (FIG. 38D), reduced HeLa cell colony formation in a clonogenic assay (FIG. 38E), and caused increased cell death (FIG. 38F) and caspase activity (FIG. 38G).

FIG. 39A-C shows model building for the 8-Marker Signature Assay. FIG. 39A shows the odds ratios (with 95% confidence interval) for individual biomarkers. Quantitative biomarker measurements were correlated with prostate cancer pathology as an endpoint. Note that effect size has been normalized. FIG. 39B shows biomarker frequency utilization in top 10% of multivariate models. Given that many models have similar performance in the bootstrapped test AUC, frequency of occurrence in the exhaustive top marker model search is used as an additional criterion to choose the ultimate markers for the diagnostic test. This figure shows how often the top eight markers occur in the top 10% of eight-marker models, when sorted by bootstrapped median test AUC. FIG. 39C shows the final marker model coefficients that were used in a logistic regression model for calculation of the risk score, provided as a continuous scale from 0 to 1. Note that a negative sign indicates a protective marker. Units of these coefficients are in the fluorescence intensity scale associated with the assay.

FIG. 40A-F illustrates the clinical validation study and its performance for prediction of favorable pathology. Sensitivity and specificity curves (FIG. 40A and FIG. 40B, respectively) may be used to identify appropriate risk classification groups. Risk score distribution relative to NCCN risk classification groups (FIG. 40C and FIG. 40D) and D'Amico risk classification groups (FIG. 40E and FIG. 40F), showing that the biomarker signature assay adds significant additional risk information within each NCCN or D'Amico level.

FIG. 40A shows that the relationship between sensitivity and associated medical decision level can be used to identify low-risk classification groups. For example, a favorable classification might be identified as patients with risk score in the interval 0 to 0.33, which corresponds to a sensitivity (P[risk score>0.33|nonfavorable pathology]) of 90% (95% CI, 82% to 94%). In this case, a patient with nonfavorable pathology would have a 10% (95% CI, 6% to 18%) chance of incorrectly receiving a favorable classification. This false negative might lead to undertreatment.

FIG. 40B shows that the relationship between specificity and associated medical decision level can likewise be used to identify nonfavorable classification groups. For example, a nonfavorable classification might be identified as patients with risk score in the interval (0.8 to 1), which corresponds to a specificity (P[risk score ≤0.80| favorable pathology]) of 95% (95% CI, 90% to 98%). In this case, a patient with favorable pathology would have a 5% (95% CI, 2% to 10%) chance of incorrectly receiving a non-favorable classification. This false positive might lead to overtreatment.

FIG. 40C shows that the median risk score derived using the biomarker signature assay at each NCCN risk level (very low, low, intermediate, high) fell between the risk score cut-off levels of 0.33 and 0.8, with the predictive value (+PV) for favorable (surgical Gleason 3+3 or 3+4 and ≤T2) pathology found in 85% at risk score cut-off <0.33. The predictive value (−PV) for nonfavorable pathology was 100% at risk score cut-off >0.9, and 76.9% at risk score >0.8. For a risk score <0.33, 95% of the patients with ‘very low’ NCCN classification had favorable pathology, while the observed frequency of favorable cases by the ‘very low’ NCCN classification alone was 80.3%. In the ‘low’ NCCN category, for a risk score <0.33, 81.5% of the patients had favorable pathology, while the observed frequency of favorable pathology by the ‘low’ NCCN criterion was 63.8%. Conversely, for a risk score >0.8, 75% of patients in the ‘very low’ NCCN category had nonfavorable pathology and 76.9% of all patients had nonfavorable pathology when the risk score was >0.8.

FIG. 40D shows that the observed frequency of favorable cases as a function of the risk score quartile. Increased risk score quartile largely correlated with decreased observed frequency of favorable cases in each NCCN category. Moreover, the observed frequency of patients with favorable pathology identified by the test versus the NCCN stratification alone increased from 0% to 23.8% at a confidence level of 81%.

FIG. 40E shows the median risk score derived using the biomarker signature assay, at each D'Amico risk level (low, intermediate, high) fell between the risk score cut-off levels of 0.33 and 0.8. The predictive value (+PV) for favorable pathology is 85% at risk score cut-off <0.33. The predictive value (−PV) for nonfavorable cases is 100% at risk score cut-off >0.9, and 76.9% at risk score >0.8. For a risk score <0.33, 87.2% of the patients with ‘low’ D'Amico classification have favorable pathology, while the observed frequency of favorable cases within the ‘low’ D'Amico group is 70.6%. In the ‘intermediate’ D'Amico category, for a risk score <0.33, 75% of the patients have favorable favorable, while the observed frequency of all patients with favorable pathology within the ‘intermediate’ D'Amico group is 41.2%. Conversely, for a risk score >0.8, 59.3% of patients within the ‘low’ D'Amico category have nonfavorable pathology and 76.9% of all patients have nonfavorable pathology when the risk score is >0.8.

FIG. 40F shows the observed frequency of favorable cases as a function of the risk score quartile. Increased risk score quartile largely correlated with decreased observed frequency of favorable cases in each D'Amico category. Moreover, the observed frequency of patients with favorable pathology identified by the test versus the D'Amico stratification alone increased from 0% to 23.8% at a confidence level of 81%.

FIG. 41A-D shows results of the clinical validation study, full cohort: performance for “GS 6” pathology (surgical Gleason=3+3 and localized ≤T3a, N=256). FIG. 41A shows sensitivity (P[risk score>threshold|“non-GS 6” pathology]) of the test, as a function of medical decision level. FIG. 41B shows specificity (P[risk score<threshold|“GS 6” pathology]) of the risk score, used to identify “non-GS 6” category. FIG. 41C and FIG. 41D show the distribution of risk scores for “GS 6” and “Non-GS 6” pathologies. FIG. 41E shows the receiver operating characteristic (ROC) curve for the model. The area under the ROC curve (AUC)=0.65 (95% confidence interval [CI], 0.58 to 0.72), P<0.001, and highest-to-lowest quartile odds ratio (OR)=4.2 (95% CI, 1.9 to 9.3). OR for quantitative risk score was 12.59 (95% CI, 3.5 to 47.2) per unit change.

FIG. 42A-C shows results of the clinical validation study, full cohort: performance for prediction of favorable pathology (surgical Gleason ≤3+4 and organ-confined <T2, N=274). FIG. 42A shows the distribution of risk scores for favorable pathology. FIG. 42B shows the distribution of risk scores for nonfavorable pathology.

FIG. 42C shows the ROC curve for the model. AUC=0.68 (95% CI, 0.61 to 0.74), P<0.0001, and highest-to-lowest quartile OR=3.3 (95% CI, 1.8 to 6.1). OR for quantitative risk score was 20.9 (95% CI, 6.4 to 68.2) per unit change.

FIG. 43A-C shows the results of the clinical validation study, cohort with National Comprehensive Cancer Network (NCCN) and D'Amico criteria: performance for favorable pathology (surgical Gleason ≤3+4 and organ-confined ≤T2, N=256). FIG. 43A shows the distribution of risk scores for favorable disease. FIG. 43B shows the distribution of risk scores for nonfavorable disease. FIG. 43C shows the ROC curve for the model. AUC=0.69 (95% CI, 0.63 to 0.73), P<0.0001, and highest-to-lowest quartile OR=5.5 (95% CI, 2.5 to 12.1). OR for quantitative risk score was 26.2 (95% CI, 7.6 to 90.1) per unit change.

FIG. 44A-B shows the Net Reclassification Index analysis illustrates how molecular signature categories of favorable (risk score ≤0.33) and nonfavorable (risk score >0.8) can supplement NCCN (FIG. 44A) and D'Amico (FIG. 44B) SOC risk classification systems. Patients with molecular risk score ≤0.33 in NCCN low, intermediate, and high, and in D'Amico intermediate and high categories can be considered at lower risk of aggressive disease than the SOC category alone indicates. Patients with molecular risk score >0.8 in NCCN very low, low, and intermediate, and in D'Amico low and intermediate categories can be considered at higher risk of aggressive disease than the SOC category alone indicates. A molecular risk score ≤0.33 for categories NCCN very low and D'Amico low would be considered confirmatory. Similarly, a molecular risk score >0.8 for categories NCCN high and D'Amico high would be considered confirmatory. Note that favorable patients in the left rectangles and nonfavorable patients in the right rectangles reflect correct risk adjustments. Among patients with favorable pathology, 78% and 53% for NCCN and D'Amico, respectively, are correctly adjusted. Among patients with nonfavorable pathology, 76% and 88% for NCCN and D'Amico, respectively, are correctly adjusted. Note also that patients in the categories NCCN very low and in D'Amico low with molecular risk score ≤0.33 are significantly enriched for favorable patients relative to the risk group overall. R.S.=Molecular risk score.

FIG. 45A shows an outline of all four quantitative multiplex immunofluorescence triplex assay formats (PBXA/B/C/D) for staining of 12 markers. Region of interest marker antibodies were directly conjugated with Alexa dyes, while biomarker antibodies in channel 568 were conjugated with fluorescein isothiocyanate (FITC). All biomarkers (primary antibodies) were detected with a sequence of secondary and tertiary antibodies, except for pS6 and PDSS2, which were directly conjugated with FITC. Each color corresponds to a specific channel. Biomarkers with asterisks (*) were used for internal tissue quality control purposes, where cases with lower than predetermined signal intensities for ACTN1, DERL1, or VDAC were automatically excluded. The eight biomarkers whose quantitative measurements in the tumor epithelium are used in the predictive algorithm are indicated in italics.

FIG. 45B shows that during the image acquisition process, an image of the entire slide is acquired initially with a mosaic of 4× monochrome 4′,6-diamidino-2-phenylindole (DAPI) filter images. A tissue-finding algorithm was used to locate tissue where re-acquisition of images was performed with both 4×DAPI and 4×FITC monochrome filters, and later another tissue-finding algorithm was used to acquire images of all 20× fields containing a sufficient amount of tissue with consecutive exposures of DAPI, FITC, tetramethylrhodamine isothiocyanate (TRITC), and Cy5 filters. Image cubes were stored for automatic unmixing into individual channels and further processing by Definiens software.

FIG. 45C shows different steps of the whole quantitative multiplex immunofluorescence assay procedure. Unprocessed slides were initially examined visually with a fluorescence microscope for the presence of stains and dyes. The presence of noticeable amounts of fluorescent dyes excluded slides from further analysis. Tissues that passed initial quality control were subjected to the multiplex staining procedure with subsequent image acquisition, Definiens analysis, and bioinformatics analysis. The image acquisition process was performed as described above for FIG. 45B. Image cubes were stored in a server, unmixed into individual channels, and processed by Definiens software. Data were collected from tumor and benign regions from each specific region of interest (ROI) using ROI biomarkers by Definiens software. A bioinformatics analysis algorithm excluded cases with lower than predetermined signal intensities for ACTN1, DERL1, or VDAC 1 before the data were analyzed further.

### DETAILED DESCRIPTION OF THE INVENTION

The present invention is based on the discovery that biomarker panels comprising two or more members from the group consisting of ACTN1, CUL2, DCC, DERL1, FUS, PDSS2, PLAG1, RpS6, SMAD2, SMAD4, VDAC1, and YBX1 (“prognosis determinants” or “PD”s; Table 1) are useful in providing molecular, evidence-based, reliable prognosis about cancer patients. By measuring the expression (e.g., protein expression) or activity levels of the biomarkers in a cancerous tissue sample from a patient, one can reliably predict the aggressiveness of a tumor, such as a tumor's ability to invade surrounding tissues or risk of progression, in cancer patients. Cancer progression is indicated by, e.g., metastasis or recurrence of a cancer). The levels can also be used to predict lethal outcome of cancer, or efficacy of a cancer therapy (e.g., surgery, radiation therapy or chemotherapy) independent of, or in addition to, traditional, established risk assessment procedures. The levels also can be used to identify patients in need of aggressive cancer therapy (e.g., adjuvant therapy such as chemotherapy given in addition to surgical treatment), or to guide further diagnostic tests. When used in context with pathway context genes or proteins, the levels can also be used to inform healthcare providers about which types of therapy a cancer patient would be most likely to benefit from, and to stratify patients for inclusion in a clinical study. The levels also can be used to identify patients who will not benefit from and/or do not need cancer therapy (e.g., surgery, radiation therapy, chemotherapy, targeted therapy, or adjuvant therapy). In other words, the biomarker panels of this invention allow clinicians to optimally manage cancer patients.

In some embodiments, a primary clinical indication of a multiplex or multivariate diagnostic method of the invention is to accurately predict whether a PCA is “aggressive” (e.g., to predict the probability that a prostate tumor is actively progressing at the time of diagnosis (i.e., “active, aggressive disease”; or will progress at some later point (i.e., “risk of progression”)), or is “indolent” or “dormant.” Another clinical indication of the method can be to accurately predict the probability that the patient will die from PCA (i.e., “lethal outcome”/“disease-specific death”). Accuracy can be measured in terms of the C-statistic. For a model that assigns risk scores to samples, the C-statistic is the ratio of the number of pairs of samples with one aggressive sample and one indolent sample where the aggressive sample has a higher risk score than the indolent sample, over the total number of such pairs of samples.

### Definitions

“Acquire” or “acquiring” as the terms are used herein, refer to obtaining possession of a physical entity (e.g., a sample), or a value, e.g., a numerical value, or image, by “directly acquiring” or “indirectly acquiring” the physical entity or value. “Directly acquiring” means performing a process (e.g., performing a synthetic or analytical method, contacting a sample with a detection reagent, or capturing a signal from a sample) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; inducing or collecting a signal, e.g., a light based signal, e.g., a fluorescent signal, or by changing the structure of a reagent, or a fragment or other derivative thereof, e.g., by breaking or forming a covalent or non-covalent bond, between a first and a second atom of the reagent. Directly acquiring a value includes methods in which a computer or detection device, e.g, a scanner is used, e.g., when a change in electronic state responsive to impingement of a photon on a detector. Directly acquiring a value includes capturing a signal from a sample.

Detection reagent, as used herein, is a reagent, typically a binding reagent, that has sufficient specificity for its intended target that it can be used to distinguish that target from others discussed herein. In embodiments a detection reagent will have no or substantially no binding to other (non-target) species under the conditions in which the method is carried out.

Region of interest (ROI), as the term is used herein, refers to one or more entities, e.g., acellular entities (e.g., a subcellular component (e.g. a nucleus or cytoplasm), tissue components, acellular connective tissue matrix, acellular collagenous matter, extracellular components such as interstitial tissue fluids), or cells, which entity comprises a region-phenotype marker, which region-phenotype marker is used in the analysis of the ROI, or a sample, tissue, or patient from which it is derived. In an embodiment the entities of a ROI are cells.

A region-phenotype marker, as that term is used herein, reflects, predicts, or is associated with, a preselected phenotype, e.g., cancer, e.g., a cancer subtype, or outcome for a patient. In an embodiment a region-phenotype marker reflects, predicts, or is associated with, inflammatory disorders (e.g., autoimmune disorders), neurological disorders, or infectious diseases. In an embodiment, the preselected phenotype is present, or exerted, in the entities or cells of the ROI. In an embodiment the preselected phenotype is the phenotype of a disease, e.g., cancer, for which ROI, sample, tissue, or patient is being analyzed.

By way of example, the ROI can include cancer cells, e.g., cancerous prostate cells, the preselected phenotype is that of a cancerous cell, the population-phenotype marker is a cancer marker, e.g., in the case of prostate cancer, a tumor marker selected from FUS, SMAD4, DERL1, YBX1, pS6, PDSS2, CUL2, and HSPA9.

As used herein, unless the context indicates otherwise, pS6 refers to a phosphorylated form of ribosomal protein S6, which is encoded by the RpS6 gene.

In an embodiment a first ROI is a cancerous ROI and a second ROI is a benign ROI.

In an embodiment a region-phenotype marker is expressed in a cell of a ROI. In an embodiment a region-phenotype marker is disposed in a cell of a ROI, but is not expressed in that cell, e.g., in an embodiment the region-phenotype marker is a secreted factor found in the stroma, thus in this example the stroma is a ROI.

A ROI can be provided in a variety of ways. By way of example, a ROI can be selected or identified by possession of:

a morphological characteristic, e.g., a first tissue or cell type having a preselected relationship with, e.g., bounded by, a second tissue or cell type;

a non-morphological characteristic, e.g., a molecular characteristic, e.g., by possession of a selected molecule, e.g., a protein, mRNA, or DNA (referred to herein as a ROI marker) marker; or

by a combination of a morphological characteristic and a non-morphological characteristic.

In an embodiment identification or selection by morphological characteristic includes the selection (e.g., by manual or automated means) and physical separation of the ROI from other cells or material, e.g., by dissection of a ROI, e.g., a cancerous region, from other tissue, e.g., noncancerous cells. In an embodiment of morphological selection, e.g., micro-dissection, the ROI is removed essentially intact from its surroundings. In an embodiment of morphological selection, e.g., micro-dissection, the ROI is removed, but the morphological structure is not maintained.

In an embodiment of selection or identification by non-morphological characteristics, a ROI can be identified or selected by virtue of inclusion of a ROI marker, e.g., a preselected molecular species associated with, e.g., in, entities, e.g., cells, of the ROI. By way of example, cell sorting, e.g., FACS, can be used to provide a ROI by a non-morphological characteristic. In an embodiment FACS is used to separate cells having a ROI marker from other cells, to provide a ROI.

In an embodiment of selection of a ROI by a combination of morphological and non-morphological selection, morphologically identifiable structures that show a preselected pattern of binding to a detection reagent for a ROI marker are used to provide a ROI.

A ROI comprises entities, typically cells, in which the population phenotype marker exerts its function. In an embodiment, a ROI is a collection of entities, typically cells, from which a signal related to, e.g., proportional to, a region-phenotype marker can be extracted. The level of region-phenotype marker in the ROI, allows evaluation of the sample. E.g., in the case of prostate cancer, the level of a region-phenotype marker, e.g., a tumor marker, e.g., one of the tumor markers described herein, allows evaluation of the sample and the patient from whom the sample was taken. In an embodiment the region-phenotype marker is selected on the fact that it exerts a function, e.g., a function relating to a disorder being evaluated, prognosed or diagnosed, in the entities or cells of the ROI. ROI markers are used, in some embodiments, to select or define the ROI.

In an embodiment a ROI, is a collection of entities, typically cells, that, e.g., in the patient, though not necessarily in the sample, form a pattern, e.g., a distinct morphological region.

Sample, as that term is used herein, is a composition comprising a cellular or acellular component from a patient. The term sample includes an unprocessed sample, e.g., biopsy, a processed sample, e.g., a fixed tissue, fractions from a tissue or other substance from a patient. An ROI is considered to be a sample.

Prognosis Determinants

A first aspect of the invention provides prognosis determinants for use in cancer treatment decisions. The terms “prognosis determinant,” “biomarker” and “marker” are used interchangeably herein and refer to an analyte (e.g., a peptide or protein) that can be objectively measured and evaluated as an indicator for a biological process. The inventors have discovered that the expression or activity levels of these biomarkers correlate reliably with the prognosis of cancer patients, for example, tumor aggressiveness or lethal outcome. The ability of these biomarkers to correlate with cancer prognosis may be amplified by using them in combination.

At least one biomarker may be a cytoskeleton gene or protein. Without being bound by theory, cytoskeleton genes and proteins may correlate with cancer prognosis because malignancy is characterized, in part, by the invasion of a tumor into adjacent tissues and the spreading of the tumor to distant tumors. Such invasion and spreading typically require cytoskeletal reorganization. Non-limiting examples of cytoskeleton genes and proteins useful as biomarkers for cancer prognosis include alpha actin, beta actin, gamma actin, alpha-actinin 1, alpha-actinin 2, alpha-actinin 3, alpha-actinin 4, vinculin, E-cadherin, vimentin, keratin 1, keratin 2, keratin 3, keratin 4, keratin 5, keratin 6, keratin 7, keratin 8, keratin 9, keratin 10, keratin 11, keratin 12, keratin 13, keratin 14, keratin 15, keratin 16, keratin 17, keratin 18, keratin 19, keratin 20, lamin A, lamin B1, lambin B2, lamin C, alpha-tubulin, beta-tubulin, gamma-tubulin, delta-tubulin, epsilon-tubulin, LMO7, LATS1 and LATS2. Preferably, the cytoskeleton gene or protein is alpha-actinin 1, alpha-actinin 2, alpha-actinin 3, or alpha-actinin 4, particularly alpha-actinin 1. Alpha-actinin 1 has been shown to interact with CDK5R1; CDK5R2; collagen, type XVII, alpha 1; GIPC1; PDLIM1; protein kinase N1; SSX2IP; and zyxin. Accordingly, these genes and proteins are considered cytoskeleton proteins for the purposes of this application.

At least one biomarker may be an ubiquitination gene or protein. Without being bound by theory, ubiquitination genes and proteins may correlate with cancer prognosis because ubiquitin can be attached to proteins and directs them to the proteasome for destruction. Because increased rates of protein synthesis are often required to support transforming events in cancer, protein control processes, such as ubiquitination, are critical in tumor progression. Non-limiting examples of ubiquitination genes and proteins useful as biomarkers for cancer prognosis include ubiquitin activating enzyme (such as UBA1, UBA2, UBA3, UBA5, UBA6, UBA7, ATG7, NAE1, and SAE1), ubiquitin conjugating enzymes (such as UBE2A, UBE2B, UBE2C, UBE2D1, UBE2D2, UBE2D3, UBE2E1, UBE2E2, UBE2E3, UBE2G1, UBE2G2, UBE2H, UBE2I, UBE2J1, UBE2L3, UBE2L6, UBE2M, UBE2N, UBE2O, UBE2R2, UBE2V1, UBE2V2, and BIRC6), ubiquitin ligases (such as UBE3A, UBE3B, UBE3C, UBE4A, UBE4B, UBOX5, UBR5, WWP1, WWP2, mdm2, APC, UBR5, SOCS, CBLL1, HERC1, HERC2, HUWE1, NEDD4, NEDD4L, PPIL2, PRPF19, PIAS1, PIAS2, PIAS3, PIAS4, RANBP2, RBX1, SMURF1, SMURF2, STUB1, TOPORS, and TRIP12), F-box proteins (such as cdc4), Skp1, cullin family members (such as CUL1, CUL2, CUL3, CUL4A, CUL4B, CUL5, CUL7, and ANAPC2), RING proteins (such as RBX1), Elongin C, and endoplasmic-reticulum-associated protein degradation (“ERAD,” such as DERL1, DERL2, DERL3, Doa10, EDEM, ER mannosidase I, VIMP, SEL1, HRD1, and HERP). Preferably, the ubiquitination gene or protein is a cullin, particularly CUL2, or an ERAD gene or protein, particularly DERL1. CUL2 has been shown to interact with DCUN1D1, SAP130, CAND1, RBX1, TCEB2, and Von Hippel-Lindau tumor suppressor. Accordingly, these genes and proteins are considered ubiquitination proteins for the purposes of this application.

At least one biomarker may be a dependence receptor gene or protein. Without being bound by theory, dependence receptor genes and proteins may correlate with cancer prognosis because of their ability to trigger two opposite signaling pathways: 1) cell survival, migration and differentiation; and 2) apoptosis. In the presence of ligand, these receptors activate classic signaling pathways implicated in cell survival, migration and differentiation. In the absence of ligand, they do not stay inactive; rather they elicit an apoptotic signal. Cell survival, migration and apoptosis are all implicated in cancer. Non-limiting examples of dependence receptor genes and proteins useful as biomarkers for cancer prognosis include DCC, neogenin, p75^{NTR}, RET, TrkC, Ptc, EphA4, ALK, MET, and a subset of integrins. Preferably, the dependence receptor gene or protein is DCC. DCC has been shown to interact with PTK2, APPL1, MAZ, Caspase 3, NTN1 and Androgen receptor. Accordingly, these genes and proteins are considered dependence receptor proteins for the purposes of this application.

At least one biomarker may be a DNA repair gene or protein. Without being bound by theory, DNA repair genes and proteins may correlate with cancer prognosis because a cell that has accumulated a large amount of DNA damage, or one that no longer effectively repairs damage incurred to its DNA can enter unregulated cell division. Non-limiting examples of DNA repair genes and proteins useful as biomarkers for cancer prognosis include homologous recombination repair genes and proteins (such as BRCA1, BRCA2, ATM, MRE11, BLM, WRN, RECQ4, FANCA, FANCB, FANCC, FANCD1, FANCD2, FANCE, FANCF, FANCG, FANCI, FANCJ, FANCL, FANCM, and FANCN), nucleotide excision repair genes and proteins (such as XPC, XPE(DDB2), XPA, XPB, XPD, XPF, and XPG), non-homologous end joining genes and proteins (such as NBS, Rad50, DNA-PKcs, Ku70 and Ku80), trans lesion synthesis genes and proteins (such as XPV(POLH)), mismatch repair genes and proteins (such as hMSH2, hMSH6, hMLH1, hPMS2), base excision repair of adenine genes and proteins (such as MUTYH), cell cycle checkpoint genes and proteins (such as p53, p21, ATM, ATR, BRCA1, MDC1, and 53BP1), and TET family genes and proteins (such as FUS, EWS, TAF15, SARF, and TLS). Preferably, the DNA repair gene or protein is a TET family member, particularly FUS. FUS has been shown to interact with FUSIP1, ILF3, PRMT1, RELA, SPI1, and TNPO1. Accordingly, these genes and proteins are considered DNA repair proteins for the purposes of this application.

At least one biomarker may be a terpenoid backbone biosynthesis gene or protein. Without being bound by theory, terpenoid backbone biosynthesis genes and proteins may correlate with cancer prognosis because the biosynthesis of some terpenoids, such as CoQ₁₀, is reportedly reduced in cancer. Non-limiting examples of terpenoid backbone biosynthesis genes and proteins useful as biomarkers for cancer prognosis include ACAT1, ACAT2, HMGCS1, HMGCS2, HMGCR, MVK, PMVK, MVD, IDI1, IDI2, FDPS, GGPS1, PDSS1, PDSS2, DHDDS, FNTA, FNTB, RCE1, ZMPSTE24, ICMT, and PCYOX1. Preferably, the terpenoid backbone biosynthesis gene or protein is PDSS2.

At least one biomarker may be a phosphatidylinositide 3-kinase (PI3K) pathway gene or protein. Without being bound by theory, PI3K genes and proteins may correlate with cancer prognosis because the pathway, in part, regulates apoptosis. Non-limiting examples of the PI3K pathway include ligands (such as insulin, IGF-1, IGF-2, EGF, PDGF, FGF, and VEGF), receptor tyrosine kinases (such as insulin receptor, IGF receptor, EGF receptor, PDGF receptor, FGF receptor, and VEGF receptor), kinases (such as PI3K, AKT, mTOR, GSK3-beta, IKK, PDK1, CDKN1B, FAK1 and S6K), phosphatases (such as PTEN and PHLPP), ribosomal proteins (such as ribosomal protein S6), adapter proteins (such as GAB2, GRB2, GRAP, GRAP2, PIK3AP1, PRAS40, PXN, SHB, SH2B1, SH2B2, SH2B3, SH2D3A, and SH2D3C) immunophilins (such as FKBP12, FKBP52, and FKBP5), and transcription factors (such as FoxO1, Hif1-alpha, DEC1 and PLAG1). Preferably, the PI3K gene or protein is a ribosomal protein, such as ribosomal protein S6, particularly phospho-rpS6, or a transcription factor gene or protein, particularly PLAG1. PLAG1 has been shown to regulate the transcription of IGF-2, as well as other target genes, including CRLF1, CRABP2, CRIP2, and PIGF. Accordingly, CRLF1, CRABP2, CRIP2, and PIGF are considered PI3K proteins for the purposes of this application.

At least one biomarker may be a transforming growth factor-beta (TGF-β) pathway gene or protein. Without being bound by theory, TGF-β genes and proteins may correlate with cancer prognosis because the TGF-β signaling pathway stops the cell cycle at G1 stage to stop proliferation and also promotes apoptosis. Disruption of TGF-β signaling increases proliferation and decreases apoptosis. Non-limiting examples of the TGF-β pathway members include ligands (such as Activin A, GDF1, GDF11, BMP2, BMP3, BMP4, BMP5, BMP6, BMP7, Nodal, TGF-β1, TGF-β2, and TGF-β3), Type I receptors (such as TGF-βR1, ACVR1B, ACVR1C, BMPR1A, and BMPR1B), Type II receptors (such as TGF-βR2, ACVR2A, ACVR2B, BMPR2B), SARA, receptor regulated SMADs (such as SMAD1, SMAD2, SMAD3, SMAD5, and SMAD9), coSMAD (such as SMAD4), apoptosis proteins (such as DAXX), and cell cycle proteins (such as p15, p21, Rb, and c-myc). Preferably, the TGF-β pathway gene or protein is a SMAD, particularly SMAD2 or SMAD4.

At least one biomarker may be a voltage-dependent anion channel gene or protein. Without being bound by theory, voltage-dependent anion channel genes and proteins may correlate with cancer prognosis because they have been shown to play a role in apoptosis. Non-limiting examples of the voltage-dependent anion channels include VDAC1, VDAC2, VDAC3, TOMM40 and TOMM40L. Preferably the voltage-dependent anion channel is VDAC1. VDAC1 has been shown to interact with Gelsolin, BCL2-like 1, PRKCE, Bc1-2-associated X protein and DYNLT3. Accordingly, these genes and proteins are considered voltage-gated anion channels for the purposes of this application.

At least one biomarker may be a RNA splicing gene or protein. Without being bound by theory, RNA splicing genes and proteins may correlate with cancer prognosis because abnormally spliced mRNAs are also found in a high proportion of cancerous cells. Non-limiting examples of RNA splicing genes and proteins include snRNPs (such as U1, U2, U4, U5, U6, U11, U12, U4atac, and U6atac), U2AF, and YBX1. Preferably the RNA splicing gene or protein is YBX1. YBX1 has been shown to interact with RBBP6, PCNA, ANKRD2, SFRS9, CTCF and P53. Accordingly, these genes and proteins are considered RNA splicing proteins for the purposes of this application.

The preferred prognosis determinants of this invention include ACTN1, FUS, SMAD2, HOXB13, DERL1, pS6, FAK1, YBX1, SMAD4, VDAC1, COX6C, FKBP5, DCC, CUL2, PLAG1, PDSS2, PXN, AKAP8, DIABLO, CD75, LATS2, DEC1, LMO7, EIF3H, CDKN1B, MTDH2, MAOA, CCND1, HSD17B4, MAP3K5, pPRAS40. More preferred prognosis determinants of this invention include ACTN1, FUS, SMAD2, DERL1, pS6, YBX1, SMAD4, VDAC1, DCC, CUL2, PLAG1, and PDSS2. The twelve more preferred biomarkers are listed in more detail in Table 1 below.

TABLE 1
Prognosis Determinants and Exemplary NCBI Reference Numbers

| | | | | | Expression |
|---|---|---|---|---|---|
| | | Entrez | | | Level |
| | Official gene | Gene | | | Change in |
| Name | name | ID | mRNA | Protein | PCA |
| ACTN1 | ACTN1 | 87 | NM_001102.3 | NP_001093.1 | Decreased |
| | | | NM_001130004.1 | NP_001123476.1 | |
| | | | NM_001130005.1 | NP_001123477.1 | |
| CUL2 | Cullin-2 | 8453 | NM_001198778 | NP_001185707.1 | Increased |
| | | | NM_001198779 | NP_001185708.1 | |
| | | | NM_003591 | NP_003582.2 | |
| | | | NM_001198777 | NP_001185706.1 | |
| DCC | deleted in | 1630 | NM_005215.3 | NP_005206.2 | Increased |
| | colorectal | | | | |
| | carcinoma | | | | |
| DERL1 | Derlin 1 | 79139 | NM_024295.5 | NP_077271.1 | Increased |
| | | | NM_001134671.2 | NP_001128143.1 | |
| FUS | Fused in | 2521 | NM_004960.3 | NP_004951.1 | Increased |
| | sarcoma | | NM_001170634.1 | NP_001164105.1 | |
| | | | NM_001170937.1 | NP_001164408.1 | |
| PDSS2 | prenyl | 57107 | NM_020381.3 | NP_065114.3 | Increased |
| | (decaprenyl) | | | | |
| | diphosphate | | | | |
| | synthase, | | | | |
| | subunit 2 | | | | |
| PLAG1 | pleiomorphic | 5324 | NM_001114634.1 | NP_001108106.1 | Increased |
| | adenoma | | NM_002655.2 | NP_002646.2 | |
| | gene 1 | | NM_001114635.1 | NP_001108107.1 | |
| RpS6 | ribosomal | 6194 | NM_001010.2 | NP_001001.2 | Decreased |
| | protein S6 | | | | |
| SMAD2 | SMAD | 4087 | NM_001003652.3 | NP_001003652.1 | Increased |
| | family | | NM_005901.5 | NP_005892.1 | |
| | member 2 | | NM_001135937.2 | NP_001129409.1 | |
| SMAD4 | SMAD | 4089 | NM_005359.5 | NP_005350.1 | Decreased |
| | family | | | | |
| | member 4 | | | | |
| VDAC1 | voltage- | 7416 | NM_003374.2 | NP_003365.1 | Increased |
| | dependent | | | | |
| | anion | | | | |
| | channel 1 | | | | |
| YBX1 | Y box | 4904 | NM_004559.3 | NP_004550.2 | Decreased |
| | binding | | | | |
| | protein 1 | | | | |

As used herein, the term “ACTN1” refers to actinin, alpha 1. ACTN1 also may be known as actinin alpha 1, alpha-actinin cytoskeletal isoform, non-muscle alpha-actinin-1, F-actin cross-linking protein, actinin 1 smooth muscle, or alpha-actinin-1. It is a F-actin cross-linking protein which may anchor actin to a variety of intracellular structures. For example, the ACTN1 protein sequence may comprise SEQ ID NO: 1 and the ACTN1 mRNA sequence may comprise SEQ ID NO: 2.

As used herein, the term “CULT” refers to Cullin-2. It is a core component of multiple cullin-RING based E3 ubiquitin-protein ligase complexes. For example, the CUL2 protein sequence may comprise SEQ ID NO: 3 and the CUL2 mRNA sequence may comprise SEQ ID NO: 4.

As used herein, the term “DCC” refers to deleted in colorectal cancer. DCC may also be known as IGDCC, colorectal tumor suppressor, colorectal cancer suppressor, deleted in colorectal cancer protein, immunoglobulin superfamily DCC subclass member 1, immunoglobulin superfamily, DCC subclass, member 1, tumor suppressor protein DCC, netrin receptor DCC2 CRC18, and CRCR1. It is a dependence receptor. It promotes axonal growth in the presence of netrin and induces apoptosis when netrin is absent. For example, the DCC protein sequence may comprise SEQ ID NO: 5 and the DCC mRNA sequence may comprise SEQ ID NO: 6.

As used herein, the term “DERL1” refers to Derlin 1. DERL1 may also be known as DER1, DER-1, DER1-like domain family, member, degradation in endoplasmic reticulum protein 1, DERtrin-1, F1113784, MGC3067, PRO2577, and Derl-like protein. It participates in in the ER-associated degradation response and retrotranslocates misfolded or unfolded proteins from the ER lumen to the

cytosol for proteasomal degradation. For example, the DERL1 protein sequence may comprise SEQ ID NO: 7 and the DERL1 mRNA sequence may comprise SEQ ID NO: 8.

As used herein, the term “FUS” refers to fused in sarcoma. FUS may also be known as TLS, ALS6, FUS1, oncogene FUS, oncogene TLS, translocated in liposarcoma protein, 75 kDa DNA-pairing protein, amyotrophic lateral sclerosis 6, hnRNP-P2, ETM4, HNRNPP2, PoMP75, fus-like protein, fusion gene in myxoid liposarcoma, heterogeneous nuclear ribonucleoprotein P2, RNA-binding protein FUS, and POMp75. It is a member of the TET family of proteins, which have been implicated in cellular processes that include regulation of gene expression, maintenance of genomic integrity and mRNA/microRNA processing. For example, the FUS protein sequence may comprise SEQ ID NO: 8 and the FUS mRNA sequence may comprise SEQ ID NO: 10.

As used herein, the term “PDSS2” refers to prenyl (decaprenyl) diphosphate synthase, subunit 2. PDSS2 may also be known as DLP1; hDLP1; COQ10D3; C6orf210; bA59I9.3; decaprenyl pyrophosphate synthetase subunit 2; decaprenyl-diphosphate synthase subunit 2; all-trans-decaprenyl-diphosphate synthase subunit 2; subunit 2 of decaprenyl diphosphate synthase; decaprenyl pyrophosphate synthase subunit 2; EC 2.5.1.91; and chromosome 6 open reading frame 210. It is an enzyme that synthesizes the prenyl side-chain of coenzyme Q or ubiquinone, a key element in the respiratory chain. For example, the PDSS2 protein sequence may comprise SEQ ID NO: 11 and the PDSS2 mRNA sequence may comprise SEQ ID NO: 12.

As used herein, the term “PLAG1” refers to pleiomorphic adenoma gene 1. PLAG1 may also be known as PSA; SGPA; ZNF912; COL1A2/PLAG1 fusion; zinc finger protein PLAG1; and pleiomorphic adenoma gene 1 protein. It is a zinc finger protein with 2 putative nuclear localization signals. For example, the PLAG1 protein sequence may comprise SEQ ID NO: 13 and the PLAG1 mRNA sequence may comprise SEQ ID NO: 14.

As used herein, the term “RpS6” refers to ribosomal protein S6. RpS6 may also be known as S6; phosphoprotein NP33; and 40S ribosomal protein S6. It is a cytoplasmic ribosomal protein that is a component of the 40S ribosome subunit. For example, the RpS6 protein sequence may comprise SEQ ID NO: 15 and the RpS6 mRNA sequence may comprise SEQ ID NO: 16.

As used herein, the term “SMAD2” refers to SMAD family member 2. SMAD2 may also be known as JV18; MADH2; MADR2; JV18-1; hMAD-2; hSMAD2; SMAD family member 2; SMAD, mothers against DPP homolog 2 (Drosophila); mother against DPP homolog 2; mothers against decapentaplegic homolog 2; Sma- and Mad-related protein 2; MAD homolog 2; Mad-related protein 2; mothers against DPP homolog 2; and MAD, mothers against decapentaplegic homolog 2 (Drosophila). It is a member of the Smad family proteins, which are signal transducers and transcriptional modulators that mediate multiple signaling pathway, such as TGF-beta pathway, cell proliferation process, apoptosis process, and differentiation process. For example, the SMAD2 protein sequence may comprise SEQ ID NO: 17 and the SMAD2 mRNA sequence may comprise SEQ ID NO: 18.

As used herein, the term “SMAD4” refers to SMAD family member 4. SMAD4 may also be known as JIP; DPC4; MADH4; MYHRS; deleted in pancreatic carcinoma locus 4; mothers against decapentaplegic homolog 4; mothers against decapentaplegic, Drosophila, homolog of, 4; deletion target in pancreatic carcinoma 4; SMAD, mothers against DPP homolog 4; MAD homolog 4; hSMAD4; MAD, mothers against decapentaplegic homolog 4 (Drosophila); mothers against DPP homolog 4; and SMAD, mothers against DPP homolog 4 (Drosophila). It is a member of the Smad family proteins and can form homomeric complexes and heteromeric complexes with other activated Smad proteins, which then accumulate in the nucleus and regulate the transcription of target genes. For example, the SMAD4 protein sequence may comprise SEQ ID NO: 19 and the SMAD4 mRNA sequence may comprise SEQ ID NO: 20.

As used herein, the term “VDAC1” refers to voltage-dependent anion channel 1. VDAC may also be known as VDAC-1; PORIN; MGC111064; outer mitochondrial membrane protein porin 1; voltage-dependent anion-selective channel protein 1; plasmalemmal porin; VDAC; Porin 31HL; hVDAC1; and Porin 31HM. It is a voltage-dependent anion channel protein that is a major component of the outer mitochondrial membrane. It can facilitate the exchange of metabolites and ions across the outer mitochondrial membrane and may regulate mitochondrial functions. For example, the VDAC1 protein sequence may comprise SEQ ID NO: 21 and the VDAC1 mRNA sequence may comprise SEQ ID NO: 22.

As used herein, the term “YBX1” refers to Y box binding protein 1. YBX1 may also be known as YB1; BP-8; YB-1; CSDA2; NSEP1; MDR-NF1; NSEP-1; nuclease sensitive element binding protein 1; DBPB; Enhancer factor I subunit A; CBF-A3; EFI-A; CCAAT-binding transcription factor I subunit A; DNA-binding protein B; Y-box transcription factor; CSDB; Y-box-binding protein 1; major histocompatibility complex, class II, Y box-binding protein 1; and nuclease-sensitive element-binding protein 1. It mediates pre-mRNA alternative splicing regulation. For example, it can bind to splice sites in pre-mRNA and regulate splice site selection. It can also bind and stabilize cytoplasmic mRNA. For example, the YBX1 protein sequence may comprise SEQ ID NO: 23 and the YBX1 mRNA sequence may comprise SEQ ID NO: 24.

Another biomarker referred to herein is HSPA9. As used herein, the term “HSPA9” refers to heat shock 70 kDa protein 9 (mortalin). HSPA9 may also be known as CSA; MOT; MOT2; GRP75; PBP74; GRP-75; HSPA9B; MTHSP75; or HEL-S-124m. The Entrez Gene ID for human HSPA9 is 3313. A human HSPA9 mRNA sequence is provided in NM_004134.6 (SEQ ID NO:26). A human HSPA9 protein sequence is provided in NP_004125.3 (SEQ ID NO:25). For example, the HSPA9 protein sequence may comprise SEQ ID NO:25. For example, the HSPA9 mRNA sequence may comprise SEQ ID NO:26.

The sequences presented herein are merely illustrative. The biomarkers of this invention encompass all forms and variants of any specifically described biomarkers, including, but not limited to, polymorphic or allelic variants, isoforms, mutants, derivatives, precursors including nucleic acids and pro-proteins, cleavage products, and structures comprised of any of the biomarkers as constituent subunits of the fully assembled structure.

Construction of Biomarker Panels

As mentioned above, ability of the PDs to correlate with cancer prognosis may be amplified by using them in combination. Accordingly, biomarker panels of this invention can be constructed with two or more of the PDs described herein. A biomarker panel of this invention may comprise two, three, four, five, six, seven, eight, nine, ten, eleven, or twelve biomarkers, wherein each biomarker is independently selected from at least one cytoskeletal gene or protein; at least one ubiquitination gene or protein; at least one dependence receptor gene or protein; at least one DNA repair gene or protein; at least one terpenoid backbone biosynthesis gene or protein; at least one PI3K pathway gene or protein; at least one TFG-beta pathway gene or protein; at least one voltage-dependent anion channel gene or protein; and at least one RNA splicing gene or protein. Preferably, the biomarker panel comprises six, seven, eight, or nine biomarkers, most preferably, seven biomarkers.

A preferred biomarker panel of this invention may comprise two, three, four, five, six, seven, eight, nine, ten, eleven, or twelve biomarkers, wherein each biomarker is independently selected from ACTN1, FUS, SMAD2, HOXB13, DERL1, pS6, FAK1, YBX1, SMAD4, VDAC1, COX6C, FKBP5, DCC, CUL2, PLAG1, PDSS2, PXN, AKAP8, DIABLO, CD75, LATS2, DEC1, LMO7, EIF3H, CDKN1B, MTDH2, MAOA, CCND1, HSD17B4, MAP3K5, and pPRAS40. A preferred biomarker panel of this invention may comprise two, three, four, five, six, seven, eight, nine, ten, eleven, or twelve biomarkers, wherein each biomarker is independently selected from ACTN1, FUS, SMAD2, DERL1, pS6, YBX1, SMAD4, VDAC1, DCC, CUL2, PLAG1, and PDSS2. Preferably, the biomarker panel comprises six, seven, eight, or nine biomarkers, most preferably, seven biomarkers. The precise combination and weight of the biomarkers may vary dependent on the prognostic information being sought.

The following combinations of biomarkers are contemplated:
1. PD1 and PD2, wherein PD1 and PD2 are different;
2. PD1, PD2, and PD3, wherein PD1, PD2, and PD3 are different;
3. PD1, PD2, PD3, and PD4, wherein PD1, PD2, PD3, and PD4 are different;
4. PD1, PD2, PD3, PD4, and PD5, wherein PD1, PD2, PD3, PD4, and PD5 are different;
5. PD1, PD2, PD3, PD4, PD5, and PD6, wherein, PD1, PD2, PD3, PD4, PD5, and PD6 are different;
6. PD1, PD2, PD3, PD4, PD5, PD6, and PD7, wherein, PD1, PD2, PD3, PD4, PD5, PD6, and PD7 are different;
7. PD1, PD2, PD3, PD4, PD5, PD6, PD7, and PD8, wherein PD1, PD2, PD3, PD4, PD5, PD6, PD7, and PD8 are different;
8. PD1, PD2, PD3, PD4, PD5, PD6, PD7, PD8, and PD9, wherein PD1, PD2, PD3, PD4, PD5, PD6, PD7, PD8, and PD9 are different;
9. PD1, PD2, PD3, PD4, PD5, PD6, PD7, PD8, PD9, and PD10, wherein PD1, PD2, PD3, PD4, PD5, PD6, PD7, PD8, PD9, and PD10 are different;
10. PD1, PD2, PD3, PD4, PD5, PD6, PD7, PD8, PD9, PD10, and PD11, wherein PD1, PD2, PD3, PD4, PD5, PD6, PD7, PD8, PD9, PD10, and PD11 are different;
11. PD1, PD2, PD3, PD4, PD5, PD6, PD7, PD8, PD9, PD10, PD11 and PD12, wherein PD1, PD2, PD3, PD4, PD5, PD6, PD7, PD8, PD9, PD10, PD11 and PD12 are different;
   wherein PD1, PD2, PD3, PD4, PD5, PD6, PD7, PD8, PD9, PD10, PD11 and PD12 are each independently selected from the group consisting of ACTN1, FUS, SMAD2, HOXB13, DERL1, pS6, FAK1, YBX1, SMAD4, VDAC1, COX6C, FKBP5, DCC, CUL2, PLAG1, PDSS2, PXN, AKAP8, DIABLO, CD75, LATS2, DEC1, LMO7, EIF3H, CDKN1B, MTDH2, MAOA, CCND1, HSD17B4, MAP3K5, and pPRAS40.

The following combinations of biomarkers are preferred:
1. PD1 and PD2, wherein PD1 and PD2 are different;
2. PD1, PD2, and PD3, wherein PD1, PD2, and PD3 are different;
3. PD1, PD2, PD3, and PD4, wherein PD1, PD2, PD3, and PD4 are different;
4. PD1, PD2, PD3, PD4, and PD5, wherein PD1, PD2, PD3, PD4, and PD5 are different;
5. PD1, PD2, PD3, PD4, PD5, and PD6, wherein, PD1, PD2, PD3, PD4, PD5, and PD6 are different;
6. PD1, PD2, PD3, PD4, PD5, PD6, and PD7, wherein, PD1, PD2, PD3, PD4, PD5, PD6, and PD7 are different;
7. PD1, PD2, PD3, PD4, PD5, PD6, PD7, and PD8, wherein PD1, PD2, PD3, PD4, PD5, PD6, PD7, and PD8 are different;
8. PD1, PD2, PD3, PD4, PD5, PD6, PD7, PD8, and PD9, wherein PD1, PD2, PD3, PD4, PD5, PD6, PD7, PD8, and PD9 are different;
9. PD1, PD2, PD3, PD4, PD5, PD6, PD7, PD8, PD9, and PD10, wherein PD1, PD2, PD3, PD4, PD5, PD6, PD7, PD8, PD9, and PD10 are different;
10. PD1, PD2, PD3, PD4, PD5, PD6, PD7, PD8, PD9, PD10, and PD11, wherein PD1, PD2, PD3, PD4, PD5, PD6, PD7, PD8, PD9, PD10, and PD11 are different;
11. PD1, PD2, PD3, PD4, PD5, PD6, PD7, PD8, PD9, PD10, PD11 and PD12, wherein PD1, PD2, PD3, PD4, PD5, PD6, PD7, PD8, PD9, PD10, PD11 and PD12 are different;
   wherein PD1, PD2, PD3, PD4, PD5, PD6, PD7, PD8, PD9, PD10, PD11 and PD12 are each independently selected from the group consisting of ACTN1, FUS, SMAD2, DERL1, pS6, YBX1, SMAD4, VDAC1, DCC, CUL2, PLAG1, and PDSS2.

Optionally, the combinations of biomarkers comprise at least ACTN1, YBX1, SMAD2, and FUS. Alternatively, the combinations of biomarkers comprise (1) at least ACTN1, YBX1, and SMAD2; (2) at least ACTN1, YBX1, and FUS; (3) at least ACTN1, SMAD2, and FUS; or (4) at least YBX1, SMAD2, and FUS. Some of the preferred combinations of biomarkers are provided in Table 6, which is disclosed in U.S. Provisional Application No. 61/792,003, filed Mar. 15, 2013, the entire content of which is incorporated by reference herein.

Tissue Samples

Tissue samples used in the methods of the invention may be tumor samples (e.g., prostate tumor samples) obtained by biopsy. A health care provider may order a biopsy (e.g., a prostate biopsy) if results from initial tests, such as a prostate-specific antigen (PSA) blood test or digital rectal exam (DRE), suggest prostate cancer. To obtain a prostate biopsy, a health care provider may use a fine needle to collect a number of tissue samples (also called “cored” samples) from the prostate gland (see also discussion infra). Tissue samples for the methods of this invention may also be obtained through surgery (e.g., prostatectomy) performed by a urologist or a robotic surgeon. The tissue sample obtained by surgery may be a whole or partial prostate and may comprise one or more lymph nodes. In one embodiment, the tissue samples may be formalin-fixed and paraffin-embedded (FFPE) in blocks. Sections may then be cut from the FFPE blocks and placed on slides by any appropriate means. Slides containing samples from multiple tumors or patients can be combined into one batch as a tissue microarray (TMA) for processing. Frozen tissues may be used as well. Suitable control slides or control cores, e.g., those prepared from cell lines that have a broad range of expression of ACTN1, CUL2, DCC, DERL1, FUS, PDSS2, PLAG1, RpS6, SMAD2, SMAD4, VDAC1, and YBX1, may be added to the batch.

A set of control cell lines that show high, intermediate, and low levels of expression for each biomarker (e.g., ACTN1, CUL2, DCC, DERL1, FUS, PDSS2, PLAG1, RpS6, SMAD2, SMAD4, VDAC1, and YBX1) can be selected. These cell lines can then be fixed with formalin, processed, and incorporated into paraffin blocks using standard histology techniques. A cell line control TMA can be established by placing a core from each cell line paraffin block into a new acceptor block. This cell line control TMA can be sectioned and the resulting sections can be stained in parallel to patient tissue samples. Since cell lines represent a homogeneous and reproducible source of biomarkers expression, such a cell line control TMA can be used as a reference point for quantitative immuno-staining assay measuring biomarkers' expression in patient tissue samples. Comparing quantitative control levels over time allows a user to determine if the equipment is trending out of calibration. If necessary, a user may also standardize patient samples against control values for absolute quantitation between batches.

Measurement of Biomarkers

The biomarkers of this invention can be measured in various forms. For example, levels of biomarkers can be measured at the genomic DNA level (e.g. measuring copy number, heterozygosity, deletions, insertions or point mutations), the mRNA level (e.g, measuring transcript level or transcript location), the protein level (e.g., protein expression level, quantification of post-translational modification, or activity level), or at the metabolite/analyte level. Methods for measuring the levels of biomarkers at the genomic DNA, mRNA, protein and metabolite/analyte levels are known in the art. Preferably, levels of biomarkers are determined at the protein level, in whole cells and/or in subcellular compartments (e.g., nucleus, cytoplasm and cell membrane). Exemplary methods for determining the levels at the protein level include, without limitation, immunoassays such as immunohistochemistry assays (IHC), immunofluorescence assays (IF), enzyme-linked immunosorbent assays (ELISA), immunoradiometric assays, and immunoenzymatic assays. In immunoassays, one may use, for example, antibodies that bind to a biomarker or a fragment thereof. The antibodies may be monoclonal, polyclonal, chimeric, or humanized. The antibodies may be bispecific. One may also use antigen-binding fragments of a whole antibody, such as single chain antibodies, Fv fragments, Fab fragments, Fab′ fragments, F(ab′)2 fragments, Fd fragments, single chain Fv molecules (scFv), bispecific single chain Fv dimers, nanobodies, diabodies, domain-deleted antibodies, single domain antibodies, and/or an oligoclonal mixture of two or more specific monoclonal antibodies.

For example, the tissue samples, e.g., the biopsy slides described above, can be assayed to measure the levels of the appropriate biomarkers, in, for example, an immunohistochemical (IHC) assay. In an IHC assay, detectably-labeled antibodies to the various biomarkers can be used to stain a prostate tissue sample and the levels of binding can be indicated by, e.g., fluorescence or luminescent emission. Colorimetric dyes (e.g., DAB, Fast Red) can be used as well. In one embodiment, the prostate tissue slides are stained with one or more of antibodies that bind respectively to ACTN1, CUL2, DCC, DERL1, FUS, PDSS2, PLAG1, RpS6, SMAD2, SMAD4, VDAC1, and YBX1. The antibodies used in the methods of the invention may be monoclonal or polyclonal. Antigen-binding portions of whole antibodies, or any other molecular entities (e.g., peptide mimetics and aptamers) that can bind specifically to the biomarkers can also be used.

Other methods to measure biomarkers at the protein level include, for example, chromatography, mass spectrometry, Luminex xMAP Technology, microfluidic chip-based assays, surface plasmon resonance, sequencing, Western blot analysis, aptamer binding, molecular imprints, peptidomimetics, affinity-based peptide binding, affinity-based chemical binding, or a combination thereof. To determine whole cell and/or subcellular levels of a biomarker, one may also use methods such as AQUA® (see, e.g., U.S. Pat. Nos. 7,219,016, and 7,709,222; Camp et al., Nature Medicine, 8(11):1323-27 (2002)), and Definiens TissueStudio™ (see, e.g., U.S. Pat. Nos. 7,873,223, 7,801,361, 7,467,159, and 7,146,380, and Baatz et al., Comb Chem High Throughput Screen, 12(9):908-16 (2009)).

In some embodiments, the measured level of a biomarker is normalized against normalizing proteins, including expression products of housekeeping genes such as GAPDH, Cynl, ZNF592, or actin, to remove sources of variation. Methods of normalization are well known in the art. See, e.g., Park et al., BMC Bioinformatics. 4:33 (2003).

Defining a Region of Interest

To improve accuracy of the assays, it may be desirable to define a region of interest and only quantify biomarkers in that region of interest. A region of interest may be defined by applying a “tumor mask” to the sample so that only biomarker levels in a tumor region are measured. A “tumor mask” refers to a combination of biomarkers that allows identification of tumor regions in a tissue of interest. For example, prostate cancer is typically a carcinoma expressing epithelial markers such as cytokeratin 8 (CK8 or KRT8) and cytokeratin 18 (CK18 or KRT18) while not expressing prostate basal markers such as cytokeratin 5 (CK5 or KRTS). Thus, a “tumor mask” for prostate cancer may entail the use of a mixture of antibodies that bind specifically to these markers. We have also found surprisingly that TRIM29, a tumor marker for some other cancers, is a basal marker, not a tumor marker, in prostate tissue; thus, anti-TRIM29 antibodies may also be used in a prostate tumor mask. For example, a prostate tumor mask useful in this invention may comprise a mixture of anti-CK5, anti-CK8, anti-CK18, and anti-TRIM29 antibodies, where a prostate tumor region is defined as a prostate tissue region bound by anti-CK8 and anti-CK18 antibodies and not bound by anti-CK5 and anti-TRIM29 antibodies. A prostate tumor region may be defined as a prostate tissue region bound by either anti-CK8 or anti-CK18 antibodies, preferably both. Similarly, a prostate tumor region may be defined as a prostate tissue region not bound by anti-CK5 antibodies or not bound by anti-TRIM29 antibodies. Preferably, the prostate tumor region is not bound by either anti-CK5 or anti-TRIM29 antibodies. A basal prostate tumor region may be defined as a prostate tissue region bound by either anti-CK5 or anti-TRIM2 antibodies, preferably both. Preferably, the basal tumor region is not bound by either anti-CK8 or anti-CK18 antibody. Alternatively, other combinations of epithelial and basal markers could be used, such as ESA antibody for epithelial and p63 antibody for basal cells. In cancers other than prostate cancer, other combinations of markers that allow tumor region identification could be used, such as S100 markers specific for malignant melanoma.

Accordingly, one aspect of the present invention provides a method for defining a region of interest in a tissue sample comprising contacting the tissue sample with one or more first reagents for specifically for identifying the region of interest. The region of interest may comprise cancer cells, such as prostate cancer cells. To identify prostate cancer cells, the one or more first reagents may comprise an anti-cytokeratin 8 antibody, an anti-cytokeratin 18 antibody, or both. The method may further comprise defining a region of the tissue sample to be excluded from the region of interest, e.g., noncancerous cells, by contacting the tissue sample with one or more second reagents for specifically for identifying the region to be excluded. For example, to exclude basal, noncancerous prostate cells, the one or more second reagents may comprise an anti-cytokeratin 5 antibody, an anti-TRIM29 antibody, or both.

To allow measurement of biomarkers in subcellular regions such as nucleus, cytoplasm, and cell membrane, it is necessary to use specific markers for those regions. Cytokeratins 8 and 18 that are used for identification of epithelial regions provide cytoplasm- and membrane-specific staining pattern and can hence be used to define this subcellular localization. To identify the nucleus area of cells, a prostate tissue sample may be stained with nuclear-specific fluorescent dyes, such as DAPI or Hoechst 33342.

After appropriate stainings have been performed, the biopsy slides can be treated to preserve signals for detection, e.g., by applying anti-fade reagents and/or cover slips on the slides. The slides can then be stored and read by an imaging machine. Images so obtained can then be processed and biomarker expression quantified. This process is also termed quantitative multiplex immunofluorescence acquisition (QMIF acquisition).

The multiplex in situ proteomics technology of this invention provides several advantages over conventional genetics platforms where gene expression, rather than protein expression/activity, is measured. First, the use of tumor mask enables procurement of marker information from tumor tissue only, without “dilution” from normal tissue, therefore enhancing accuracy of the test. The current technology also enables quantitation of markers in different regions of tumor tissue, which is known to be quite heterogeneous. Readout from the most aggressive region of a tumor provides a more accurate outlook on the patient's clinical outcome, and therefore is more useful in helping physicians to determine the best course of treatment for the patient. In addition, the multivariate diagnostic methods of this invention have been designed to predict outcome even on less representative tumor regions, alleviating problems caused by random sampling error due to tumor heterogeneity. Furthermore, the use of activation-state antibodies and sub-cellular localization of the markers enables quantification of functionally active markers, further enhancing the accuracy of the test.

Data Processing

Images obtained from immunofluorescence of the tumor samples may be exported into pattern recognition software that uses an algorithm suitable for automated quantitative analysis of data acquired from the images (e.g., an algorithm developed using Definiens Developer XD™ or other image analysis software such as INFORM (PerkinElmer). In some embodiments of the invention, such an algorithm measures the presence and/or levels of antibody staining for one or more of ACTN1, CUL2, DCC, DERL1, FUS, PDSS2, PLAG1, RpS6, SMAD2, SMAD4, VDAC1, and YBX1. The algorithm may be used to focus this measurement on the tumor regions defined by presence of CK8 and CK18 staining and the absence of CK5 and TRIM29 staining. In some embodiments, the algorithm is used to generate heat maps of maximum aggressiveness areas for one or more of ACTN1, CUL2, DCC, DERL1, FUS, PDSS2, PLAG1, RpS6, SMAD2, SMAD4, VDAC1, and YBX1. The algorithm also may be used to measure tumor volume.

Data obtained from image processing of the tissue samples are used to calculate a risk score. The risk score may measure the aggressiveness of the tumor (e.g., the prostate tumor). For example, the risk score may predict the probability that the tumor (e.g., the prostate tumor) is actively progressing or indolent/dormant at the time of diagnosis. The risk score may also predict the probability that the tumor (e.g., the prostate tumor) will progress at some later point after the time of diagnosis. The risk score may also indicate the lethal outcome/disease-specific death (DSD) of the cancer (e.g., the prostate cancer), i.e., the probability that a patient with the tumor will die from the cancer (e.g., the number of years of expected survival), or the risk that a tumor (e.g., a prostate tumor) will progress or metastasize. These probabilities may obtained by evaluating the model/classifier trained to predict this risk, at the marker values measured in the sample. Several probabilistic binary classifiers can be used and are known to the skilled in the art such as random forests or logistic regression. In the Examples presented below, logistic regression was used. The risk score may also be used to detect cells with metastatic potential in a tumor tissue sample. The risk score may also incorporate other diagnostic results or cancer parameters, for example digital rectal examination (DRE) results, prostate-specific antigen (PSA) levels, PSA kinetics, the Gleason score, tumor stage, tumor size, age of onset, and lymph node status. The risk score may be communicated to the health care provider and/or patient and used to determine a treatment regimen for the patient (for example, surgery).

Clinical Applications

The present diagnostic methods are useful for a health care provider to determine the most appropriate treatment for a cancer patient (e.g., prostate cancer patient). When a health care provider suspects cancer (e.g., prostate cancer) in a patient based on medical history, DRE, and/or PSA levels, he or she may order a biopsy (e.g., a prostate biopsy). To perform a biopsy, a general practitioner or urologist may use a transurethral ultrasound (TRUS)-guided core needle to obtain multiple (e.g., 8-18) cored samples, each about ½ inch long and 1/16 inch wide. If cancerous cells are found by morphological examination, further tests (e.g., imaging tests such as bone scan, CT scan, and MRI Prostastint™ Scan) can be done to help stage the cancer. The diagnostic methods of this invention can then be performed to further predict the aggressiveness, risk of progression, or outcome of the cancer. If the methods predict 1) active progression of tumor; 2) a high risk of progression; or 3) a lethal outcome, a health care provider may decide to use aggressive treatment. For example, in addition to prostatectomy, a physician may use radiation therapy (e.g., external beam radiation, proton therapy, and brachytherapy), hormonal therapy (e.g., orchiectomy, LHRH agonists or antagonists, and anti-androgens), chemotherapy, and other appropriate treatments (e.g., Sipuleucel-T (PROVENGE®) therapy, cryosurgery, and high intensity laser therapy). If, however, a patient is prognosticated to have indolent PCA, then he can be referred to active surveillance and be subject to repeat biopsies, without the need to undergo radical treatment.

Accordingly, one aspect of the present invention provides methods for predicting the prognosis of a cancer patient. The method may comprise measuring, in a sample obtained from a patient, the levels of two or more PDs selected from at least one cytoskeletal gene or protein; at least one ubiquitination gene or protein; at least one dependence receptor gene or protein; at least one DNA repair gene or protein; at least one terpenoid backbone biosynthesis gene or protein; at least one PI3K pathway gene or protein; at least one TFG-beta pathway gene or protein; at least one voltage-dependent anion channel gene or protein; and at least one RNA splicing gene or protein; wherein the measured levels are indicative of the prognosis of the cancer patient. Optionally, the two or more PDs are selected from the group consisting of ACTN1, FUS, SMAD2, HOXB13, DERL1, RpS6, FAK1, YBX1, SMAD4, VDAC1, COX6C, FKBP5, DCC, CUL2, PLAG1, PDSS2, PXN, AKAP8, DIABLO, CD75, LATS2, DEC1, LMO7, EIF3H, CDKN1B, MTDH2, MAOA, CCND1, HSD17B4, MAP3K5, and pPRAS40. Preferably, the two or more PDs are elected from the group consisting of ACTN1, CUL2, DCC, DERL1, FUS, PDSS2, PLAG1, RpS6, SMAD2, SMAD4, VDAC1, and YBX1. The method may further comprise the step of obtaining the sample from the patient. The prognosis may be that the cancer is an aggressive form of cancer, that the patient is at risk for having an aggressive form of cancer or that the patient is at risk of having a cancer-related lethal outcome. The cancer may be prostate cancer.

Another aspect of the present invention provides a method for identifying a cancer patient in need of adjuvant therapy, comprising obtaining a tissue sample from the patient; and measuring, in the sample, the levels of two or more PDs selected from at least one cytoskeletal gene or protein; at least one ubiquitination gene or protein; at least one dependence receptor gene or protein; at least one DNA repair gene or protein; at least one terpenoid backbone biosynthesis gene or protein; at least one PI3K pathway gene or protein; at least one TFG-beta pathway gene or protein; at least one voltage-dependent anion channel gene or protein; and at least one RNA splicing gene or protein; wherein the measured levels indicate that the patient is in need of adjuvant therapy. Optionally, the two or more PDs are selected from the group consisting of ACTN1, FUS, SMAD2, HOXB13, DERL1, RpS6, FAK1, YBX1, SMAD4, VDAC1, COX6C, FKBP5, DCC, CUL2, PLAG1, PDSS2, PXN, AKAP8, DIABLO, CD75, LATS2, DEC1, LMO7, EIF3H, CDKN1B, MTDH2, MAOA, CCND1, HSD17B4, MAP3K5, and pPRAS40. Preferably, the two or more PDs are elected from the group consisting of ACTN1, CUL2, DCC, DERL1, FUS, PDSS2, PLAG1, RpS6, SMAD2, SMAD4, VDAC1, and YBX1.

An additional aspect of the present invention provides a method for treating a cancer patient, comprising measuring the levels of two or more PDs selected from the group consisting of at least one cytoskeletal gene or protein; at least one ubiquitination gene or protein; at least one dependence receptor gene or protein; at least one DNA repair gene or protein; at least one terpenoid backbone biosynthesis gene or protein; at least one PI3K pathway gene or protein; at least one TFG-beta pathway gene or protein; at least one voltage-dependent anion channel gene or protein; and at least one RNA splicing gene or protein; and treating the patient with an adjuvant therapy if the measured levels indicate that the patient has actively progressing cancer, or a risk of cancer progression, or a risk of having a cancer-related lethal outcome. Optionally, the two or more PDs are selected from the group consisting of ACTN1, FUS, SMAD2, HOXB13, DERL1, RpS6, FAK1, YBX1, SMAD4, VDAC1, COX6C, FKBP5, DCC, CUL2, PLAG1, PDSS2, PXN, AKAP8, DIABLO, CD75, LATS2, DEC1, LMO7, EIF3H, CDKN1B, MTDH2, MAOA, CCND1, HSD17B4, MAP3K5, and pPRAS40. Preferably, the two or more PDs are elected from the group consisting of ACTN1, CUL2, DCC, DERL1, FUS, PDSS2, PLAG1, RpS6, SMAD2, SMAD4, VDAC1, and YBX1. Alternatively, the method comprises identifying patient with level changes in at least two PDs, wherein the level changes are selected from the group consisting of up-regulation of one or more of CUL2, DCC, DERL1, FUS, PDSS2, PLAG1, SMAD2, and VDAC land down-regulation of one or more of ACTN1, RpS6, SMAD4, and YBX1; and treating the patient with an adjuvant therapy. The patient may have prostate cancer.

The adjuvant therapy may be selected from the group consisting of radiation therapy, chemotherapy, immunotherapy, hormone therapy, and targeted therapy. In some embodiments, the targeted therapy targets a component of a signaling pathway in which one or more of the selected PD is a component and wherein the targeted component is different from the selected PD. Alternatively, the targeted therapy targets one or more of the selected PD. The patient may have been subjected to a standard of care therapy, such as surgery, radiation, chemotherapy, or androgen ablation.

A further aspect of the present invention provides a method of identifying a compound capable of reducing the risk of cancer progression, or delaying or slowing the cancer progression, comprising providing a cell expressing a PD selected from the group consisting of ACTN1, CUL2, DCC, DERL1, FUS, PDSS2, PLAG1, RpS6, SMAD2, SMAD4, VDAC1, and YBX1; contacting the cell with a candidate compound; and determining whether the candidate compound alters the expression or activity of the selected PD; whereby the alteration observed in the presence of the compound indicates that the compound is capable of reducing the risk of cancer progression, or delaying or slowing the cancer progression.

Another aspect of the present invention provides a method for treating a cancer patient, comprising measuring the levels of two or more PDs selected from the group consisting of at least one cytoskeletal gene or protein; at least one ubiquitination gene or protein; at least one dependence receptor gene or protein; at least one DNA repair gene or protein; at least one terpenoid backbone biosynthesis gene or protein; at least one PI3K pathway gene or protein; at least one TFG-beta pathway gene or protein; at least one voltage-dependent anion channel gene or protein; and at least one RNA splicing gene or protein; and administering an agent that modulates the level of the selected PD. Optionally, the two or more PDs are selected from the group consisting of ACTN1, FUS, SMAD2, HOXB13, DERL1, RpS6, FAK1, YBX1, SMAD4, VDAC1, COX6C, FKBP5, DCC, CUL2, PLAG1, PDSS2, PXN, AKAP8, DIABLO, CD75, LATS2, DEC1, LMO7, EIF3H, CDKN1B, MTDH2, MAOA, CCND1, HSD17B4, MAP3K5, and pPRAS40. Preferably, the two or more PDs are elected from the group consisting of ACTN1, CUL2, DCC, DERL1, FUS, PDSS2, PLAG1, RpS6, SMAD2, SMAD4, VDAC1, and YBX1. Alternatively, the method comprises identifying patient with level changes in at least two PDs, wherein the level changes are selected from the group consisting of up-regulation of one or more of CUL2, DCC, DERL1, FUS, PDSS2, PLAG1, SMAD2, and VDAC1 and down-regulation of one or more of ACTN1, RpS6, SMAD4, and YBX1; and administering an agent that modulates the level of at least one of the PDs.

In any of the methods above, the levels of at least three, four, five, six, seven, eight, nine, ten, eleven, or twelve PDs may be measured. Optionally, the levels of six PDs consisting of PD1, PD2, PD3, PD4, PD5, and PD6 are measured, wherein PD1, PD2, PD3, PD4, PD5, and PD6 are different and are independently selected from the group consisting of ACTN1, FUS, SMAD2, HOXB13, DERL1, RpS6, FAK1, YBX1, SMAD4, VDAC1, COX6C, FKBP5, DCC, CUL2, PLAG1, PDSS2, PXN, AKAP8, DIABLO, CD75, LATS2, DEC1, LMO7, EIF3H, CDKN1B, MTDH2, MAOA, CCND1, HSD17B4, MAP3K5, and pPRAS40. Preferably, PD1, PD2, PD3, PD4, PD5, and PD6 are different and are independently selected from the group consisting of ACTN1, CUL2, DCC, DERL1, FUS, PDSS2, PLAG1, RpS6, SMAD2, SMAD4, VDAC1, and YBX1.

Optionally, the levels of seven PDs consisting of PD1, PD2, PD3, PD4, PD5, PD6, and PD7 are measured, wherein PD1, PD2, PD3, PD4, PD5, PD6, and PD7 are different and are independently selected from the group consisting of ACTN1, FUS, SMAD2, HOXB13, DERL1, RpS6, FAK1, YBX1, SMAD4, VDAC1, COX6C, FKBP5, DCC, CUL2, PLAG1, PDSS2, PXN, AKAP8, DIABLO, CD75, LATS2, DEC1, LMO7, EIF3H, CDKN1B, MTDH2, MAOA, CCND1, HSD17B4, MAP3K5, and pPRAS40. Preferably, PD1, PD2, PD3, PD4, PD5, PD6, and PD7 are different and are independently selected from the group consisting of ACTN1, CUL2, DCC, DERL1, FUS, PDSS2, PLAG1, RpS6, SMAD2, SMAD4, VDAC1, and YBX1. The method may further comprise measuring the levels of one or more PDs selected from the group consisting of HOXB13, FAK1, COX6C, FKBP5, PXN, AKAP8, DIABLO, CD75, LATS2, DEC1, LMO7, EIF3H, CDKN1B, MTDH2, MAOA, CCND1, HSD17B4, MAP3K5, and pPRAS40.

The measured level of at least one PD may be up-regulated relative to a reference value. Preferably, the up-regulated PD is selected from the group consisting of CUL2, DCC, DERL1, FUS, PDSS2, PLAG1, SMAD2, and VDAC1. Further, the measured level of at least one PD may be down-regulated relative to a reference value. Preferably, the down-regulated PD is selected from the group consisting of ACTN1, RpS6, SMAD4, and YBX1. Accordingly the measured level of at least one PD may be up-regulated relative to a reference value and the measured of at least one PD may be down-regulated relative to a reference value. The reference value may be the measured level of the PD in noncancerous cells.

Any of the methods above may comprise measuring the genomic DNA levels, the mRNA levels or the protein levels of the each PD. For example, the method may comprise contacting the sample with an oligonucleotide, aptamer or antibody specific for each PD. The levels of PDs may be measured separately or concurrently, for example, using a multiplex reaction. Preferably, the protein level of each PD is measured. Antibodies or antibody fragments may be used to measure protein levels, for example by immunohistochemistry or immunofluorescence. When more than one PD is measured from a single sample, antibodies or fragments thereof may each be labeled or bound by a different fluorophore. Signals from the different fluorophores can be detected concurrently by an automated imaging machine.

The protein levels of the PDs may be measured in specific subcellular compartments. For example, a DAPI stain can be used to identify the nucleus of each cell so the amount of each PD in the nucleus and/or the cytoplasm can be measured.

Similarly, the levels of the PDs may be measured only in a defined region of interest. In cancer, for example, cancer cells would be included in the region of interest, while noncancer cells may be excluded from the region of interest. In the prostate, cancer cells express cytokeratin-8 and cytokeratin-18 and basal (noncancer) cells express cytokeratin-5 and TRIM29. Accordingly, the region of interest may defined by anti-cytokeratin 8 antibody and anti-cytokeratin 18 antibody staining and further defined by lack of anti-cytokeratin 5 antibody and anti-TRIM29 antibody staining. The exclude region may be defined by anti-cytokeratin 5 antibody and anti-TRIM29 antibody staining and further defined by lack of anti-cytokeratin 8 antibody and anti-cytokeratin 18 antibody staining.

In any of the methods above, the sample is a solid tissue sample or a blood sample, preferably a solid tissue sample. The solid tissue sample may be a formalin-fixed paraffin-embedded tissue sample, a snap-frozen tissue sample, an ethanol-fixed tissue sample, a tissue sample fixed with an organic solvent, a tissue sample fixed with plastic or epoxy, a cross-linked tissue sample, a surgically removed tumor tissue, or a biopsy sample, such as a core biopsy, and excisional tissue biopsy or an incisional tissue biopsy. Preferably, the sample is a cancerous tissue sample. The sample may be a prostate tissue sample, for example a formalin-fixed paraffin-embedded (FFPE) prostate tumor sample. Accordingly, the above methods may further comprise contacting a cross-section of the FFPE prostate tumor sample with an anti-cytokeratin 8 antibody, an anti-cytokeratin 18 antibody, an anti-cytokeratin 5 antibody, and an anti-TRIM29 antibody, wherein the measuring step is conducted in an area in the cross section that is bound by the anti-cytokeratin 8 and anti-cytokeratin 18 antibodies and is not bound by the anti-cytokeratin 5 and anti-TRIM29 antibodies.

Any of the methods above may further comprise measuring at least one standard parameter associated with the cancer. Standard parameters include, but are not limited to, Gleason score, tumor stage, tumor grade, tumor size, tumor visual characteristics, tumor location, tumor growth, lymph node status, tumor thickness (Breslow score), ulceration, age of onset, PSA level, and PSA kinetics.

Additional Prognostic Factors

The biomarker panels of this invention may be used in conjunction with additional biomarkers, clinical parameters, or traditional laboratory risk factors known to be present or associated with the clinical outcome of interest. One or more clinical parameters may be used in the practice of the invention as a biomarker input in a formula or as a pre-selection criterion defining a relevant population to be measured using a particular biomarker panel and formula. One or more clinical parameters may also be useful in the biomarker normalization and pre-processing, or in biomarker selection, panel construction, formula type selection and derivation, and formula result post-processing. A similar approach can be taken with the traditional laboratory risk factors. Clinical parameters or traditional laboratory risk factors are clinical features typically evaluated in the clinical laboratory and used in traditional global risk assessment algorithms. Clinical parameters or traditional laboratory risk factors for tumor metastasis may include, for example, tumor stage, tumor grade, tumor size, tumor visual characteristics, tumor location, tumor growth, lymph node status, histology, tumor thickness (Breslow score), ulceration, proliferative index, tumor-infiltrating lymphocytes, age of onset, PSA level, or Gleason score. Other traditional laboratory risk factors for tumor metastasis are known to those skilled in the art.

In some embodiments, the biomarker scores obtained by the present methods may be used in conjunction with Gleason score to obtain better predictive results. A Gleason score is given to prostate cancer based on the prostate tissue's microscopic appearance, and it has been used clinically to predict PCA prognosis. To obtain a Gleason score, a prostate tissue sample may be stained with hematoxylin and eosin (H&E) and examined under a microscope by a pathologist. Prostate tumor patterns in the sample are graded on a scale of 1-5, with 5 being the least differentiated and most invasive. The grade of most common pattern (more than 50% of the tumor) is added with the grade of second most common pattern (less than 50% but more than 5%) to form a tumor Gleason score. A score of 2-6 indicates low-grade PCA with low recurrence risk. A score of 7 (3+4 or 4+3) indicates intermediate-grade PCA with intermediate recurrence risk, where a score of 4+3 is worse than a score of 3+4. A score of 8-10 indicates high-grade PCA with high recurrence risk. The risk score as determined by the methods described herein can be used together with Gleason score and can improve predictive abilities of Gleason score. For example, intermediate Gleason score of 7 (3+4) does not give a good prediction of patient risk of PCA recurrence. But addition of the risk score as calculated by the methods described herein will improve predictive power of that intermediate Gleason score.

Kits for Detecting Biomarkers

Another aspect of the present invention is the ability to generate kits for measuring the levels of two or more PDs selected from the group consisting of at least one cytoskeletal gene or protein; at least one ubiquitination gene or protein; at least one dependence receptor gene or protein; at least one DNA repair gene or protein; at least one terpenoid backbone biosynthesis gene or protein; at least one PI3K pathway gene or protein; at least one TFG-beta pathway gene or protein; at least one voltage-dependent anion channel gene or protein; and at least one RNA splicing gene or protein; comprising reagents for specifically measuring the levels of the selected PDs. Optionally, the two or more PDs are selected from the group consisting of ACTN1, FUS, SMAD2, HOXB13, DERL1, RpS6, FAK1, YBX1, SMAD4, VDAC1, COX6C, FKBP5, DCC, CUL2, PLAG1, PDSS2, PXN, AKAP8, DIABLO, CD75, LATS2, DEC1, LMO7, EIF3H, CDKN1B, MTDH2, MAOA, CCND1, HSD17B4, MAP3K5, and pPRAS40. Preferably, the two or more PDs are elected from the group consisting of ACTN1, CUL2, DCC, DERL1, FUS, PDSS2, PLAG1, RpS6, SMAD2, SMAD4, VDAC1, and YBX1.

The reagents may measure genomic DNA levels, mRNA transcript levels, or protein levels of the selected PDs. Preferably the reagents comprise one or more antibodies or fragments thereof, oligonucleotides, or apatmers.

Methods for Selecting Biomarkers

Another aspect of the present invention a method for identifying prognosis determinants for a disease of interest comprising a biological step; a technical step; a performance step; and a validation step.

The biological step may comprise generating a candidate list is compiled for the disease of interest from publically available data, including scientific literature, databases, and presentations at meetings; and prioritizing the candidate list based on biological relevance, in silico analysis, known expression information, and commercial availability of requisite monoclonal antibodies.

The technical step may comprise obtaining antibodies for candidate prognosis determinants; testing the antibodies in an immunohistochemistry assay using 3,3′-Diaminobenzidine (DAB) staining to evaluate staining specificity and intensity; and testing antibodies with sufficient staining specificity and intensity with DAB in an immunofluorescence (IF) assay to determine IF specificity, signal intensity and dynamics to identify antibodies that pass the technical requirements.

The performance step may comprise contacting a mini tissue microarray (TMA) with the antibodies that pass the technical requirements, wherein the mini TMA comprises several samples at different stages of the disease of interest; quantifying the immunofluorescent intensity for each antibody; correlating the immunofluorescent intensity for each antibody for the prognosis of each sample in the mini TMA; and determining which antibodies demonstrate univariate performance on the mini TMA for correlation with he prognosis of disease of interest. Optionally, the performance step further comprises contacting a larger TMA with the antibodies that pass the technical requirements, wherein the larger TMA comprises several samples at different stages of the disease of interest; quantifying the immunofluorescent intensity for each antibody; correlating the immunofluorescent intensity for each antibody for the prognosis of each sample in the larger TMA; and determining which antibodies demonstrate univariate performance on the larger TMA for correlation with he prognosis of disease of interest. In some embodiments, the performance step further comprises performing bioinformatics analysis to identify combinations of antibodies for PDs that are correlate with the prognosis of the disease of interest.

The validation step may comprise obtaining tissue samples from patients suffering from the disease of interest; contacting the tissue samples with antibodies for PDs or combinations of antibodies for PDs for the disease of interest; quantifying the immunofluorescent intensity for each antibody or combination of antibodies; and correlating the immunofluorescent intensity for each antibody or combination of antibodies with the subject's prognosis for the disease of interest.

Example Computer System

Various aspects and functions described herein in accord with the present disclosure may be implemented as hardware, software, or a combination of hardware and software on one or more computer systems. There are many examples of computer systems currently in use. Some examples include, among others, network appliances, personal computers, workstations, mainframes, networked clients, servers, media servers, application servers, database servers, web servers, and virtual servers. Other examples of computer systems may include mobile computing devices, such as cellular phones and personal digital assistants, and network equipment, such as load balancers, routers and switches. Additionally, aspects in accord with the present disclosure may be located on a single computer system or may be distributed among a plurality of computer systems connected to one or more communication networks.

For example, various aspects and functions may be distributed among one or more computer systems configured to provide a service to one or more client computers, or to perform an overall task as part of a distributed system. Additionally, aspects may be performed on a client-server or multi-tier system that includes components distributed among one or more server systems that perform various functions. Thus, the disclosure is not limited to executing on any particular system or group of systems. Further, aspects may be implemented in software, hardware or firmware, or any combination thereof. Thus, aspects in accord with the present disclosure may be implemented within methods, acts, systems, system placements and components using a variety of hardware and software configurations, and the disclosure is not limited to any particular distributed architecture, network, or communication protocol. Furthermore, aspects in accord with the present disclosure may be implemented as specially-programmed hardware and/or software.

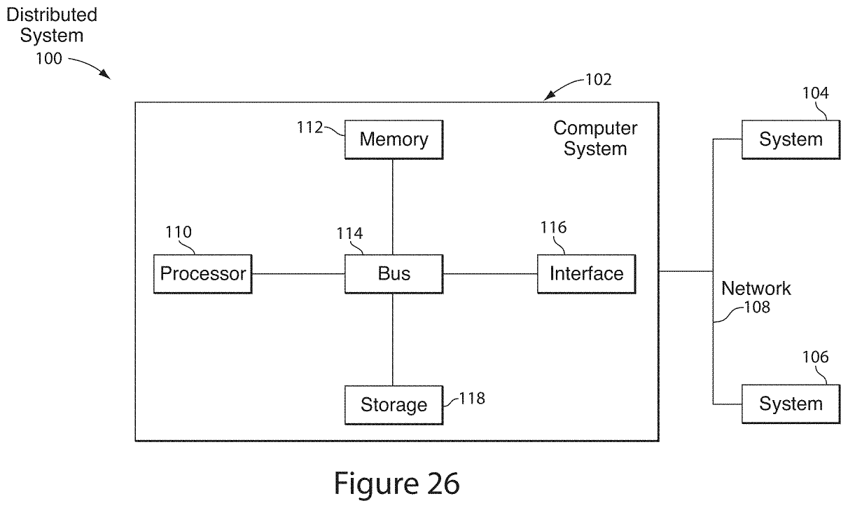

FIG. 26 shows a block diagram of a distributed computer system 100, in which various aspects and functions in accord with the present disclosure may be practiced. The distributed computer system 100 may include one more computer systems. For example, as illustrated, the distributed computer system 100 includes three computer systems 102, 104 and 106. As shown, the computer systems 102, 104 and 106 are interconnected by, and may exchange data through, a communication network 108. The network 108 may include any communication network through which computer systems may exchange data. To exchange data via the network 108, the computer systems 102, 104 and 106 and the network 108 may use various methods, protocols and standards including, among others, token ring, Ethernet, Wireless Ethernet, Bluetooth, TCP/IP, UDP, HTTP, FTP, SNMP, SMS, MMS, SS7, JSON, XML, REST, SOAP, CORBA HOP, RMI, DCOM, and Web Services. To ensure data transfer is secure, the computer systems 102, 104 and 106 may transmit data via the network 108 using a variety of security measures including TSL, SSL, or VPN, among other security techniques. While the distributed computer system 100 illustrates three networked computer systems, the distributed computer system 100 may include any number of computer systems, networked using any medium and communication protocol.

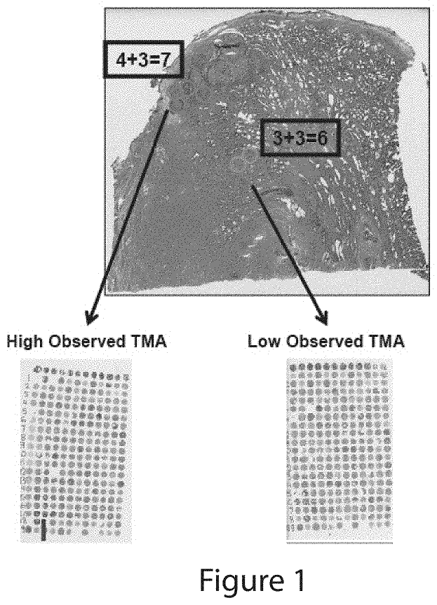

Various aspects and functions in accord with the present disclosure may be implemented as specialized hardware or software executing in one or more computer systems including the computer system 102 shown in FIG. 1. As depicted, the computer system 102 includes a processor 110, a memory 112, a bus 114, an interface 116 and a storage system 118. The processor 110, which may include one or more microprocessors or other types of controllers, can perform a series of instructions that manipulate data. The processor 110 may be a well-known, commercially available processor such as an Intel Pentium, Intel Atom, ARM Processor, Motorola PowerPC, SGI MIPS, Sun UltraSPARC, or Hewlett-Packard PA-RISC processor, or may be any other type of processor or controller as many other processors and controllers are available. The processor 110 may be a mobile device or smart phone processor, such as an ARM Cortex processor, a Qualcomm Snapdragon processor, or an Apple processor. As shown, the processor 110 is connected to other system placements, including a memory 112, by the bus 114.

The memory 112 may be used for storing programs and data during operation of the computer system 102. Thus, the memory 112 may be a relatively high performance, volatile, random access memory such as a dynamic random access memory (DRAM) or static memory (SRAM). However, the memory 112 may include any device for storing data, such as a disk drive or other non-volatile storage device, such as flash memory or phase-change memory (PCM). Various embodiments in accord with the present disclosure can organize the memory 112 into particularized and, in some cases, unique structures to perform the aspects and functions disclosed herein.

Components of the computer system 102 may be coupled by an interconnection element such as the bus 114. The bus 114 may include one or more physical busses (for example, busses between components that are integrated within a same machine), and may include any communication coupling between system placements including specialized or standard computing bus technologies such as IDE, SCSI, PCI and InfiniBand. Thus, the bus 114 enables communications (for example, data and instructions) to be exchanged between system components of the computer system 102.

Computer system 102 also includes one or more interface devices 116 such as input devices, output devices, and combination input/output devices. The interface devices 116 may receive input, provide output, or both. For example, output devices may render information for external presentation. Input devices may accept information from external sources. Examples of interface devices include, among others, keyboards, mouse devices, trackballs, microphones, touch screens, printing devices, display screens, speakers, network interface cards, etc. The interface devices 116 allow the computer system 102 to exchange information and communicate with external entities, such as users and other systems.

Storage system 118 may include a computer-readable and computer-writeable nonvolatile storage medium in which instructions are stored that define a program to be executed by the processor. The storage system 118 also may include information that is recorded, on or in, the medium, and this information may be processed by the program. More specifically, the information may be stored in one or more data structures specifically configured to conserve storage space or increase data exchange performance. The instructions may be persistently stored as encoded signals, and the instructions may cause a processor to perform any of the functions described herein. A medium that can be used with various embodiments may include, for example, optical disk, magnetic disk, or flash memory, among others. In operation, the processor 110 or some other controller may cause data to be read from the nonvolatile recording medium into another memory, such as the memory 112, that allows for faster access to the information by the processor 110 than does the storage medium included in the storage system 118. The memory may be located in the storage system 118 or in the memory 112. The processor 110 may manipulate the data within the memory 112, and then copy the data to the medium associated with the storage system 118 after processing is completed. A variety of components may manage data movement between the medium and the memory 112, and the disclosure is not limited thereto.

Further, the disclosure is not limited to a particular memory system or storage system. Although the computer system 102 is shown by way of example as one type of computer system upon which various aspects and functions in accord with the present disclosure may be practiced, aspects of the disclosure are not limited to being implemented on the computer system, shown in FIG. 1. Various aspects and functions in accord with the present disclosure may be practiced on one or more computers having different architectures or components than that shown in FIG. 1. For instance, the computer system 102 may include specially-programmed, special-purpose hardware, such as for example, an application-specific integrated circuit (ASIC) tailored to perform a particular operation disclosed herein. Another embodiment may perform the same function using several general-purpose computing devices running MAC OS System X with Motorola PowerPC processors and several specialized computing devices running proprietary hardware and operating systems.

The computer system 102 may include an operating system that manages at least a portion of the hardware placements included in computer system 102. A processor or controller, such as processor 110, may execute an operating system which may be, among others, a Windows-based operating system (for example, Windows NT, Windows 2000/ME, Windows XP, Windows 7, or Windows Vista) available from the Microsoft Corporation, a MAC OS System X operating system available from Apple Computer, one of many Linux-based operating system distributions (for example, the Enterprise Linux operating system available from Red Hat Inc.), a Solaris operating system available from Sun Microsystems, or a UNIX operating systems available from various sources. The operating system may be a mobile device or smart phone operating system, such as Windows Mobile, Android, or iOS. Many other operating systems may be used, and embodiments are not limited to any particular operating system. The computer system 102 may include a virtualization feature that hosts the operating system inside a virtual machine (e.g., a simulated physical machine). Various components of a system architecture could reside on individual instances of operating systems inside separate “virtual machines”, thus running somewhat insulated from each other.

The processor and operating system together define a computing platform for which application programs in high-level programming languages may be written. These component applications may be executable, intermediate (for example, C# or JAVA bytecode) or interpreted code which communicate over a communication network (for example, the Internet) using a communication protocol (for example, TCP/IP). Similarly, functions in accord with aspects of the present disclosure may be implemented using an object-oriented programming language, such as SmallTalk, JAVA, C++, Ada, or C# (C-Sharp). Other object-oriented programming languages may also be used. Alternatively, procedural, scripting, or logical programming languages may be used.

Additionally, various functions in accord with aspects of the present disclosure may be implemented in a non-programmed environment (for example, documents created in HTML, XML or other format that, when viewed in a window of a browser program, render aspects of a graphical-user interface or perform other functions). Further, various embodiments in accord with aspects of the present disclosure may be implemented as programmed or non-programmed placements, or any combination thereof. For example, a web page may be implemented using HTML while a data object called from within the web page may be written in C++. Thus, the disclosure is not limited to a specific programming language and any suitable programming language could also be used.

A computer system included within an embodiment may perform functions outside the scope of the disclosure. For instance, aspects of the system may be implemented using an existing product, such as, for example, the Google search engine, the Yahoo search engine available from Yahoo! of Sunnyvale, Calif., or the Bing search engine available from Microsoft of Seattle Wash. Aspects of the system may be implemented on database management systems such as SQL Server available from Microsoft of Seattle, Wash.; Oracle Database from Oracle of Redwood Shores, Calif.; and MySQL from Sun Microsystems of Santa Clara, Calif.; or integration software such as WebSphere middleware from IBM of Armonk, N.Y. However, a computer system running, for example, SQL Server may be able to support both aspects in accord with the present disclosure and databases for sundry applications not within the scope of the disclosure.

In addition, the method described herein may be incorporated into other hardware and/or software products, such as a web publishing product, a web browser, or an internet marketing or search engine optimization tool.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and embodiments, the word “comprise,” or variations such as “comprises” or “comprising” will be understood to imply the inclusion of a stated integer or group of integers but not the exclusion of any other integer or group of integers. The materials, methods, and examples are illustrative only and not intended to be limiting.

### EXAMPLES

Further details of the invention are described in the following non-limiting Examples. It should be understood that these examples, while indicating some preferred embodiments of the invention, are given by way of illustration only, and should not be construed as limiting the appended Embodiments or Claims. From the present disclosure and these examples, one skilled in the art can ascertain certain characteristics of this invention, and without departing from the spirit and scope thereof, can make changes and modifications of these examples to adapt them to various usages and conditions.

### Example 1: Preparation of Tumor Microarrays

The experiments described in Examples 2-4 utilized four different tumor microarrays (TMA): a cell line control TMA, a mini TMA, a high observed Gleason TMA and a low observed Gleason TMA.

A. Preparation of cell line control TMAs

A set of cell line controls was selected to measure the reliability and reproducibility of the multiplex immunofluorescence assay. These cell lines had a range of expression levels for the tumor markers that would be analyzed in the multiplex immunofluorescence assay. The cell lines and their levels of biomarker expression are described in Table 2 below.

TABLE 2
Cell line control treatment and tumor marker expression patterns.

| | | BIOMARKERS | | | | | |
|---|---|---|---|---|---|---|---|
| | | | | | | CK8 | |
| Cell line | Source | CCND1 | PTEN | SMAD4 | SPP1 | &CK18 | AKT pathway |
| DU-145 | ATCC | High | High | Present | Medium | positive | Active |
| PC-3 | ATCC | Medium | Negative | Present | Low | positive | Very active |
| DU-145 | ATCC | | | | | positive | Very low activity |
| LY294002- | | | | | | | |
| treated | | | | | | | |
| PC3 | ATCC | | | | | positive | Low activity |
| LY294002- | | | | | | | |
| treated | | | | | | | |
| DU-145 | Metamark | Low | | | | positive | |
| shRNA for | | | | | | | |
| CCND1 | | | | | | | |
| DU-145 | Metamark | | | Low | | positive | |
| shRNA for | | | | | | | |
| SMAD4 | | | | | | | |
| RWPE-1 | Metamark | Low | High | High | Negative | positive | Low activity |
| BxP3 | Metamark | | | negative | | negative | |
| SK-MEL-5 | ATCC | | Medium | Medium | High | negative | Active |
| WM266-4 | ATCC | | | | | | Active |
| RPMI7951 | ATCC | High | | | | | Active |

Selected cell lines were grown under standard conditions, and if necessary, treated with PI3K kinase inhibitors (see Table 2). Cells were washed with PBS, fixed directly on plates with 10% formalin for 5 min, scraped and collected in fixative, with continued fixation at room temperature for 1 hour total. Cells were then spun down and washed twice with PBS. Cell pellets were resuspended in warm Histogel at 70° C. and quickly spun down in an Eppendorf tube to form a condensed cell-Histogel pellet. The pellets were embedded in paraffin, placed into standard paraffin blocks, and used as donor blocks for tissue microarray (TMA) construction.

TMA blocks were prepared using a modified agarose block procedure (Yan et al., J Histochem Cytochem 55(1): 21-24 (2007). Briefly, 0.7% agarose blocks were embedded into paraffin blocks and used as TMA acceptor blocks. Using a TMA MASTER (3DHISTECH) instrument, acceptor blocks were pre-drilled for 1 mm cores. One mm cores were removed from donor blocks of cell line controls and placed in the TMA acceptor blocks to create a cell line control TMA. Then cores were aligned by pressing the TMA blocks face down onto glass slides and placing them on a 65° C. hot plate for 15 min, so that the paraffin would melt and completely fuse the cores within the block. Slides with blocks were cooled, TMA blocks removed from slides, trimmed and 5 μm serial sections were cut from the TMA blocks.

B. Preparation of Mini TMAs

To generate mini TMAs, we used formalin-fixed, paraffin-embedded (FFPE) prostate tumor sample blocks from an annotated cohort of patients who had undergone radical prostatectomies and had their Gleason scores determined. The cohort consisted of about 40 indolent tumors (Gleason ≤3+3) and about 40 aggressive tumors (Gleason ≥4+3).

TMA blocks were prepared using a modified agarose block procedure (Yan et al., supra). Briefly, 0.7% agarose blocks were embedded into paraffin blocks and used as TMA acceptor blocks. Using a TMA MASTER (3DHISTECH) instrument, acceptor blocks were pre-drilled for 1 mm cores. One mm cores were removed from about 80 cohort donor blocks and placed in the TMA acceptor blocks to create a mini TMA. Cell line controls were interspersed with the cohort samples to serve as controls for intra-slide or core-to-core staining reproducibility, slide-to-slide staining reproducibility, and day-to-day staining reproducibility. Then cores were aligned by pressing the TMA blocks face down onto glass slides and placing them on a 65° C. hot plate for 15 min, so that the paraffin would melt and completely fuse the cores within the block. Slides with blocks were cooled, TMA blocks removed from slides, trimmed and 5 μm serial sections were cut from the TMA blocks.

C. Preparation of High and Low Observed Gleason TMAs

Formalin-fixed, paraffin-embedded (FFPE) prostate tumor sample blocks from an annotated cohort of patients who had undergone radical prostatectomies were obtained from Folio Biosciences (Powell, Ohio).

A series of 5 μm sections was cut from each FFPE block and the sections used for tissue quality control processing and subsequent Gleason score annotation. Some sections underwent immunofluorescent staining to determine whether the tissue quality was suitable for further study and to ensure that the tissue contained sufficient tumor regions for further study. Briefly, these control FFPE sections were processed for immunofluorescent staining, and stained with anti-phospho STAT3-T705 rabbit monoclonal antibody (mAb), anti-STAT3 mouse mAb, Alexa 488-conjugated anti-cytokeratin 8 mouse mAb, Alexa 488-conjugated anti-cytokeratin 18 mouse mAb, Alexa 555-conjugated anti-cytokeratin 5 mAb, and Alexa 555-conjugated anti-TRIM mAb (see Table 1). Slides were visually examined for staining by each antibody under a fluorescent microscope (Vectra System, PerkinElmer). Based on the staining intensities and autofluorescence, the sections and their corresponding FFPE blocks were graded into four categories that indicated the quality of the tissue as shown in Table 2. Tumor regions were defined as prostate epithelial structures devoid of basal cell markers. Anti-cytokeratin 8 and anti-cytokeratin 18 mAbs were used to indicate epithelial-specific staining. Anti-cytokeratin 5 and anti-TRIM29 mAbs were used to indicate basal cell staining. Only FFPE blocks that contained sufficient amounts of tumor areas and that fell into the top two quality categories were used in further studies.

A 5 μm section that was the last to be cut from each FFPE block was stained with hematoxylin and eosin (H&E) and scanned using an Aperio XT system (Aperio, Vista, Calif.). The scanned images were deposited into a SPECTRUM database (Aperio, Vista, Calif.). Images of H&E-stained sections were remotely reviewed and Gleason score annotated in a blinded manner by American Board of Pathology-Certified anatomical pathologists at Brigham and Women's Hospital (Boston, Mass.) and Johns Hopkins University (Baltimore, Md.) via ImageScope software (Aperio, Vista, Calif.). The pathologists placed annotated circles corresponding to 1 mm cores over four areas of highest and two areas of lowest Gleason score patterns on each section image (see, e.g., FIG. 1). One highest Gleason section and one lowest Gleason section were selected for inclusion in the high and low observed Gleason TMAs, respectively. In cases where the tumors were relatively uniform, the high and low sections were roughly identical.

TMA blocks were prepared using a modified agarose block procedure (Yan et al., supra). Briefly, 0.7% agarose blocks were embedded into paraffin blocks and used as TMA acceptor blocks. Using a TMA Master (3DHistech) instrument, acceptor blocks were pre-drilled for 1 mm cores. One mm cores were removed from donor blocks of cell line controls (described above) and placed in three separate regions of the acceptor blocks: top, middle and bottom portions. In this arrangement, cell line controls could serve as controls for intra-slide or core-to-core staining reproducibility, slide-to-slide staining reproducibility, and day-to-day staining reproducibility. One important feature of cell line controls was that they were consistent between distant sections of TMA block. Tissue samples change as cores were cut into sections, while cell line controls were uniform mixtures of cells all along the depth of cores and do not change.

FFPE blocks of prostate tumor samples that passed quality control were selected as patient sample donor blocks. These donor blocks were cored in areas corresponding to the selected high and low observed Gleason sections as per pathologist annotation. The order of patient sample placement into the acceptor block was randomized. As duplicate cores were taken from each donor block (i.e., one high observed Gleason core and one low observed Gleason core), and placed into one of two separate acceptor blocks, the second core was placed in a position randomized relative to the position of the first core. In other words, the high observed Gleason TMA was randomized separately from the low observed Gleason TMA. Thus, the resulting two duplicate TMA blocks were identical in terms of patient sample composition but their positions were randomized. Then cores were aligned by pressing the TMA blocks face down onto glass slides and placing them on a 65° C. hot plate for 15 min, so that the paraffin would melt and completely fuse the cores within the block. Slides with blocks were cooled, TMA blocks removed from slides, trimmed and 5 μm serial sections were cut from the TMA blocks. Each core obtained from the prostate tumor samples was then annotated by pathologists to give an observed Gleason score (based only on the isolated core, separate from the whole tumor “actual” Gleason score obtained previously). For example, a core selected from an aggressive tumor and placed on the LTMA may have been annotated as having an “observed” Gleason score of 3+3, even though the tumor's “actual” surgical Gleason score was greater than 4+3.

### Example 2: Engines for Biomarker Selection

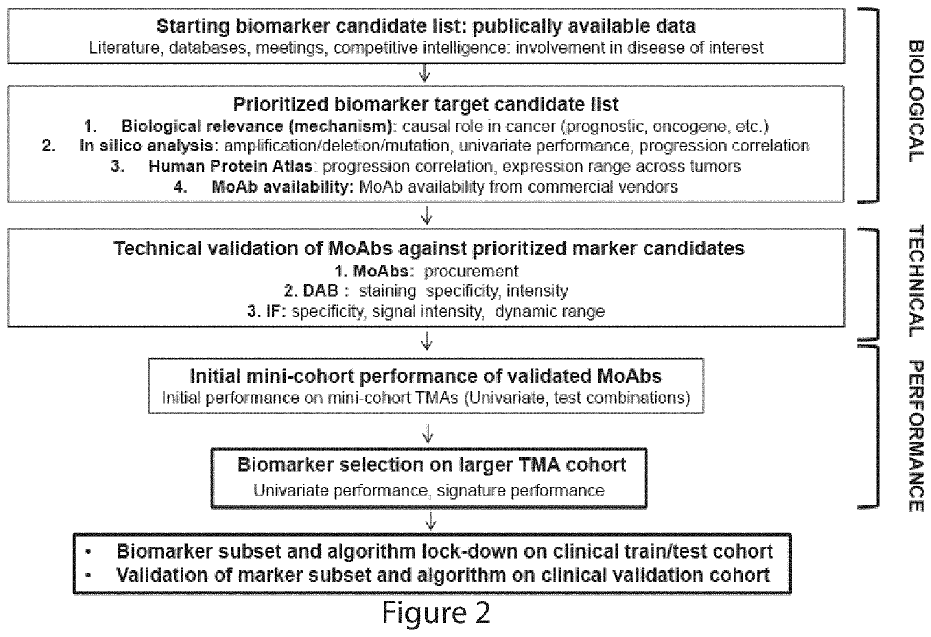

We developed a biomarker selection and validation engine that can be used to identify biomarkers for any disease or condition (FIG. 2). The engine has four main stages: a biological stage, a technical stage, a performance stage, and a validation stage.

In the biological stage, a starting biomarker candidate list is compiled for the disease of interest from publically available data, including scientific literature, databases, and presentations at meetings. The biomarker list is then prioritized based on biological relevance, in silico analysis, review of the Human Protein Atlas, and commercial availability of requisite monoclonal antibodies. Biological relevance review is based on its mechanism of action in the cell and, in particular, in the disease. In silico analysis is based on previously known gene amplifications, deletions and mutations, and univariate performance or progression correlation between these genetic alterations and the disease. The Human Protein Atlas provides protein expression levels in various tissues across disease states. Biomarkers are ranked based on whether or not they are expressed at a range of expression levels across healthy and disease states.

In the technical stage, commercial antibodies are obtained from vendors and tested for their ability to detect markers from clinical samples. First, the antibodies are tested in an immunohistochemistry assay using classical 3,3′-Diaminobenzidine (DAB) staining to evaluate staining specificity and intensity. As DAB is more sensitive than immunofluorescent staining, it is important to identify markers that are detected by DAB with sufficient intensity to also be detected by immunofluorescence. Antibodies and markers that meet the DAB criteria are then evaluated by immunofluorescence (IF) to determine specificity, signal intensity and dynamics (i.e., range of expression). Antibodies and markers that meet the IF criteria are advanced to the performance stage.

In the performance stage, antibodies are tested on mini TMAs. Performance is evaluated for a univariate correlation between expression and disease state. The antibodies and markers that demonstrate univariate correlation between expression and disease state are then evaluated on a larger TMA cohort for both univariate correlation and performance in combination with other markers. Leading biomarker combinations are then validated using a clinical validation cohort.

### Example 3: Selection of Prostate Cancer Biomarkers

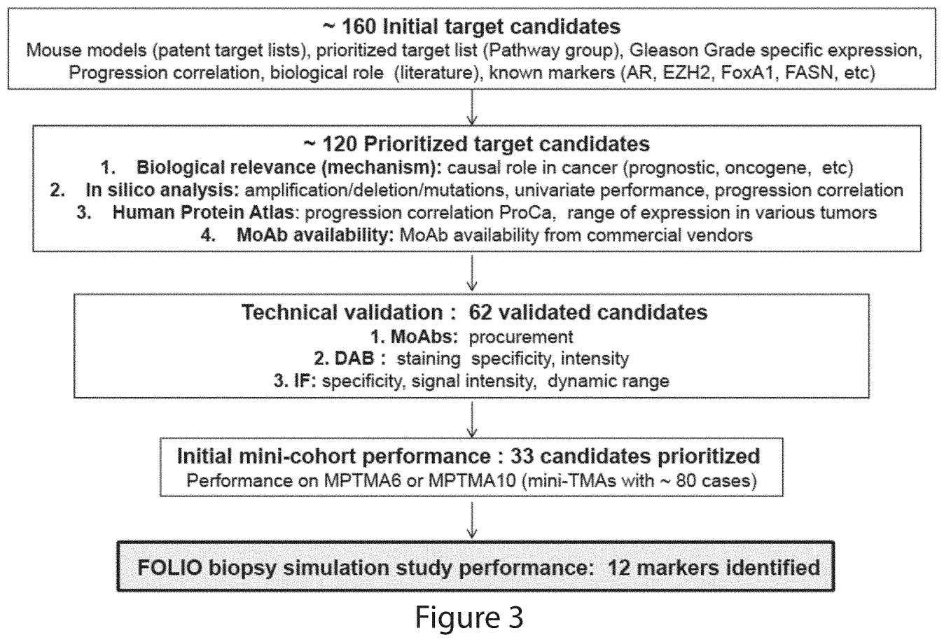

Using the biomarker selection engine described in Example 2, biomarkers for identification of indolent and aggressive prostate cancer were tested and selected as shown in FIG. 3.

A. Biological Stage

An initial target candidate list was compiled based on a review of prostate cancer literature to identify markers that are associated with prostate cancer in mouse models, Gleason grade-specific expression, progression correlation, a biological role in prostate cancer, and/or known prostate cancer markers. As several of the identified markers were part of one or more signaling pathways, other members of those signaling pathways were included in the initial candidate list. In total, 160 potential markers were included in the initial candidate target list.

The initial target list was prioritized based on biological relevance, in silico analysis, the Human Protein Atlas (available at www.proteinatlas.org/), and antibody availability. In evaluating biological relevance, oncogenes and tumor suppressor genes were considered less important for prognosis because they were less likely to be associated with tumor grade. Similarly, genes that were identified with univariate performance and progression correlation in an in silico analysis were prioritized. In prostate cancer, however, the correlation between gene and protein expression is poor. Accordingly, most prioritization of prostate cancer markers was based on the Human Protein Atlas, which shows the spatial distribution of proteins in 46 different normal human tissues and 20 different cancer types, as well as 47 different human cell lines. In particular, proteins whose expression level varied in various tumors were prioritized because their expression level may more closely correlate with tumor stage. After these analyses, a list of about 120 prioritized candidates moved into the technical validation stage.

B. Technical Stage

Antibodies for the 120 prioritized candidates were obtained from commercial vendors and were validated by immunohistochemistry. Sections from a variety of benign and cancerous prostate FFPE tissue samples were stained with candidate antibodies using a standard DAB protocol with the universal polymeric DAB detection kit (ThermoFisher). Roughly half of the test antibodies demonstrated specific staining patterns with strong intensity and were thus selected for evaluation by immunofluorescence.

Sections from a variety of benign and cancerous prostate FFPE tissue samples were stained with candidate antibodies using an immunofluorescent protocol described below with a control cell line TMA. Antibodies that demonstrated specific staining patterns were selected for further studies.

Prostate cancer is typically a carcinoma expressing epithelial markers such as cytokeratin 8 (CK8 or KRT8) and cytokeratin 18 (CK18 or KRT18) while not expressing prostate basal markers such as cytokeratin 5 (CK5 or KRT5). We have also found surprisingly that TRIM29, a tumor marker for some other cancers, is a basal marker, not a tumor marker, in prostate tissue; thus, anti-TRIM29 antibodies may also be used in a prostate tumor mask. We evaluated tumor sections using a mixture of anti-CK5, anti-CK8, anti-CK18, and anti-TRIM29 antibodies, where a prostate tumor region is defined as a prostate tissue region bound by anti-CK8 and anti-CK18 antibodies and not bound by anti-CK5 and anti-TRIM29 antibodies.

Five μm sections were cut from cell line control TMA blocks and placed on HISTOGRIP (Life Technologies) coated slides. Slides were baked at 65° C. for 30 min, de-paraffinized through serial incubations in xylene, and rehydrated through a series of graded alcohols. Antigen retrieval was done in a 0.05% Citraconic anhydride solution at pH 7.4 for 40 min at 95° C.

Immunofluorescent staining was done using a LabVision Autostainer, with all incubations at room temperature, all washes with TBS-T (TBS+0.05% Tween 20), and all antibodies diluted with TBS-T+0.1% BSA solution. Slides were first blocked with Biotin Block (Life Technologies) solution A for 20 min, washed, then solution B for 20 min, washed, and then blocked with Background Sniper (Biocare Medical) for 20 min and washed again. Either a mouse or a rabbit primary antibody was applied and incubated for 1 hour. In some cases, a mouse primary antibody for a first biomarker and a rabbit primary antibody for a second biomarker were applied to the slide and incubated for an hour.

After extensive washes, either a biotin-conjugated anti-mouse IgG or a FITC-conjugated anti-rabbit IgG was applied for 45 min. In cases where two biomarkers were detected on the same slide, both a biotin-conjugated anti-mouse IgG and a FITC-conjugated anti-rabbit IgG were applied for 45 min. After extensive washes, a mixture of Alexa fluorophore-conjugated reagents was applied that consisted of streptavidin-Alexa 633, anti-FITC mAb-Alexa 568, and a Tumor Mask cocktail (anti-cytokeratin 8 mAb Alexa 488, anti-cytokeratin 18 mAb Alexa 488, anti-cytokeratin 5 mAb Alexa 555, anti-TRIM29 mAb Alexa555).

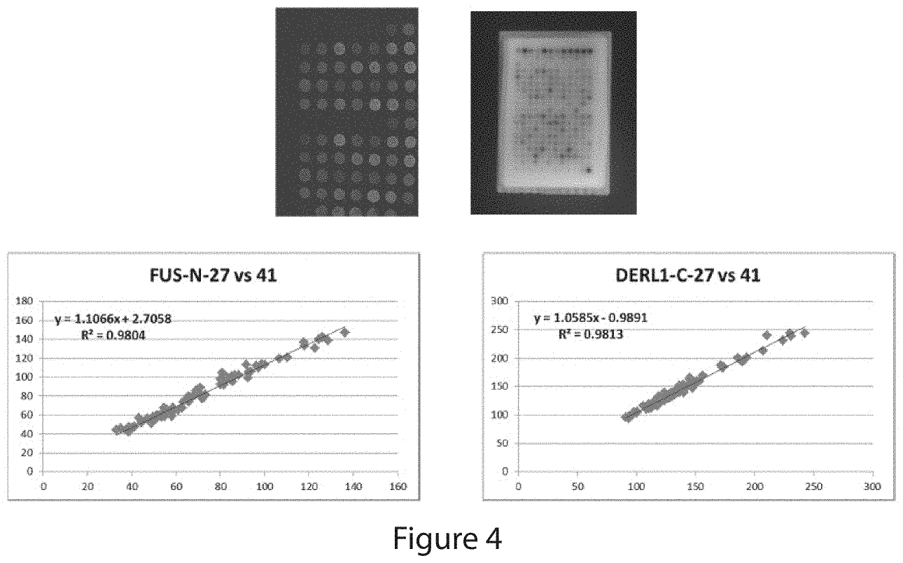

To enable automated image analysis of prostate cancer tumor tissue, we utilized a combination of antibodies for prostate epithelial and basal markers (Tumor Mask) and object recognition based on Definiens Developer XD (described below). Tumor regions were defined as prostate epithelial structures devoid of basal markers. A cocktail of Alexa 488-conjugated anti-cytokeratin 8 and anti-cytokeratin 18-specific mouse mAbs was used to obtain epithelial-specific staining. Staining of basal cells was based on a cocktail of Alexa 555-conjugated anti-cytokeratin 5 and anti-TRIM29-specific mAbs. The slides were incubated for 1 hour with these Alexa fluorophore-conjugated reagents. After extensive washes, a DAPI solution (100 ng/ml DAPI in TBS-T) was applied for 3 min. After several washes, slides were mounted in Prolong Gold anti-fade reagent (Life Technologies). Slides were left overnight at −20° C. in the dark to “cure” and were stored long term in the dark at −20° C. to minimize fading. The amount of immunofluorescence for each marker was evaluated. FIG. 4, for example, shows quantitative immunofluorescence for two different markers (FUS and DERL1) on two different sections (sections 27 and 41) of a control cell line TMA. The amount of immunofluorescence detected for each cell line in section 27 is displayed on the x-axis, while the amount of immunofluorescence detected for each cell line in section 41 is displayed on the y-axis. The linear relationship of the amount of immunofluorescence in the two cell lines and the high R² values demonstrate the reproducibility of the quantitative immunofluorescence assay between experiments.

Next we tested the range of marker expression. For optimal dilutions of marker antibodies in our staining assays and for reproducibility, we prepared a “titration” TMA. We selected 40 FFPE blocks of prostate cancer samples with a range of Gleason scores. Then the “titration” TMA was generated using the modified agarose block procedure described above with duplicate cores from each donor sample. Immunofluorescent staining with single markers and with tumor region recognition anti-cytokeratin 8, anti-cytokeratin 18, anti-cytokeratin 5, and anti-TRIM29 antibodies was performed. As discussed above, for detection of mouse monoclonal candidate antibodies, we used anti-mouse-biotin secondary and Streptavidin-Alexa 633 tertiary antibodies. For rabbit monoclonal candidate antibodies, anti-rabbit-FITC secondary and anti-FITC mAb-Alexa 568 tertiary antibodies were used. Images were captured with Vectra systems as described below and marker expression was quantified using Inform 1.3 software. Based on marker specificity, signal intensity and the dynamic range of the markers, 62 validated candidates were advanced to the performance stage.

C. Performance Stage

Mini Cohort Screening

The 62 validated candidates were tested on mini TMAs, which were prepared as described in Example 1. Quantitative immunofluorescent assays were performed using mouse and rabbit primary antibodies as described above in the Technical Stage. The 62 biomarkers were quantitated and differences in expression levels were determined between the about 40 indolent tumor samples and the about 40 aggressive tumor samples. Of the 62 markers, 33 demonstrated univariate performance for correlation with indolent or aggressive tumor status.

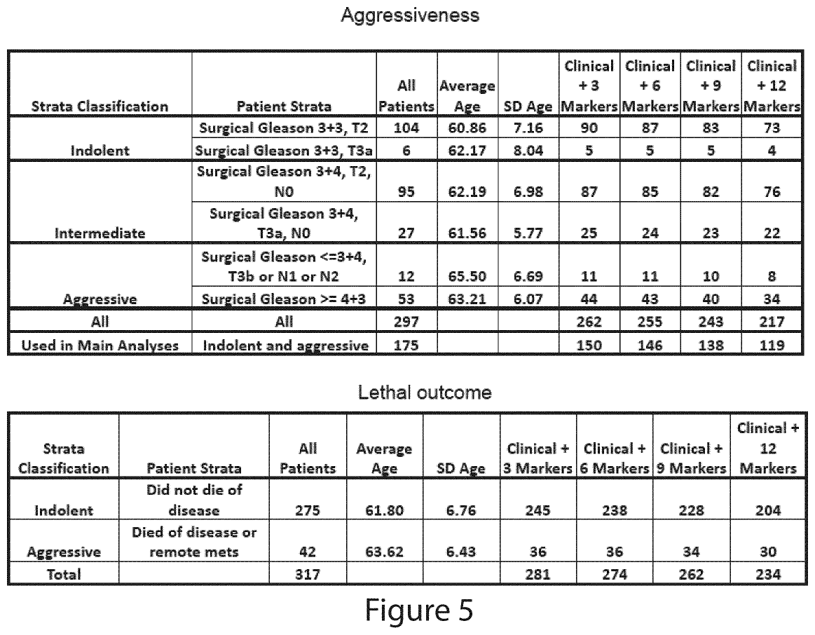

The 33 univariate performing markers were tested in an expanded biopsy simulation study using high and low observed Gleason TMAs (HLTMAs). Because the observed Gleason score for each core on the high and low TMAs may differ from the actual Gleason score for the tumor from which the core was derived (based on the entire surgically removed tumor), it is possible to identify biomarkers that are predictive of the true Gleason score, and therefore aggressiveness, independent of the sample's location in the tumor. In other words, we hoped to identify biomarkers that would minimize sampling bias caused by heterogeneity within the tumor. For example, indolent, intermediate, and aggressive tumors were each represented on the low observed TMA (see, e.g., FIG. 5 for a summary of the actual Gleason scores of the cores on the low observed TMA).

For the 33 biomarkers with univariate performance in the mini TMA experiments, quantitative immunofluorescent assays were performed using mouse and rabbit primary antibodies as described above in the Technical Stage. Two of the markers were discarded due to technical difficulties during the HLTMA immunostaining and detection. Thus, data was obtained and analyzed for 31 of the biomarkers with univariate performance in the mini TMA experiments.

D. Image Acquisition

Two Vectra Intelligent Slide Analysis Systems (PerkinElmer) were used for quantitative multiplex immunofluorescence (QMIF) image acquisition. TMA acquisition protocols were run according to manufacturer's instructions with minor modifications. The same exposure times were used for all slides. To minimize inter-TMA variability, TMA slides stained with the same antibody combinations were processed on the same Vectra microscope.

DAPI, FITC, TRITC and Cy5 long pass emission filter cubes were obtained from Semrock. TRITC and Cy5 filter cubes were optimized to allow maximum spectral separation between the Alexa 555, Alexa 568, and Alexa 633 dyes. DAPI, FITC, TRITC and Cy5 long pass emission filter cubes were obtained from Semrock. TRITC and Cy5 filter cubes were optimized to allow maximum spectral separation between the Alexa 555, Alexa 568, and Alexa 633 dyes.

DAPI band acquisition was done with 20 nm steps. FITC, TRITC and Cy5 bands acquisition was done with 10 nm steps. Two 20× image cubes per core were obtained with sequential collection of images in DAPI, FITC, TRITC and Cy5 bands. Spectral libraries were prepared according to manufacturer instructions, and Inform 1.4 software (PerkinElmer) was used to unmix image cubes into floating TIFF files with individual fluorophore signals and autofluorescence signals. Two channels were created for autofluorescence, one for general tissue autofluorescence and another for erythrocytes and bright granules scattered across prostatic tissue. After image unmixing, sets of TIFF files were analyzed further with Definiens Developer software. For analysis of data from a smaller “titration” TMA, Inform 1.3 software (PerkinElmer) was used to unmix image cubes and to quantify markers expression.

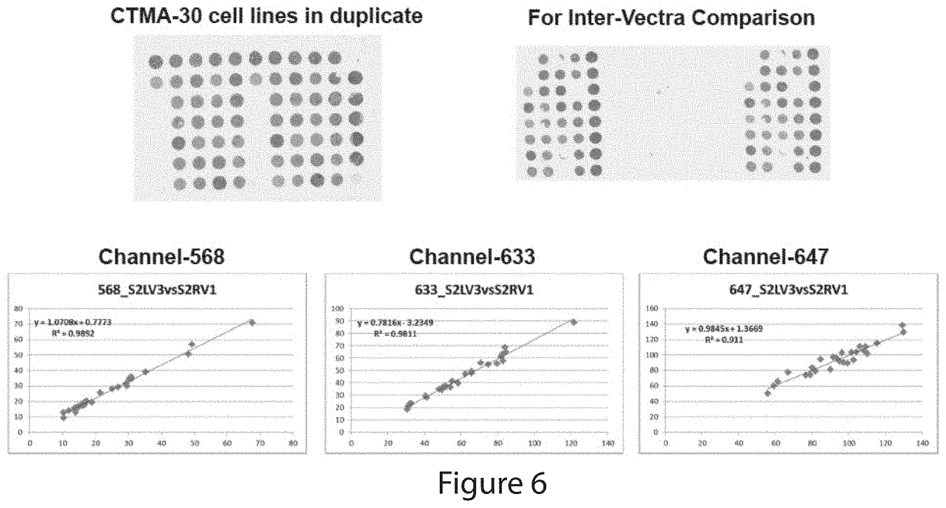

To determine if any inter-instrument variation existed between the two Vectra Intelligent Slide Analysis Systems (PerkinElmer), we analyzed CTMAs in parallel on the two machines for detection of Alexa-568, Alexa-633 and Alexa-647. As shown in FIG. 6, the two systems differed in Alexa-647 detection by less than 2% and in Alexa-568 detection by about 7%. The detection of Alexa-633, however, was about 20% different between the two machines. Using these data, we were able to establish inter-instrument conversion factors for each channel.

E. Image Analysis

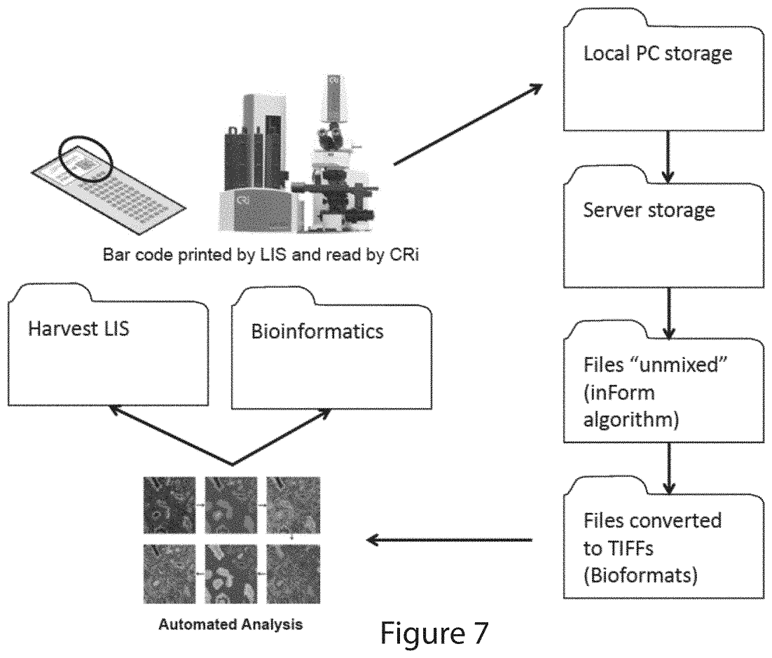

A fully automated image analysis algorithm was generated using Definiens Developer XD™ (Definiens, Inc., Parsippany, N.J.) for tumor identification and biomarker quantification (see, e.g., FIG. 7). For each tissue microarray (TMA) core, two 20×1.0 mm image fields were acquired. The Vectra multispectral image files were first converted into multilayer TIFF format using inForm, and then converted to single layer TIFF files using BioFormats. The single layer TIFF files were imported into the Definiens workspace using a customized import algorithm. For each TMA core both of the image field TIFF files were loaded as “maps” within a single “scene” per manufacturer's instructions.

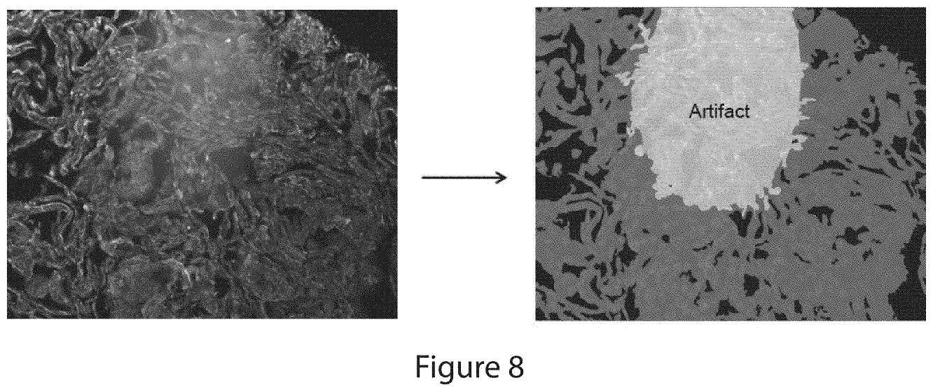

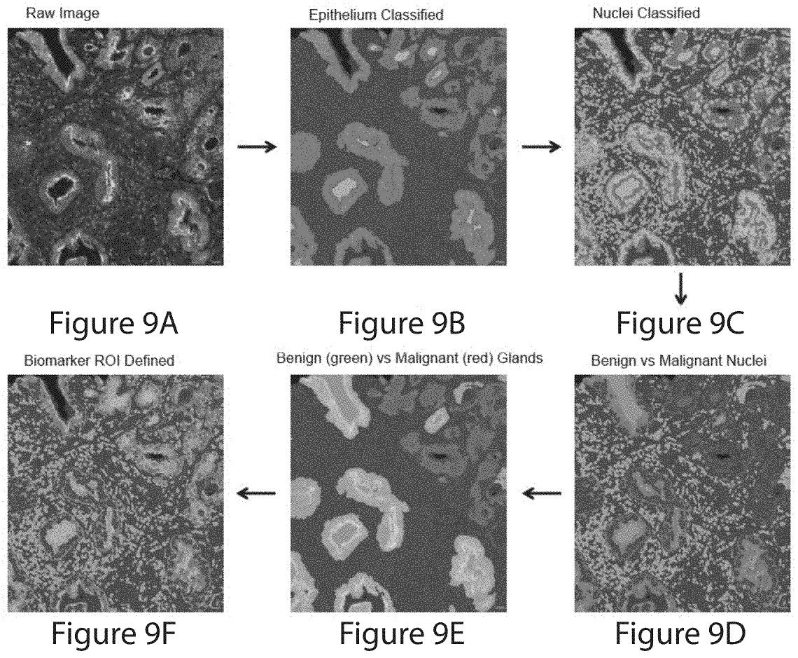

Built-in auto-adaptive thresholding was used to define fluorescent cut-offs for tissue segmentation in each individual tissue sample in our image analysis algorithm. Cell line controls were identified automatically based on pre-defined core locations. The tissue samples were segmented using the fluorescent epithelial and basal cell markers, along with DAPI, for classification into epithelial cells, basal cells, and stroma and further compartmentalized into cytoplasm and nuclei. The cell line controls were segmented using the autofluorescence channel. Fields with artifact staining, insufficient epithelial tissue, and out of focus were removed by a rigorous multi-parameter quality control algorithm (see, e.g., FIG. 8). Individual gland regions in tissue samples were further classified as malignant or benign based on the relational features between basal cells and adjacent epithelial structures combined with object-related features, such as gland thickness (see, e.g., FIG. 9).

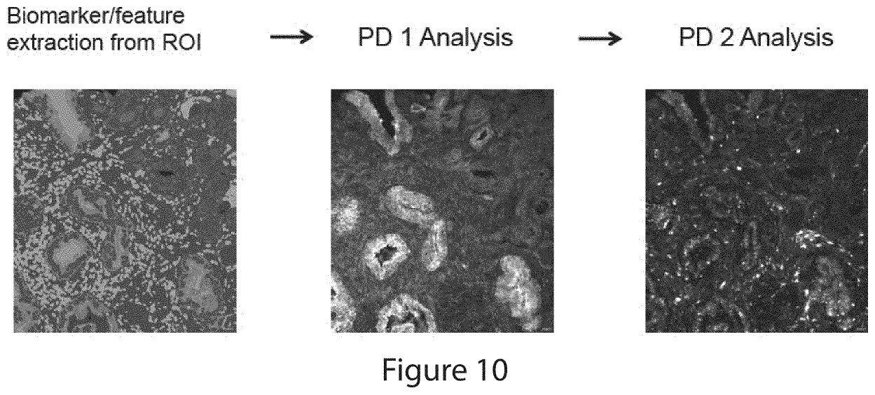

Epithelial marker and DAPI intensities were quantified in malignant and nonmalignant epithelial regions as quality control measurements. Biomarker values were measured independently in the malignant tissue cytoplasm, nucleus, or whole cell based on predetermined subcellular localization (see, e.g., FIG. 10). The mean biomarker pixel intensity in the malignant compartments was averaged across the maps with acceptable quality parameters to yield a single value for each tissue sample and cell line control core.

Data obtained from the Definiens analysis were exported for bioinformatics analysis or Clinical Lab Improvement Amendment Laboratory Information System (LIS) analysis.

F. Data Analysis

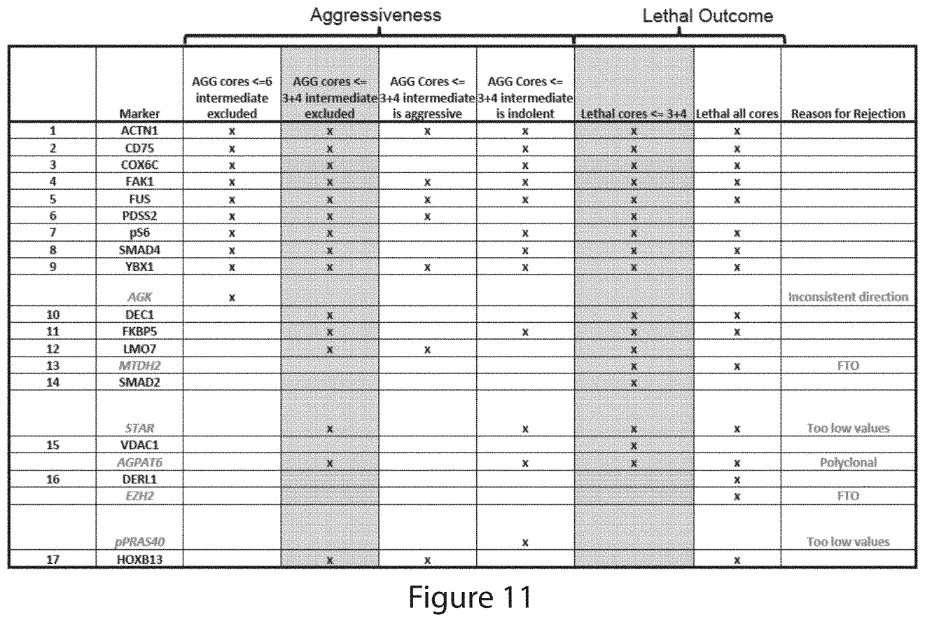

The mean biomarker values obtained for the 31 biomarkers with univariate performance in the mini TMA experiments were examined for their correlation with tumor aggressiveness and lethality. As discussed above, indolent, intermediate, and aggressive tumors were each represented on the both the high and low observed TMAs (see, e.g., FIG. 5 for the breakdown of each category on the low observed TMA). For aggressiveness studies, we correlated biomarker expression with aggressiveness in four different sample sets: (1) all cores with an observed Gleason score ≤3+3; (2) all cores with an observed Gleason score ≤3+4 wherein cores with a surgical intermediate Gleason score are excluded; (3) all cores with an observed Gleason score ≤3+4 wherein cores with a surgical intermediate Gleason score are counted as aggressive; and (4) all cores with an observed Gleason score ≤3+4 wherein cores with a surgical intermediate Gleason score are counted as indolent (FIG. 11). For lethal outcome studies, we correlated biomarker expression with lethal aggression in two different sample sets: (1) all cores with an observed Gleason score ≤3+4; and (2) all cores (FIG. 10). Biomarker values were correlated on a univariate basis using T test, Wilcoxson test, and Permutation test. Of the 31 biomarkers tested, 17 biomarkers demonstrated univariate performance in both aggressiveness and lethal outcome determinations (FIG. 11).

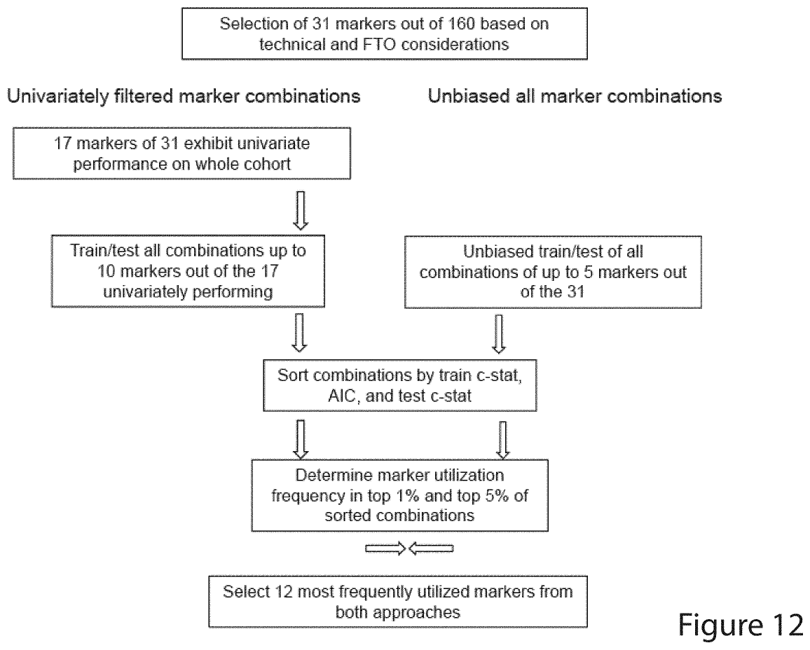

We next evaluated whether combinations of biomarkers correlated with tumor aggressiveness using two different approaches: (1) looking at combinations of the 17 biomarkers that demonstrated univariate performance in both aggressiveness and lethal outcome determinations; and (2) unbiased analysis of combinations of all 31 biomarkers tested in the HLTMA analysis (FIG. 12). Combinations between three and ten biomarkers selected from the 17 univariately performing biomarkers were analyzed, and combinations between three and five biomarkers from the set of 31 biomarkers were analyzed. For each marker combination, 500 training sets were generated by bootstrap (i.e., random sampling with replacement) and the associated test sets were obtained. Models were derived by Logistic Regression on training sets were tested on the associated test sets. Training and test C-statistic (i.e., area under the curve) and training Akaike information criterion (AIC) were obtained each round. Median and 95% confidence intervals were obtained for all three statistics. The top-ranking models for tumor aggressiveness in the combinations preselected for univariate performance for each method of analysis are listed in Table 3.

TABLE 3
Top Combinations of 17 Univariately Performing Biomarkers

| | Num- | | | | | | | Low | High | | Low | High |
|---|---|---|---|---|---|---|---|---|---|---|---|---|
| | ber | | | | | | | 95% | 95% | | 95% | 95% |
| | of | | | | AIC | AIC | Median | c- | C- | Median | C- | C- |
| Ranked | Mark- | Per- | | | Low | High | C-stat | Stat | stat | C-stat | stat | stat |
| By | ers | centile | Markers | AIC | 95% | 95% | train | Train | train | Test | test | Test |
| Train | 10 | 0.001 | ACTN1, COX6C, FUS, pS6, | 101.08 | 70.99 | 127.04 | 0.936 | 0.853 | 0.985 | 0.849 | 0.642 | 0.970 |
| | | | SMAD4, YBX1, FKBP5, VDAC1, | | | | | | | | | |
| | | | DERL1, SMAD2 | | | | | | | | | |
| | 9 | 0.007 | ACTN1, COX6C, FUS, pS6, | 97.61 | 64.50 | 123.14 | 0.932 | 0.840 | 0.988 | 0.857 | 0.658 | 0.979 |
| | | | SMAD4, YBX1, DERL1, HOXB13, | | | | | | | | | |
| | | | SMAD2 | | | | | | | | | |
| | 8 | 0.150 | ACTN1, FUS, pS6, SMAD4, YBX1, | 100.83 | 68.72 | 125.25 | 0.924 | 0.831 | 0.983 | 0.855 | 0.658 | 0.976 |
| | | | DERL1, HOXB13, SMAD2 | | | | | | | | | |
| | 7 | 1.494 | ACTN1, FUS, pS6, SMAD4, | 116.75 | 84.30 | 144.34 | 0.914 | 0.828 | 0.972 | 0.856 | 0.676 | 0.969 |
| | | | FKBP5, DERL1, SMAD2 | | | | | | | | | |
| | 6 | 7.893 | ACTN1, FUS, SMAD4, YBX1, | 111.30 | 81.98 | 134.50 | 0.900 | 0.797 | 0.967 | 0.849 | 0.656 | 0.973 |
| | | | DERL1, SMAD2 | | | | | | | | | |
| | 5 | 19.030 | ACTN1, FUS, pS6, SMAD4, | 126.70 | 99.31 | 150.61 | 0.884 | 0.793 | 0.952 | 0.848 | 0.673 | 0.963 |
| | | | SMAD2 | | | | | | | | | |
| | 4 | 29.339 | ACTN1, FUS, SMAD4, SMAD2 | 133.03 | 104.06 | 156.82 | 0.868 | 0.768 | 0.944 | 0.837 | 0.649 | 0.960 |
| AIC | 10 | 0.001 | ACTN1, COX6C, FUS, pS6, | 96.58 | 63.28 | 124.25 | 0.933 | 0.842 | 0.988 | 0.839 | 0.619 | 0.971 |
| | | | SMAD4, YBX1, DEC1, DERL1, | | | | | | | | | |
| | | | HOXB13, SMAD2 | | | | | | | | | |
| | 9 | 0.003 | ACTN1, COX6C, FUS, pS6, | 97.61 | 64.50 | 123.14 | 0.932 | 0.840 | 0.988 | 0.857 | 0.658 | 0.979 |
| | | | SMAD4, YBX1, DERL1, HOXB13, | | | | | | | | | |
| | | | SMAD2 | | | | | | | | | |
| | 8 | 0.023 | ACTN1, FUS, pS6, SMAD4, YBX1, | 100.83 | 68.72 | 125.25 | 0.924 | 0.831 | 0.983 | 0.855 | 0.658 | 0.976 |
| | | | DERL1, HOXB13, SMAD2 | | | | | | | | | |
| | 7 | 0.357 | ACTN1, FUS, pS6, SMAD4, YBX1, | 105.67 | 73.83 | 130.47 | 0.913 | 0.820 | 0.973 | 0.861 | 0.667 | 0.980 |
| | | | DERL1, SMAD2 | | | | | | | | | |
| | 6 | 1.939 | ACTN1, pS6, SMAD4, YBX1, | 110.71 | 81.92 | 138.26 | 0.896 | 0.783 | 0.968 | 0.858 | 0.651 | 0.981 |
| | | | DERL1, SMAD2 | | | | | | | | | |
| | 5 | 5.317 | ACTN1, SMAD4, YBX1, DERL1, | 115.48 | 89.56 | 140.31 | 0.884 | 0.771 | 0.957 | 0.848 | 0.658 | 0.971 |
| | | | SMAD2 | | | | | | | | | |
| | 4 | 12.715 | ACTN1, YBX1, DERL1, SMAD2 | 121.94 | 92.75 | 144.96 | 0.861 | 0.748 | 0.944 | 0.830 | 0.633 | 0.960 |
| Test | 7 | 0.001 | ACTN1, FUS, pS6, SMAD4, YBX1, | 105.67 | 73.83 | 130.47 | 0.913 | 0.820 | 0.973 | 0.861 | 0.667 | 0.980 |
| | | | DERL1, SMAD2 | | | | | | | | | |
| | 6 | 0.002 | ACTN1, FUS, pS6, SMAD4, | 122.68 | 91.59 | 145.62 | 0.898 | 0.807 | 0.966 | 0.861 | 0.679 | 0.972 |
| | | | DERL1, SMAD2 | | | | | | | | | |
| | 9 | 0.003 | ACTN1, COX6C, FUS, pS6, | 103.11 | 70.64 | 129.52 | 0.928 | 0.840 | 0.983 | 0.860 | 0.669 | 0.977 |
| | | | SMAD4, YBX1, FKBP5, DERL1, | | | | | | | | | |
| | | | SMAD2 | | | | | | | | | |
| | 8 | 0.004 | ACTN1, COX6C, FUS, pS6, | 115.88 | 88.32 | 142.45 | 0.919 | 0.833 | 0.973 | 0.859 | 0.690 | 0.965 |
| | | | SMAD4, FKBP5, DERL1, SMAD2 | | | | | | | | | |
| | 10 | 0.043 | ACTN1, COX6C, FUS, pS6, | 98.98 | 67.44 | 124.73 | 0.934 | 0.845 | 0.988 | 0.852 | 0.649 | 0.970 |
| | | | SMAD4, YBX1, DERL1, HOXB13, | | | | | | | | | |
| | | | MTDH2, SMAD2 | | | | | | | | | |
| | 5 | 0.082 | ACTN1, FUS, SMAD4, DERL1, | 128.74 | 93.09 | 153.75 | 0.883 | 0.785 | 0.954 | 0.849 | 0.668 | 0.963 |
| | | | SMAD2 | | | | | | | | | |
| | 4 | 0.659 | ACTN1, FUS, SMAD4, SMAD2 | 133.03 | 104.06 | 156.82 | 0.868 | 0.768 | 0.944 | 0.837 | 0.649 | 0.960 |

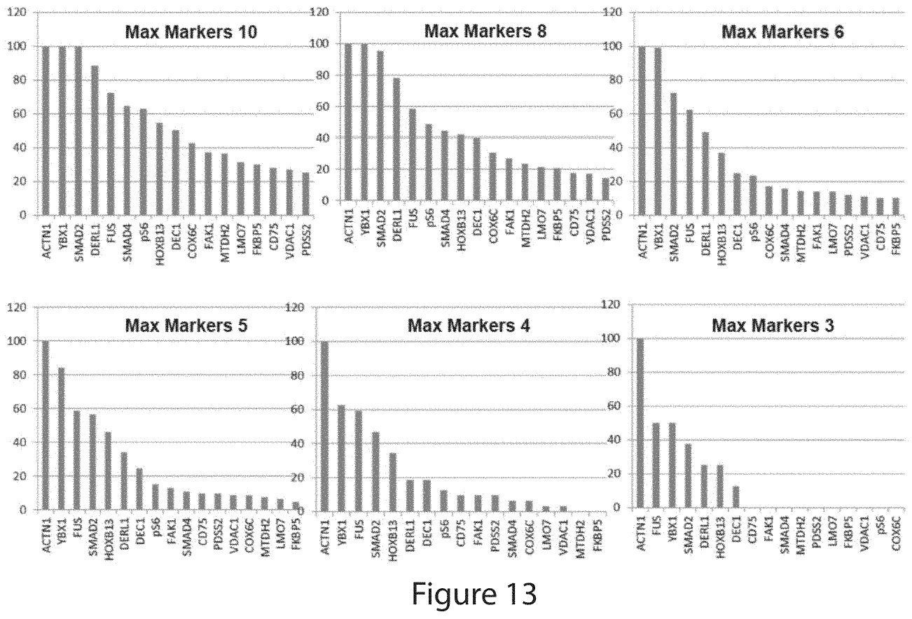

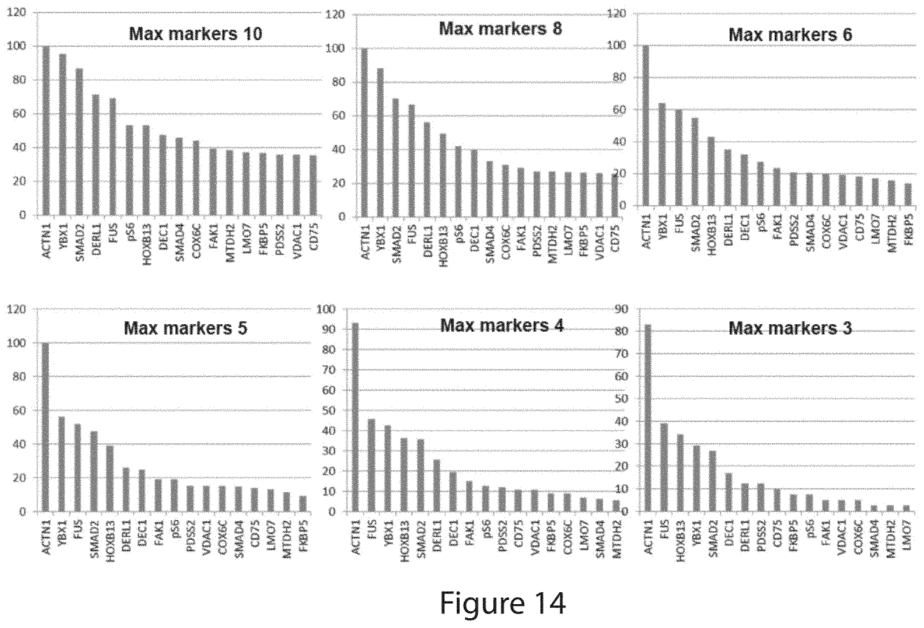

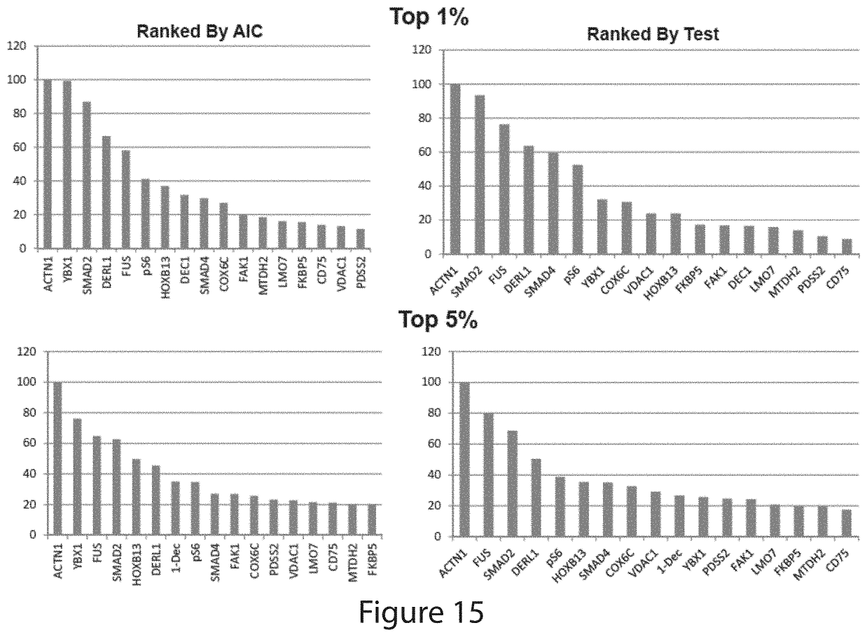

As expected, when the data were sorted based on training data or AIC, the correlation of the various combinations with aggressiveness increased as the size of the combination increased. In other words, the 10-member combinations were more predictive of aggressiveness than 9-member combinations, and so on. This is expected because with each additional member in the combination, an additional degree of freedom is added to the training analysis. When the data were sorted based on the test data, however, combinations with seven members or six members were more correlative than combinations with eight, nine, and ten members because as the data are trained with more degrees of freedom, it becomes more difficult to generalize to the test data. Accordingly, combinations of six or seven biomarkers may in some cases be more useful in predicting the aggressiveness of tumors in a clinical assay. The frequency with which each biomarker appeared in the top combinations for each AIC and test data was determined. See, FIG. 13 for the top biomarkers in the top 1% of 3- to 10-member combinations sorted by AIC; FIG. 14 for the top biomarkers in the top 5% of 3- to 10-member combinations sorted by AIC; and FIG. 15 for the top biomarkers in the top 1% and top 5% of seven-member combinations sorted by AIC and test C-stat data. The top 5% of seven-member combinations sorted by test data are presented in Table 6, which is disclosed in U.S. Provisional Application No. 61/792,003, filed Mar. 15, 2013, the entire content of which is incorporated by reference herein.

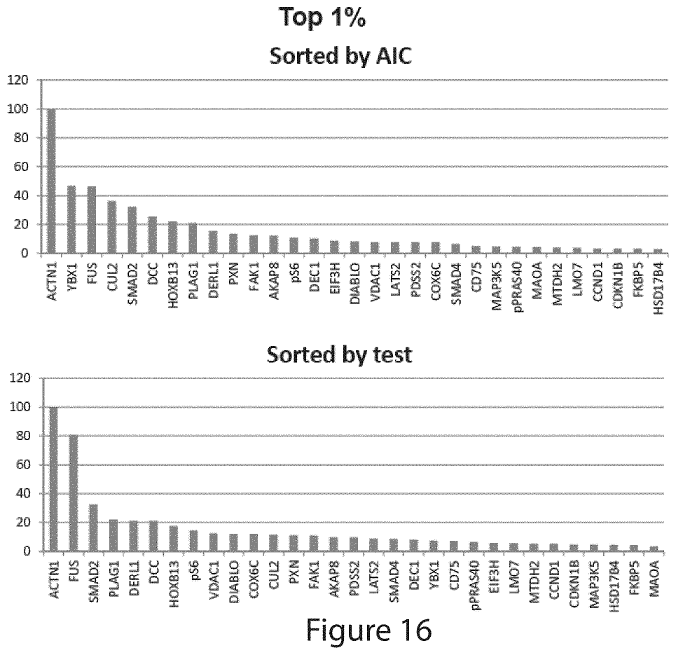

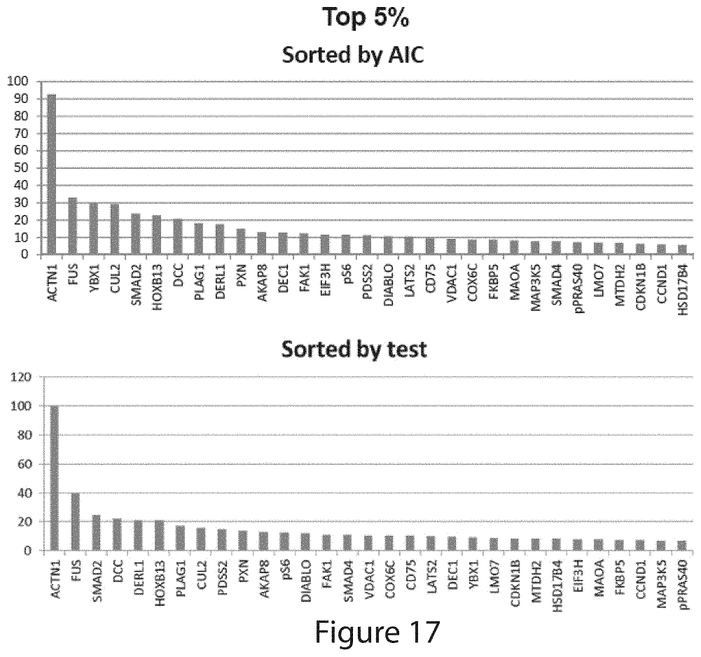

The top-ranking models for tumor aggressiveness in the combinations not preselected for univariate performance for each method of analysis are listed in Table 4. The frequency with which each biomarker appeared in the top combinations for each AIC and test data was determined. See, FIG. 16 for the frequency with which biomarkers appear in the top 1% of 5-member combinations sorted by AIC and test data. See, FIG. 17 for the frequency with which biomarkers appear in the top 5% of 5-member combinations sorted by AIC and test data. The flat tails of FIGS. 16 and 17 suggest that many of these biomarkers are interchangeable and may provide little added performance.

TABLE 4
Top Combinations of 31 HLTMA-Tested Biomarkers

| | | | | | | | | Low | High | | Low | |
|---|---|---|---|---|---|---|---|---|---|---|---|---|
| | | | | | Low | High | Median | 95% | 95% | | 95% | High |
| Sorted | Max | | Markers in | Median | 95% | 95% | Train c- | train c- | train c- | Median | test c- | 95% test |
| By | Markers | Percentile | Model | AIC | AIC | AIC | stat | stat | stat | test c-stat | stat | c-stat |
| Train | 5 | 0.0005 | ACTN1, FUS, | 124.95 | 98.61 | 150.52 | 0.892 | 0.796 | 0.956 | 0.856 | 0.676 | 0.965 |
| | | | PLAG1, | | | | | | | | | |
| | | | SMAD2, | | | | | | | | | |
| | | | SMAD4 | | | | | | | | | |
| | 4 | 0.1197 | ACTN1, FUS, | 133.56 | 104.26 | 159.09 | 0.869 | 0.769 | 0.943 | 0.833 | 0.649 | 0.957 |
| | | | SMAD2, | | | | | | | | | |
| | | | SMAD4 | | | | | | | | | |
| AIC | 5 | 0.0005 | ACTN1, | 112.33 | 86.22 | 135.58 | 0.873 | 0.756 | 0.954 | 0.813 | 0.596 | 0.958 |
| | | | CUL2, DERL1, | | | | | | | | | |
| | | | SMAD2, | | | | | | | | | |
| | | | YBX1 | | | | | | | | | |
| | 4 | 0.0344 | ACTN1, FUS, | 121.41 | 94.94 | 146.58 | 0.859 | 0.743 | 0.942 | 0.828 | 0.627 | 0.960 |
| | | | SMAD2, | | | | | | | | | |
| | | | YBX1 | | | | | | | | | |
| Test | 5 | 0.0005 | ACTN1, FUS, | 124.95 | 98.61 | 150.52 | 0.892 | 0.796 | 0.956 | 0.856 | 0.676 | 0.965 |
| | | | PLAG1, | | | | | | | | | |
| | | | SMAD2, | | | | | | | | | |
| | | | SMAD4 | | | | | | | | | |
| | 4 | 0.0170 | ACTN1, | 134.54 | 106.12 | 157.19 | 0.861 | 0.753 | 0.940 | 0.840 | 0.654 | 0.961 |
| | | | AKAP8, | | | | | | | | | |
| | | | PLAG1, | | | | | | | | | |
| | | | SMAD2 | | | | | | | | | |

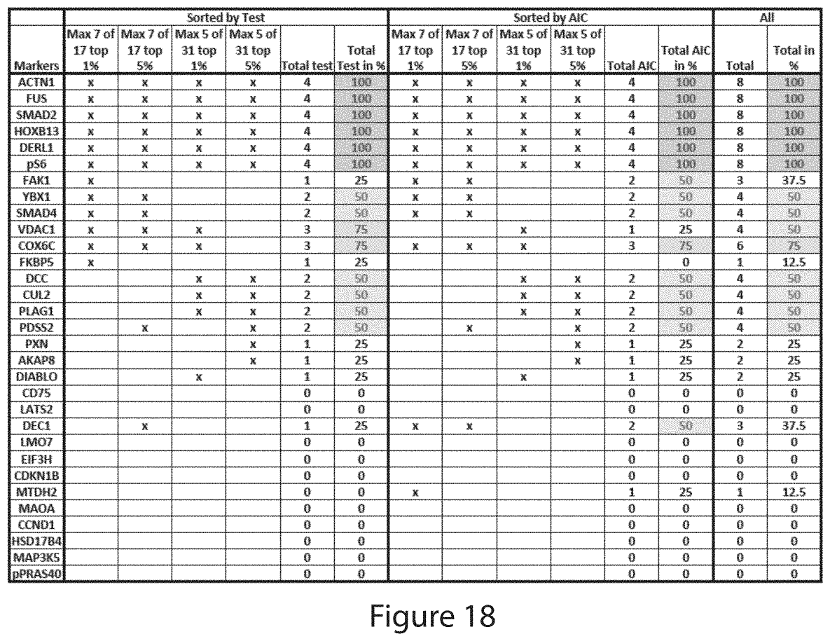

When all of the above data are combined, a core set of seven markers (i.e., ACTN1, FUS, SMAD2, HOXB13, DERL1, RpS6, and CoX6C) that consistently appear in all selection approaches for prostate tumor aggressiveness can be identified (see, markers with 100% or 75% in the right most column of FIG. 18). A secondary set of seven markers for prostate tumor aggressiveness (i.e., YBX1, SMAD4, VDAC1, DCC, CUL2, PLAG1, and PDSS2) can also be readily identified (see, markers with 50% in the right most column of FIG. 18).

We also evaluated whether combinations of biomarkers correlated with lethal outcome using combinations of the 17 biomarkers that demonstrated univariate performance in both aggressiveness and lethal outcome determinations. Combinations between three and ten biomarkers selected from the 17 univariately performing biomarkers were analyzed by logistic regression. Train/test cohorts utilizing bootstrapping (i.e. random sampling with replacement) and multiple rounds of cross-validation were analyzed by C-stat, AIC and 95% confidence intervals. The top-ranking models for lethal outcome in the combinations preselected for univariate performance for each method of analysis are listed in Table 5.

TABLE 5
Top Combinations for Lethal Outcome

| | Num- | | | | | | | Low | High | | Low | High |
|---|---|---|---|---|---|---|---|---|---|---|---|---|
| | ber | | | | AIC | AIC | Median | 95% | 95% | Median | 95% | 95% |
| Sorted | of | Per- | | | Low | High | C-stat | c-Stat | C-stat | C-stat | C-stat | C-stat |
| By | Markers | centile | Markers | AIC | 95% | 95% | train | Train | train | Test | test | Test |
| Train | 10 | 0.001 | ACTN1, COX6C, FAK1, PDSS2, | 134.62 | 99.16 | 168.69 | 0.902 | 0.796 | 0.965 | 0.784 | 0.553 | 0.947 |
| | | | SMAD4, YBX1, LMO7, MTDH2, | | | | | | | | | |
| | | | HOXB13, SMAD2 | | | | | | | | | |
| | 9 | 0.101 | ACTN1, CD75, COX6C, FAK1, | 135.41 | 96.08 | 170.21 | 0.895 | 0.798 | 0.961 | 0.795 | 0.566 | 0.946 |
| | | | PDSS2, YBX1, MTDH2, HOXB13, | | | | | | | | | |
| | | | SMAD2 | | | | | | | | | |
| | 8 | 0.340 | ACTN1, COX6C, FAK1, PDSS2, | 138.07 | 103.43 | 171.97 | 0.891 | 0.793 | 0.957 | 0.804 | 0.554 | 0.953 |
| | | | YBX1, MTDH2, HOXB13, SMAD2 | | | | | | | | | |
| | 7 | 1.717 | ACTN1, COX6C, PDSS2, YBX1, | 141.52 | 103.64 | 181.16 | 0.882 | 0.769 | 0.951 | 0.816 | 0.583 | 0.955 |
| | | | MTDH2, HOXB13, SMAD2 | | | | | | | | | |
| | 6 | 5.378 | ACTN1, COX6C, YBX1, MTDH2, | 145.17 | 107.13 | 181.49 | 0.869 | 0.767 | 0.944 | 0.817 | 0.616 | 0.948 |
| | | | HOXB13, SMAD2 | | | | | | | | | |
| | 5 | 17.298 | ACTN1, COX6C, YBX1, MTDH2, | 155.88 | 123.37 | 188.20 | 0.851 | 0.754 | 0.925 | 0.805 | 0.606 | 0.938 |
| | | | HOXB13 | | | | | | | | | |
| | 4 | 35.653 | ACTN1, COX6C, MTDH2, | 173.35 | 133.01 | 207.87 | 0.831 | 0.726 | 0.914 | 0.802 | 0.621 | 0.931 |
| | | | HOXB13 | | | | | | | | | |
| AIC | 10 | 0.001 | ACTN1, COX6C, FAK1, PDSS2, | 131.54 | 96.08 | 166.01 | 0.894 | 0.797 | 0.963 | 0.775 | 0.529 | 0.938 |
| | | | YBX1, DEC1, FKBP5, MTDH2, | | | | | | | | | |
| | | | HOXB13, SMAD2 | | | | | | | | | |
| | 9 | 0.004 | ACTN1, COX6C, FAK1, PDSS2, | 133.20 | 98.31 | 168.91 | 0.889 | 0.784 | 0.959 | 0.792 | 0.546 | 0.944 |
| | | | YBX1, DEC1, MTDH2, HOXB13, | | | | | | | | | |
| | | | SMAD2 | | | | | | | | | |
| | 8 | 0.039 | ACTN1, CD75, FAK1, PDSS2, | 135.48 | 101.98 | 170.86 | 0.883 | 0.787 | 0.952 | 0.792 | 0.568 | 0.946 |
| | | | YBX1, MTDH2, HOXB13, SMAD2 | | | | | | | | | |
| | 7 | 0.453 | ACTN1, COX6C, FAK1, YBX1, | 139.52 | 108.55 | 176.18 | 0.878 | 0.779 | 0.948 | 0.808 | 0.598 | 0.949 |
| | | | MTDH2, HOXB13, SMAD2 | | | | | | | | | |
| | 6 | 1.424 | ACTN1, FAK1, YBX1, MTDH2, | 142.23 | 109.59 | 175.41 | 0.865 | 0.766 | 0.939 | 0.797 | 0.585 | 0.940 |
| | | | HOXB13, SMAD2 | | | | | | | | | |
| | 5 | 9.126 | ACTN1, CD75, YBX1, DEC1, | 150.41 | 117.30 | 186.36 | 0.838 | 0.732 | 0.917 | 0.788 | 0.587 | 0.928 |
| | | | HOXB13 | | | | | | | | | |
| | 4 | 18.742 | ACTN1, CD75, YBX1, HOXB13 | 156.38 | 121.57 | 188.52 | 0.825 | 0.721 | 0.908 | 0.784 | 0.596 | 0.920 |
| Test | 7 | 0.001 | ACTN1, COX6C, YBX1, LMO7, | 145.21 | 112.25 | 181.32 | 0.878 | 0.766 | 0.949 | 0.823 | 0.609 | 0.951 |
| | | | MTDH2, HOXB13, SMAD2 | | | | | | | | | |
| | 8 | 0.002 | ACTN1, COX6C, PDSS2, YBX1, | 140.73 | 99.27 | 174.15 | 0.890 | 0.775 | 0.963 | 0.817 | 0.586 | 0.956 |
| | | | LMO7, MTDH2, HOXB13, SMAD2 | | | | | | | | | |
| | 6 | 0.003 | ACTN1, COX6C, YBX1, MTDH2, | 145.17 | 107.13 | 181.49 | 0.869 | 0.767 | 0.944 | 0.817 | 0.616 | 0.948 |
| | | | HOXB13, SMAD2 | | | | | | | | | |
| | 9 | 0.004 | ACTN1, COX6C, PDSS2, YBX1, | 141.51 | 106.10 | 178.76 | 0.891 | 0.779 | 0.958 | 0.816 | 0.586 | 0.955 |
| | | | LMO7, DERL1, MTDH2, HOXB13, | | | | | | | | | |
| | | | SMAD2 | | | | | | | | | |
| | 5 | 0.007 | ACTN1, COX6C, LMO7, MTDH2, | 169.58 | 127.52 | 206.74 | 0.847 | 0.739 | 0.927 | 0.816 | 0.630 | 0.937 |
| | | | HOXB13 | | | | | | | | | |
| | 10 | 0.067 | ACTN1, COX6C, PDSS2, pS6, | 141.96 | 101.04 | 175.82 | 0.894 | 0.783 | 0.966 | 0.806 | 0.585 | 0.951 |
| | | | YBX1, LMO7, VDAC1, MTDH2, | | | | | | | | | |
| | | | HOXB13, SMAD2 | | | | | | | | | |
| | 4 | 0.124 | ACTN1, COX6C, MTDH2, | 173.35 | 133.01 | 207.87 | 0.831 | 0.726 | 0.914 | 0.802 | 0.621 | 0.931 |
| | | | HOXB13 | | | | | | | | | |

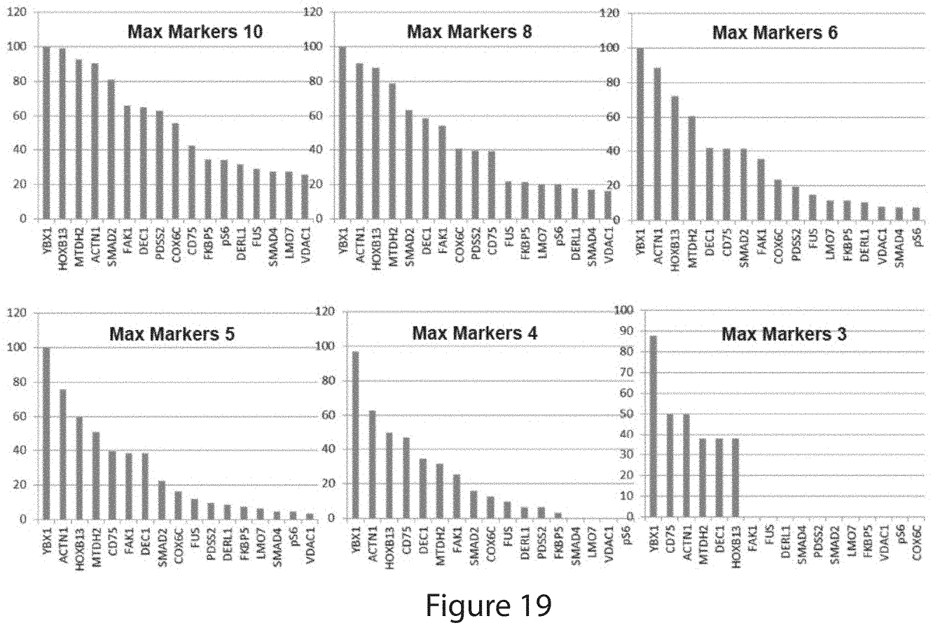

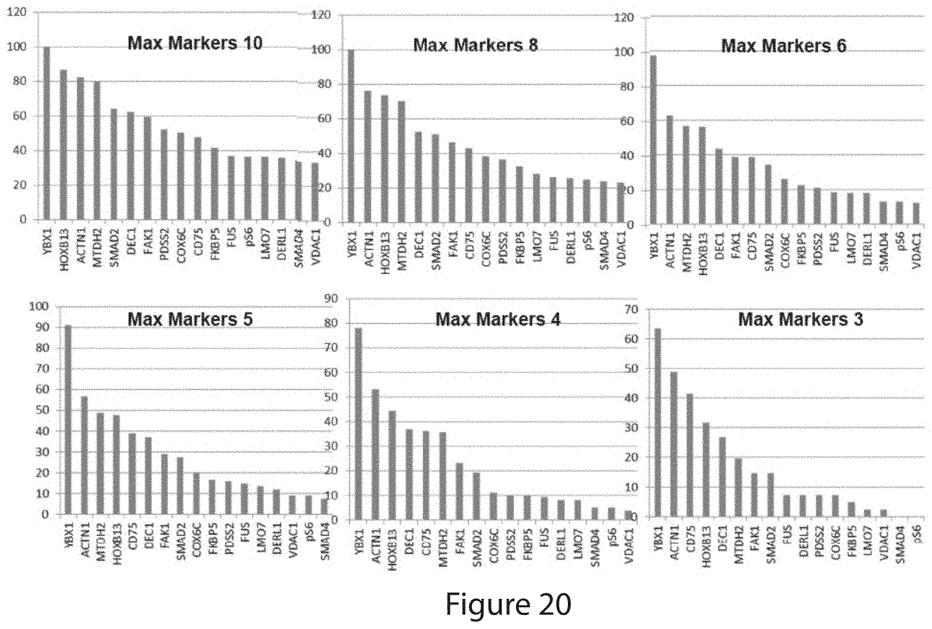

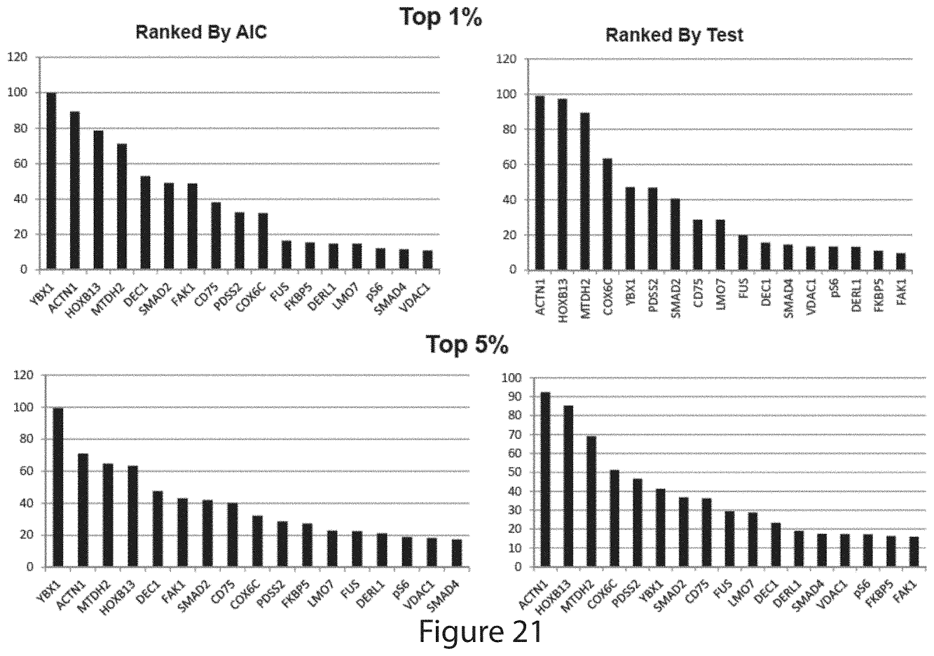

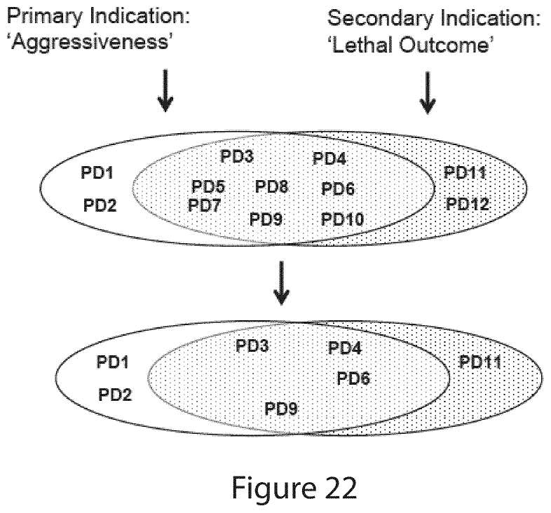

Similar to the tumor aggressiveness model above, combinations of six or seven biomarkers may be the most useful in predicting lethal outcome in a clinical assay. The frequency with which each biomarker appeared in the top combinations for each AIC and test data was determined for lethal outcome analysis. See, FIG. 19 for the top biomarkers in the top 1% of 3- to 10-member combinations sorted by AIC; FIG. 20 for the top biomarkers in the top 5% of 3- to 10-member combinations sorted by AIC; and FIG. 21 for the top biomarkers in the top 1% and top 5% of seven-member combinations sorted by AIC and test data. Interestingly, when comparing FIGS. 15 and 21, 8 biomarkers appear in the top 12 biomarkers for both tumor aggression and lethal outcome. Accordingly, it is possible to select a set of biomarkers that partially overlap in their ability to predict tumor aggression and lethal outcome (FIG. 22).

### Example 4: Clinical Verification of Biomarker Combinations

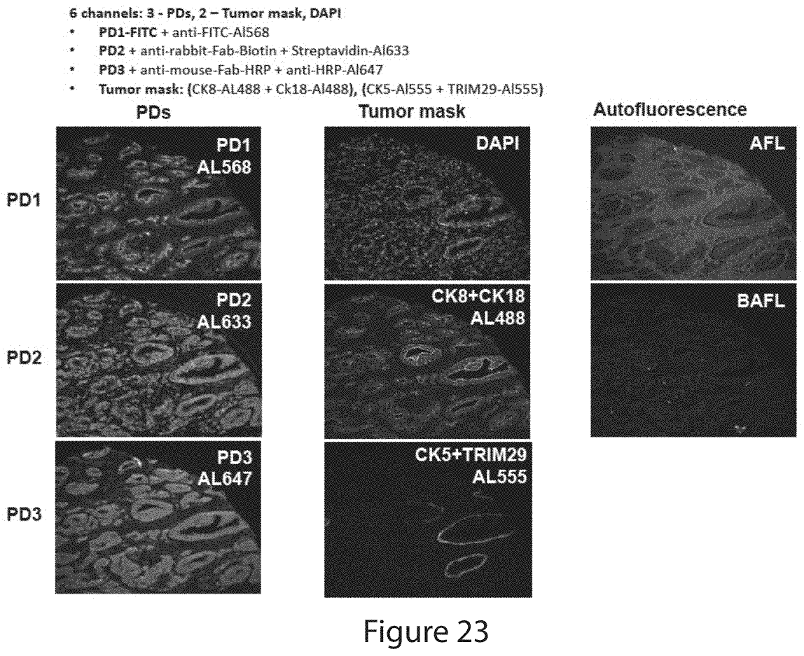

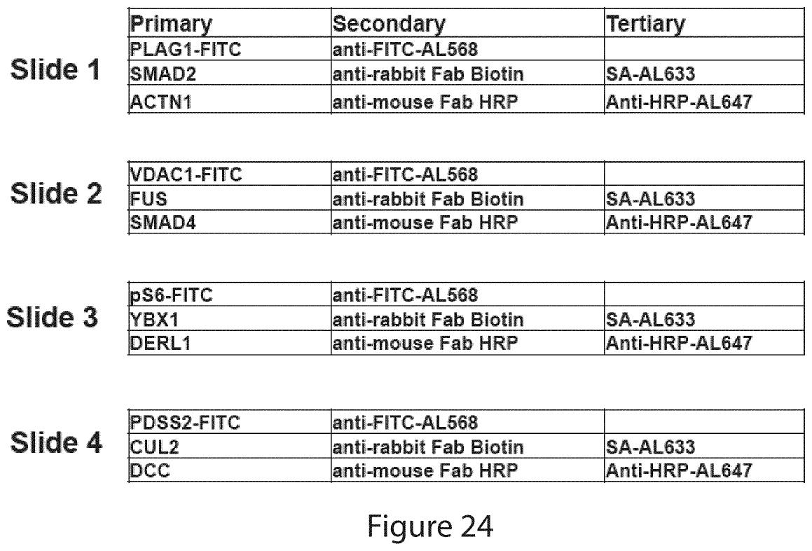

Using the combinations of the top biomarkers identified in Example 3 above, we designed an assay for evaluating clinical tumor samples for tumor aggression and verifying the results of our models above. Image acquisition hardware can detected up to six different fluorescent channels. Accordingly, it is possible to detect up to three biomarkers (or prognosis determinants, “PD”) along with two tumor mask signals and a nuclear stain (e.g., DAPI), i.e., Triplex staining. FIG. 23, for example, shows the detection of six different fluorescent signals from a single slide for three biomarkers (HSD17B4, FUS, and LATS2), two tumor mask signals (CK8+CK18-Alexa 488 and CK5+TRIM29-Alexa 555) and nuclear staining (DAPI). For example, the first channel may be used to detect a first biomarkers (e.g., PD1), whose primary antibody is conjugated to a FITC molecule and can be detected by anti-FITC-Alexa-568. The second channel may be used to detect a second biomarker (e.g., PD2), whose primary antibody is a rabbit antibody that can be detected using anti-rabbit Fab conjugated to biotin and streptavidin conjugated to Alexa-633. The third channel may be used to detect a third biomarker (e.g., PD3), whose primary antibody is a mouse antibody that can be detected with anti-mouse Fab conjugated to horseradish peroxidase (HRP) and anti-HRP conjugated to Alexa-647. The fourth channel may be used to detect epithelial markers of a carcinoma, such as cytokeratin 8 (CK8 or KRT8) and cytokeratin 18 (CK18 or KRT18). For example, a combination of anti-CK8-Alexa-488 and anti-CK18-Alexa-488 can be used to define the tumor regions of a sample. The fifth channel may be used to detect basal epithelial markers such as cytokeratin 5 (CK5 or KRT5) and TRIM29. For example, a combination of anti-CK5-Alexa-555 and anti-TRIM29-Alexa-555 can be used to define the non-tumor regions of a sample. The sixth channel may be used to detect a cellular structure, such as detecting a nucleus with DAPI staining. From the a core set of seven markers and the secondary set of seven markers, we identified 12 commercially available antibodies for these markers suitable for Triplex staining on a set of 4 slides (see, FIG. 24).

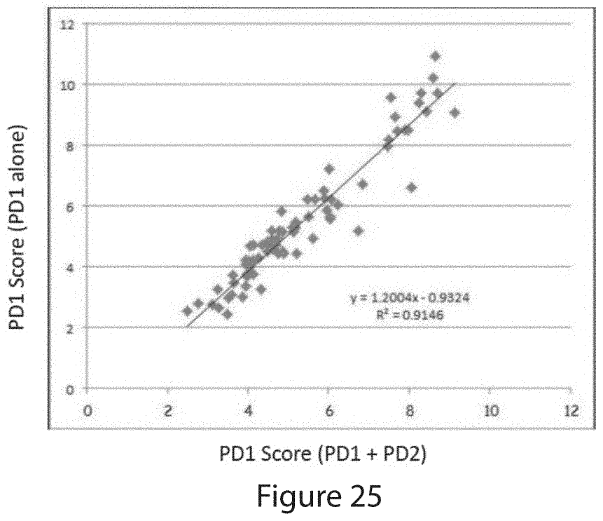

To confirm that staining with multiple antibodies in Triplex staining would not affect the detection of those antibodies, we compared the signal from an antibody for a biomarker (PD1) in an assay by itself or in the presence of the antibody for another biomarker (PD2) on a background of tumor mask markers. As shown in FIG. 25, the addition of antibodies for a second biomarker has minimal impact on the detection of the first marker. Using this analysis, we confirmed that the combinations of markers listed in FIG. 24 could be used with minimal interference in the detection of each biomarker.

We next obtained two cohorts of prostate cancer tumor samples: a cohort of 350 tumors from the Cleveland Clinic and a cohort of 180 tumors from Harvard School of Public Health. We isolated five 5 μm serial sections from each tumor sample in the cohort. Four of the sections were used for biomarker detection, as described in FIG. 24. The fifth section was used to determine the quality of the tumor sample by evaluating autofluroescence. Two channels were evaluated for autofluorescence, one for general tissue autofluorescence (AFL) and another for erythrocytes and bright granules (BAFL) scattered across prostatic tissue. See, e.g., FIG. 23.

Specifically, five μm sections were cut from paraffin-embedded tumor sample blocks and placed on Histogrip (Life Technologies) coated slides. Slides were baked at 65° C. for 30 min, de-paraffinized through serial incubations in xylene, and rehydrated through a series of graded alcohols. Antigen retrieval was done in a 0.05% Citraconic anhydride solution at pH 7.4 for 40 min at 95° C.

Immunofluorescent staining was done using a LabVision Autostainer, with all incubations at room temperature, all washes with TBS-T (TBS+0.05% Tween 20), and all antibodies diluted with TBS-T+0.1% BSA solution. Slides were first blocked with Biotin Block (Life Technologies) solution A for 20 min, washed, then solution B for 20 min, washed, and then blocked with Background Sniper (Biocare Medical) for 20 min and washed again. Mixtures of FITC-conjugated, mouse and rabbit primary antibodies (see FIG. 23) were applied and incubated for 1 hour.

After extensive washes, a mixture of biotin-conjugated anti-rabbit IgG and HRP conjugated anti-rabbit IgG was applied for 45 min. After extensive washes, a mixture of Alexa fluorophore-conjugated reagents was applied that consisted of streptavidin-Alexa 633, anti-FITC mAb-Alexa 568, anti-HRP mAb-Alexa 647 and a Tumor Mask cocktail (anti-cytokeratin 8 mAb Alexa 488, anti-cytokeratin 18 mAb Alexa 488, anti-cytokeratin 5 mAb Alexa 555, anti-TRIM29 mAb Alexa555). As described above, we utilized a combination of antibodies directed against prostate epithelial and basal markers (Tumor Mask) and object recognition based on Definiens Developer XD to enable automated image analysis of prostate cancer tumor tissue. Tumor regions were defined as prostate epithelial structures devoid of basal markers. A cocktail of Alexa 488-conjugated anti-cytokeratin 8 and anti-cytokeratin 18-specific mouse mAbs was used to obtain epithelial-specific staining. Staining of basal cells was based on a cocktail of Alexa 555-conjugated anti-cytokeratin 5 and anti-TRIM29-specific mAbs. The slides were incubated for 1 hour with these Alexa fluorophore-conjugated reagents. After extensive washes, a DAPI solution (100 ng/ml DAPI in TBS-T) was applied for 3 min. After several washes, slides were mounted in Prolong Gold anti-fade reagent (Life Technologies). Slides were left overnight at −20° C. in the dark to “cure” and were stored long term in the dark at −20° C. to minimize fading. Images were acquired and analyzed as described in Example 3.

### Example 5: Development of an Automated Quantitiative Multiplex Proteomics In Situ Imaging Platform and Application in Prediction of Prostate Cancer Lethal Outcome

### SUMMARY

There has been significant progress in gene-based approaches to cancer prognostication, promising early intervention for high-risk patients and avoidance of overtreatment for low-risk patients. There has been less advancement in proteomics approaches, even though perturbed protein levels and post-translational modifications are more directly linked with phenotype. Most current, gene expression-based platforms require tissue lysis resulting in loss of structural molecular information, and hence are blind to tumor heterogeneity and morphological features. Presented here is an automated, integrated multiplex proteomics in situ imaging platform that quantitatively measures protein biomarker levels and activity states in defined intact tissue regions where the biomarkers of interest exert their phenotype. As proof-of-concept, it was confirmed thatfour previously reported prognostic markers, PTEN, SMAD4, CCND1 and SPP1, can predict lethal outcome of human prostate cancer. Furthermore, it was shown that the mechanism-based power of protein expression by demonstrating that PTEN can be replaced by two PI3K pathway-regulated protein activities. In summary, the platform can reproducibly and simultaneously quantify and assess multiple functional activities of oncogenes and tumor-suppressor genes in intact tissue. The platform is broadly applicable and well suited for prognostication at early stages of disease where key signaling protein levels and activities are perturbed.

### INTRODUCTION

While tests for recurrent, validated gene mutations have great prognostic and predictive value¹⁻⁵, these mutations are relatively rare in early stage cancers. Multivariate gene-based tests require homogenized tissue with variable ratios of tumor and benign tissue resulting in less accurate biomarker measurements^{6,7}. In these types of tests, phenotype must be inferred from genetic and mutational patterns. In contrast, direct in situ measurement of protein levels and post-translational modifications should more directly reflect the status of oncogenic signaling pathways. Thus, it is reasonable to expect a protein-based approach to be valuable for prognostication.

Other issues complicate prognostic testing. In prostate cancer, tumor heterogeneity is pronounced, and sampling error can contribute to incorrect predictions. Pathologist discordance in Gleason grading and tumor staging also renders prognostication in this multifocal disease difficult. To address these shortcomings, we developed an automated quantitative multiplex proteomics imaging platform for intact tissue that integrates morphological object recognition and molecular biomarker measurements from defined, relevant tissue regions at the individual slide level. This system was used to predict lethal outcome from radical prostatectomy tissue using four previously reported markers, PTEN, SMAD4, CCND1 and SPP1⁸. Importantly, here it is also demonstrated thatquantitative measurements of protein activity states reflective of PI3K/AKT and MAPK signaling status can substitute for PTEN, a highly validated prognostic marker which itself regulates PI3K/AKT pathway signaling⁹⁻¹³. Together these data identified a novel lethal outcome predictive signature: SMAD4, CCND1, SPP1, phospho-PRAS40 (pPRAS40)-T246 and phospho-ribosomal S6 (pS6)-S235/236.

Materials and Methods

Reagents and Antibodies

All antibodies and reagents used in this study were procured from commercially available sources as described in Table 7. Anti-FITC MAb-Alexa568, anti-CK8-Alexa488, anti-CK18-Alexa488, anti-CK5-Alexa555 and anti-Trim29-Alexa555 were conjugated with Alexa dyes, in-house using appropriate protein conjugation kits, according to manufacturer's instructions (LifeTechnologies, Grand Island, N.Y.).

TABLE 7
Antibodies

| | Antibody | | | |
|---|---|---|---|---|
| Antigen | Type | Source | Clone | Catalog # |
| KRT8 | mouse mAb | Santa-Cruz | C51 | sc-8020 |
| KRT18 | mouse mAb | Santa-Cruz | C-04 | sc-51582 |
| KRT5 | mouse mAb | Santa-Cruz | RCK103 | sc-32721 |
| ATDC1 | mouse mAb | Santa-Cruz | A-5 | sc-166718 |
| (Trim29) | | | | |
| PTEN | rabbit mAb | Cell | D4.3 | 9188 |
| | | Signaling | | |
| CCND1 | rabbit mAb | Spring Bio | SP4 | M3044 |
| SMAD4 | mouse mAb | Santa-Cruz | B-8 | sc-7966 |
| SPP1 | rabbit mAb | Abcam | EPR3688 | ab91655 |
| PRAS40 pT246 | rabbit mAb | Cell | C77D7 | 2997 |
| | | Signaling | | |
| S6 | rabbit mAb | Cell | D57.2.2E | 4858 |
| pS235/pS236 | | Signaling | | |
| RSK1 | rabbit mAb | Abcam | E238 | 2006-1 |
| pT359/pS363 | | | | |
| Foxo3a | rabbit mAb | Abcam | EP1949Y | ab53287 |

Acquisition, processing and quality control of formalin-fixed, paraffin-embedded (FFPE) prostate cancer tissue blocks.

We acquired a cohort of FFPE human prostate cancer tissue blocks with clinical annotations and long-term patient outcome information from Folio Biosciences (Powell, Ohio). Samples had been collected with appropriate IRB approval and all patient records were de-identified. We included a number of FFPE human prostate cancer tissue blocks from other commercial sources (BioOptions, Brea, Calif.; Cureline, So. San Francisco, Calif.; ILSBio, Chestertown, Md.; OriGene, Rockville, Md.) to validate individual antibody and combined multiplex staining format staining intensities, to develop quality control procedures, to assess intra-experiment reproducibility studies, and to confirm specificity of staining on prostate tumor tissue.

Between 10 to 12 sections (5 μm cuts) were produced from each FFPE block. The last section was stained with hematoxylin and eosin (H&E) and scanned with an Aperio (Vista, Calif.) XT system. H&E stained images were deposited into the Spectrum database (Aperio, Vista, Calif.) for remote reviewing and centralized Gleason annotation in a blinded manner by expert Board-Certified anatomical pathologists using ImageScope software (Aperio, Vista, Calif.). Annotated circles corresponding to 1 mm cores were placed over four areas of highest and two areas of lowest Gleason patterns on each prostatectomy sample using current criteria¹⁴.

Tissue Quality Control Procedure

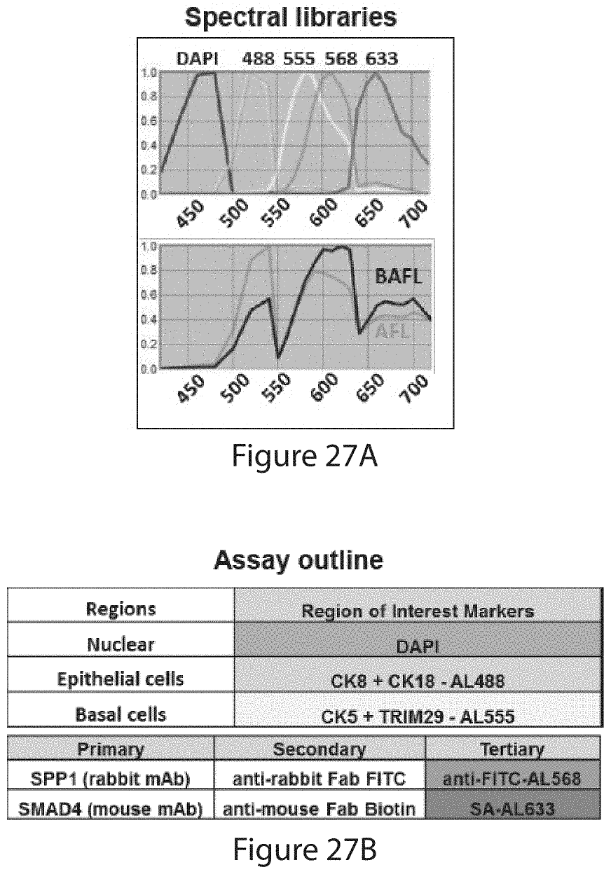

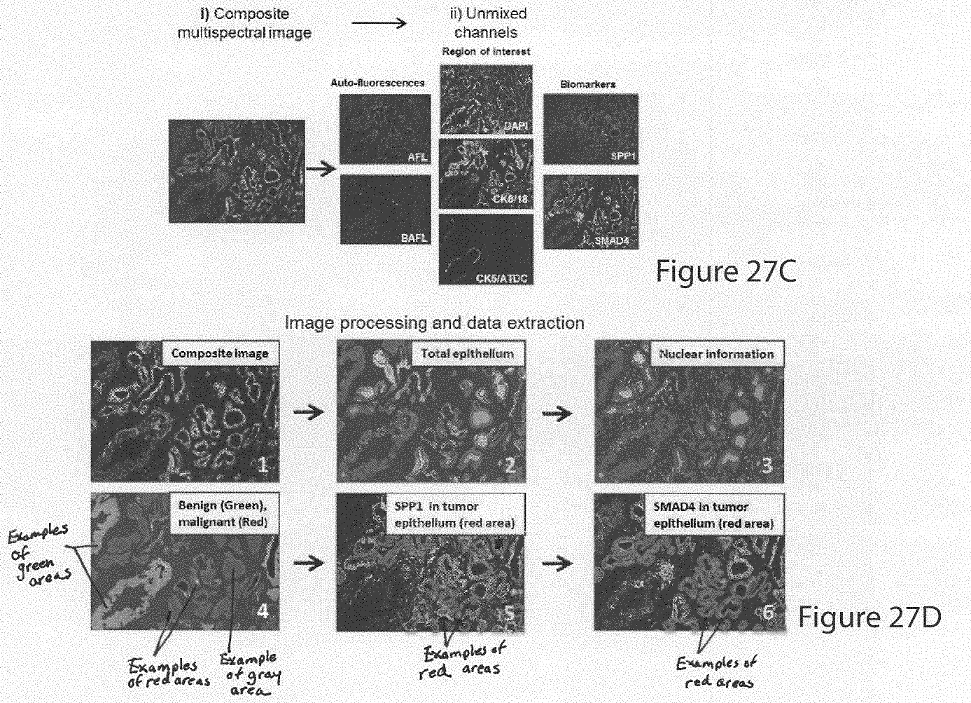

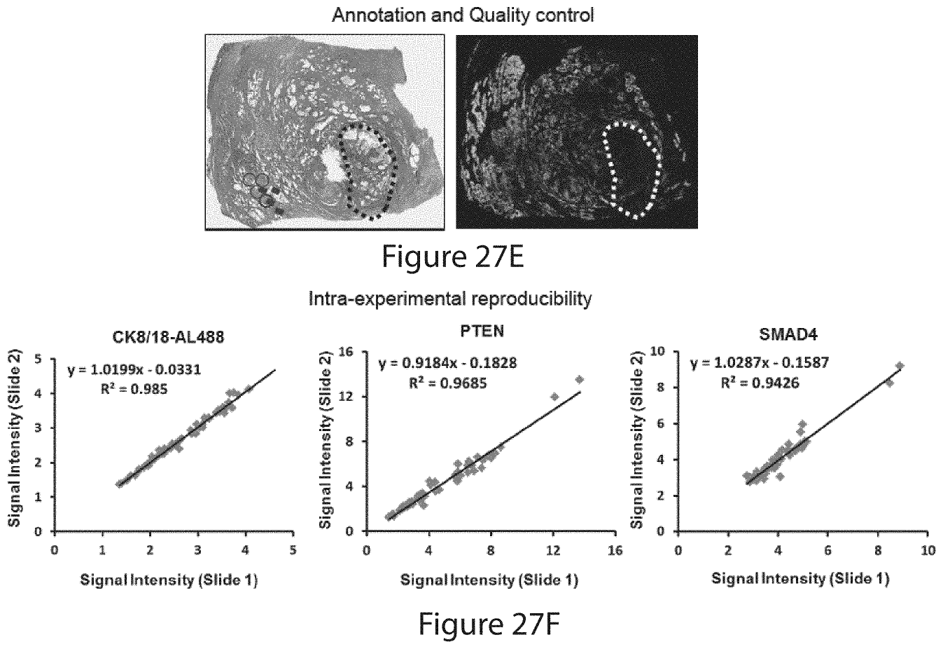

A 5 μm section from each FFPE block was stained with anti-phospho STAT3T705 rabbit monoclonal antibody (mAb), anti-STAT3 mouse mAb and region of interest markers, as described below. Slides were examined under a fluorescence microscope. Based on staining intensities and autofluorescence, tissues were qualitatively graded into four categories as shown in Table 8 and FIG. 27E. FFPE blocks belonging to the top two quality categories were included for the study.

TABLE 8
Tissue Grading

| | Signal for | |
|---|---|---|
| | CK8-Al488 + | Signal for |
| Tissue | Ck18-Al488 | pSTAT3 in |
| category | in epithelial cells | endothelial cells |
| 1 | High | High |
| 2 | High | Low |
| 3 | Low | Low/Absent |
| 4 | Absent | Absent |

### Definitions

High—bright fluorescent staining. Uniform for CK8 and 18
Low—barely visible staining, partially at background level
Absent—no staining observed

Cell Line Controls

Selected cell lines to be used as positive and negative controls were grown under standard conditions and treated with drugs and inhibitors before harvesting as indicated (Table 10). Cells were washed with PBS, fixed directly on plates with 10% formalin for 5 min, then scraped and collected into PBS. Next, cells were washed twice with phosphate buffered saline (PBS), resuspended in Histogel (Thermo Scientific, Waltham, Mass.) at 70° C., and spun for 5 minutes (10,000 g) to form a condensed cell-Histogel pellet. Pellets were embedded in paraffin, placed into standard paraffin blocks, and used as donor blocks for tumor microarray construction.

Generation of Tumor Microarray (TMA) Blocks

TMA blocks were prepared using a modified agarose block procedure¹⁵. Briefly, 0.7% agarose blocks were embedded into paraffin and used as TMA acceptor blocks. Using a TMA Master (3DHistech, Budapest, Hungary) instrument, two 1 mm diameter cores were drilled into donor blocks from areas corresponding to the highest Gleason pattern according to pathologist annotation. One of these cores was placed in a randomized position in one acceptor block while the position of the other core in a second acceptor block was randomized relative to the first core. This was repeated with 91, 170 and 157 annotated prostate tumor samples (Table 9) to form 3 pairs of TMA blocks (MPTMAF1A and 1B, 2A and 2B, 3A and 3B) respectively. The resulting paired blocks were identical in terms of patient sample composition but randomized in terms of sample position. Cell line control cores were added to top, middle and bottom portions of these acceptor blocks. Once loaded, TMA blocks were placed face down on glass slides at 65° C. for 15 min to enable fusion of TMA cores into host paraffin. Paraffin blocks were then cut into 5 μm serial sections. A smaller test TMA was generated from commercially available FFPE prostate tumor cases with only limited (Gleason score) annotation. This TMA was used to compare PTEN values with phosphomarkers prior to the main cohort study and to confirm reproducibility. Reproducibility was demonstrated by comparing individual marker signals on consecutive sections of the test TMA (Table 9 and FIG. 27F).

TABLE 9
TMA Maps
Block MPTMAF1a

| | | | | | | | | |
|---|---|---|---|---|---|---|---|---|
| 10 | 11 | 12 | 13 | 14 | 15 | 16 | 17 | 18 |
| 19 | 20 | 21 | 22 | 23 | 24 | 25 | 26 | 27 |
| 28 | 29 | 30 | 31 | 32 | 33 | 34 | 35 | 36 |
| 37 | 38 | 39 | 40 | 41 | 42 | 43 | 44 | 45 |
| 46 | 47 | 48 | 49 | 50 | 51 | 52 | 53 | 54 |
| 55 | 56 | 57 | 58 | 59 | 60 | 61 | 62 | 63 |
| 64 | 65 | 66 | 67 | 68 | 69 | 70 | 71 | 72 |
| 73 | 74 | 75 | 76 | 77 | 78 | 79 | 80 | 81 |
| 82 | 83 | 84 | 85 | 86 | 87 | 88 | 89 | 90 |
| 91 | 92 | 93 | 94 | 95 | 96 | 97 | 98 | 99 |
| 100 | Mcell- | Mcell- | Mcell- | Mcell- | Mcell- | Mcell- | Mcell- | Mcell- |
| | 042 | 043 | 047 | 044 | 046 | 045 | 049 | 055 |
| Mcell- | Mcell- | Mcell- | Mcell- | Mcell- | | | | |
| 050 | 051 | 056 | 048 | 057 | | | | |

Block MPTMAF1B

| | | | | | | | | |
|---|---|---|---|---|---|---|---|---|
| 10 | 11 | 12 | 13 | 14 | 15 | 16 | 17 | 18 |
| 19 | 20 | 21 | 22 | 23 | 24 | 25 | 26 | 27 |
| 28 | 29 | 30 | 31 | 32 | 33 | 34 | 35 | 36 |
| 37 | 38 | 39 | 40 | 41 | 42 | 43 | 44 | 45 |
| 46 | 47 | 48 | 49 | 50 | 51 | 52 | 53 | 54 |
| 55 | 56 | 57 | 58 | 59 | 60 | 61 | 62 | 63 |
| 64 | 65 | 66 | 67 | 68 | 69 | 70 | 71 | 72 |
| 73 | 74 | 75 | 76 | 77 | 78 | 79 | 80 | 81 |
| 82 | 83 | 84 | 85 | 86 | 87 | 88 | 89 | 90 |
| 91 | 92 | 93 | 94 | 95 | 96 | 97 | 98 | 99 |
| 100 | Mcell- | Mcell- | Mcell- | Mcell- | Mcell- | Mcell- | Mcell- | Mcell- |
| | 042 | 043 | 047 | 044 | 046 | 045 | 049 | 055 |
| Mcell- | Mcell- | Mcell- | Mcell- | Mcell- | | | | |
| 050 | 051 | 056 | 048 | 057 | | | | |

Block MPTMAF2A

| | | | | | | | | | | | |
|---|---|---|---|---|---|---|---|---|---|---|---|
| Mcell- | 130 | Mcell- | 323 | Mcell- | 400 | Mcell- | 276 | Mcell- | 420 | Mcell- | Mcell- |
| 042 | | 043 | | 047 | | 049 | | 050 | | 056 | 048 |
| 138 | Mcell- | 327 | Mcell- | 248 | Mcel- | 315 | Mcell- | 443 | Mcell- | 296 | Mcell- |
| | 044 | | 046 | | 045 | | 055 | | 051 | | 057 |
| 124 | 131 | 347 | 243 | 215 | 345 | 433 | 348 | 379 | 246 | 240 | 263 |
| 226 | 319 | 180 | 417 | 453 | 336 | 109 | 256 | 367 | 267 | 203 | 122 |
| 370 | 115 | 282 | 150 | 297 | 356 | 423 | 291 | 112 | 451 | 114 | 197 |
| 172 | 158 | 199 | 338 | 148 | 376 | 257 | 395 | 229 | 274 | 154 | 209 |
| 162 | 133 | 174 | 269 | 314 | 146 | 374 | 431 | 294 | 361 | 141 | 202 |
| 349 | Mcell- | 334 | Mcell- | 411 | Mcell- | 301 | Mcell- | 307 | Mcell- | 333 | Mcell- |
| | 042 | | 047 | | 046 | | 049 | | 050 | | 056 |
| Mcell- | 389 | Mcell- | 237 | Mcell- | 232 | Mcel- | 279 | Mcell- | 448 | Mcell- | Mcell- |
| 057 | | 043 | | 044 | | 045 | | 055 | | 051 | 048 |
| 242 | 212 | 249 | 450 | 143-2 | 205 | 128-2 | 344 | 186 | 401 | 455 | 381 |
| 273 | 357 | 183 | 328 | 445 | 457 | 136-1 | 129 | 217-2 | 308 | 305 | 383 |
| 214 | 350 | 117 | 375 | 173 | 452 | 325 | 234 | 275 | 449 | 119 | 371 |
| 454-2 | 346 | 166 | 372 | 321-1 | 225 | 144 | 369 | 413-1 | 107 | 368 | 447 |
| 285 | 439-2 | 181 | 380 | 298 | 385 | 386 | 391 | 409 | 251 | 425-2 | 306 |
| 272 | 429 | 382 | 288 | 434 | 283 | 394 | 277 | 330 | 182 | 177 | 387 |
| 281 | 116 | 142 | 427 | 260 | 218 | 165 | 120-1 | 247 | 309 | 262 | 456 |
| Mcell- | 343 | Mcell- | 157 | Mcell- | 377 | Mcell- | 223 | Mcell- | Mcell- | Mcell- | Mcell- |
| 042 | | 047 | | 046 | | 049 | | 050 | 048 | 056 | 057 |
| Empty | Mcell- | Empty | Mcell- | Empty | Mcell- | Empty | Mcell- | Empty | Mcell- | Empty | Empty |
| | 043 | | 044 | | 045 | | 055 | | 051 | | |

Block MPTMAF2B

| | | | | | | | | | | | |
|---|---|---|---|---|---|---|---|---|---|---|---|
| Mcell- | 420 | Mcell- | 240 | Mcell- | 371 | Mcell- | 395 | Mcell- | 298 | Mcell- | Mcell- |
| 042 | | 043 | | 047 | | 049 | | 050 | | 056 | 048 |
| 350 | Mcell- | 209 | Mcell- | 162 | Mcel- | 377 | Mcell- | 457 | Mcell- | 370 | Mcell- |
| | 044 | | 046 | | 045 | | 055 | | 051 | | 057 |
| 411 | 177 | 174 | 413-1 | 314 | 183 | 246 | 275 | 256 | 223 | 431 | 429 |
| 131 | 307 | 217-2 | 276 | 387 | 327 | 451 | 450 | 237 | 130 | 165 | 348 |
| 242 | 157 | 417 | 452 | 202 | 383 | 356 | 439-2 | 248 | 234 | 197 | 306 |
| 251 | 323 | 367 | 205 | 296 | 136-1 | 120-1 | 154 | 445 | 338 | 119 | 186 |
| 269 | 142 | 301 | 225 | 173 | 148 | 455 | 212 | 291 | 381 | 109 | 277 |
| 454-2 | Mcell- | 386 | Mcell- | 345 | Mcell- | 391 | Mcell- | 344 | Mcell- | 319 | Mcell- |
| | 042 | | 047 | | 046 | | 049 | | 050 | | 056 |
| Mcell- | 226 | Mcell- | 172 | Mcell- | 330 | Mcel- | 368 | Mcell- | 199 | Mcell- | Mcell- |
| 057 | | 043 | | 044 | | 045 | | 055 | | 051 | 048 |
| 443 | 380 | 379 | 144 | 243 | 288 | 114 | 325 | 433 | 456 | 112 | 334 |
| 232 | 294 | 267 | 449 | 249 | 263 | 305 | 336 | 328 | 257 | 375 | 349 |
| 124 | 425-2 | 141 | 229 | 448 | 262 | 133 | 321-1 | 357 | 400 | 382 | 453 |
| 158 | 214 | 394 | 389 | 297 | 129 | 347 | 218 | 146 | 374 | 150 | 285 |
| 272 | 273 | 138 | 385 | 423 | 117 | 401 | 333 | 215 | 427 | 361 | 409 |
| 369 | 122 | 447 | 282 | 315 | 247 | 115 | 128-2 | 281 | 203 | 308 | 143-2 |
| 434 | 166 | 376 | 181 | 107 | 279 | 260 | 346 | 274 | 283 | 180 | 372 |
| Mcell- | 116 | Mcell- | 182 | Mcell- | 309 | Mcell- | 343 | Mcell- | Mcell- | Mcell- | Mcell- |
| 042 | | 047 | | 046 | | 049 | | 050 | 048 | 056 | 057 |
| Empty | Mcell- | Empty | Mcell- | Empty | Mcel- | Empty | Mcell- | Empty | Mcell- | Empty | Empty |
| | 043 | | 044 | | 045 | | 055 | | 051 | | |

Block MPTMAF3A

| | | | | | | | | | | | |
|---|---|---|---|---|---|---|---|---|---|---|---|
| Mcell- | 236-1 | Mcell- | 179 | Mcell- | 222 | Mcell- | 426 | Mcell- | 341 | Mcell- | Mcell- |
| 042 | | 047 | | 046 | | 049 | | 050 | | 056 | 048 |
| 266 | Mcell- | 290 | Mcell- | 360 | Mcell- | 362 | Mcell- | 313 | Mcell- | 384 | Mcell- |
| | 043 | | 044 | | 045 | | 055 | | 051 | | 057 |
| 139 | 331 | 378 | 118 | 312 | 134 | 268 | 168 | 340 | 163 | 196 | 137 |
| 446 | 329 | 310 | 259 | 198 | 161 | 176 | 460 | 504 | 185 | 489 | 280 |
| 264 | 169 | 365 | 482 | 265 | 473-1 | 363 | 151 | 359 | 502 | 494 | 352 |
| 175 | 326 | 200 | 479 | 278 | 125 | 497 | 220 | 245-1 | 184 | 481-1 | 469 |
| 147 | 155 | 432-3 | 424 | 508 | 219 | 471 | 398 | 464 | 492 | 414-2 | 465 |
| 201 | 422 | 238 | 252 | 505 | 160 | 355 | 470 | 399-2 | 339 | 438-2 | 474 |
| Mcell- | 484 | Mcell- | 178 | Mcell- | 300 | Mcell- | 250 | Mcell- | 461 | Mcell- | Mcell- |
| 042 | | 047 | | 046 | | 049 | | 050 | | 056 | 048 |
| 407 | Mcell- | 303 | Mcell- | 351 | Mcell- | 402 | Mcell- | 503 | Mcell- | 270 | Mcell- |
| | 043 | | 044 | | 045 | | 055 | | 051 | | 057 |
| 153 | 480 | 487 | 358 | 472 | 293 | 113 | 145 | 430 | 261 | 416 | 292 |
| 506 | 410 | 126 | 228 | 123 | 459 | 495 | 501 | 289 | 437 | 488-2 | 441 |
| 302 | 132 | 418 | 188 | 498 | 486-2 | 404 | 317 | 191 | 481-2 | 463 | 170 |
| 189 | 373 | 396 | 490 | 477 | 187 | 299 | 467 | 311 | 241 | 318 | 354 |
| 476 | 156 | 316 | 366 | 507 | 108 | 444 | 287 | 193 | 392 | 254 | 500 |
| 227 | 195 | 493 | Empty | Empty | Empty | Empty | Empty | Empty | Empty | Empty | Empty |
| Mcell- | Empty | Mcell- | Empty | Mcell- | Empty | Mcell- | Empty | Mcell- | Mcell- | Mcell- | Mcell- |
| 042 | | 047 | | 046 | | 049 | | 050 | 048 | 056 | 057 |
| Empty | Mcell- | Empty | Mcell- | Empty | Mcell- | Empty | Mcell- | Empty | Mcell- | Empty | Empty |
| | 043 | | 044 | | 045 | | 055 | | 051 | | |

Block MPTMAF3B

| | | | | | | | | | | | |
|---|---|---|---|---|---|---|---|---|---|---|---|
| Mcell- | 118 | Mcell- | 469 | Mcell- | 505 | Mcell- | 507 | Mcell- | 201 | Mcell- | Mcell- |
| 042 | | 047 | | 046 | | 049 | | 050 | | 056 | 048 |
| 278 | Mcell- | 341 | Mcell- | 156 | Mcell- | 352 | Mcell- | 318 | Mcell- | 176 | Mcell- |
| | 043 | | 044 | | 045 | | 055 | | 051 | | 057 |
| 184 | 303 | 151 | 474 | 424 | 280 | 396 | 503 | 479 | 238 | 265 | 193 |
| 326 | 270 | 290 | 191 | 178 | 268 | 410 | 384 | 287 | 293 | 137 | 153 |
| 139 | 227 | 399-2 | 378 | 404 | 329 | 407 | 505 | 163 | 302 | 222 | 426 |
| 472 | 502 | 355 | 477 | 489 | 495 | 464 | 497 | 354 | 365 | 123 | 147 |
| 259 | 312 | 500 | 175 | 261 | 236-1 | 292 | 487 | 362 | 488-2 | 245-1 | 161 |
| 441 | 470 | 473-1 | 299 | 155 | 463 | 494 | 438-2 | 126 | 264 | 392 | 250 |
| Mcell- | 170 | Mcell- | 132 | Mcell- | 430 | Mcell- | 108 | Mcell- | 340 | Mcell- | Mcell- |
| 042 | | 047 | | 046 | | 049 | | 050 | | 056 | 048 |
| 311 | Mcell- | 506 | Mcell- | 196 | Mcell- | 317 | Mcell- | 366 | Mcell- | 198 | Mcell- |
| | 043 | | 044 | | 045 | | 055 | | 051 | | 057 |
| 465 | 486-2 | 168 | 501 | 200 | 228 | 310 | 373 | 113 | 359 | 444 | 422 |
| 484 | 220 | 476 | 169 | 460 | 418 | 480 | 313 | 490 | 339 | 241 | 331 |
| 508 | 179 | 358 | 481-1 | 467 | 482 | 195 | 351 | 504 | 185 | 402 | 498 |
| 316 | 363 | 145 | 160 | 360 | 481-2 | 432-3 | 125 | 188 | 266 | 493 | 446 |
| 471 | 254 | 134 | 461 | 300 | 398 | 187 | 414-2 | 416 | 219 | 252 | 492 |
| Empty | 289 | 437 | 459 | Empty | 189 | Empty | Empty | Empty | Empty | Empty | Empty |
| Mcell- | Empty | Mcell- | Empty | Mcell- | Empty | Mcell- | Empty | Mcell- | Mcell- | Mcell- | Mcell- |
| 042 | | 047 | | 046 | | 049 | | 050 | 048 | 056 | 057 |
| Empty | Mcell- | Empty | Mcell- | Empty | Mcell- | Empty | Mcell- | Empty | Mcell- | Empty | Empty |
| | 043 | | 044 | | 045 | | 055 | | 051 | | |

Block MPTMA 1

| | | | | | | | |
|---|---|---|---|---|---|---|---|
| ILS28857 | I-7644 | I-7653 | I-7663 | I-7666 | I-3219 | I-3219 | I-6012 |
| I-6018 | I-3220 | I-3221 | I-3223 | I-6016 | I-6016 | I-3218 | I-3218 |
| I-7648 | I-7648 | I-7649 | I-7650 | I-7659 | ILS22883 | ILS28720 | ILS25346 |
| ILS23325 | ILS23330 | ILS28870 | ILS22171 | ILS28878 | ILS23426 | ILS25693 | ILS25693 |
| ILS28848 | ILS23510 | ILS23510 | ILS23659 | ILS23659 | ILS28860 | ILS28860 | ILS24983 |
| ILS28857 | I-7644 | I-7653 | I-7663 | I-7666 | I-3219 | I-3219 | I-6012 |
| I-6018 | I-3220 | I-3221 | I-3223 | I-6016 | I-6016 | I-3218 | I-3218 |
| I-7648 | I-7648 | I-7649 | I-7650 | I-7659 | ILS22883 | ILS28720 | ILS25346 |
| ILS23325 | ILS23330 | ILS28870 | ILS22171 | ILS28878 | ILS23426 | ILS25693 | ILS25693 |
| ILS28848 | ILS23510 | ILS23510 | ILS23659 | ILS23659 | ILS28860 | ILS28860 | ILS24983 |

Slide Processing and Quantitative Multiplex Immunofluorescence (QMIF) Staining Protocol

TMA sections were cut at 5 urn thickness and placed on Histogrip (LifeTechnologies, Grand Island, N.Y.) coated slides. Slides were baked at 65° C. for 30 min, deparaffinized through serial incubations in xylene, and rehydrated through a series of graded alcohols. Antigen retrieval was performed in 0.05% citraconic anhydride solution for 45 min at 95° C. using a PT module (Thermo Scientific, Waltham, Mass.). Autostainers 360 and 720 (Thermo Scientific, Waltham, Mass.) were used for staining.

The staining procedure involved two blocking steps followed by four incubation steps with appropriate washes in between. Blocking consisted of a biotin step followed by Sniper reagent (Biocare Medical, Concord, Calif.). The first incubation step included anti-biomarker 1 mouse mAb and anti-biomarker 2 rabbit mAb. The second step included anti-rabbit IgG Fab-FITC and anti-mouse IgG Fab-biotin, followed by a third “visualization” step that included anti-FITC MAb-Alexa568, streptavidin-Alexa633 and fluorophor-conjugated region of interest antibodies (anti-CK8-Alexa488, anti-CK18-Alexa488, anti-CK5-Alexa555 and anti-Trim29-Alexa555). Finally, sections were incubated with DAPI for nuclear staining (for a staining format outline, see FIG. 27B). Slides were mounted with ProlongGold (LifeTechnologies, Grand Island, N.Y.) and coverslipped. Slides were kept at −20° C. overnight before imaging and for long-term storage. A full set of 6 MPTMAF slides were stained in a single staining session for the various antibody combinations encompassing all biomarkers tested.

Antibody Validation

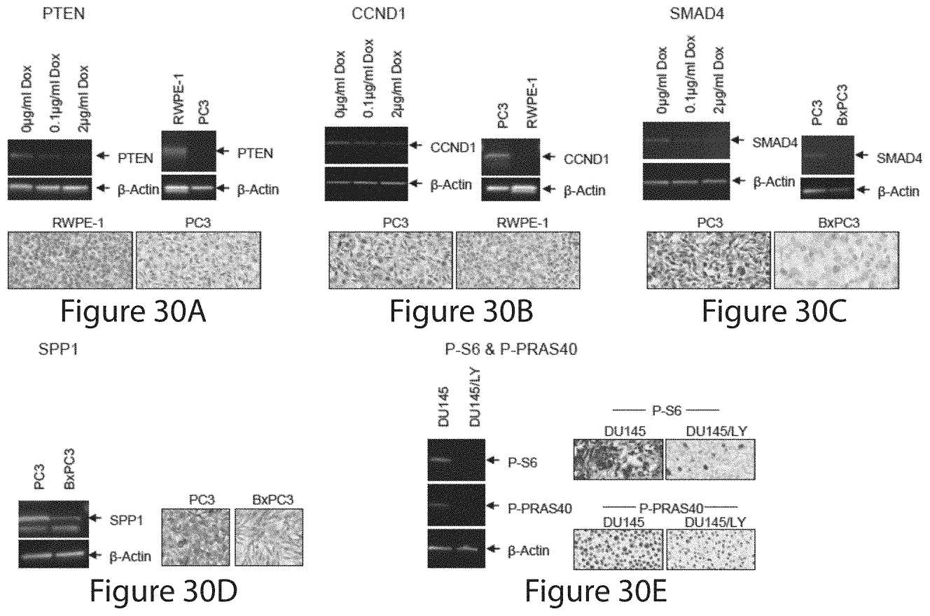

Testing by Western Blotting before and after knock down: To test specificity of mAbs against PTEN, SMAD4 and CCND1, we employed inducible shRNA knockdown of the protein marker of interest. Briefly, DU145 cells with inducible shRNA were generated by transducing naïve DU145 cells with a virus carrying pTRIPZ (Thermo Scientific, Waltham, Mass.). Cells were stably selected using 2 μg/ml puromycin for a week. Subsequently, cells were induced with either 0.1 μg/ml or 2 μg/ml of doxycycline for 72 hours. Cells were trypsinized and processed either for RNA extraction or cell lysate generation. The best shRNA for each protein marker was confirmed first by RT-PCR and then by Western blot. Antibodies were considered specific when the expected molecular band size decreased upon shRNA induction on Western blot. To test mAb against SPP1, we used cell lines with high or low SPP expression. Lysates from these cell lines (as shown in FIG. 30) were also used for Western blotting. To test anti-phospho antibodies against members of the AKT signaling pathway, DU145 cells were serum starved overnight, treated with the PI3K inhibitor LY294002 at 10 μM for 2 hours, and lysed. Lysates from cells treated with inhibitor were used as negative controls for Western blots; lysates from cells grown in standard conditions were used as positive controls.

20 μg of cell lysates were run on a 4-15% Criterion TGX precast gel (Bio-Rad, Hercules, Calif.). Afterwards, the gel was transferred onto nitrocellulose membrane using iBlot (LifeTechnologies, Grand Island, N.Y.). The primary antibody dilution was used according to product data sheet recommendation. The membrane was developed using SuperSignal West Femto Maximum Sensitivity Substrate (Thermo Scientific, Waltham, Mass.). Images were captured using the FluroChem Q system (Protein Simple, Santa Clara, Calif.). Images were processed using AlphaView (Protein Simple, Santa Clara, Calif.) and ImageJ¹⁶

Testing by Immunohistochemistry before and after target knock down: FFPE cell pellets from cell lines treated as described above were assembled together in a TMA block. 5 μm sections were cut and dried at 60° C. for an hour before de-paraffinization in three changes of Xylene and rehydration in a series of descending Ethanol washes. The slides were heated in 0.05% Citraconic Anhydride (Sigma, Saint Louis, Mo.) at 95° C. for 40 min for antigen retrieval. Slides were stained using the Lab Vision™ UltraVision™ LP Detection System: HRP Polymer/DAB Plus Chromogen Kit (Thermo Scientific, Waltham, Mass.) as per manufacturer's instructions. Slides were scanned with an Aperio Scanscope AT Turbo system (Aperio, Vista, Calif.). Images were analyzed with Aperio ImageScope software (Aperio, Vista, Calif.).

Image Acquisition

Two Vectra Intelligent Slide Analysis Systems (Perkin-Elmer, Waltham, Mass.) were used for automated image acquisition. DAPI, FITC, TRITC and Cy5 long pass filter cubes were optimized to allow maximum spectral resolution and minimize cross-interference between fluorophores. Vectra 2.0 and Nuance 2.0 software packages (Perkin Elmer, Waltham, Mass.) were used for automated image acquisition and development of the spectral library, respectively.

TMA acquisition protocols were run in an automated mode according to manufacturer instructions (Perkin-Elmer, Waltham, Mass.). Two 20× fields per core were imaged using a multispectral acquisition protocol that included consecutive exposures with DAPI, FITC, TRITC and Cy5 filters. To ensure reproducibility of biomarker quantification, light source intensity was calibrated with the X-Cite Optical Power Measurement System (Lumen Dynamics, Mississauga, ON, Canada) prior to image acquisition for each TMA slide. Identical exposure times were used for all slides containing the same antibody combination. To minimize intra-experiment variability, TMA slides stained with the same antibody combinations were imaged on the same Vectra microscope.

A spectral profile was generated for each fluorescent dye as well as for FFPE prostate tissue autofluorescence. Interestingly, two types of autofluorescence were observed in FFPE prostate tissue. A typical autofluorescence signal was common in both benign and tumor tissue, whereas atypical “bright” autofluorescence was specific for bright granules present mostly in epithelial cells of benign tissue. A spectral library containing a combination of these two spectral profiles was used to separate or “unmix” individual dye signals from autofluorescent background (FIG. 27A and FIG. 27C).

Image Analysis

We developed an automated image analysis algorithm using Definiens Developer XD (Definiens AG, Munich, Germany) for tumor identification and biomarker quantification. For each 1.0 mm TMA core, two 20× image fields were acquired. Vectra multispectral image files were first converted into multilayer TIFF format using inForm (PerkinElmer, Waltham, Mass.) and a customized spectral library, then converted to single layer TIFF files using BioFormats (OME¹⁷). Single layer TIFF files were imported into the Definiens workspace using a customized import algorithm so that for each TMA core, both of the image field TIFF files were loaded and analyzed as “maps” within a single “scene”.

Autoadaptive thresholding was used to define fluorescent intensity cut-offs for tissue segmentation in each individual tissue sample. Tissue samples were segmented using DAPI along with fluorescent epithelial and basal cell markers to allow classification as epithelial cells, basal cells and stroma, and were further compartmentalized into cytoplasm and nuclei. Benign prostate glands contain basal cells and luminal cells, whereas prostate cancer glands lack basal cells and have smaller luminal profiles. Therefore, individual gland regions were classified as malignant or benign based on the relational features between basal cells and adjacent epithelial structures combined with object-related features, such as gland size (see FIG. 27D). Fields with artifactual staining, insufficient epithelial tissue or out-of-focus images were removed prior to scoring.

Epithelial marker and DAPI intensities were quantitated in benign and malignant epithelial regions as quality control measurements. Biomarker intensity levels were measured in the cytoplasm, nucleus or whole cancer cell based on predetermined subcellular localization criteria. Mean biomarker pixel intensity in the cancer compartments was averaged across maps with acceptable quality parameters to yield a single value for each tissue sample and cell line control core.

Patient Cohort Composition

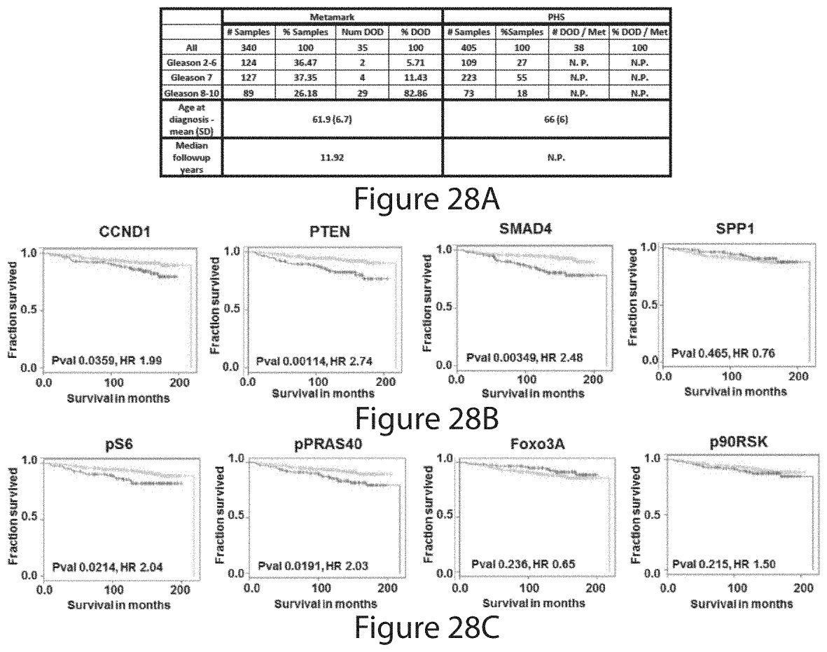

FIG. 28A describes the FOLIO cohort composition used in the current study and includes a comparison with the PHS cohort⁸.

Marker Value Determination

As each sample was represented by two cores, we generated an aggregate score for each marker based on correlation direction. For markers correlated positively with lethality we used the core with the highest value; for negatively-correlated markers we used the core with the lowest value. For example, for the tumor suppressor SMAD4, which was present on all stained sections, we used the lowest core value for the three cores.

Univariate Analyses

Univariate cox models were trained for each biomarker. For each marker, the hazard ratio and log rank p-value were calculated to compare the populations consisting of the top one-third and bottom two-thirds of the risk scores for positively correlated markers, and populations consisting of the bottom one-third and top two-thirds of risk scores for negatively correlated markers (FIGS. 28B and C).

Multivariate Analyses

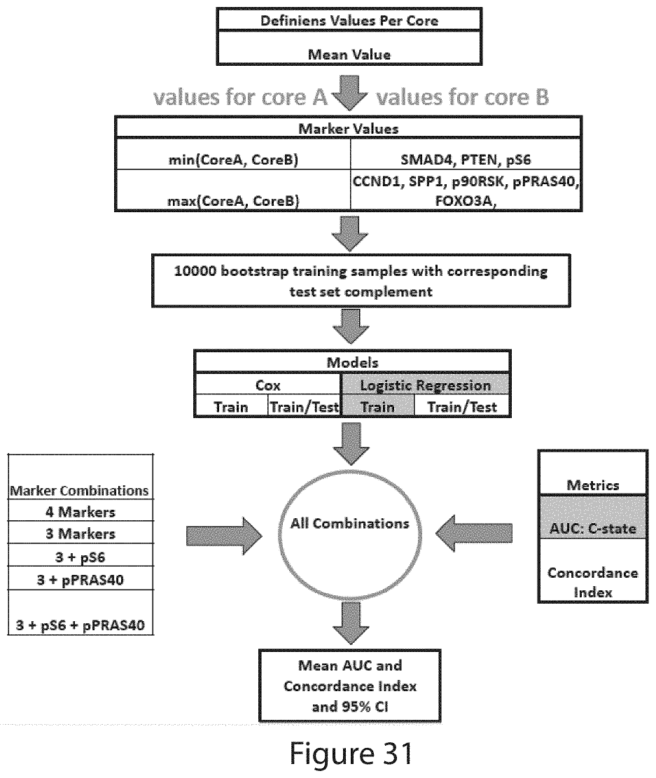

We used multivariate analyses to determine the ability of the marker set to predict lethal outcome. We leveraged two modeling approaches and two metrics. Specifically, 10,000 bootstrap training samples were generated, and both multivariate Cox models and logistic regression models were trained on each training sample. Testing was performed on the complement set. Concordance index (CI) and area under the curve (AUC) were used to estimate model performance. Kaplan-Meyer curves were generated to compare the population with the bottom two-thirds of risk scores to the population with the top one-third of risk scores. Receiver operating characteristic (ROC) curves were generated for the whole cohort based on the risk scores from the logistic regression model. The marker combinations tested in our models were as follows: 1) PTEN, SMAD4, CCND1 and SPP1, and 2) SMAD4, CCND1, SPP1 and one of the following combinations of the phospho markers: pS6, pPRAS40, and pS6+pPRAS40. FIG. 31 presents an outline of the multivariate analysis approaches.

Results

Platform Development

Developing an automated multiplex proteomics imaging platform required meeting a number of technical requirements: 1) ability to quantitate multiple markers in a defined region of interest (i.e. in tumor versus surrounding benign tissue), 2) rigorous tissue quality controls, 3) balanced multiplex assay staining format, and 4) experimental reproducibility.

To address the first, we optimized long-pass DAPI, FITC, TRITC and Cy5 filter sets to have sufficient excitation energy and emission bandpass with minimal interference between channels. We further separated biomarker signals from endogenous autofluorescence through spectral unmixing of images (FIG. 27A;¹⁸). In order to quantitatively measure biomarkers in tumor epithelium only, we needed to achieve “tissue segmentation”, distinguishing tumor from benign areas. Segmentation was achieved using a combination of feature extraction and protein co-localization algorithms. Total epithelium was stained using Alexa488 conjugated anti-CK8 and CK18 antibodies, while Alexa555 conjugated anti-CK5 and Trim29 antibodies stained basal epithelium^{19,20} Using automated Definiens (Munich, Germany) image analysis, epithelial structures with an outer layer of basal cells were considered benign, while those lacking basal cells were considered cancer²⁰. Non-epithelial areas were considered stroma. Ultimately, quantitative biomarker values were extracted only from cancer epithelium (the ‘region of interest’; FIG. 27B-D).

To evaluate tissue sample quality for study inclusion, we assessed staining intensities of several protein markers in benign tissue. Examination of a large number of prostate tissue blocks of variable quality revealed that Cytokeratin 8 and 18 and pSTAT3 intensities in benign epithelial regions and capillary endothelium, respectively, varied from ‘high’ to ‘low’ or ‘absent’ (data not shown; Massimo Loda, personal communication). On this basis, we categorized formalin-fixed, paraffin-embedded (FFPE) prostate cancer tissue blocks into four quality groups (FIG. 27E and Table 8). Only blocks from the best two groups were used to generate tumor microarray blocks (TMA), thereby controlling for biospecimen degradation and variability due to pre-analytic variation²¹⁻²³. In total, we procured and tested 508 unique prostatectomy samples with lethal outcome annotation available (Folio Biosciences, Powell, Ohio). Of these, 418 passed quality testing and were used for our TMA (Table 10).

TABLE 10
Cell line controls

| | Block ID | Cell Line | shRNA Knockdown or treatment |
|---|---|---|---|
| 1 | MCELL-11- | DU-145 | None |
| | 042 | | |
| 2 | MCELL-11- | PC-3 | None |
| | 043 | | |
| 4 | MCELL-11- | WM266-4 | None |
| | 044 | | |
| 6 | MCELL-11- | RPMI7951 | None |
| | 045 | | |
| 5 | MCELL-11- | BxPC-3 | None |
| | 046 | | |
| 3 | MCELL-11- | RWPE-1 | None |
| | 047 | | |
| 13 | MCELL-11- | SK-MEL-5 | None |
| | 048 | | |
| 7 | MCELL-11- | DU-145 | LY treated for 1 hr |
| | 049 | | |
| 9 | MCELL-11- | DU-145 | Smad4 knockdown, 4H1, 0 ug/ml Dox |
| | 050 | | |
| 10 | MCELL-11- | DU-145 | Smad4 knockdown, 4H1, 1 ug/ml Dox |
| | 051 | | |
| 8 | MCELL-11- | PC-3 | LY treated for 1 hr |
| | 055 | | |
| 11 | MCELL-11- | DU-145 | CCND1/, Dox 0 μg/ml |
| | 056 | | |
| 12 | MCELL-11- | DU-145 | CCND1/, Dox 1 μg/ml |
| | 057 | | |

To balance biomarker signal levels in our multiplex assay format, proteins with high expression levels, like cytokeratins and Trim29 were visualized with directly conjugated antibodies, while biomarkers with lower expression levels required signal amplification through use of secondary and tertiary antibodies. Using a test prostate TMA containing low- and high-grade tumor material, dilutions of each antibody were optimized to minimize background and maximize specificity, and to ensure a dynamic range of at least 3-fold difference between low and high signal values (FIG. 28B). Signals from consecutive TMA sections showed high reproducibility with typical R² correlation values above 0.9 and differences in absolute values typically less than 10% (FIG. 28B and data not shown).

Ability to Predict Lethal Outcome

We tested the platform using a four-protein signature reported in a recent study published by Ding et al⁸. Using a TMA comprised of 405 cases derived from the Physician's Health Study (PHS), the authors had demonstrated that a multivariate model based on semi-quantitative, pathologist-evaluated protein levels of PTEN, SMAD4, CCND1 and SPP1 could predict lethal outcome. We asked whether we could predict lethal outcome by evaluating protein levels in an independent prostatectomy cohort using our automated platform instead of a pathologist. Out of the 418 qualified cases in our TMA, 340 were found useful for analysis, attrition primarily being due to cores displaced during sectioning (see FIG. 28A for cohort description and comparison with the PHS cohort). Quantitative tumor epithelium biomarker levels were extracted from each sample and values were subjected to univariate analyses. PTEN, SMAD4 and CCND1 were all found to be individually lethal outcome-predictive with hazard ratios (HRs) of 2.74, 2.48 and 1.99, respectively, while SPP1 did not have significant predictive performance (FIG. 28B).

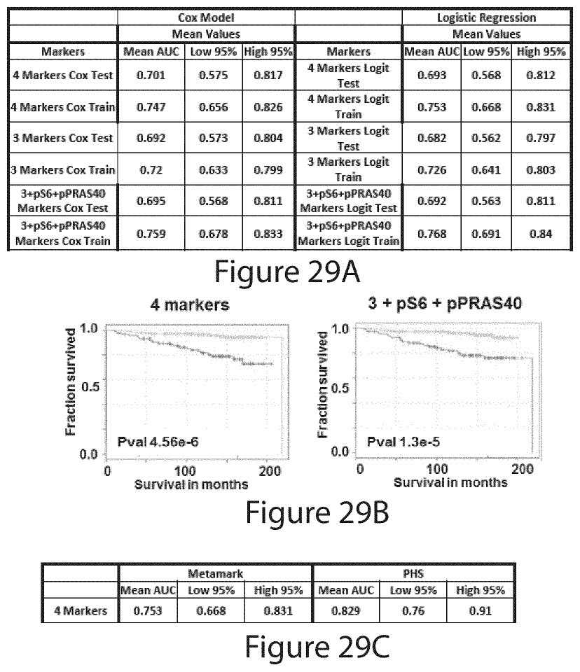

Next, multivariate Cox and logistic regression analyses were conducted. The performance of the four-marker model was determined as an area under the curve (AUC) and a concordance index (CI) (FIG. 29A and Table 11, respectively). For logistic regression analyses, cases were defined as patients that died from prostate cancer. The AUC was approximately 0.75 for the four markers in training mode, and 0.69 to 0.70 in test mode by logistic regression and Cox analyses, respectively (FIG. 29A). A Kaplan-Meier curve comparing the top one-third to bottom two-thirds of risk scores based on the four markers was generated by a Cox model trained on the whole cohort. This curve shows a clear survival difference between risk groups (FIG. 29B). FIG. 29B presents a comparison between our results and those of the PHS study. Our mean AUC of 0.75 [95% confidence interval (0.67, 0.83)] is comparable with performance of the PHS mean AUC of 0.83 [95% confidence interval (0.76, 0.91)]. Note the large overlap in confidence intervals.

TABLE 11
Concordance index

| | Cox Model | | | | Logistic Regression | | |
|---|---|---|---|---|---|---|---|
| | Mean | | | | Mean | | |
| | Concordance | Low | High | | Concordance | Low | High |
| Markers | Index | 95% | 95% | Markers | Index | 95% | 95% |
| 4 Markers Cox | 0.688 | 0.592 | 0.827 | 4 Markers Logit | 0.686 | 0.571 | 0.812 |
| Test | | | | Test | | | |
| 4 Markers Cox | 0.715 | 0.605 | 0.792 | 4 Markers Logit | 0.707 | 0.588 | 0.787 |
| Train | | | | Train | | | |
| 3 + pS6 + pPRAS40 | 0.693 | 0.57 | 0.807 | 3 + pS6 + pPRAS40 | 0.699 | 0.579 | 0.811 |
| Markers Cox | | | | Markers Logit | | | |
| Test | | | | Test | | | |
| 3 + pS6 + pPRAS40 | 0.759 | 0.685 | 0.829 | 3 + pS6 + pPRAS40 | 0.758 | 0.684 | 0.827 |
| Markers Cox | | | | Markers Logit | | | |
| Train | | | | Train | | | |

Incorporation of Protein Activity States as Part of Multivariate Signature

Since protein activity states reflect functional events in the tumors that are associated with aggressive behavior, we tested whether our platform could quantitatively measure not just protein levels but protein activity states as reflected by post-translational modifications or altered sub-cellular localization. Phosphorylation is a particularly well-studied example of post-translational modification; the stoichiometry of protein phosphorylation at a particular site is an indirect measure of the activity state of the parent signaling pathway^{24,25}. Specifically, we examined whether the activity state of one or more signaling molecules in the core PTEN-regulated signaling pathways PI3K/Akt and MAPK could substitute for PTEN in the four-marker model. PTEN protein, in contrast to the PI3K/AKT pathway, is only altered in a subset of prostate cancers^{11,26}, so our goal was to identify replacement phosphomarkers that could be more broadly informative about PI3K/Akt pathway activity states^{26,27}. To this end, we obtained a number of phospho-specific monoclonal antibodies (P-mAb) directed against key phosphoproteins and tested them for technical suitability (Table 7). Testing included specificity analysis through knock down in cell lines, signal intensity in human prostate cancer tissue, and, importantly, epitope stability^{23,27} based on signal preservation across prostate cancer FFPE samples (FIG. 30 and data not shown). We included phospho-markers because PI3K/AKT pathway activity is often independent of PTEN protein status^{12,13} Based on these criteria, the following phospho-specific antibodies were selected and tested for univariate and multivariate lethal outcome predictive performance: p90RSK-T359/5363, pPRAS40-T246, pS6-5235/236 and pGSK3-521/9 (Cell Signaling Technology, Danvers, Mass.;²⁷). We also selected an anti-Foxo3A antibody for testing since it is excluded from the nucleus when the PI3K pathway is activated²⁸. Markers were subjected to univariate analysis in a Kaplan-Meier plot. pPRAS40 and pS6 had significant univariate performance with HRs around 2 when comparing signal values of the top one-third to bottom two-thirds (FIG. 28C).

We then examined the performance of the four original markers without PTEN (FIG. 29A). The AUC (train) dropped from 0.75 to 0.72-0.73. Addition of pS6 (in essence substituting pS6 for PTEN) increased the CI and AUC to between 0.75 and 0.76, respectively, while substitution with pPRAS40 did not result in a significant increase of the AUC and CI (data not shown). Finally, substitution of PTEN with both pS6 and pPRAS40 increased AUC (train) values to between ˜0.76 and −0.77 (FIG. 29A). The corresponding Kaplan-Meier curve for the three markers together with pS6+pPRAS40 is shown in FIG. 29B. These results demonstrate that we can successfully replace PTEN, a known lethal outcome-predictive marker, with two phospho-markers, pS6 and pPRAS40, while maintaining the ability to predict lethal outcome.

### DISCUSSION

This work established an automated imaging platform that accurately and reproducibly integrates morphological and proteomic information. We assessed platform performance through direct comparison with a previous study by using the same 4 markers reported to predict lethal outcome. A simple meta-analysis of the two studies estimated a non-significant difference in mean AUC of 0.08 [95% confidence interval (−0.03, 0.19)]. Differences in performance may be due to methodological differences between the two studies. First, we used monoclonal antibodies validated for specificity through siRNA oligo-mediated knock down in Western blotting and immunohistochemistry (FIG. 30), while two of the antibodies used in the PHS study were polyclonal and thus not ideal for continued prospective application. Moreover, the quantitative measurements in this study were fully automated, while theirs relied on pathologist interpretation, and hence overall would be expected to be slightly less reproducible. Finally, our cohort included a higher proportion of Gleason ≤6 cases for which lethal outcome would be more difficult to predict than for higher grade cases and lethal outcome prediction was further limited by a median follow-up of 11.92 years which is not long enough to capture all deaths. Given these methodological distinctions and the assessment of difference in AUCs, our results are comparable, demonstrating for the usefulness of this fully automated platform and prognostication independent of human interpretation.

In embodiments featured herein, robust tissue segmentation algorithm and quantitative biomarker measurements are achieved in tumor epithelium regions by combining Vectra multispectral image decomposition with the programmable Definiens Tissue Developer. The methods provided herein provide an automated approach that is highly sensitive, operates without subjective intervention, and can successfully evaluate very small amounts of cancer tissue.

An important application of the present platform is the ability to incorporate protein activation states as biomarkers. It is demonstrated here that p-mAbs measuring activity states of signaling molecules in the core PI3K and MAPK pathways can substitute for PTEN, a highly outcome-predictive marker. The tumor suppressor PTEN is altered in only 15-20% of early stage prostate cancers, yet is often functionally inactivated through a variety of other mechanisms that would be reflected in altered PI3K/Akt pathway activityl². Without wishing to be bound by theory, it may be that PI3K/AKT pathway activity state measurements are more informative in early prostate cancer lesions than PTEN. We show here the lethal outcome predictive performance of a new five-marker signature for radical prostatectomy: SMAD4, CCND1, SPP1, pPRAS40 and pS6.

In summary, we have developed a multiplex proteomics in situ imaging platform with automated, objective biomarker measurements able to predict lethal outcome using prostatectomy tissue independent of pathologist interpretation. Importantly, we demonstrated the ability to incorporate quantitative measurements of protein activity states, as reflected by post-translational modifications, into a multivariate protein predictor of lethal outcome. This platform is broadly applicable across disease states. In particular, we have already applied it to develop a prognostic prostate cancer biopsy test for early stage lesions where tissue amounts are often limited.

### References Cited in Example 5

1. Hudson T J: Genome variation and personalized cancer medicine, J Intern Med 2013, 274:440-450
2. Liehr T, Weise A, Hamid A B, Fan X, Klein E, Aust N, Othman M A, Mrasek K, Kosyakova N: Multicolor FISH methods in current clinical diagnostics, Expert Rev Mol Diagn 2013, 13:251-255
3. Cheng S, Koch W H, Wu L: Co-development of a companion diagnostic for targeted cancer therapy, N Biotechnol 2012, 29:682-688
4. Maxwell K N, Domchek S M: Cancer treatment according to BRCA1 and BRCA2 mutations, Nature reviews Clinical oncology 2012, 9:520-528
5. Kwak E L, Bang Y J, Camidge D R, Shaw A T, Solomon B, Maki R G, Ou S H, Dezube B J, Janne P A, Costa D B, Varella-Garcia M, Kim W H, Lynch T J, Fidias P, Stubbs H, Engelman J A, Sequist L V, Tan W, Gandhi L, Mino-Kenudson M, Wei G C, Shreeve S M, Ratain M J, Settleman J, Christensen J G, Haber D A, Wilner K, Salgia R, Shapiro G I, Clark J W, lafrate AJ: Anaplastic lymphoma kinase inhibition in non-small-cell lung cancer, The New England journal of medicine 2010, 363:1693-1703
6. Cuzick J, Swanson G P, Fisher G, Brothman A R, Berney D M, Reid J E, Mesher D, Speights V O, Stankiewicz E, Foster C S, Moller H, Scardino P, Warren J D, Park J, Younus A, Flake D D, 2nd, Wagner S, Gutin A, Lanchbury J S, Stone S, Transatlantic Prostate G: Prognostic value of an RNA expression signature derived from cell cycle proliferation genes in patients with prostate cancer: a retrospective study, The lancet oncology 2011, 12:245-255
7. Kaklamani V: A genetic signature can predict prognosis and response to therapy in breast cancer: Oncotype DX, Expert Rev Mol Diagn 2006, 6:803-809
8. Ding Z, Wu C J, Chu G C, Xiao Y, Ho D, Zhang J, Perry S R, Labrot E S, Wu X, Lis R, Hoshida Y, Hiller D, Hu B, Jiang S, Zheng H, Stegh A H, Scott K L, Signoretti S, Bardeesy N, Wang Y A, Hill D E, Golub T R, Stampfer M J, Wong W H, Loda M, Mucci L, Chin L, DePinho R A: SMAD4-dependent barrier constrains prostate cancer growth and metastatic progression, Nature 2011, 470:269-273
9. McMenamin M E, Soung P, Perera S, Kaplan I, Loda M, Sellers W R: Loss of PTEN expression in paraffin-embedded primary prostate cancer correlates with high Gleason score and advanced stage, Cancer research 1999, 59:4291-4296
10. Yoshimoto M, Cunha I W, Coudry R A, Fonseca F P, Torres C H, Soares F A, Squire J A: FISH analysis of 107 prostate cancers shows that PTEN genomic deletion is associated with poor clinical outcome, British journal of cancer 2007, 97:678-685
11. Cuzick J, Yang Z H, Fisher G, Tikishvili E, Stone S, Lanchbury J S, Camacho N, Merson S, Brewer D, Cooper C S, Clark J, Berney D M, Moller H, Scardino P, Sangale Z: Prognostic value of PTEN loss in men with conservatively managed localised prostate cancer, British journal of cancer 2013, 108:2582-2589
12. Song M S, Salmena L, Pandolfi P P: The functions and regulation of the PTEN tumour suppressor, Nat Rev Mol Cell Biol 2012, 13:283-296
13. Yuan T L, Cantley L C: PI3K pathway alterations in cancer: variations on a theme, Oncogene 2008, 27:5497-5510
14. Epstein J I, Allsbrook W C, Jr., Amin M B, Egevad L L: The 2005 International Society of Urological Pathology (ISUP) Consensus Conference on Gleason Grading of Prostatic Carcinoma, Am J Surg Pathol 2005, 29:1228-1242
15. Yan P, Seelentag W, Bachmann A, Bosman F T: An agarose matrix facilitates sectioning of tissue microarray blocks, The journal of histochemistry and cytochemistry: official journal of the Histochemistry Society 2007, 55:21-24
16. Schneider C A, Rasband W S, Eliceiri K W: NIH Image to ImageJ: 25 years of image analysis, Nature methods 2012, 9:671-675
17. Linkert M, Rueden C T, Allan C, Burel J M, Moore W, Patterson A, Loranger B, Moore J, Neves C, Macdonald D, Tarkowska A, Sticco C, Hill E, Rossner M, Eliceiri K W, Swedlow J R: Metadata matters: access to image data in the real world, The Journal of cell biology 2010, 189:777-782
18. Mansfield J R, Hoyt C, Levenson R M: Visualization of microscopy-based spectral imaging data from multi-label tissue sections, Curr Protoc Mol Biol 2008, 14:
19. Kristiansen G: Diagnostic and prognostic molecular biomarkers for prostate cancer, Histopathology 2012, 60:125-141
20. Brimo F, Epstein J I: Immunohistochemical pitfalls in prostate pathology, Human pathology 2012, 43:313-324
21. Portier B P, Wang Z, Downs-Kelly E, Rowe J J, Patil D, Lanigan C, Budd G T, Hicks D G, Rimm D L, Tubbs R R: Delay to formalin fixation ‘cold ischemia time’: effect on ERBB2 detection by in-situ hybridization and immunohistochemistry, Modern pathology: an official journal of the United States and Canadian Academy of Pathology, Inc 2013, 26:1-9
22. Hicks D G, Boyce B F: The challenge and importance of standardizing pre-analytical variables in surgical pathology specimens for clinical care and translational research, Biotech Histochem 2012, 87:14-17
23. Holzer T R, Fulford A D, Arkins A M, Grondin J M, Mundy C W, Nasir A, Schade A E: Ischemic time impacts biological integrity of phospho-proteins in PI3K/Akt, Erk/MAPK, and p38 MAPK signaling networks, Anticancer research 2011, 31:2073-2081
24. Hunter T: Signaling—2000 and beyond, Cell 2000, 100:113-127
25. Blume-Jensen P, Hunter T: Oncogenic kinase signalling, Nature 2001, 411:355-365
26. Taylor B S, Schultz N, Hieronymus H, Gopalan A, Xiao Y, Carver B S, Arora V K, Kaushik P, Cerami E, Reva B, Antipin Y, Mitsiades N, Landers T, Dolgalev I, Major J E, Wilson M, Socci N D, Lash A E, Heguy A, Eastham J A, Scher H I, Reuter V E, Scardino P T, Sander C, Sawyers C L, Gerald W L: Integrative genomic profiling of human prostate cancer, Cancer cell 2010, 18:11-22
27. Andersen J N, Sathyanarayanan S, Di Bacco A, Chi A, Zhang T, Chen A H, Dolinski B, Kraus M, Roberts B, Arthur W, Klinghoffer R A, Gargano D, Li L, Feldman I, Lynch B, Rush J, Hendrickson R C, Blume-Jensen P, Paweletz C P: Pathway-based identification of biomarkers for targeted therapeutics: personalized oncology with PI3K pathway inhibitors, Science translational medicine 2010, 2:43ra55
28. Yang J Y, Hung M C: A new fork for clinical application: targeting forkhead transcription factors in cancer, Clinical cancer research: an official journal of the American Association for Cancer Research 2009, 15:752-757
29. Camp R L, Chung G G, Rimm D L: Automated subcellular localization and quantification of protein expression in tissue microarrays, Nature medicine 2002, 8:1323-1327
30. Donovan M J, Hamann S, Clayton M, Khan F M, Sapir M, Bayer-Zubek V, Fernandez G, Mesa-Tejada R, Teverovskiy M, Reuter V E, Scardino P T, Cordon-Cardo C: Systems pathology approach for the prediction of prostate cancer progression after radical prostatectomy, Journal of clinical oncology: official journal of the American Society of Clinical Oncology 2008, 26:3923-3929
31. Teverovskiy M, Vengrenyuk Y, Tabesh A, Sapir M, Fogarasi S, Ho-Yuen P, Khan F M, Hamann S, Capodieci P, Clayton M, Kim R, Fernandez G, Mesa-Tejada R, Donovan M J: Automated localization and quantification of protein multiplexes via multispectral fluorescence imaging. Edited by 2008, p. pp. 300-303

### Example 6: Identification and Clinical Assessment of Proteomic Biomarkers Predicting Prostate Cancer Aggressiveness Despite Biopsy Sampling Error

### SUMMARY

This study describes the identification and clinical evaluation of intact tissue protein biomarkers that are predictive of prostate cancer aggressiveness and lethal outcome despite sampling error.

Determination of prostate cancer aggressiveness and appropriate therapy are based on clinical pathological parameters, including biopsy Gleason grading and extent of tumor involvement, prostate-specific antigen (PSA) levels, and patient age. Key challenges for prediction of tumor aggressiveness based on biopsy Gleason grading include heterogeneity of prostate cancer, biopsy-sampling error, and variations in biopsy interpretation. The resulting uncertainty in risk assessment leads to significant over-treatment, with associated costs and morbidity. We developed a performance-based strategy to identify protein biomarkers tailored to more accurately reflect true prostate cancer aggressiveness despite biopsy sampling variation. Prostatectomy samples with pathological and lethal outcome annotation from a large patient cohort with long follow-up were blindly assessed by expert pathologists who identified the tissue regions with the highest and lowest Gleason grade from each patient. To simulate biopsy-sampling error, a core from a high and a low Gleason area from each patient sample was used to generate a ‘High’ and a Tow′ tumor microarray, respectively. Using a quantitative in situ proteomics approach we identified from 160 candidates 12 biomarkers, mostly novel, that predicted prostate cancer aggressiveness (Surgical Gleason score and pathological TNM stage) and lethal outcome robustly in both high and low Gleason areas. Conversely, a previously reported lethal outcome-predictive marker signature for prostatectomy tissue was unable to perform under circumstances of maximal sampling error. Our work provides for cancer biomarker discovery in general and for a clinical test predictive of prostate cancer pathology at the time of biopsy, resistant to biopsy-sampling error.

### INTRODUCTION

Prostate cancer accounts for 27% of incident cancer diagnosed in men in the USA and the American Cancer Society estimates that, nationally, 233,000 new diagnoses of prostate cancer will be made in 2014 (1). Although the risk of death due to prostate cancer has fallen significantly as a result of earlier detection and improved treatment options (1), there are concerns around the over-diagnosis and over-treatment of this common cancer (2, 3). Of all newly diagnosed cases of prostate cancer, only about one in seven will progress to metastatic disease over a lifetime, whereas approximately half of men newly diagnosed with prostate cancer have localized disease that has a very low risk of progression (1, 4). Despite this low risk, as many as 90% of men diagnosed with low risk prostate cancer in the USA undergo radical treatment, usually radical prostatectomy or ablative radiation therapy (5). For a disease that is unlikely to become clinically apparent, such treatments may be excessive and often result in long-term adverse events, including urinary incontinence and erectile and bowel dysfunction (2, 6, 7).

Current guidance and accepted standards of care for the diagnosis and management of prostate cancer recommend the use of clinical and pathological parameters to assess the disease grade and stage on biopsy (8, 9). Pathological evaluation of tissue obtained by needle biopsy is essential both to confirm a prostate cancer diagnosis and to grade the cancer. Grade, as determined by biopsy Gleason score (GS) is the most important predictor of outcome, and has been deemed to be the most informative for guiding management decisions. Approximately 80-85% of all prostate cancer biopsies have a GS of 3+3=6 or 3+4=7, representing a spectrum of cases with low to intermediate to high risk of progression (10). Patients deemed to have indolent disease are candidates for active surveillance (3, 8, 9). However, current methods of biopsy evaluation are often unable to place individual patients accurately along this spectrum (5, 10).

There are two recognized factors that affect the accuracy of biopsy-based Gleason scoring: one is sampling variation (i.e. failing to sample the area with the highest Gleason grade), and the second is pathologist discordance in Gleason scoring (10-12). Despite the current standard practice of multicore biopsy sampling, the most aggressive area of the tumor is frequently underrepresented or overrepresented (11, 13). Indeed, 25-50% of cases of prostate cancer need to be either upgraded or downgraded from their initial biopsy score to a more accurate surgical GS after analysis and grading of prostatectomy tissue (10, 14, 15). Discordance between pathologists in Gleason grading derives from subjective aspects of the Gleason scoring system that particularly apply to small samples. Such discordance adds to the difficulty of ensuring uniform and accurate prognostication and can be as high as 30% (16, 17).

Several clinical and pathological risk stratification systems have been developed to improve prediction of prostate cancer aggressiveness, including the D'Amico classification system, the Cancer of the Prostate Risk Assessment (CAPRA) score, and the National Comprehensive Cancer Network (NCCN) guidelines (9, 18-20). All such systems recognize the biopsy GS as the single most powerful variable in risk assessment. The GS is comprised of two Gleason patterns, with the more prevalent pattern specified first. The two are summed to determine the Gleason score. According to a 2005 consensus on Gleason scoring, only three patterns (3, 4, and 5) are typically recognized on biopsy (21). The accepted prognostic categories of GS are 3+3=6, 3+4=7, 4+3=7, 8, and 9-10. Importantly, although 3+4=7 and 4+3=7 have equivalent Gleason sums, the latter has significantly worse prognosis, based on higher amount of pattern 4 (16, 22). Importantly, all of the risk stratification systems used to guide clinical management depend upon effective and consistent Gleason scoring and are therefore vulnerable to sampling variation and discordant scoring by pathologists.

Enhanced biopsy strategies have been proposed as one means to overcome sampling variation and errors. Among these, increasing the number or density of sampled cores might ensure more representative capture of tumor tissue. However, increasing the number of biopsy samples collected to more than the 12 currently recommended could increase the risk of adverse events from oversampling, and there is little evidence that this improves pathological classification (23, 24). There has also been interest in novel forms of image-guided biopsy. Currently, MRI-guided biopsy appears to improve detection of aggressive cancers, but long term studies will be needed to determine whether MRI can improve patient selection for active surveillance (AS) (25).

Using a quantitative multiplex proteomics in situ imaging system which enables accurate biomarker measurements from the intact tumor epithelium (26), we here report the identification and evaluation of 12 biomarkers that are able to predict prostate cancer aggressiveness (defined by prostatectomy (Surgical) Gleason score and pathological TNM stage) and lethal outcome. The markers were specifically selected to be robust to sampling error. The study was performed on prostatectomy tissue, and involved a simulation of biased biopsy sampling error based on coring from areas of high and low GS from each patient. Using this approach, biomarkers were selected based on their ability to reflect the true prostate pathology as determined by prostatectomy GS and pathological stage, regardless of whether they were measured in a high or a low score Gleason area. In addition to reflecting aggressive pathology, the biomarker candidates were also evaluated for their ability to predict prostate cancer-specific mortality across low- and high-grade areas of heterogeneous cancers. This performance-based approach identified novel biomarkers and confirmed known biomarkers predictive of prostate cancer aggressiveness and lethal outcome.

Results

Biopsy Simulation

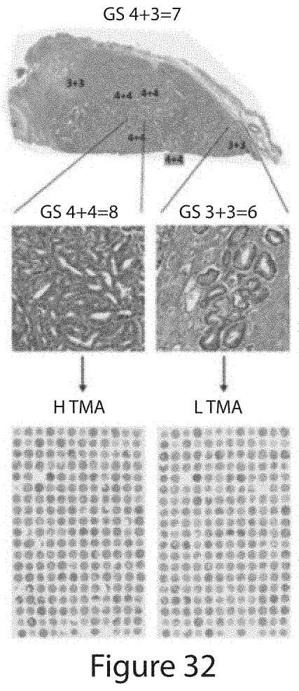

A biopsy-sampling model was developed to simulate and exaggerate the biopsy sample variation observed in clinical practice. For this purpose, we embedded cores from annotated prostatectomy tissue into tissue microarrays (TMAs). Based on centralized Gleason grading by expert urologic pathologists, a core was taken for each patient from the area with the least aggressive tumor (low GS) and embedded in a low grade tissue microarray (L TMA); in parallel, a core was taken from the area with the most aggressive tumor based on Gleason grading (high GS) and embedded in a high grade tissue microarray (H TMA) (FIG. 32). Thus, we developed paired tissue TMAs with samples biased in two directions, representing both more and less aggressive tumor areas from each patient. Table 12a describes the clinical features for the multi-institution cohort of 380 patients for whom paired TMAs were prepared. Table 12b describes the subset of 301 cases with core Gleason of 3+3 or 3+4 on L TMA along with their corresponding core Gleason on H TMA and their Surgical (prostatectomy) Gleason.

Table 12a and 12b show clinical features of the cohort used to create L and H TMAs.

TABLE 12a
A single cohort of 380 patients provided samples for the two TMAs. For
technical reasons, only 360 samples on the L TMA and 363 samples on the H TMA were
usable. TMA, tissue microarray.

| | L TMA | | | H TMA |
|---|---|---|---|---|
| Patients with | 360 of 380 | | | 363 of 380 |
| survival and | | | | |
| biomarker | | | | |
| information | | | | |
| Mean age | 62.2 (6.76) | | | 62.1 (6.83) |
| (SD), years | | | | |
| Lethal | 60 (16.67) | | | 59 (16.25) |
| events, n (%) | | | | |
| Mean length | 11.55 (3.96) | | | 11.52 (3.98) |
| of follow-up | | | | |
| (SD), years | | | | |
| Pathological | | | | |
| tumor stage, | | | | |
| n (%) | | | | |
| T2 | 244 (67.8) | | | 250 (68.9) |
| T3 | 112 (31.1) | | | 109 (20.0) |
| T4 | 2 (0.56) | | | 2 (0.55) |
| Missing | 2 (0.56) | | | 2 (0.55) |
| Core Gleason | | | | |
| score | n | % | n | % |
| ≤6 | 233 | 64.7 | 177 | 48.8 |
| 3 + 4 | 68 | 18.9 | 98 | 27.0 |
| 4 + 3 | 15 | 4.2 | 31 | 8.5 |
| 8 − 10 | 27 | 7.4 | 47 | 13 |
| Total | 343 | 95 | 353 | 97 |

| | Surgical | | Disease deaths | | | Surgical | | Disease deaths | | |
|---|---|---|---|---|---|---|---|---|---|---|
| Surgical | Gleason | | | % of | % of | Gleason | | | % of | % of |
| Gleason | score | | | disease | Gleason | score | | | disease | Gleason |
| score | N | % | N | Deaths | strata | N | % | N | Deaths | strata |
| ≤6 | 108 | 30.0 | 2 | 3.3 | 1.9 | 112 | 30.9 | 3 | 5.1 | 2.7 |
| 3 + 4 | 169 | 46.9 | 20 | 33.3 | 11.8 | 168 | 46.3 | 17 | 28.8 | 10.1 |
| 4 + 3 | 30 | 8.3 | 9 | 15.0 | 30.0 | 30 | 8.3 | 10 | 17.0 | 33.3 |
| 8 − 10 | 53 | 14.7 | 29 | 48.3 | 54.7 | 53 | 14.6 | 29 | 49.1 | 54.7 |
| Total | 360 | 100 | 60 | 100 | | 363 | 100 | 59 | 100 | |

TABLE 12b
The distribution of H TMA core Gleason scores and
Surgical Gleason scores amongst the 301 patients
with L TMA core Gleason of 3 + 3 or 3 + 4.

| Core | L TMA: | Core | H TMA: | Surgical | n of |
|---|---|---|---|---|---|
| Gleason | n of | Gleason | n of | Gleason | pa- |
| score | patients | score | patients | score | tients |
| 3 + 3 = 6 | 233 | 3 + 3 = 6 | 149 | 3 + 3 = 6 | 93 |
| | | 3 + 4 = 7 | 58 | 3 + 4 = 7 | 112 |
| | | ≥4 + 3 = 7 | 26 | ≥4 + 3 = 7 | 30 |
| 3 + 4 = 7 | 68 | 3 + 3 = 6 | 23 | 3 + 3 = 6 | 14 |
| | | 3 + 4 = 7 | 27 | 3 + 4 = 7 | 32 |
| | | ≥4 + 3 = 7 | 18 | ≥4 + 3 = 7 | 22 |

Sampling for the L TMA was specifically designed to underestimate disease severity. As shown in Table 12a and Table 12b, 64.7% of L TMA samples had a core GS less than or equal to 6, while only 30% of these L TMA samples came from patients with a surgical GS less than or equal to 6. The probability of upgrade (Table 12b) for samples in the L TMA from cases with core GS of ≤3+4 to a higher surgical GS was 0.64 (95% Wilson confidence interval [CI]: 0.59-0.69). This probability of upgrade is higher than that seen in clinical practice (12), as expected from the sampling method and patient cohort used. Thus, by exaggerating sample variation expected in clinical practice, this biopsy simulation procedure provided a useful model to identify biomarkers that reliably predict prostate cancer aggressiveness, regardless of sample variation.

Effect of Sampling Error on Known Biomarker Model Performance

To assess the effect of sampling variation on prognostic marker performance, we initially tested an established biomarker combination reported to be prognostic for lethal outcome when used on prostatectomy tissue for its ability to predict lethal outcome and aggressive disease when used on the biopsy simulation tissue. Prior studies have demonstrated that radical prostatectomy (RP) GS of 7 or higher and extension of prostate cancer beyond the prostate gland are significant predictors of metastasis and prostate cancer-specific mortality (27-29). Accordingly, we defined ‘aggressive disease’ based on the prostate pathology as surgical GS of at least 3+4 or pT3b (seminal vesicle invasion), N+, or M+. We tested the four-biomarker model (SMAD4, CCND1, SPP1, PTEN) previously reported by Ding et al. (30) for its ability to predict both disease specific death and disease aggressiveness in our sampling variation TMA cohort. Patient cores in the L or H TMA were separated into independent “training” and “testing” data sets, and logistic regression models were used to estimate marker coefficients using the training data set. We estimated area under the curve (AUC) from the resulting receiver operating characteristic (ROC) in the testing set and then repeated the process for additional sampling. As shown in Table 13, when measured on H TMA the 4-marker signature was able to predict disease-specific death with a median test AUC of 0.65 (95% CI of 0.59-0.74). However, when measured on L TMA, representing biased under-estimation of the Surgical GS, the 4-marker model showed a non-significant median test AUC of 0.49 (95% CI of 0.42-0.58). Moreover, the ability of the 4-marker signature to predict aggressive disease when measured in either H or L TMA also did not reach significance (median test AUC of 0.56 [95% CI of 0.44-0.64] and of 0.56 [95% CI of 0.46-0.65], respectively). These results illustrate the impact of sampling error on prognostic marker performance and the importance of identifying alternative biomarker combinations that can predict outcomes accurately despite such sampling variation.

TABLE 13
Sampling variation reduces the performance of an established lethal
outcome-predictive biomarker signature. The combination PTEN +
SMAD4 + CCND1 + SPP1has previously been shown
to be prognostic for lethal outcome when measured on prostatectomy
tissue. We confirmed that these markers are indeed predictive of
lethal outcome when measured in the high Gleason biopsy simulation
tissue (H TMA). However, these markers are unable to predict lethality
in the low Gleason simulation biopsy (L TMA). The markers do not show
statistically significant predictive performance for aggressive disease
regardless whether measured in high (H TMA) or low (L TMA) Gleason
tissue areas. AIC, Akaike information criterion; C statistic, area under
receiver operating characteristic (ROC) curve; TMA, tissue microarray.

| Markers: | Mean | Median train | Median test |
|---|---|---|---|
| PTEN SMAD4 | AIC (2.5%, | AUC (2.5%, | AUC (2.5%, |
| CCND1 SPP1 | 97.5%) | 97.5%) | 97.5%) |
| H TMA Lethal | 282.2 | 0.67 | 0.65 |
| | (275.9, 293.2) | (0.64, 0.70) | (0.59, 0.74) |
| L TMA Lethal | 301.3 | 0.6 | 0.49 |
| | (288.6, 316.8) | (0.58, 0.63) | (0.42, 0.58) |
| H TMA Aggressiveness | 350.1 | 0.62 | 0.56 |
| | (330.4, 367.4) | (0.56, 0.68) | (0.44, 0.64) |
| L TMA Aggressiveness | 381.6 | 0.61 | 0.56 |
| | (353.0, 400.7) | (0.55, 0.68) | (0.46, 0.65) |

Biomarker Identification

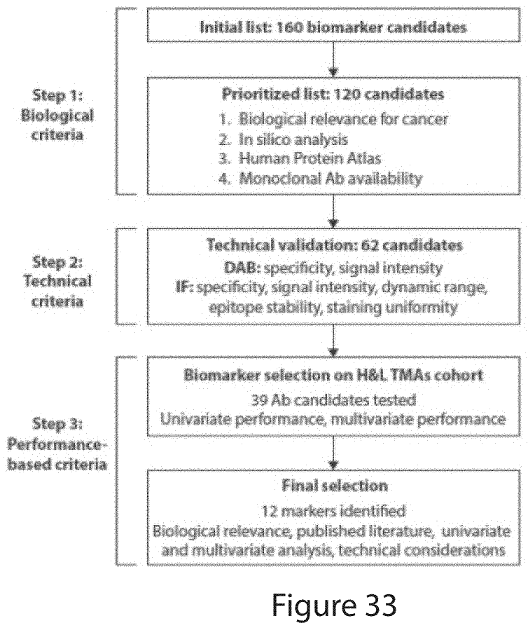

After showing that the biased biopsy simulation TMAs did indeed reflect an extreme sampling error scenario, and that such sampling variation rendered a known predictive marker signature unable to perform reproducibly, we next pursued the primary objective of identifying biomarkers that would robustly predict cancer aggressiveness regardless of biopsy-sampling variation. By taking advantage of prostatectomy tissue samples with rich clinical and pathological annotation from a large cohort of patients with long-term follow-up, we established a performance-based strategy to select potential markers. The stepwise approach involved: 1) identification of candidate biomarkers, 2) evaluation of their biological and technical suitability, and 3) analysis of performance in H and L TMA cohorts (FIG. 33).

The process began with a search of published literature and publicly available gene expression data sets, which identified 160 biomarker candidates based on biological relevance for prostate cancer (30-48). We further prioritized 120 of these based on availability of appropriate monoclonal antibodies (MAbs) (see Table 14 for a comprehensive biomarker candidate list). Candidates included well-characterized markers relevant for prostate cancer aggressiveness, such as EZH2, MTDH, FOXA1 (49-51), and the markers PTEN, SMAD4, Cyclin D1, SPP1, phospho-PRAS40-T246 (pPRAS40), and phospho-S6-Ser235/236 (pS6) previously identified as predictive of lethal outcome on prostatectomy tissue (26, 30).

TABLE 14
Candidate biomarkers identified from published literature and gene expression
databases. Notes: DAB staining and specificity. Passed: antibodies that
with DAB-based immunohistochemical staining demonstrated signal intensity
and staining pattern of benign and tumor prostate tissue commensurate
with published literature. Immunofluorescence signal and specificity.
Passed: antibodies that with immunofluorescent staining exhibited high
level of signal and with staining pattern of benign and tumor prostate
tissue commensurate with published literature. Marker stability in tissue.
Passed: antibodies that showed signal intensities correlating with epithelial
marker staining intensities across tissue areas of variable quality.
MPTMA 10. Passed: antibodies that demonstrated correlation between expression
and Surgical (prostatectomy) Gleason score.

| | | Immuno- | | | |
|---|---|---|---|---|---|
| | | fluorescence | Marker | | |
| | DAB staining | signal and | Stability | MPTMA | Tested on H |
| Biomarker | and specificity | specificity | in tissue | 10 | and L TMAs |
| PIK3R1 | Failed | | | | |
| PHLPP1 (Poly) | Passed | Passed | Passed | Failed | |
| CDKN1B (p27kip1) | Passed | Passed | Passed | Passed | Yes |
| SPRY2 | Failed | | | | |
| NCOR2 | Passed | Failed | | | |
| E2F1 | Failed | | | | |
| Top2A | Failed | | | | |
| IGF1 | Failed | | | | |
| EGR1 | Failed | | | | |
| SRF | Passed | Failed | | | |
| CTGF | Failed | | | | |
| CCL2 | Failed | | | | |
| FUS | Passed | Passed | Passed | Passed | Yes |
| LKB1 (STK11) | Passed | Failed | | | |
| CD142 | Passed | Failed | | | |
| MTHFD1L | Failed | | | | |
| SHMT2 | MAb not | | | | |
| | available | | | | |
| KRT6A | Failed | | | | |
| LOX | Failed | | | | |
| CD53 | Passed | Failed | | | |
| CUL2 | Passed | Passed | Passed | Passed | Yes |
| MBD2 | Failed | | | | |
| MTERF | MAb not | | | | |
| | available | | | | |
| PARD3 | Failed | | | | |
| RBL2 | Passed | Passed | Passed | Failed | |
| SMAD2 | Passed | Passed | Passed | Passed | Yes |
| SMAD7 | Failed | | | | |
| DSC2 | Passed | Passed | Failed | | |
| EMD | Passed | Passed | Passed | Failed | |
| PRMT1 | Passed | Passed | Passed | Failed | |
| REV1 | Passed | Failed | | | |
| StAR | Passed | Passed | Passed | Passed | Yes |
| CPNE3 | MAb not | | | | |
| | available | | | | |
| CML66 | Passed | Passed | Passed | Failed | |
| GRINA | Passed | Failed | | | |
| SPAG1 | MAb not | | | | |
| | available | | | | |
| ANPTL4 | MAb not | | | | |
| | available | | | | |
| TGS1 | MAb not | | | | |
| | available | | | | |
| WWP1 | Passed | Passed | Passed | Failed | |
| ATF2 | Passed | Failed | | | |
| COPB2 | Passed | Passed | Passed | Failed | |
| DERL1 | Passed | Passed | Passed | Passed | Yes |
| FAM91A1 | MAb not | | | | |
| | available | | | | |
| FOLH1 | Passed | Failed | | | |
| KIF5C | Passed | Passed | Passed | Failed | |
| NPC2 | Failed | | | | |
| OXCT1 | MAb not | | | | |
| | available | | | | |
| RAB18 | Failed | | | | |
| RHOA | Passed | Passed | Passed | Failed | |
| UNC13B | Failed | | | | |
| YIPF6 | MAb not | | | | |
| | available | | | | |
| ST6GAL1 (CD75) | Passed | Passed | Passed | Passed | Yes |
| BHLHE40 (Dec1) | Passed | Passed | Passed | Passed | Yes |
| BHLHE41 (Dec2) | Passed | Failed | | | |
| EIF2C2 | Passed | Failed | | | |
| PUF60 | Failed | | | | |
| WDR67 | MAb not | | | | |
| | available | | | | |
| SQLE | Passed | Failed | | | |
| RNF19A | Failed | | | | |
| UBR5 | Passed | Failed | | | |
| PABPC1 | Passed | Passed | Passed | Failed | |
| EIF3H | Passed | Passed | Passed | Passed | Yes |
| ARMC1 | Failed | | | | |
| WDYHV1 | MAb not | | | | |
| | available | | | | |
| ANKRD46 | MAb not | | | | |
| | available | | | | |
| AKAP9 | Failed | | | | |
| AKAP8 | Passed | Passed | Passed | Passed | Yes |
| EEF1D | Failed | | | | |
| TMEM68 | MAb not | | | | |
| | available | | | | |
| SRI | Passed | Failed | | | |
| HOXB13 | Passed | Passed | Passed | Passed | Yes |
| NCOA2 (clone 29) | Passed | Passed | Passed | Passed | Yes |
| SLC2A4/GLUT4 | Failed | | | | |
| GRIP-1 | Passed | Passed | Passed | Passed | Yes |
| SCRIB | Passed | Passed | Failed | Failed | |
| PXN | Passed | Passed | Passed | Passed | Yes |
| ARHGEF7 | Passed | Failed | | | |
| RAVER1 | Failed | | | | |
| PTBP1 | Passed | Passed | Failed | Failed | |
| KHDRBS2 | MAb not | | | | |
| | available | | | | |
| KHDRBS3 | Passed | Passed | Passed | Failed | |
| UBE2L3 | Failed | | | | |
| UBE2L6 | Failed | | | | |
| SNCG | Passed | Failed | | | |
| MT-CO2 | Passed | Passed | Passed | Failed | |
| RTN4 | Failed | | | | |
| COMT | Passed | Passed | Passed | Failed | |
| PNMT | Failed | | | | |
| ABL2 | Failed | | | | |
| ACTN1 | Passed | Passed | Passed | Passed | Yes |
| CDC7 | Failed | | | | |
| CPNE3 | MAb not | | | | |
| | available | | | | |
| DAB2 | Failed | | | | |
| FKBP5 | Passed | Passed | Passed | Passed | Yes |
| HMMR | Failed | | | | |
| ITGB3BP | Failed | | | | |
| KIAA0196 | MAb not | | | | |
| | available | | | | |
| KIF11 | Passed | Passed | Passed | Failed | |
| MAP2K6 | Failed | | | | |
| MRPL37 | MAb not | | | | |
| | available | | | | |
| MTHFD2 | Failed | | | | |
| NRP1 | Failed | | | | |
| OXCT1 | MAb not | | | | |
| | available | | | | |
| ST14 | Passed | Passed | Passed | Failed | |
| PDSS2 | Passed | Passed | Passed | Passed | Yes |
| DIABLO | Passed | Passed | Passed | Passed | Yes |
| ATP6V1F | Failed | | | | |
| AZGP1 | Passed | Failed | | | |
| CAPZA2 | Passed | Failed | | | |
| COX6C | Passed | Passed | Passed | Passed | Yes |
| DAD1 | Failed | | | | |
| HSD17B4 | Passed | Passed | Passed | Passed | Yes |
| PRDX5 | Passed | Failed | | | |
| SLC22A3 | Passed | Passed | Failed | | |
| YBX1 | Passed | Passed | Passed | Passed | Yes |
| MAOA | Passed | Passed | Passed | Passed | Yes |
| SHMT2 | Failed | | | | |
| ECHS1 | Failed | | | | |
| TMEM16G | Failed | | | | |
| VCAN | Failed | | | | |
| PDIA3 | Passed | Passed | Failed | | |
| MAP3K5 | Passed | Passed | Passed | Passed | Yes |
| ANXA5 | Failed | | | | |
| TRAF4 | Passed | Failed | | | |
| VCP | Failed | | | | |
| VDAC1 | Passed | Passed | Passed | Passed | Yes |
| COL1A2 | Failed | | | | |
| SSTR1 | Failed | | | | |
| LACTB2 | Passed | Failed | | | |
| XKR9 | Passed | Failed | | | |
| PEBP4 | Failed | | | | |
| PPP3CC | Failed | | | | |
| SLC39A14 | Passed | Failed | | | |
| LATS2 | Passed | Passed | Passed | Passed | Yes |
| PLAG1 | Passed | Passed | Passed | Passed | Yes |
| Stat 5 | Failed | | | | |
| cMyc | Passed | Failed | | | |
| ANO7 | Passed | Passed | Failed | | |
| AGPAT6 | Passed | Passed | Passed | Passed | Yes |
| ROCK1 | Passed | Failed | | | |
| RAD21 | Passed | Failed | | | |
| FASN | Passed | Passed | Failed | | |
| PECI | Passed | Failed | | | |
| Stathmin | Failed | | | | |
| SLC16A1 | Passed | Passed | Failed | | |
| TGM2 | Failed | | | | |
| Ubc2H10 | Passed | Failed | | | |
| EZH2 | Passed | Passed | Passed | Passed | Yes |
| AR | Passed | Passed | Failed | | |
| FOXA1 | Passed | Passed | Failed | | |
| HSPA9 | Passed | Passed | Passed | Passed | Yes |
| FAK1 | Passed | Passed | Passed | Passed | Yes |
| LMO7 | Passed | Passed | Passed | Passed | Yes |
| MTDH2 | Passed | Passed | Passed | Passed | Yes |
| AGK | Passed | Passed | Passed | Passed | Yes |
| CDH10 | Passed | Passed | Passed | Failed | |
| COBP2 | Passed | Passed | Passed | Failed | |
| CRLF1 | Passed | Passed | Passed | Failed | |
| RASSF1 | Passed | Passed | Passed | Failed | |
| RRM2 | Passed | Passed | Passed | Failed | |
| PRMT16 | Passed | Passed | Passed | Failed | |
| pS6 | N/A | N/A | | | Yes |
| SMAD4 | N/A | N/A | | | Yes |
| CCND1 | N/A | N/A | | | Yes |
| pPRAS40 | N/A | N/A | | | Yes |
| PTEN | N/A | N/A | | | Yes |
| SPP1 | N/A | N/A | | | Yes |

N/A: not applicable

We next procured and tested MAbs against the 120 prioritized candidate biomarkers for specificity and suitability for quantitative multiplex immunofluorescence (QMIF) assay. Candidate MAbs were selected for further analysis on the basis of signal intensity and specific immunofluorescence (IF) staining patterns, as described elsewhere (26). We prioritized MAbs that preferentially stained cancer cells over stromal cells. Based on a large number of stained samples, we observed that IF staining intensities of epithelial markers were low in seemingly badly fixed or preserved tissue. Candidate biomarker antibodies were selected based on signals that were more stable relative to those of epithelial markers.

In the third step, we tested the 62 MAbs that passed the previous steps and determined their dynamic range as well as their predictive performance. Using a small test TMA designed to represent the least aggressive areas from prostate tumors with high and low overall GSs, biomarkers were selected based on correlation of signal intensity with Surgical GS. Specifically, we required a three-fold difference of signals between lowest and highest expression values, in addition to demonstrated difference in signal value distributions between nonaggressive and aggressive cases. The final 39 candidate MAbs that fulfilled these criteria were tested on the clinical cohort represented by H and L TMA blocks described above.

Univariate Analysis

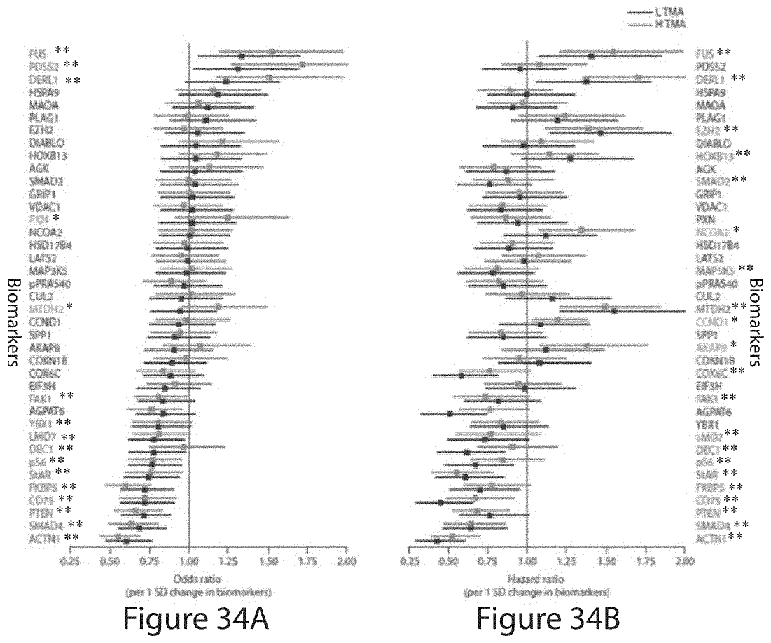

Our next goal was to evaluate the candidate biomarkers further based on univariate prognostic capability and analytical performance under circumstances of sampling error. Each of the 39 biomarkers identified above were tested for their ability to predict disease aggressiveness (Surgical GS ≥3+4 or pathological stage pT3b, and/or N+ or M+) and death from disease (survival analysis) when measured in both low and high Gleason areas. The individual markers shown with two asterisks demonstrated predictive value (P<0.1) for aggressive disease or death from prostate cancer based on increased or decreased expression regardless whether they were measured in low or high Gleason areas (FIG. 34). This result suggests that these markers are resistant to varying degrees of sampling error. There were 2 markers that were predictive of aggressiveness and 3 markers of lethal outcome only when measured in high, but not in low Gleason areas, indicating that these markers are not robust to sampling error. Conversely, we identified no markers that had predictive performance when measured in low, but not high Gleason areas. Of note, the strong link between aggressive disease and lethal outcome was revealed by the finding that, of the 14 markers with significant univariate performance for aggressiveness, 12 of them also exhibited significant univariate performance for lethal outcome. As further validation of the performance-based biomarker selection approach we confirmed the strong correlation between lethal outcome and expression of three known prostate cancer progression markers, EZH2, HoxB13, and MTDH2, as previously reported (49-51). In conclusion, we identified a number of marker candidates with univariate performance for both aggressive disease and lethal outcome that are also resistant to sampling error. Moreover, we identified markers that were only predictive in situations of minimal sampling error (performance in H, but not L TMA).

Multivariate Analysis: Biomarkers Predicting Tumor Aggressiveness

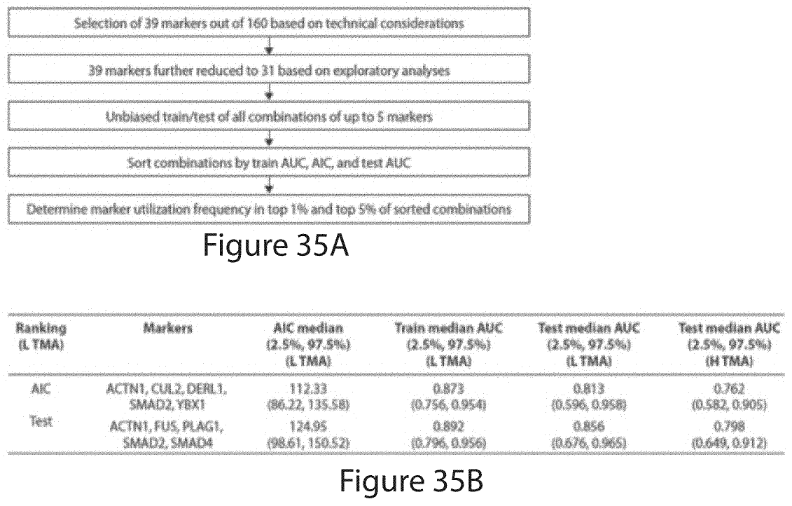

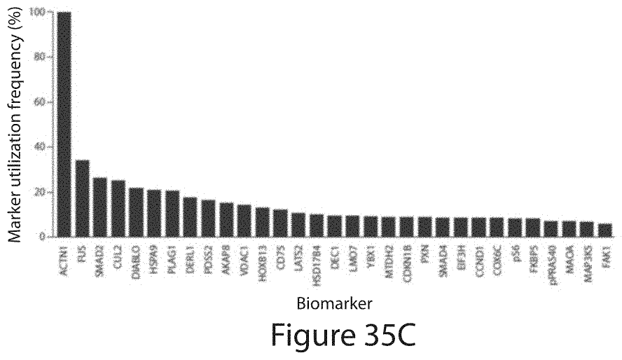

To explore the best multivariate biomarker combinations to predict disease aggressiveness, we exhaustively searched all possible models with combinations up to and including five biomarkers (FIG. 35A). Multivariate analyses focused on 31 biomarkers, refined from the original set of 39 based on technical criteria including MAb detection signal intensity, dynamic range, and specificity (see Materials and Methods). Initially, an ‘extreme’ model approach was used for the multivariate analysis, which included removal of the intermediate samples (GS=3+4, ≤T3a and NO) for the model building and testing. We separated patient cores in the L TMA into independent training and test sets and tested the resulting models on both L and H TMAs for multivariate performance across sampling variation. For this purpose, we used logistic regression models to estimate biomarker coefficients using the training data set, estimated AUC from the resulting ROC in the testing set, and then repeated the process for another sampling.

In each case, the most frequently occurring biomarkers in the top 5% or 1% of the models, sorted by AIC (Akaike information criterion) (52) and test-set AUC, were determined. A final tally was generated for ranking by test, ranking by AIC and both rankings (see

FIG. 35B for representative example of a five-biomarker model ranked by AIC and test). We observed a high degree of conservation of biomarker order in the top-performing biomarker models (see FIG. 35C and Table 15). The following biomarkers appeared among the top markers in at least 50% of the ranked lists: ACTN1, FUS, SMAD2, DERL1, YBX1, DEC1, pS6, HSPA9, HOXB13, PDSS2, SMAD4, CD75. In addition, CUL2 was present in a number of highly ranked models. (See Table 15 for further details of the ranking results)

TABLE 15
Performance-based biomarker ranking: aggressiveness. Combinations
of up to five biomarkers were generated and tested for their
ability to predict severe disease (aggressiveness). The frequency
of each biomarker in the best models was used for ranking.

| | |
|---|---|
| Sort by AIC | Sort by Test |

| | | | |
|---|---|---|---|
| YBX1 | 70.80 | ACTN1 | 99.94 |
| CUL2 | 65.72 | FUS | 34.18 |
| ACTN1 | 44.09 | SMAD2 | 26.13 |
| AKAP8 | 20.74 | CUL2 | 25.00 |
| SMAD2 | 17.43 | DIABLO | 21.59 |
| DEC1 | 16.37 | HSPA9 | 20.79 |
| DIABLO | 15.29 | PLAG1 | 20.4 |
| CD75 | 15.12 | DERL1 | 17.42 |
| FUS | 14.31 | PDSS2 | 16.21 |
| HOXB13 | 14.17 | AKAP8 | 14.94 |
| PLAG1 | 14.02 | VDAC1 | 14.08 |
| HSPA9 | 13.76 | HOXB13 | 12.84 |
| PDSS2 | 13.29 | CD75 | 11.93 |
| EIF3H | 11.93 | LATS2 | 10.44 |
| PXN | 11.65 | HSD17B4 | 10.00 |
| DERL1 | 11.20 | DEC1 | 9.40 |
| LATS2 | 10.80 | LMO7 | 9.20 |
| pS6 | 10.51 | YBX1 | 9.18 |
| pPRAS40 | 10.38 | MTDH2 | 8.76 |
| HSD17B4 | 10.33 | CDKN1B | 8.67 |
| MAOA | 9.10 | PXN | 8.65 |
| FAK1 | 8.71 | SMAD4 | 8.49 |
| VDAC1 | 8.35 | EIF3H | 8.48 |
| FKBP5 | 8.14 | CCND1 | 8.39 |
| MTDH2 | 7.45 | COX6C | 8.37 |
| MAP3K5 | 7.42 | pS6 | 8.28 |
| CCND1 | 7.25 | FKBP5 | 8.22 |
| LMO7 | 7.14 | pPRAS40 | 6.97 |
| COX6C | 6.50 | MAOA | 6.81 |
| CDKN1B | 6.13 | MAP3K5 | 6.75 |
| SMAD4 | 5.57 | FAK1 | 5.83 |

Multivariate Analysis: Biomarkers Predicting Lethal Outcome

A similar modeling analysis was performed for lethal outcome (Table 16). Biomarkers appearing among top markers in at least 50% of the ranked lists included: MTDH2, ACTN1, COX6C, YBX1, SMAD2, DERL1, CD75, FUS, LMO7, PDSS2, FAK1, SMAD4, DEC1. (See Table 16 for further details of the ranking results.)

TABLE 16
Performance-based biomarker ranking: lethal outcome. Combinations
of up to five markers were generated and tested for their ability
to predict lethal outcome (lethality). The frequency of each
biomarker in the best models was used for ranking.

| | |
|---|---|
| Sort by AIC (%) | Sort by Test (%) |

| | | | |
|---|---|---|---|
| ACTN1 | 95.20 | ACTN1 | 97.55 |
| PLAG1 | 41.99 | PLAG1 | 40.62 |
| MTDH2 | 37.97 | MTDH2 | 32.79 |
| DERL1 | 21.86 | HOXB13 | 29.65 |
| HOXB13 | 20.76 | DERL1 | 16.26 |
| CD75 | 17.49 | PDSS2 | 16.18 |
| PDSS2 | 16.69 | CD75 | 15.56 |
| FAK1 | 16.19 | COX6C | 13.85 |
| FUS | 12.99 | FAK1 | 13.30 |
| AKAP8 | 12.39 | FUS | 12.72 |
| COX6C | 11.51 | AKAP8 | 11.97 |
| SMAD4 | 11.06 | CUL2 | 11.29 |
| MAP3K5 | 10.90 | pS6 | 10.96 |
| pS6 | 10.25 | EIF3H | 10.04 |
| LMO7 | 10.20 | CCND1 | 9.62 |
| FKBP5 | 9.97 | DIABLO | 9.41 |
| CUL2 | 9.67 | YBX1 | 9.36 |
| EIF3H | 9.57 | HSPA9 | 9.32 |
| VDAC1 | 9.54 | pPRAS40 | 9.27 |
| CDKN1B | 9.28 | HSD17B4 | 9.26 |
| MAOA | 9.23 | LATS2 | 9.21 |
| pPRAS40 | 8.95 | SMAD4 | 9.16 |
| YBX1 | 8.90 | PXN | 9.08 |
| HSPA9 | 8.78 | CDKN1B | 9.06 |
| DEC1 | 8.76 | MAP3K5 | 8.84 |
| DIABLO | 8.63 | DEC1 | 8.78 |
| SMAD2 | 8.29 | LMO7 | 8.77 |
| LATS2 | 8.24 | SMAD2 | 8.46 |
| CCND1 | 8.24 | MAOA | 8.33 |
| HSD17B4 | 7.95 | FKBP5 | 8.12 |
| PXN | 7.81 | VDAC1 | 7.54 |

Final Biomarker Set

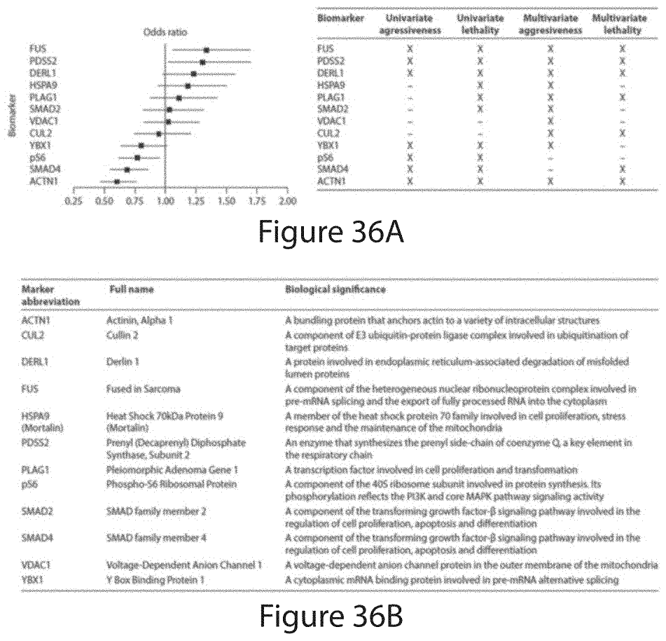

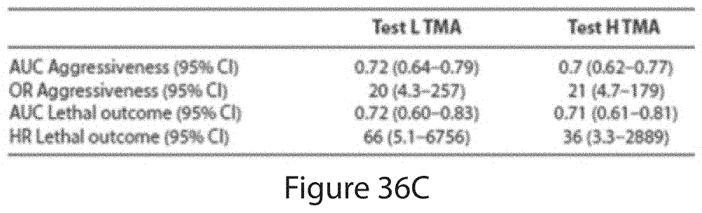

We chose a final set of 12 biomarkers based on careful integration of univariate and multivariate performance, and analytical considerations, including minimally a 3-fold dynamic signal intensity range across tumor samples for all antibodies. FIG. 36A shows the estimated odds ratios (ORs) associated with these 12 biomarkers for univariate prediction of aggressiveness, and also summarizes the basis for choice of each biomarker based on both univariate and multivariate analyses. FIG. 36B provides a biological summary of the selected biomarkers. The final biomarker set was comprised of: FUS, PDSS2, DERL1, HSPA9, PLAG1, SMAD2, VDAC1, CUL2, YXB1, pS6, SMAD4, ACTN1.

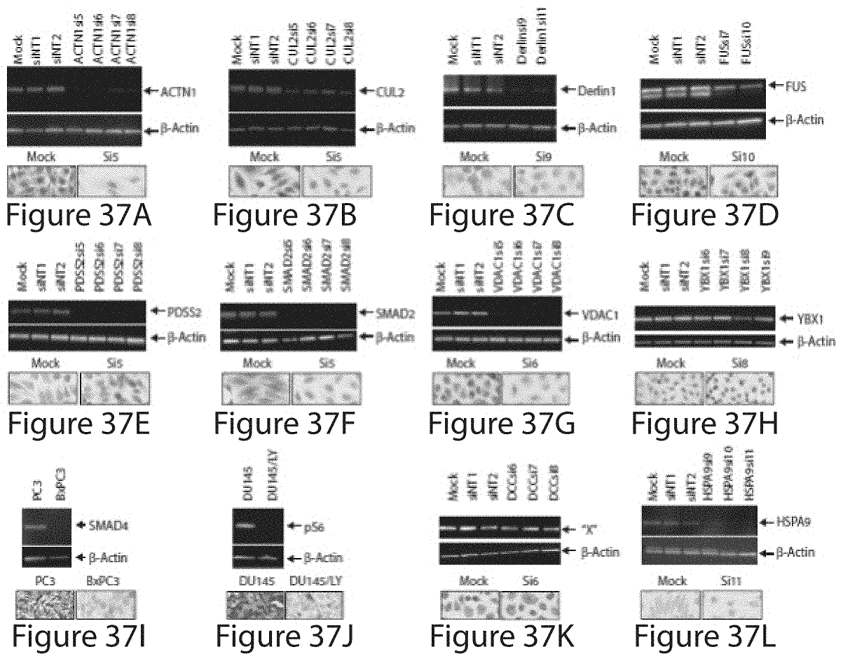

Each of the 12 marker antibodies was rigorously validated by specificity analyses including Western blotting (WB) and immunohistochemistry (IHC) assay before and after target-specific knockdown, as shown in FIG. 37. Interestingly, through this process we found that the MAb sold as specific for DCC did not detect DCC, but rather HSPA9 (also known as Mortalin)(FIG. 38). Since DCC knockdown did not result in disappearance of the only band on WB we undertook mass spectrometry sequencing analysis to identify the protein as HSPA9. The fact that this protein has a well-described role in cancer progression and survival(53), further validates the performance and function-based biomarker identification approach.

We next used the previously described modelling approach to assess the predictive potential of the final 12-biomarker set for both disease aggressiveness and disease-specific death on the entire patient cohort. Data from the L TMA and H TMA were randomly partitioned into training and test sets, logistic regression was performed on the L TMA training set, performance was evaluated on the L TMA and H TMA test sets, and the process was repeated to develop a 12-marker model for disease aggressiveness. As shown in FIG. 36C, this resulted in an L TMA test AUC of 0.72 (95% CI: 0.64-0.79) and a corresponding OR for aggressive disease of 20 per unit change in risk score (95% CI: 4.3-257). To confirm the ability to generalize across sampling error, the model derived from the L TMA training set was also tested on H TMA for prediction of aggressive disease with consistent results (FIG. 36C). Without any further changes to the aggressiveness model we examined its performance on lethal outcome prediction by correlating the aggressiveness risk scores with death from disease. Of note, we found a similar AUC for lethal outcome as for aggressiveness on both L and H TMA of 0.72 (95% CI: 0.60-0.83) and 0.71 (95% CI: 0.61-0.81), respectively. The corresponding HRs for lethal outcome on L and H TMAs were 66 per unit change (95% CI: 5.1-6756) and 36 (95% CI: 3.3-2889), respectively. We conclude that the 12 identified biomarkers are robust to sampling error and likely predictive of both disease aggressiveness and lethal outcome.

### DISCUSSION

There is a continuing clinical need to assess prostate cancer aggressiveness more accurately at the time of initial diagnosis and as part of the ongoing follow-up of patients, including those assigned to active patient surveillance as well as those receiving active treatment for this disease (4, 29, 54). Currently, in men with early disease, a biopsy GS of 3+4=7 or more is one of the prognostic factors that serves to indicate the need for active treatment (9, 55) but, as discussed, biopsy-sampling error resulting from tumor heterogeneity and discordant Gleason scoring can affect the accuracy and reliability of assessing a patient's risk of cancer progression, aggressiveness and lethality. This uncertainty has contributed to a situation where prostate cancer is significantly overtreated, as the prognosis for patients with biopsies of Gleason grade 3+3 or 3+4 is difficult to accurately predict (2, 5, 10, 54, 56, 57).

Biomarkers Predictive of Prostate Cancer Aggressiveness and Lethality

Described herein is the successful development of a performance-based method to identify and evaluate biomarkers predictive of prostate cancer aggressiveness and lethal outcome, even under circumstances of extreme sampling variation, an issue typically encountered during prostate biopsy taking. Using a large cohort (N=380) of annotated clinical prostatectomy samples with long-term follow up for lethal outcome, the areas of highest and lowest GS on each prostatectomy tissue were marked by expert pathologists in blinded manner. By coring these ‘high’ and ‘low’ regions from each patient sample we generated paired TMAs representing the entire cohort, thereby simulating biopsies with sampling error for each patient. Using these paired TMAs, we assessed a large number of biomarker candidates for the ability to predict aggressive prostate pathology and lethal outcome when measured in either low or high grade cancer regions from each patient. We specifically first selected for biomarkers with performance against aggressiveness and lethal outcome when measured in L TMA tissue, to identify those most robust to extreme sampling error. For this purpose, we only included L TMA samples with core Gleason≤3+4 as clinically relevant, since biopsies with GS 4+3 or higher inevitably will be aggressive therapy candidates. Biomarker candidates were quantified using an integrated multiplex proteomics in situ imaging platform, which provides automated, objective biomarker measurements (26). Based on univariate analyses, most of the identified biomarkers were predictive of both disease aggressiveness and prostate cancer-specific mortality regardless whether measured in L or H TMA tissue samples, and hence robust to sampling variation (FIG. 34). Moreover, several prostate cancer biomarkers previously reported to be predictive of progression risk and lethal outcome, including SMAD4, EZH2, MTDH2, HoxB13, and PTEN all confirmed positive for lethal outcome, supporting the validity of the approach.

As part of specificity validation of our antibodies we learned through target knockdown analyses and mass spectrometry-based protein sequencing analysis that a MAb sold as anti-DCC actually recognized the unrelated protein HSPA9, or Mortalin. We found that HSPA9 was predictive as part of multivariate models and hence was included in the final 12 marker set. When subjected to functional analyses we did indeed find that HSPA9 was involved in clonogenic cell colony assay formation and cell proliferation, consistent with previous findings (see FIG. 38 and (53)). This further supports the validity of the unbiased, performance-based marker selection approach.

Based on univariate performance as well as frequency of marker appearance in multivariate models for disease aggressiveness and lethal outcome 12 biomarkers were selected (FIG. 36A). A multivariate model based on these 12 markers showed similar predictive performance for aggressiveness across tissue sampling variation (FIG. 36C). Interestingly, the risk scores generated based on the 12-marker aggressiveness model were equally predictive for the separate endpoint of lethal outcome across tissue sampling variation (FIG. 36C). The fact that the markers predictive of prostate cancer aggressiveness, defined by prostate Gleason grade and stage, were also predictive of lethal outcome strongly supports the linkage of aggressive features on surgical pathology with lethality. More importantly, it validates the usage of our pathologic endpoint for building our biomarker panel as relevant for long-term patient outcome. The 12 identified biomarkers are relevant for prediction of tumor behaviour and provide the basis for a clinical, evidence-based multivariate biopsy test for assessing prostate cancer aggressiveness. Biopsies play a key role at initial diagnosis and in monitoring disease status in patients undergoing active surveillance (8, 9). As such, there a multivariate biopsy test, as described here, can inform early decision-making steps in managing patients with prostate cancer.

Biomarkers Robust to Sampling Error

The present study identified and selected markers that are highly robust to sampling error. One of the key reasons for biopsy sampling error is the heterogeneity of prostate cancer. The inability to consistently acquire tissue from the most aggressive parts of the tumor leads to frequent under-estimation of tumor aggressiveness and progression risk. By coring into the highest and lowest Gleason area from each patient we generated paired TMAs of the entire cohort study designed to simulate two biopsies from each patient, one with ‘maximal’ sampling error (L TMA), and the other with minimal sampling error (H TMA). We focused on L TMAs with core Gleason ≤3+4, as these represent the clinically relevant cases where standard of care is insufficient for accurate prognosis. We found that ˜54% of these L TMA cases were upgraded to a higher Surgical Gleason score, which is higher than observed in clinical practice(12), confirming that our approach provided a biased sampling error model (Table 12b).

The need for identification of biomarkers that are resistant to sampling error was underscored by examining a well-established 4-marker signature based on Cyclin D1, SMAD4, PTEN, and SPP1 previously reported to be predictive of lethal outcome based on prostatectomy cohorts (30). While the model was predictive for lethal outcome in H TMA, representing a situation of minimal sampling error, the model was not lethal outcome-predictive at all in our L TMA tissue cores, representing maximal sampling error (Table 13). This finding is consistent with a recent report that the 4-marker signature is unable to predict lethal outcome in low Gleason score prostate tumors (58).

Based on univariate marker analyses we identified 14 and 18 markers with sampling error-robust performance across L and H TMA samples for disease aggressiveness and lethal outcome, respectively (markers marked with ** on FIG. 34). Most of these univariately selected markers were predictive of both indications across sampling variation, again supporting the correlation between disease aggressiveness and lethal outcome, as discussed above for multivariate analyses described above. Interestingly, while all markers that showed univariate performance for disease aggressiveness and lethal outcome on L TMA also were predictive on H TMA, 2 markers (PXN and MTDH2) and 3 markers (NCOA2, CCND1 (Cyclin D1), and AKAP8) were predictive of aggressive disease and lethal outcome, respectively, only when measured on H TMA, but not on L TMA (FIG. 34). This indicates that these markers are predictive primarily in situations of minimal sampling error. Indeed, all these 5 markers have been shown as important regulators of cellular proliferation, migration and oncogenesis (see e.g. (30, 51, 59-61)). The observation that Cyclin D1 is predictive of lethal outcome only in H TMA, but not L TMA is consistent with the finding that the 4-marker signature reported by Ding et al was not predictive of lethal outcome in our L TMA tissue, as well as on low grade prostate cancer samples (58). The fact that no markers were predictive for aggressive disease or lethal outcome on only L TMA, but not H TMA, is interesting given that we primarily selected for markers that can predict either aggressiveness or lethal outcome in L TMA, to reflect maximal sampling error robustness. This indicates that the identified markers likely reflect field effects from more aggressive tumor regions, consistent with their similar performance in L and H TMA tissue.

Genetic and Proteomic Approaches

In the search to find new and better biomarkers in prostate cancer, there has been great interest and advances made in identifying possible genetic markers that might inform clinical risk prognostication (31, 32, 39, 48, 62, 63). However, for many of the genes identified, there are conflicting or poor results regarding the reliability of such markers in disease prognostication. For example, although TMPRSS2-ERG gene fusions are reported to be associated with high-risk tumors, more recent studies with large cohorts report no strong correlation between these fusions and patient outcome (64). A multivariate gene expression-based test has recently been reported to predict metastatic disease and lethal outcome based on a conservatively managed cohort of patients from the UK (65), as well as biochemical recurrence after treatment in actively managed cohorts in the US (66, 67). The influence of sampling variation on this test has yet to be established.

The results of the present study demonstrate that taking a proteomic approach, which measures proteins from only the tumor region of intact tissue, can improve accurate risk classification at the biopsy stage. The rationale for this idea is two-fold. First, because prostate cancer is a heterogeneous, multifocal disease, biopsies frequently contain only lower-grade components, and pathologists may classify them as low-risk cancers. However, higher-grade molecular features, not reflected morphologically, have been reported to extend throughout the cancer, (68, 69) and therefore are measurable in seemingly lower grade-containing biopsies. Through a proteomic approach measuring proteins only from intact tissue tumor regions, it is possible to accurately and sensitively assess such high grade molecular features in situ, even in tissue samples with variable amounts of tumor versus benign components. This is an advantage to gene expression-based technologies requiring tissue homogenization, resulting in variable dilution of the higher grade molecular features depending on the amount of intermixed benign tissue. Second, Gleason grading on biopsy is subjective, with expert pathologists disagreeing on up to 30% of cases (16, 17). Molecular features that can be objectively measured will improve risk classification.

The 12 biomarkers identified in this study represent proteins with a range of functions, including transcription, protein synthesis, and regulation of cell proliferation and apoptosis, as well as cell structure (30). The fact that the biomarkers are able to perform despite biopsy sampling error indicates that protein-based biomarkers can further improve upon Gleason-based risk classification as a means to guide initial management of prostate cancer treatment.

### CONCLUSION

There is an urgent need for a reliable and accurate prognostic test for patients with prostate cancer, given the difficulties of predicting survival outcomes for patients diagnosed with early-stage cancer and the resulting overtreatment. The identification strategy for protein biomarkers described herein can also be applied to other tumor types and allows for performance-based selection of biomarkers that can be used to develop prognostic or predictive tests for other tumors where histological assessment is pivotal to risk stratification and prognostication.

Materials and Methods

Reagents and Antibodies

All antibodies and reagents used in this study were procured from commercially available sources as described in Table 17. Anti-fluroescein isothopcyanate (FITC) MAb-Alexa 568, anti-CK8-Alexa 488, anti-CK18-Alexa 488, anti-CK5-Alexa 555 and anti-Trim29-Alexa 555 were conjugated with Alexa dyes using the appropriate protein conjugation kits (Life Technologies).

TABLE 17
Antibody sources.

| Protein | H and L TMAs | Source | Cat # | Clonality | Host | Clone ID |
|---|---|---|---|---|---|---|
| CDKN1B (p27kip1) | Yes | Epitomics | 1591-1 | Mono | Rabbit | Y236 |
| FUS | Yes | Epitomics/ | 5321-1/ab133571 | Mono | Rabbit | EPR5813 |
| | | Abcam | | | | |
| CUL2 | Yes | Invitrogen | 700179 | Mono | Rabbit | 50H17L12 |
| SMAD2 | Yes | Invitrogen | 700048 | Mono | Rabbit | 31H15L54 |
| StAR | Yes | Santa Cruz | sc-166821 | Mono | Mouse | D-2 |
| DERL1 | Yes | Sigma | SAB4200148 | Mono | Mouse | Derlin1-1 |
| ST6GAL1 (CD75) | Yes | Novus | NB100-78091 | Mono | Mouse | LN1 |
| BHLHE40 (Dec1) | Yes | Santa Cruz | sc-101023 | Mono | Mouse | S-8 |
| EIF3H | Yes | Cell Signaling | 3413 | Mono | Rabbit | |
| AKAP8 | Yes | Epitomics | 6620-1 | Mono | Rabbit | EPR8978(B) |
| HOXB13 | Yes | Santa Cruz | sc-28333 | Mono | Mouse | F-9 |
| NCOA2 (clone 29) | Yes | Santa Cruz | 81280 | Mono | Mouse | |
| GRIP-1 | Yes | Santa Cruz | 136244 | Mono | Mouse | clone29 |
| PXN | Yes | Epitomics | 1500-1 | Mono | Rabbit | Y113 |
| ACTN1 | Yes | Santa Cruz | sc-17829 | Mono | Mouse | H-2 |
| FKBP5 | Yes | Epitomics | 5532-1 | Mono | Rabbit | EPR6617 |
| PDSS2 | Yes | Abcam | ab119768 | Mono | Mouse | 1D12 |
| DIABLO | Yes | Epitomics | 1012-1 | Mono | Rabbit | Y12 |
| COX6C | Yes | Santa Cruz | sc-65240 | Mono | Mouse | 3G5 |
| HSD17B4 | Yes | Santa Cruz | sc-365167 | Mono | Mouse | A-6 |
| YBX1 | Yes | Epitomics/ | 2397-1/76149 | Mono | Rabbit | EP2708Y |
| | | Abcam | | | | |
| MAOA | Yes | Epitomics | 5530-1 | Mono | Rabbit | EPR7101 |
| MAP3K5 | Yes | Epitomics | 1772-1 | Mono | Rabbit | EP553Y |
| VDAC1 | Yes | Santa Cruz | sc-58649 | Mono | Mouse | 20B12 |
| LATS2 | Yes | Abcam | ab54073 | Mono | Mouse | |
| PLAG1 | Yes | Sigma | SAB1404215 | Mono | Mouse | |
| AGPAT6 | Yes | Sigma/ | SAB1403460/16762- | Mono | Mouse | |
| | | Protein Tech | 1-AP | | | |
| EZH2 | Yes | Cell Signaling | 5246 | Mono | Rabbit | DC29 |
| DCC (HSPA9) | Yes | Leica | NCL-DCC | Mono | Mouse | DM51 |
| | | (Novocastra) | | | | |
| FAK1 | Yes | Epitomics | 2146-1 | Mono | Rabbit | EP1831Y |
| LMO7 | Yes | Santa Cruz | sc-365515 | Mono | Mouse | C-5 |
| MTDH2 | Yes | Epitomics | 3674-1 | Mono | Rabbit | EP4445 |
| AGK | Yes | Santa Cruz | sc-374390 | Mono | Mouse | F-3 |
| pS6 (POC) | Yes | Epitomics/ | 2268-1/ab157359 | Mono | Rabbit | EP1338(2)Y |
| | | Abcam | | | | |
| SMAD4 (POC) | Yes | Santa Cruz | sc-7966 | Mono | Mouse | B-8 |
| CCND1 (POC) | Yes | Spring Bio | M3044 | Mono | Rabbit | SP4 |
| pPRAS40 (POC) | Yes | Cell Signaling | 2997 | Mono | Rabbit | C77D7 |
| PTEN (POC) | Yes | Cell Signaling | 9188 | Mono | Rabbit | D4.3 |
| SPP1 (POC) | Yes | Abcam | ab91655 | Mono | Rabbit | EPR3688 |

Slide Processing and Staining Protocol

From TMA blocks, 5 μm sections were cut, placed on Histogrip (Life Technologies)-coated slides and processed as described previously (Supplementary Materials). Briefly, after deparaffinization, antigen retrieval was performed in 0.05% citraconic anhydride

solution for 45 min at 95° C. using a Lab Vision PT module (Thermo Scientific). Staining was performed either manually or in automated fashion with an Autostainer 360 or 720 (Thermo Scientific).

The QMIF staining procedure that combined two anti-biomarker antibodies with region-of-interest markers was performed as previously described (see Supplementary Materials and Methods). For diaminobenzidine (DAB)-based IHC staining, slides with tissue were processed as described above, blocked with Sniper Reagent™ (Biocare Medical) and incubated with primary antibody solution. UltraVision (Thermo Scientific) was used as a secondary reagent. Finally, tissue was counterstained with hematoxylin and coverslips were added.

Acquisition, Processing, Quality Control, and Annotation of FFPE Prostate Cancer Tissue Blocks

A set of FFPE human prostate cancer tissue blocks with clinical annotations and long-term patient outcome information was acquired from Folio Biosciences. Samples had been collected with appropriate institutional review board approval and all patient records were de-identified. For evaluation of candidate biomarker antibodies, FFPE human prostate cancer tissue blocks with limited clinical annotation were acquired from other commercial sources.

A series of 5 μm sections was cut from each FFPE block. For annotation, a 5 μm section that was the last to be cut from each FFPE block was stained with hematoxylin and eosin (H&E) and scanned using a ScanScope XT system (Aperio). The scanned images were remotely reviewed and annotated for GS in a blinded manner by expert clinical board-certified anatomical pathologists. Circles corresponding to 1 mm diameter cores were placed over four areas of highest and two areas of lowest Gleason patterns (see FIG. 32, top).

Generation of TMA Blocks

TMA blocks were prepared using a modified agarose block procedure(70). To generate the test TMA (MPTMA10), we selected 72 FFPE tissue blocks of prostatectomy samples with available annotations for GS and pathological stage. Of these, 37 had a GS of 3+3=6 with T2 stage, while 35 had a GS of 4+3=7 or a GS of either 3+3=6 or 3+4=7 with T3b stage. One 1 mm core per patient sample was taken from areas of lowest Gleason pattern and placed into an acceptor block.

For construction of H and L TMAs, we used the cohort of FFPE human prostate cancer tissue blocks with clinical annotations and long-term patient outcome information. For each patient sample, a core was taken from an area with the highest Gleason pattern and deposited into an H acceptor block. A second core was then taken from an area with the lowest Gleason pattern and put into an L acceptor block. The order of sample core placement into H block was randomized, and core positions in the L block were identical to those in the H block. In addition, cores from FFPE blocks of cell-line controls (Table 18) were placed in the upper and lower parts of all H and L TMA blocks. Upon completion, 5 μm serial sections were cut from each block and representative sections were stained with H&E and scanned with the ScanScope XT system. Images of H&E-stained cores were then independently annotated for observed Gleason pattern by a board-certified anatomical pathologist in a blinded manner.

TABLE 18
Cell-line controls. The cell lines listed were included as samples
on the TMA to provide positive controls for the antibodies used.

| Cell line | shRNA knockdown or treatment |
|---|---|
| DU145 | None |
| PC-3 | None |
| WM266-4 | None |
| RPMI7951 | None |
| BxPC-3 | None |
| RWPE-1 | None |
| SK-MEL-5 | None |
| DU145 | SMAD4 knockdown; 0 μg/ml Dox |
| DU145 | LY-treated for 1 hour |
| DU145 | SMAD4 knockdown; 1 μg/ml Dox |
| PC-3 | LY treated for 1 hour |
| DU145 | CCND1 knockdown; 0 μg/ml Dox |
| DU145 | CCND1 knockdown; 1 μg/ml Dox |

(Dox = doxycycline)

The resulting H and L TMA blocks were identical for a set of patient samples, but differed in observable Gleason pattern (FIG. 32, bottom). For this study, two pairs of TMA blocks (MPTMAF5H and 5L, 6H and 6L) were generated with cores from 380 patient samples.

Biomarker Selection

To identify biomarkers for prostate cancer aggressiveness, we developed a selection and evaluation process that could be broadly applicable across diseases and conditions. The process, shown in FIG. 33, had biological, technical, performance and validation stages.

In the biological stage, an initial list of potential biomarkers for prostate cancer aggressiveness was compiled from publically available data. The list was then prioritized based on biological relevance, in silico analysis, review of the Human Protein Atlas (www.proteinatlas.org), and commercial availability of requisite MAbs. Biological relevance review was based on mechanism of action in cells and, in particular, in the disease. In silico analysis was based on previously known gene amplifications, deletions and mutations, and univariate performance or progression correlation between these genetic alterations and the disease. The Human Protein Atlas contains data on protein expression levels in various tissues across disease states.

In the technical stage, commercial MAbs were obtained and tested for their ability to detect biomarkers from clinical samples. Initially, we stained samples of malignant and benign prostatic tissue using a DAB-based IHC staining procedure and selected candidate antibodies that exhibited a good signal:noise ratio and were specific for epithelial cell staining. We further tested successful candidates on malignant and benign prostatic tissue samples using IF along with region-of-interest markers, epithelial cytokeratins CK8 and CK18 and basal markers CK5 and Trim29, as described (Supplementary Materials and Methods). Antibodies and biomarkers that met the IF criteria were taken forward to the performance stage.

In the performance stage, MAbs were tested on TMAs. Performance was evaluated for a univariate correlation between tumor epithelium expression and disease state. The MAbs and biomarkers that demonstrated univariate correlation between expression and disease state were then evaluated on a larger H and L TMA set for both univariate correlation and performance in combination with other markers.

Image Acquisition

Two Vectra Intelligent Slide Analysis Systems (PerkinElmer) were used for automated image acquisition as described (Supplementary Materials and Methods). Multispectral images were processed into images for each separate fluorophore signal and sent for analysis with Definiens Developer script (Definiens AG).

Definiens Automated Image Analysis

We developed an automated image analysis algorithm using Definiens Developer XD for tumor identification and biomarker quantification. For each 1.0 mm TMA core, two 20× image fields were acquired. The Vectra multispectral image files were first converted into multilayer TIFF files using inForm (PerkinElmer) and a customized spectral library, and then converted to single-layer TIFF files using BioFormats (OME). The single-layer TIFF files were imported into the Definiens workspace using a customized import algorithm so that, for each TMA core, both of the image field TIFF files were loaded and analyzed as “maps” within a single “scene”.

Autoadaptive thresholding was used to define fluorescent intensity cut-offs for tissue segmentation in each individual tissue sample in our image analysis algorithm. Cell-line control cores within the TMA were automatically identified in the Definiens algorithm based on predefined core coordinates. The tissue samples were segmented using the fluorescent epithelial and basal cell markers, along with 4′, 6-diamidino-2-phenylindole (DAPI) for classification into epithelial cells, basal cells, and stroma, and further compartmentalized into cytoplasm and nuclei. Individual gland regions were classified as malignant or benign based on the relational features between basal cells and adjacent epithelial structures combined with object-related features, such as gland thickness. Epithelial markers are not present in all cell lines, therefore the cell-line controls were segmented into tissue versus background using the autofluorescence channel. Fields with artifact staining, insufficient epithelial tissue, or out-of-focus images were removed by a rigorous multi-parameter quality-control algorithm.

Epithelial marker and DAPI intensities were quantified in malignant and nonmalignant epithelial regions as quality-control measurements. Biomarker intensity levels were measured in the cytoplasm, nucleus, or whole cell in the malignant tissue based on predetermined subcellular localization criteria. The mean biomarker pixel intensity in the malignant compartments was averaged across the maps with acceptable quality parameters, to yield a single value for each tissue sample and cell line control core.

Data Stratification and Endpoints in the Analysis

Expression of 39 biomarkers was examined for correlation with tumor aggressiveness and lethality using the H and L TMAs. Disease aggressiveness was defined based on prostate pathology (aggressive disease=Surgical Gleason ≥3+4 or T3b, N+, or M+). For aggressiveness analyses, we examined marker correlation based on measurements in both L TMA samples with core Gleason ≤3+4 and the corresponding, matched H TMA samples.

For lethal outcome analyses, we created two different sample sets: (1) all cores with an observed GS≤3+4; and (2) all cores.

Cohort Composition

Table 12a presents the cohort composition. Only those samples that had a complete set of clinical information were included. When performing an analysis using a certain set of biomarkers, only samples with values for those markers were considered. Hence, the numbers in the table are upper bounds.

Univariate Analysis of Aggressiveness and Lethality

Our objectives for univariate analysis were twofold: to characterize univariate behavior as a performance assessment for potential inclusion in the final marker set, and to provide a reduced set of markers for exhaustive multivariable model exploration. All modeling was done in R 3.0 using standard functions and packages, including glm, survival, KMsurv, binom, and pROC. Biomarkers were assessed based on two outcomes: prediction of Surgical GS and prediction of death (lethality). Prediction of Surgical GS, categorized as indolent or more severe, was modeled with both ORs (logistic regression) and biomarker means (linear regression). Lethality was modeled using HRs (traditional Cox proportional hazards), ORs (logistic regression), and marker means (linear regression). In addition, to provide nonparametric and robust assessments, Wilcoxon and permutation tests were applied.

FIG. 34A-B show the key results. Univariate results were also directly considered in selection of the final marker set, as seen in FIG. 36A.

Biomarker Ranking for Aggressiveness Via Exhaustive Search of Multimarker Models

We rankED the biomarkers by importance in multimarker models; 31 biomarkers, refined from the original set of 39 to improve technical performance further, were used in an exhaustive biomarker search. We considered all combinations of up to five biomarkers from the 31 biomarkers tested in the L TMA in the H and L TMA analysis. For each biomarker combination, 500 training sets were generated by bootstrapping, and associated complementary test sets were obtained. A logistic regression model was applied to each training set and then tested on each of the associated test sets. Training and test AUC (i.e. C statistic) and training AIC were obtained in each round. Medians and 95% CIs were obtained for all three statistics.

We then considered biomarker selection frequency in the models and sorted them by their AIC and, separately, by their test AUC. For each of the resulting rankings of the models, the frequency of biomarker utilization in the top 1% and the top 5% of the lists was determined. The biomarkers that were included in at least 50% of models were then identified.

Table 15 shows biomarker frequency in the prediction of aggression assessment. The performance of the top-ranking models was similar. Moreover, the number of biomarkers in the top-ranking models varied. To resolve this issue, which appeared to relate to model size, we considered the top 1% of the models sorted by test AUC. We studied the resulting distributions for a number of different population assumptions, including cases where intermediate core GSs were excluded from analysis, or were included with indolent scores, or were included with high scores. In the final analysis, we concluded that an eight-biomarker model provided the best trade-off between performance and complexity in this experimental data set.

Biomarker Ranking for Lethality Via Exhaustive Search of Multimarker Models

The same model-building approach was followed for the biomarker ranking for prediction of lethality. Table 16 shows frequency of biomarker utilization (top 5%) for lethality.

Integration of Results in the Final Biomarker Set

The choice of the final set of 12 biomarkers needed to reflect their biological significance, as assessed in the univariate and multivariate analysis of patient sample measurements. Complicating and tempering the final choice were considerations of the technical limitations of the specific MAbs available for study. The final biomarker set selection is described in FIG. 36.

### SUPPLEMENTARY APPENDIX

Results

The twelve markers identified in this study were taken forward into another independent study of prostate cancer FFPE biopsy samples to develop a locked down model for clinical use (manuscript submitted). In this new study, we identified the best marker subset of the 12 markers and locked the resulting 8-marker model down, containing the following biomarkers: SMAD4, FUS, CUL2, YBX1, DERL1, PDSS2, HSPA9 and pS6. In the interest of completeness, we analyzed this set of markers on the TMA samples in this study, with the understanding that the TMA cohort contributed to the marker selection process. We again used the same patient partition, and trained on the L TMA followed by testing on both L TMA and H TMA samples. We analyzed 268 patients containing 40 dead-from-disease events. The resulting test AUC based on L TMA for prediction of aggressive disease was 0.64 (95% CI: 0.56-0.71) with a test odds ratio for aggressive disease of 13 per unit change in risk score (95% CI: 2.3-341). The test hazard ratio for lethal outcome prediction was 14 per unit change in risk score (95% CI: 1.3-393). To confirm the ability to generalize across sampling error, the model derived from L TMA train was also tested on the test H TMA with consistent results for both indications. The H TMA test AUC was 0.70 (95% CI: 0.62-0.78) with an odds ratio for aggressive disease of 46 per unit change in risk score (95% CI: 5.6-1290). The H TMA test hazard ratio for prediction of lethal outcome was 19 per unit change in risk score (95% CI: 1.4-620).

Materials and Methods

The Quantitative Multiplex Immunofluorescence (QMIF) Staining Procedure

The QMIF was composed of two initial blocking steps followed by four MAb incubation steps with appropriate washes in between. Blocking consisted of biotin blocking steps followed by treatment with Sniper reagent (Biocare Medical), according to the manufacturer's instructions. The first MAb incubation step consisted of a mixture of anti-biomarker 1 mouse MAb and anti-biomarker 2 rabbit MAb, followed by a second step containing a mixture of anti-mouse IgG Fab-fluorescein isothiocyanate (FITC) and anti-rabbit IgG Fab-biotin. A third “visualization” step included a mixture of anti-FITC MAb-Alexa 568, streptavidin-Alexa 633, as well as MAbs against epithelium (anti-CK8-Alexa 488 and anti-CK18-Alexa 488) and basal epithelium (anti-CK5-Alexa 555 and anti-Trim29-Alexa 555), respectively. A final, fourth step comprised a brief incubation with 4′, 6-diamidino-2-phenylindole (DAPI) for nuclear staining. After final washes, slides were mounted with Prolong Gold™ (Life Technologies) before coverslips were added. Slides were kept permanently at −20° C. before and after imaging.

FFPE Tissue Block Quality Evaluation

A 5 μm section from each FFPE block was manually stained with anti-phospho STAT3(T705) rabbit MAb, anti-STAT3 mouse MAb and region-of-interest markers, as described above. Slides were visually examined under a fluorescence microscope. Based on the staining intensities and autofluorescence, the sections and their corresponding FFPE blocks were graded into four quality categories.

Image Acquisition

Two Vectra Intelligent Slide Analysis Systems (PerkinElmer) were used for automated image acquisition as described elsewhere. DAPI, FITC, tetramethylrhodamine isothiocyanate (TRITC) and Cy5 long pass filter cubes were optimized for maximal multiplexing capability. Vectra 2.0 and Nuance 2.0 software packages (PerkinElmer) were used for automated image acquisition and development of the spectral library, respectively.

TMA acquisition protocols were run in an automated mode according to the manufacturer's instructions (PerkinElmer). Two 20× fields per core were imaged using a multispectral acquisition protocol that included consecutive exposures with DAPI, FITC, TRITC and Cy5 filters. For maximal reproducibility, light source intensity was adjusted with the help of an X-Cite Optical Power Measurement System (Lumen Dynamics) before image acquisition for each TMA slide. Identical exposure times were used for all slides containing the same antibody combination. A set of TMA slides stained with the same antibody combinations was imaged on the same Vectra microscope.

A spectral profile was generated for each fluorescent dye as well as for FFPE prostate tissue autofluorescence. Interestingly, two types of autofluorescence were observed in FFPE prostate tissue. A typical autofluorescence signal was common in both benign and tumor tissue, whereas an atypical “bright” type of autofluorescence was specific for bright granules present mostly in epithelial cells of benign tissue. A spectral library containing a combination of these two spectral profiles was used to separate or “unmix” individual dye signals from the autofluorescent background.

FFPE Cell-Line Controls

Selected cell lines were grown in standard conditions with and without treatment before harvesting as indicated (Table 18). Cells were washed with phosphate-buffered saline (PBS), fixed directly on plates with 10% formalin for 5 min, then scraped and collected in PBS with continued fixation at room temperature for 1 hour. Cells were washed twice with PBS, resuspended in Histogel (Thermo Scientific) at 70° C. and quickly spun down in a 1.5 ml microfuge tube to form a condensed cell-Histogel pellet. The pellets were then embedded in paraffin and placed into standard paraffin blocks that served as donor blocks for TMA construction. DU145 cells with inducible knock down of CCND1 and SMAD4 were established according to manufacturer's instructions using the ‘Tet-one’ system (Clontech).

Antibody Specificity Assays

Several MAbs, including anti-ACTN1, anti-CUL2, anti-Derlin1, anti-FUS, anti-PDSS2, anti-SMAD2, anti-VDAC1, anti-YBX1, and anti-HSPA9, were validated by Western blotting (WB) and immunohistochemistry (IHC) assay of target-specific knockdown and control cells (FIG. 37). Details of the small interfering RNA (siRNA) sequences and host cell lines are listed in Table 19. Cells were seeded into 12-well plates and transfected with 25 nM of siRNAs and DharmaFect transfection reagent (Thermo Scientific Dharmacon); mock transfection included only the transfection reagent. Cells transfected with two nontargeting sequences were also included as controls.

TABLE 19
siRNA sequences used for antibody validation. siRNAs were used to reduce
expression of the expected targets of the antibodies used to detect biomarkers.
Sequences for the siRNAs used in validation are given.

| | | | | | SEQ | |
|---|---|---|---|---|---|---|
| Gene | Gene | Cell | | | ID | Antibody |
| name | ID | line | Catalog no. | siRNA sequences | NO: | source |
| ACTN1 | 87 | HeLa | LQ-011195 | si5: GAGACAGCCGACACAGAUA | 27 | Santa Cruz |
| | | | | si6: UGACUUACGUGUCUAGCUU | 28 | sc-17829 |
| | | | | si7: GAACUGCCCGACCGGAUGA | 29 | |
| | | | | si8: GAAUACGGCUUUUGACGUG | 30 | |
| CUL2 | 8453 | HeLa | LQ-007277 | si5: GGAAGUGCAUGGUAAAUUU | 31 | Invitrogen |
| | | | | si6: CAUCCAAGUUCAUAUACUA | 32 | 700179 |
| | | | | si7: GCAGAAAGACACACCACAA | 33 | |
| | | | | si8: UGGUUUACCUCAUAUGAUU | 34 | |
| Derlin1 | 79139 | DU145 | LQ-010733 | si9: GGGCCAGGGCUUUCGACUU | 35 | Sigma |
| | | | | si11: CAACAAUCAUAUUCACGUU | 36 | SAB4200148 |
| FUS | 2521 | A375 | LQ-009497 | si7: GAUCAAUCCUCCAUGAGUA | 37 | Epitomics |
| | | | | si10: GAGCAGCUAUUCUUCUUAU | 38 | 5321-1 |
| PDSS2 | 57107 | HeLa | LQ-018550 | si5: GGAAGAGAUUUGUGGAUUA | 39 | Abcam |
| | | | | si6: GGCCAGAUCUGCUUUAGAA | 40 | ab119768 |
| | | | | si7: GAAUAUGGCAUUUCAGUAU | 41 | |
| | | | | si8: GAAGAUUGGACUAUGCUAA | 42 | |
| SMAD2 | 4087 | HeLa | LQ-003561 | si5: GAAUUGAGCCACAGAGUAA | 43 | Invitrogen |
| | | | | si6: GGUUUACUCUCCAAUGUUA | 44 | 700048 |
| | | | | si7: UCAUAAAGCUUCACCAAUC | 45 | |
| | | | | si8: ACUAGAAUGUGCACCAUAA | 46 | |
| VDAC1 | 7416 | A549 | LQ-019764 | si5: UAACACGCGCUUCGGAAUA | 47 | Abcam |
| | | | | si6: GAAACCAAGUACAGAUGGA | 48 | ab139752 |
| | | | | si7: GAGUACGGCCUGACGUUUA | 49 | |
| | | | | si8: CCUGAUAGGUUUAGGAUAC | 50 | |
| YBX1 | 4904 | A375 | LQ-010213 | si6: CUGAGUAAAUGCCGGCUUA | 51 | Epitomics |
| | | | | si7: CGACGCAGACGCCCAGAAAA | 52 | 2397-1 |
| | | | | si8: GUAAGGAACGGAUAUGGUU | 53 | |
| | | | | si9: GCGGAGGCAGCAAAUGUUA | 54 | |
| DCC | 1630 | A549 | LQ-003880 | si6: GGAAGCAACUUACGGAUAC | 55 | Leica |
| | | | | si7: GAUUCUGGCUCAAUUAUUA | 56 | NCL-DCC |
| | | | | si8: GAAGUCAGAUGAAGGCUUU | 57 | |
| | | | | si9: GUGAACAAAUGGGAAGUUU | 58 | |
| HSPA9 | 3313 | HeLa | LQ-004750 | si9: GGAAUGGCCUUAGUCAUGA | 59 | Santa Cruz |
| | | | | si10: CCAAUGGGAUAGUACAUGU | 60 | Sc-13967 |
| | | | | si11: CCUAUGGUCUAGACAAAUC | 61 | |
| SMAD4 | 4089 | | | | | Santa Cruz |
| | | | | | | sc-7966 |
| pS6 | | | | | | Epitomics |
| | | | | | | 2268-1 |
| | | | D-001810-01 | NT1: ON-TARGETplus Non- | | |
| | | | | targeting siRNA1 | | |
| | | | D-001810-02 | NT2: ON-TARGETplus Non- | | |
| | | | | targeting siRNA2 | | |

For WB assay, transfected cells were harvested at 72 hours and lyzed with Pierce RIM buffer (Thermo Scientific) supplemented with Halt protease inhibitor cocktail (Thermo Scientific). Protein concentration was measured using Pierce BCA reagent (Thermo Scientific). Samples were adjusted to equal protein concentrations and then mixed with sample buffer (Boston BioProducts) and run on precast Criterion TGX 4-15% SDS-PAGE gels (Bio-Rad). The samples were transferred onto PVDF or nitrocellulose membranes using the IBlot apparatus (Life Technologies), and immunoblotted with antibodies at 4° C. overnight, followed by incubation with secondary mouse or rabbit MAbs (Sigma Aldrich). The blots were developed with SuperSignal West Femto reagents (Thermo Scientific), and visualized by exposure to the FluorChem Q system (Protein Simple).

For the IHC assay, cells grown on coverslips in a 12-well plate were fixed with methanol on ice for 20 min at 72 hours post-transfection. This was followed by permeabilization with 0.2% Triton X-100 on ice for 10 min. UltraVision LP Detection System HRP Polymer/DAB Plus Chromogen Kit (Thermo Scientific) was used for the subsequent IHC assay according to the manufacturer's instructions.

The SMAD4 antibody was validated by WB and IHC assays of the SMAD4-positive cell line PC3 and the SMAD4-negative cell line BxPC3. The phospho-S6 antibody was validated by WB and IHC of naïve and LY294002-treated DU145 cells.

Cell Proliferation Assay

HeLa cells were transiently transfected with two nontargeting siRNAs as well as si9-11, specific for HSPA9 (see Table 19 for details of siRNA sequences). Cells were replated 48 hours after transfection and seeded in triplicate at 1000 cells per well in a 96-well plate. Cell proliferation was monitored using a CellTiter-Glo® Luminescent Cell Viability Kit (Promega) according to the manufacturer's instructions at 0, 24, 72 and 120 hours after replating.

Clonogenic Assay

At 48 hours post-transfection, HeLa cells were replated at 500 cells per well in a 6-well plate with 2 ml of cell medium. The cells were fixed with Crystal Violet Solution (Sigma) 7 days after plating. The images of each well were captured using AlphaView software in the FluorChem Q system (Protein Simple) and processed using ImageJ software.

Cell Vitality Assay

HeLa cells were harvested at 120 hours post-transfection. Cells were collected using trypsin. The cell pellets from each well of a 12-well plate were suspended in 500 μl of cell medium. Cell suspension (95 μl) was mixed with 5 μl of Solution 5 (VB-48/PI/AO), and 30 μl of the mixture was loaded onto an NC-Slide A2 (both from ChemoMetec). Cell vitality was measured by a NucleoCounter NC3000™ (ChemoMetec) according to the manufacturer's instructions.

Caspase Assay

HeLa cells were harvested at 120 hours after siRNA transfection using trypsin. Cells were suspended at 2×10⁶ cells/ml. An aliquot of 93 μl of the cell suspension was mixed with 5 μl diluted FLICA reagent (ImmunoChemistry Technologies) and 2 μl of Hoechst 33342 (Life Technologies). The mixture was incubated at 37° C. for 1 hour. HeLa cells were washed twice with 1× Apoptosis Buffer (ImmunoChemistry Technologies). The cell pellets were suspended in 100 μl 1× Apoptosis Buffer and 2 μl of propidium iodide. A 30 μl aliquot of the mixture was loaded onto an NC-Slide A2 and read using NucleoCounter NC-3000 software for caspase assay. Cells positive for FLICA staining were counted as apoptotic cells.

Identification of HSPA9 (Mortalin)

For identification of the Leica “anti-DCC” antibody target (FIG. 37), a preparative immunoprecipitation was performed. Ten p100 plates of confluent A549 cells were harvested with 5 ml of RIPA buffer (Thermo Scientific) with added protease inhibitors. The cell lysate was spun down at 14,000 rpm for 5 min; the supernatant was heated for 5 min at 80° C., then chilled on ice, and spun down again at 14,000 rpm for 5 min. Supernatant was collected and, after addition of 50 μl of Protein A/G beads (Thermo Scientific) with 2 μg of pre-bound “anti-DCC” antibody, was incubated with rocking at 4° C. for 2 hours. Beads were washed three times with TBS+1% Triton X100, and boiled with 30 μl of 1×SDS-PAGE loading buffer. Supernatant was loaded onto a 10% SDS-PAGE gel, and separated under standard SDS-PAGE conditions. The gel was stained with a silver stain kit for mass spectrometry (Thermo Scientific); the specific band was cut out, digested with trypsin, and subjected to MS/MS sequencing mass spectrometry at the Taplin Mass Spectrometry Facility (Harvard Medical School). Identified peptides were aligned with Human Protein reference databases. The identified protein HSPA9 was further validated as described.

### REFERENCES

1. R. Siegel, J. Ma, Z. Zou, A. Jemal, Cancer statistics, 2014. CA Cancer J Clin 64, 9-29 (2014).
2. S. Loeb, M. A. Bjurlin, J. Nicholson, T. L. Tammela, D. F. Penson, H. B. Carter, P. Carroll, R. Etzioni, Overdiagnosis and Overtreatment of Prostate Cancer. Eur Urol [Epub ahead of print], (2014).
3. J. L. Mohler, A. J. Armstrong, R. R. Bahnson, B. Boston, J. E. Busby, A. V. D'Amico, J. A. Eastham, C. A. Enke, T. Farrington, C. S. Higano, E. M. Horwitz, P. W. Kantoff, M. H. Kawachi, M. Kuettel, R. J. Lee, G. R. MacVicar, A. W. Malcolm, D. Miller, E. R. Plimack, J. M. Pow-Sang, M. Roach, 3rd, E. Rohren, S. Rosenfeld, S. Srinivas, S. A. Strope, J. Tward, P. Twardowski, P. C. Walsh, M.

Ho, D. A. Shead, Prostate cancer, Version 3.2012: featured updates to the NCCN guidelines. J Natl Compr Canc Netw 10, 1081-1087 (2012).
4. H. B. Carter, A. W. Partin, P. C. Walsh, B. J. Trock, R. W. Veltri, W. G. Nelson, D. S. Coffey, E. A. Singer, J. I. Epstein, Gleason score 6 adenocarcinoma: should it be labeled as cancer? J Clin Oncol 30, 4294-4296 (2012).
5. M. R. Cooperberg, J. M. Broering, P. R. Carroll, Time trends and local variation in primary treatment of localized prostate cancer. J Clin Oncol 28, 1117-1123 (2010).
6. V. A. Moyer, Screening for prostate cancer: U.S. Preventive Services Task Force recommendation statement. Ann Intern Med 157, 120-134 (2012).
7. T. J. Wilt, R. MacDonald, I. Rutks, T. A. Shamliyan, B. C. Taylor, R. L. Kane, Systematic review: comparative effectiveness and harms of treatments for clinically localized prostate cancer. Ann Intern Med 148, 435-448 (2008).
8. NCCN, Prostate cancer early detection, Version 2.2012, Available at www.NCCN.org. Accessed 14 Feb. 2014. (2012).
9. NCCN, NCCN Clinical Practice Guidelines in Oncology: Prostate Cancer, Version 3.2012; available at: https://www.nccn.org/store/login/login.aspx?ReturnURL=http://www.nccn.org/pr ofessionals/physician_gls/pdf/prostate.pdf Accessed 14-February 2014. (2012).
10. J. I. Epstein, Z. Feng, B. J. Trock, P. M. Pierorazio, Upgrading and downgrading of prostate cancer from biopsy to radical prostatectomy: incidence and predictive factors using the modified Gleason grading system and factoring in tertiary grades. Eur Urol 61, 1019-1024 (2012).
11. N. M. Corcoran, C. M. Hovens, M. K. Hong, J. Pedersen, R. G. Casey, S. Connolly, J. Peters, L. Harewood, M. E. Gleave, S. L. Goldenberg, A. J. Costello, Underestimation of Gleason score at prostate biopsy reflects sampling error in lower volume tumours. BJU Int 109, 660-664 (2012).
12. S. P. Porten, J. M. Whitson, J. E. Cowan, M. R. Cooperberg, K. Shinohara, N. Perez, K. L. Greene, M. V. Meng, P. R. Carroll, Changes in prostate cancer grade on serial biopsy in men undergoing active surveillance. J Clin Oncol 29, 2795-2800 (2011).
13. M. A. Bjurlin, S. S. Taneja, Standards for prostate biopsy. Curr Opin Urol 24, 155-161 (2014).
14. J. D. Davies, M. A. Aghazadeh, S. Phillips, S. Salem, S. S. Chang, P. E. Clark, M. S. Cookson, R. Davis, S. D. Herrell, D. F. Penson, J. A. Smith, Jr., D. A. Barocas, Prostate size as a predictor of Gleason score upgrading in patients with low risk prostate cancer. J Urol 186, 2221-2227 (2011).
15. R. Kvale, B. Moller, R. Wahlqvist, S. D. Fossa, A. Berner, C. Busch, A. E. Kyrdalen, A. Svindland, T. Viset, O. J. Halvorsen, Concordance between Gleason scores of needle biopsies and radical prostatectomy specimens: a population-based study. BJU Int 103, 1647-1654 (2009).
16. M. Goodman, K. C. Ward, A. O. Osunkoya, M. W. Datta, D. Luthringer, A. N. Young, K. Marks, V. Cohen, J. C. Kennedy, M. J. Haber, M. B. Amin, Frequency and determinants of disagreement and error in gleason scores: a population-based study of prostate cancer. Prostate 72, 1389-1398 (2012).
17. J. K. McKenney, J. Simko, M. Bonham, L. D. True, D. Troyer, S. Hawley, L. F. Newcomb, L. Fazli, L. P. Kunju, M. M. Nicolas, F. Vakar-Lopez, X. Zhang, P. R. Carroll, J. D. Brooks, The potential impact of reproducibility of Gleason grading in men with early stage prostate cancer managed by active surveillance: a multi-institutional study. J Urol 186, 465-469 (2011).
18. M. R. Cooperberg, D. J. Pasta, E. P. Elkin, M. S. Litwin, D. M. Latini, J. Du Chane, P. R. Carroll, The University of California, San Francisco Cancer of the Prostate Risk Assessment score: a straightforward and reliable preoperative predictor of disease recurrence after radical prostatectomy. J Urol 173, 1938-1942 (2005).
19. A. V. D'Amico, R. Whittington, S. B. Malkowicz, D. Schultz, K. Blank, G. A. Broderick, J. E. Tomaszewski, A. A. Renshaw, I. Kaplan, C. J. Beard, A. Wein, Biochemical outcome after radical prostatectomy, external beam radiation therapy, or interstitial radiation therapy for clinically localized prostate cancer. JAMA 280, 969-974 (1998).
20. A. Vellekoop, S. Loeb, Y. Folkvaljon, P. Stattin, Population-based study of predictors of adverse pathology among candidates for active surveillance with Gleason 6 prostate cancer. J Urol 191, 350-357 (2014).
21. J. I. Epstein, W. C. Allsbrook, Jr., M. B. Amin, L. L. Egevad, The 2005 International Society of Urological Pathology (ISUP) Consensus Conference on Gleason Grading of Prostatic Carcinoma. Am J Surg Pathol 29, 1228-1242 (2005).
22. A. C. Reese, J. E. Cowan, J. S. Brajtbord, C. R. Harris, P. R. Carroll, M. R. Cooperberg, The quantitative Gleason score improves prostate cancer risk assessment. Cancer 118, 6046-6054 (2012).
23. K. Eichler, S. Hempel, J. Wilby, L. Myers, L. M. Bachmann, J. Kleijnen, Diagnostic value of systematic biopsy methods in the investigation of prostate cancer: a systematic review. J Urol 175, 1605-1612 (2006).
24. N. B. Delongchamps, G. de la Roza, R. Jones, M. Jumbelic, G. P. Haas, Saturation biopsies on autopsied prostates for detecting and characterizing prostate cancer. BJU Int 103, 49-54 (2009).
25. N. L. Robertson, M. Emberton, C. M. Moore, MRI-targeted prostate biopsy: a review of technique and results. Nat Rev Urol 10, 589-597 (2013).
26. M. Shipitsin, C. Small, E. Giladi, S. Siddiqui, S. Choudhury, S. Hussain, Y. E. Huang, H. Chang, D. L. Rimm, D. Berman, T. P. Nifong, P. Blume-Jensen, Development of an automated quantitative multiplex proteomics in situ imaging platform and application in prediction of prostate cancer lethal outcome. Submitted, (2014).
27. S. Shikanov, S. E. Eggener, Hazard of prostate cancer specific mortality after radical prostatectomy. J Urol 187, 124-127 (2012).
28. H. M. Ross, O. N. Kryvenko, J. E. Cowan, J. P. Simko, T. M. Wheeler, J. I. Epstein, Do adenocarcinomas of the prostate with Gleason score (GS)</=6 have the potential to metastasize to lymph nodes? Am J Surg Pathol 36, 1346-1352 (2012).
29. F. Brimo, R. Montironi, L. Egevad, A. Erbersdobler, D. W. Lin, J. B. Nelson, M. A. Rubin, T. van der Kwast, M. Amin, J. I. Epstein, Contemporary grading for prostate cancer: implications for patient care. Eur Urol 63, 892-901 (2013).
30. Z. Ding, C. J. Wu, G. C. Chu, Y. Xiao, D. Ho, J. Zhang, S. R. Perry, E. S. Labrot, X. Wu, R. Lis, Y. Hoshida, D. Hiller, B. Hu, S. Jiang, H. Zheng, A. H. Stegh, K. L. Scott, S. Signoretti, N. Bardeesy, Y. A. Wang, D. E. Hill, T. R. Golub, M. J. Stampfer, W. H. Wong, M. Loda, L. Mucci, L. Chin, R. A. DePinho, SMAD4-dependent barrier constrains prostate cancer growth and metastatic progression. Nature 470, 269-273 (2011).
31. J. Lapointe, C. Li, C. P. Giacomini, K. Salari, S. Huang, P. Wang, M. Ferrari, T. Hernandez-Boussard, J. D. Brooks, J. R. Pollack, Genomic profiling reveals alternative genetic pathways of prostate tumorigenesis. Cancer Res 67, 8504-8510 (2007).
32. J. Lapointe, C. Li, J. P. Higgins, M. van de Rijn, E. Bair, K. Montgomery, M. Ferrari, L. Egevad, W. Rayford, U. Bergerheim, P. Ekman, A. M. DeMarzo, R. Tibshirani, D. Botstein, P. O. Brown, J. D. Brooks, J. R. Pollack, Gene expression profiling identifies clinically relevant subtypes of prostate cancer. Proc Natl Acad Sci USA 101, 811-816 (2004).
33. E. K. Markert, H. Mizuno, A. Vazquez, A. J. Levine, Molecular classification of prostate cancer using curated expression signatures. Proc Natl Acad Sci USA 108, 21276-21281 (2011).
34. Z. Ding, C. J. Wu, M. Jaskelioff, E. Ivanova, M. Kost-Alimova, A. Protopopov, G. C. Chu, G. Wang, X. Lu, E. S. Labrot, J. Hu, W. Wang, Y. Xiao, H. Zhang, J. Zhang, B. Gan, S. R. Perry, S. Jiang, L. Li, J. W. Homer, Y. A. Wang, L. Chin, R. A. DePinho, Telomerase reactivation following telomere dysfunction yields murine prostate tumors with bone metastases. Cell 148, 896-907 (2012).
35. G. P. Swanson, D. Quinn, Using molecular markers to help predict who will fail after radical prostatectomy. Prostate Cancer 2011, 290160 (2011).
36. L. True, I. Coleman, S. Hawley, C. Y. Huang, D. Gifford, R. Coleman, T. M. Beer, E. Gelmann, M. Datta, E. Mostaghel, B. Knudsen, P. Lange, R. Vessella, D. Lin, L. Hood, P. S. Nelson, A molecular correlate to the Gleason grading system for prostate adenocarcinoma. Proc Natl Acad Sci USA 103, 10991-10996 (2006).
37. A. E. Ross, L. Marchionni, M. Vuica-Ross, C. Cheadle, J. Fan, D. M. Berman, E. M. Schaeffer, Gene expression pathways of high grade localized prostate cancer. Prostate 71, 1568-1578 (2011).
38. T. Nakagawa, T. M. Kollmeyer, B. W. Morlan, S. K. Anderson, E. J. Bergstralh, B. J. Davis, Y. W. Asmann, G. G. Klee, K. V. Ballman, R. B. Jenkins, A tissue biomarker panel predicting systemic progression after PSA recurrence post-definitive prostate cancer therapy. PLoS One 3, e2318 (2008).
39. J. C. Cheville, R. J. Karnes, T. M. Therneau, F. Kosari, J. M. Munz, L. Tillmans, E. Basal, L. J. Rangel, E. Bergstralh, I. V. Kovtun, C. D. Savci-Heijink, E. W. Klee, G. Vasmatzis, Gene panel model predictive of outcome in men at high-risk of systemic progression and death from prostate cancer after radical retropubic prostatectomy. J Clin Oncol 26, 3930-3936 (2008).
40. D. V. Makarov, S. Loeb, R. H. Getzenberg, A. W. Partin, Biomarkers for prostate cancer. Annu Rev Med 60, 139-151 (2009).
41. J. R. Graff, B. W. Konicek, R. L. Lynch, C. A. Dumstorf, M. S. Dowless, A. M. McNulty, S. H. Parsons, L. H. Brail, B. M. Colligan, J. W. Koop, B. M. Hurst, J. A. Deddens, B. L. Neubauer, L. F. Stancato, H. W. Carter, L. E. Douglass, J. H. Carter, eIF4E activation is commonly elevated in advanced human prostate cancers and significantly related to reduced patient survival. Cancer Res 69, 3866-3873 (2009).
42. I. P. Gorlov, K. Sircar, H. Zhao, S. N. Maity, N. M. Navone, O. Y. Gorlova, P. Troncoso, C. A. Pettaway, J. Y. Byun, C. J. Logothetis, Prioritizing genes associated with prostate cancer development. BMC Cancer 10, 599 (2010).
43. I. Cima, R. Schiess, P. Wild, M. Kaelin, P. Schuffler, V. Lange, P. Picotti, R. Ossola, A. Templeton, O. Schubert, T. Fuchs, T. Leippold, S. Wyler, J. Zehetner, W. Jochum, J. Buhmann, T. Cerny, H. Moch, S. Gillessen, R. Aebersold, W. Krek, Cancer genetics-guided discovery of serum biomarker signatures for diagnosis and prognosis of prostate cancer. Proc Natl Acad Sci USA 108, 3342-3347 (2011).
44. M. Chen, C. P. Pratt, M. E. Zeeman, N. Schultz, B. S. Taylor, A. O'Neill, M. Castillo-Martin, D. G. Nowak, A. Naguib, D. M. Grace, J. Murn, N. Navin, G. S. Atwal, C. Sander, W. L. Gerald, C. Cordon-Cardo, A. C. Newton, B. S. Carver, L. C. Trotman, Identification of PHLPP1 as a tumor suppressor reveals the role of feedback activation in PTEN-mutant prostate cancer progression. Cancer Cell 20, 173-186 (2011).
45. G. Kristiansen, Diagnostic and prognostic molecular biomarkers for prostate cancer. Histopathology 60, 125-141 (2012).
46. N. C. Pressinotti, H. Klocker, G. Schafer, V. D. Luu, M. Ruschhaupt, R. Kuner, E. Steiner, A. Poustka, G. Bartsch, H. Sultmann, Differential expression of apoptotic genes PDIA3 and MAP3K5 distinguishes between low- and high-risk prostate cancer. Mol Cancer 8, 130 (2009).
47. B. S. Taylor, N. Schultz, H. Hieronymus, A. Gopalan, Y. Xiao, B. S. Carver, V. K. Arora, P. Kaushik, E. Cerami, B. Reva, Y. Antipin, N. Mitsiades, T. Landers, I. Dolgalev, J. E. Major, M. Wilson, N. D. Socci, A. E. Lash, A. Heguy, J. A. Eastham, H. I. Scher, V. E. Reuter, P. T. Scardino, C. Sander, C. L. Sawyers, W. L. Gerald, Integrative genomic profiling of human prostate cancer. Cancer Cell 18, 11-22 (2010).
48. S. A. Tomlins, R. Mehra, D. R. Rhodes, X. Cao, L. Wang, S. M. Dhanasekaran, S. Kalyana-Sundaram, J. T. Wei, M. A. Rubin, K. J. Pienta, R. B. Shah, A. M. Chinnaiyan, Integrative molecular concept modeling of prostate cancer progression. Nature genetics 39, 41-51 (2007).
49. Y. A. Yang, J. Yu, EZH2, an epigenetic driver of prostate cancer. Protein Cell 4, 331-341 (2013).
50. I. G. Mills, HOXB13, RFX6 and prostate cancer risk. Nat Genet 46, 94-95 (2014).
51. G. Hu, Y. Wei, Y. Kang, The multifaceted role of MTDH/AEG-1 in cancer progression. Clin Cancer Res 15, 5615-5620 (2009).
52. J. K. Lindsey, B. Jones, Choosing among generalized linear models applied to medical data. Stat Med 17, 59-68 (1998).
53. Z. Flachbartova, B. Kovacech, Mortalin—a multipotent chaperone regulating cellular processes ranging from viral infection to neurodegeneration. Acta Virol 57, 3-15 (2013).
54. C. H. Bangma, M. J. Roobol, Defining and predicting indolent and low risk prostate cancer. Crit Rev Oncol Hematol 83, 235-241 (2012).
55. A. Heidenreich, P. J. Bastian, J. Bellmunt, M. Bolla, S. Joniau, T. van der Kwast, M. Mason, V. Matveev, T. Wiegel, F. Zattoni, N. Mottet, EAU guidelines on prostate cancer. part 1: screening, diagnosis, and local treatment with curative intent-update 2013. Eur Urol 65, 124-137 (2014).
56. G. S. Sandhu, G. L. Andriole, Overdiagnosis of prostate cancer. J Natl Cancer Inst Monogr 2012, 146-151 (2012).
57. S. A. Boorjian, R. J. Karnes, P. L. Crispen, L. J. Rangel, E. J. Bergstralh, T. J. Sebo, M. L. Blute, The impact of discordance between biopsy and pathological Gleason scores on survival after radical prostatectomy. J Urol 181, 95-104; discussion 104 (2009).
58. S. Irshad, M. Bansal, M. Castillo-Martin, T. Zheng, A. Aytes, S. Wenske, C. Le Magnen, P. Guarnieri, P. Sumazin, M. C. Benson, M. M. Shen, A. Califano, C. Abate-Shen, A molecular signature predictive of indolent prostate cancer. Sci Transl Med 5, 3006408 (2013).
59. J. Xu, Q. Li, Review of the in vivo functions of the p160 steroid receptor coactivator family. Mol Endocrinol 17, 1681-1692 (2003).
60. M. Sattler, E. Pisick, P. T. Morrison, R. Salgia, Role of the cytoskeletal protein paxillin in oncogenesis. Crit Rev Oncog 11, 63-76 (2000).
61. D. A. Canton, J. D. Scott, Anchoring proteins encounter mitotic kinases. (Cell Cycle. 2013 Mar. 15; 12(6):863-4. doi: 10.4161/cc.24192. Epub 2013 Mar. 5.).
62. J. Cuzick, G. P. Swanson, G. Fisher, A. R. Brothman, D. M. Berney, J. E. Reid, D. Mesher, V. O. Speights, E. Stankiewicz, C. S. Foster, H. Moller, P. Scardino, J. D. Warren, J. Park, A. Younus, D. D. Flake, 2nd, S. Wagner, A. Gutin, J. S. Lanchbury, S. Stone, G. Transatlantic Prostate, Prognostic value of an RNA expression signature derived from cell cycle proliferation genes in patients with prostate cancer: a retrospective study. The lancet oncology 12, 245-255 (2011).
63. H. I. Scher, M. J. Morris, S. Larson, G. Heller, Validation and clinical utility of prostate cancer biomarkers. Nat Rev Clin Oncol 10, 225-234 (2013).
64. A. Gopalan, M. A. Leversha, J. M. Satagopan, Q. Zhou, H. A. Al-Ahmadie, S. W. Fine, J. A. Eastham, P. T. Scardino, H. I. Scher, S. K. Tickoo, V. E. Reuter, W. L. Gerald, TMPRSS2-ERG gene fusion is not associated with outcome in patients treated by prostatectomy. Cancer Res 69, 1400-1406 (2009).
65. J. Cuzick, D. M. Berney, G. Fisher, D. Mesher, H. Moller, J. E. Reid, M. Perry, J. Park, A. Younus, A. Gutin, C. S. Foster, P. Scardino, J. S. Lanchbury, S. Stone, Prognostic value of a cell cycle progression signature for prostate cancer death in a conservatively managed needle biopsy cohort. Br J Cancer 106, 1095-1099 (2012).
66. J. T. Bishoff, S. J. Freedland, L. Gerber, P. Tennstedt, J. Reid, W. Welbourn, M. Graefen, Z. Sangale, E. Tikishvili, J. Park, A. Younus, A. Gutin, J. S. Lanchbury, G. Sauter, M. Brawer, S. Stone, T. Schlomm, Prognostic utility of the CCP score generated from biopsy in men treated with prostatectomy. J Urol 6, 00248-00241 (2014).
67. M. R. Cooperberg, J. P. Simko, J. E. Cowan, J. E. Reid, A. Djalilvand, S. Bhatnagar, A. Gutin, J. S. Lanchbury, G. P. Swanson, S. Stone, P. R. Carroll, Validation of a cell-cycle progression gene panel to improve risk stratification in a contemporary prostatectomy cohort. Journal of clinical oncology: official journal of the American Society of Clinical Oncology 31, 1428-1434 (2013).
68. L. K. Boyd, X. Mao, Y. J. Lu, The complexity of prostate cancer: genomic alterations and heterogeneity. Nat Rev Urol 9, 652-664 (2012).
69. A. G. Sowalsky, H. Ye, G. J. Bubley, S. P. Balk, Clonal progression of prostate cancers from Gleason grade 3 to grade 4. Cancer Res 73, 1050-1055 (2013).
70. P. Yan, W. Seelentag, A. Bachmann, F. T. Bosman, An agarose matrix facilitates sectioning of tissue microarray blocks. J Histochem Cytochem 55, 21-24 (2007).

### Example 7: Clinical Validation of a Proteomic In Situ Biopsy Test for Discriminating Favorable from Nonfavorable Prostate Cancer

### SUMMARY

Prostate cancer aggressiveness and appropriate therapy are determined following biopsy sampling. Current clinical and pathologic parameters are insufficient for accurate risk prediction, leading primarily to overtreatment but also missed opportunities for curative therapy.

An 8-biomarker proteomic assay for intact tissue biopsies predictive of prostate pathology was defined in a study of 381 patient biopsies with matched prostatectomy specimens and validated in a subsequent blinded study of 276 patient cases. The ability to distinguish pathologically ‘favorable’ versus ‘nonfavorable’ disease profiles based on prostatectomy was determined relative to current standards of care (SOC) for risk classification.

The validation study met its two predefined endpoints, separating favorable from nonfavorable pathology (AUC, 0.68, P<0.0001, odds ratio=20.9). Favorable (risk score ≤0.33) and nonfavorable (risk score >0.80) patient categories were defined based on ‘false negative’ and ‘false positive’ rates of 10% and 5%, respectively. At a risk score ≤0.33, predictive values for favorable patients in very-low- and low-risk NCCN and low-risk D'Amico groups were 95%, 81.5%, and 87.2%, respectively, higher than for SOC risk groups themselves (80.3%, 63.8%, and 70.6%, respectively). The predictive value for nonfavorable patients was 76.9% at risk scores >0.8 across all risk groups. Increased risk scores correlated with decreased frequency of favorable cases across all risk groups. The Net Reclassification Index for NCCN was 0.34 (P<0.00001) and for D'Amico was 0.24 (P=0.0001).

The 8-biomarker test provided individualized, independent, and complementary information to that of SOC risk stratification systems, and can aid clinical decision-making at time of biopsy.

### INTRODUCTION

In 2014, there will be an estimated 233,000 new diagnoses of prostate cancer in the USA.¹ The majority of patients have early-stage, clinically localized disease.¹⁻⁵ Given the marked heterogeneity of prostate cancer and concerns regarding its overtreatment,⁶⁻⁸ it is important, after biopsy and before definitive treatment, to distinguish indolent cases with good prognosis from more aggressive cases with poor survival.⁹ Pathologic evaluation of tissue obtained by needle biopsy is essential both to confirm a prostate cancer diagnosis and to determine a patient's risk category.¹⁰ A number of classification systems have been developed that combine available clinical and pathological parameters.^{9,11} However, all classification systems are imperfect, and none are designed to ascribe an individualized risk score.¹²⁻¹⁴

Approximately 25-30% of patients considered at diagnosis to have low-risk disease subsequently have their tumor pathology upgraded.¹⁴⁻¹⁶ Indeed, a significant proportion of patients will have upgrading or downgrading from an initial ‘biopsy’ Gleason score to a more accurate ‘surgical’ or pathologic Gleason score after analysis of radical prostatectomy tissue.¹⁶ These revisions may reflect initial biopsy sampling error,¹⁷ or pathologist discordance in tumor grading,¹⁸ both of which can contribute to overtreatment or undertreatment of disease.⁷ There are particular concerns around over-calling or under-calling Gleason pattern 4 in needle biopsy samples,^{19 16,20,21} and a continuing need to be able to determine in patients with low- to intermediate-grade disease on biopsies whether the cancer is organ-confined, or non-organ-confined with ultimate metastatic potential.

Advances have been made in identifying genetic markers informing clinical risk prognostication, one such example being the expression of a set of cell cycle progression genes used to predict risk of death.²²⁻²⁵ There has also been focus on identifying in situ protein biomarkers that, under circumstances of tumor heterogeneity, enable measurements from the most aggressive tumor areas, even if from only few cancer cells.²⁶⁻²⁸ Using a quantitative multiplex proteomics in situ imaging (QMPI) approach we identified in a large clinical independent study 12 biopsy biomarker candidates tailored to be resistant to sampling error, that predict both prostate pathology aggressiveness and lethal outcome (see Example 6 and Supplementary Appendix below).

Here the model development and subsequent blinded validation of an eight-biomarker signature derived from these 12 markers in two separate clinical biopsy studies, each with matched annotated prostatectomy specimens, is reported. The first study was designed to define and lock down the biomarker signature model and the QMPI assay (ProMark™) through logistic regression (train-test) analyses to yield a risk score for potential disease aggressiveness. The blinded clinical validation study evaluated the ability of the biopsy assay to predict the clinically relevant dichotomous endpoint of favorable versus nonfavorable pathology at prostatectomy. The differential information provided by the assay and risk score was compared with two risk stratification systems, the D'Amico system and the NCCN guideline categories,^{9,11} and considered for its potential to provide additional accuracy in predicting prognosis for the individual patient as a potential aid in decision-making.

Methods

The QMPI approach for protein in situ measurements was as described in the Supplementary Appendix below.

Clinical Model Building Study and Assay Lockdown

A noninterventional, retrospective clinical model development study using biopsy case tissue samples was devised to define the best marker subset signature out of 12 previously identified biomarker candidates shown to correlate with both prostate pathology aggressiveness and lethal outcome. The study goal was to define a model able to distinguish between prostate pathology with a surgical Gleason 3+3 and ≤T3a (“GS 6”) versus surgical Gleason ≥3+4 or non-localized >T3a, N, or M (“non-GS 6”), based on studies showing that tumors with surgical Gleason 3+3 at prostatectomy do not metastasize.^{29,30} The study protocol was approved by Institutional Review Boards (IRBs), and patient consent was obtained or waived accordingly.

To develop a robust assay, multiple institutions were recruited representing typical US patient cohorts: Urology Austin, Chesapeake Urology Associates, Cleveland Clinic, Michigan Urology, and Folio Biosciences. Biopsy sample inclusion/exclusion criteria matched those that would be in place during routine clinical use of the assay (Supplementary Appendix). Patients with biopsy Gleason ≥4+3 were excluded, except for a limited number of biopsies that had been discordantly graded as both 3+4 and 4+3 by two expert pathologists. Annotation including information on matched biopsy and prostatectomy pathology reports was required. All samples were blinded during laboratory processing.

The biomarker signature was optimized as a logistic regression model to estimate probability of “non-GS 6”, determined by bootstrap analysis of independent training and testing sets. Models were characterized by the area under the receiver operating characteristic (ROC) curve (AUC), and sorted by increasing value of Akaike information criterion (AIC),³¹ decreasing value of the AUC on the training set, and decreasing value of the AUC on the testing set. The frequency of marker usage was then determined in the 10% most highly ranked models to finalize the biomarker set. A risk score, a continuous number between 0 and 1, was computed to estimate the likelihood of “non-GS 6” pathology. Sensitivity analyses were performed to confirm the defined, locked-down assay.

Clinical Validation Study

A noninterventional, blinded, prospectively designed, retrospectively collected clinical study was conducted to validate the performance of the eight-biomarker biopsy assay in predicting prostate pathology on its own and relative to current standards of care (SOC) for patient risk categorization. The cohort comprised biopsy samples with matched prostatectomy annotation from patients managed at the University of Montreal, Canada. Consent criteria and IRB approval steps were as for the clinical development study. Inclusion criteria were biopsies with a centralized Gleason score 3+3 or 3+4 (biopsies with discordant grading by two expert pathologists of 3+4 and 4+3 were included as well), and matched prostatectomy with pathologic TNM staging, PSA level, and Gleason score. Performance of the assay was assessed using ROCs and corresponding AUCs for the diagnostic risk score.

Two co-primary endpoints for prostate pathology were validated by the biopsy assay-derived risk score, as assessed by AUC:
1. ‘Favorable’ pathology—surgical Gleason ≤3+4 and organ-confined disease (≤T2) versus
   ‘Nonfavorable’ pathology—surgical Gleason ≥4+3 or non-organ-confined disease (T3a, T3b, N, or M) and
2. “GS 6” pathology—surgical Gleason of 3+3 and localized disease (≤T3a) versus “Non-GS 6” pathology—surgical Gleason ≥3+4 or nonlocalized disease (T3b, N, or M)

Favorable versus nonfavorable pathology was chosen for final patient categorization throughout the validation study. It reflects the increasing awareness that organ-confined disease with minimal Gleason 4 pattern is likely to remain harmless with a significantly better long-term prognosis than higher-grade (dominant Gleason 4 pattern) or non-organ-confined disease^{30,32,33}

Secondary analyses included odds ratios (ORs) for the highest quartile versus lowest quartile of risk score, and OR (point estimate) for the continuous scale. We compared the risk outcomes from our diagnostic test with the SOC risk classification categories as defined by D'Amico and the NCCN,^{9,11} using positive predictive values (PPVs). Definition and statistical analysis of the Net Reclassification Index (NRI) was done as described by Pencina.³⁴

See the Supplementary Appendix for the statistical plan for both clinical studies.

Results

Clinical Model Building Study and Assay Lockdown

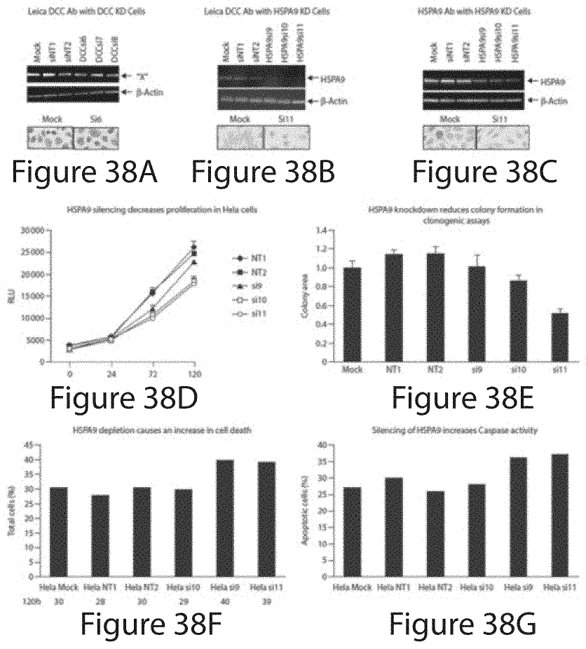

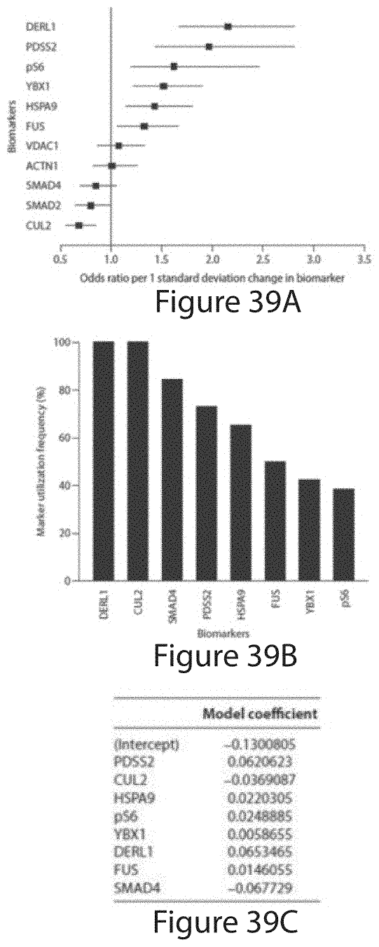

Tumor characteristics of the 381 patients included in the model development study are shown in Table 20. FIG. 39A-C illustrates the model optimization process. FIG. 39A shows the univariate OR associated with the biomarkers evaluated. Model performance was assessed and several high-performing models, e.g. test AUC of 0.79 (95% confidence interval [CI], 0.72 to 0.84), were identified. FIG. 39B shows the resulting biomarker frequencies for all models with a maximum of eight biomarkers. The resulting locked-down signature is shown in FIG. 39C.

TABLE 20
Summary of the Clinical Patient Cohorts (Tumor Characteristics
at Biopsy and at Radical Prostatectomy/Surgical Gleason)
in the Clinical Development and Validation Studies.

| | Biomaker | | Clinical | |
|---|---|---|---|---|
| | Lockdown | | Validation | |
| | Study | | Study | |
| Total (N) | 381 | | 276 | |
| Age, mean (SD), years | 60.6 (7.0) | | 59.9 (5.7) | |
| PSA, mean (SD), ng/ml | 7.7* (9.6) | | 6.7 (5.7) | |
| | N | % | N | % |
| Biopsy Gleason score | | | | |
| 3 + 3 | 162 | 42.5 | 191 | 69.2 |
| 3 + 4 | 191 | 50.1 | 68 | 24.6 |
| 4 + 3 | 28 | 7.3 | 17 | 6.2 |
| Surgical Gleason score | | | | |
| 3 + 3 = 6 | 138 | 36.2 | 80 | 29 |
| 3 + 4 | 97 | 25.5 | 150 | 54.3 |
| 4 + 3 | 119 | 31.2 | 39 | 14.1 |
| 8 | 14 | 3.7 | 4 | 1.5 |
| 9 | 13 | 3.4 | 3 | 1.1 |
| Pathologic stage | | | | |
| T1 | 2 | 0.6 | | |
| T2 | 269 | 70.5 | 168 | 60.8 |
| T3a | 72† | 18.9 | 91 | 33 |
| T3b-c | 37 | 9.7 | 15 | 5.5 |
| T4 | 1 | 0.3 | 0 | 0 |
| Missing | 0 | 0 | 2 | 0.7 |
| Source Institution | | | | |
| University of | — | — | 276 | 100 |
| Montreal | | | | |
| Urology Austin | 147 | 38.6 | — | — |
| Chesapeake Urology | 8 | 2.1 | — | — |
| Associates | | | | |
| Cleveland Clinic | 71 | 18.6 | — | — |
| Folio Biosciences | 36 | 9.4 | — | — |
| Michigan Urology | 119 | 31.2 | | |

Note that, in the clinical validation study, preoperative PSA was missing for eight patients, and clinical staging was missing for 12 patients.
SD denotes standard deviation.
*Excludes two patients with PSA reported at diagnosis of 791 and 600 ng/ml, which is atypical of newly diagnosed patients.
†Includes four samples annotated only as T3

Clinical Validation Study

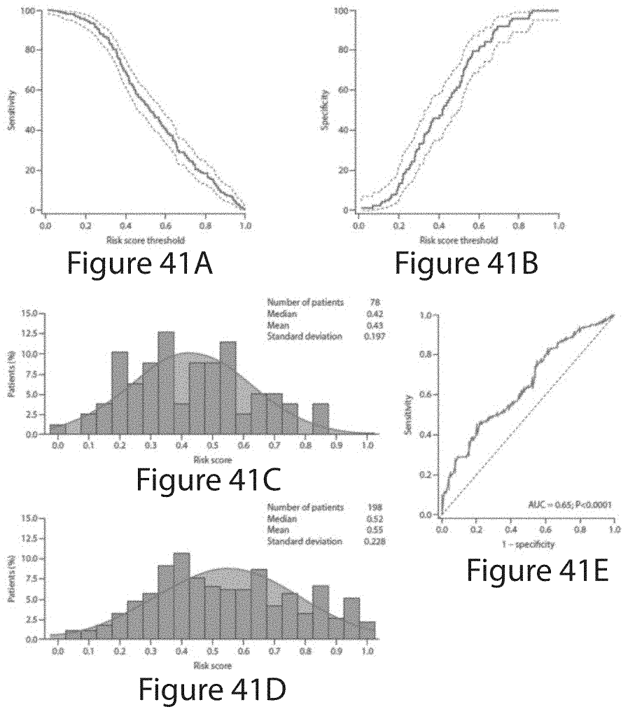

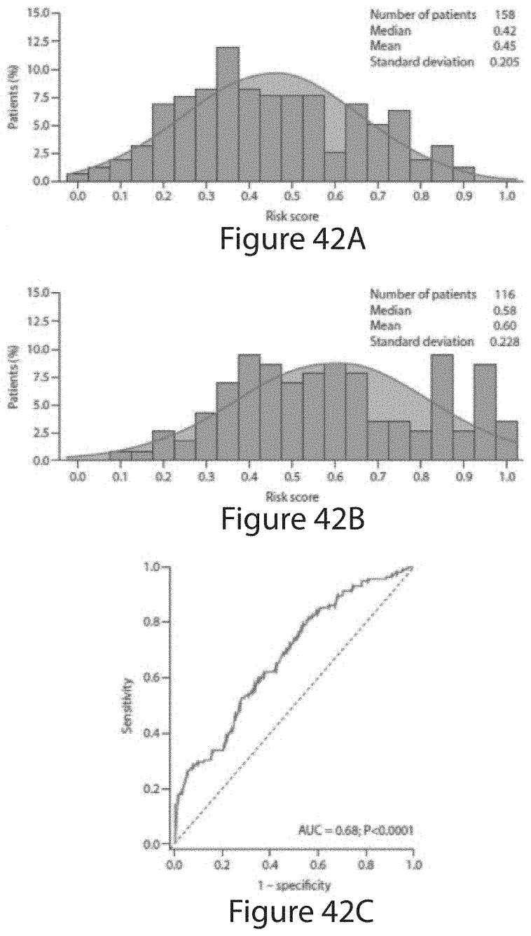

Table 20 summarizes the tumor characteristics of the 276 samples in the clinical validation study. As shown in Table 21, the study met its two co-primary endpoints and validated the assay for both endpoints (favorable pathology: AUC, 0.68 [95% CI, 0.61 to 0.74]; P<0.0001; OR for risk score, 20.9 per unit change; “GS 6” pathology: AUC, 0.65 [95% CI, 0.58 to 0.72]; P<0.0001; OR for risk score, 12.6 per unit change). Further details are shown in FIGS. 41 and 42.

TABLE 21
Clinical Validation Study: Prognostic Test Performance against
the two Co-primary Endpoints for the Eight-biomarker Signature.

| | | | | | |
|---|---|---|---|---|---|
| | | | | OR Lowest- to | OR as Point |
| | | | P Value | Highest-risk | Estimate for |
| | | | (Bonferroni- | Score Quartile | Continuous Range |
| Population (N) | Endpoint Definition | AUC (95% CI) | Adjusted) | (95% CI) | of Risk Scores (95% CI) |

Co-primary endpoints

| | | | | | |
|---|---|---|---|---|---|
| (N = 274) | Favorable pathology-Surgical | 0.68 (0.61 to 0.74). | <0.0001 | 3.3 (1.8 to 6.1) | 20.9 (6.4 to 68.2) |
| | Gleason ≤3 + 4 and organ- | | | | |
| | confined (≤T2) vs. | | | | |
| | nonfavorable- surgical | | | | |
| | Gleason ≥4 + 3 or non-organ- | | | | |
| | confined (T3a, T3b, N, or M) | | | | |
| (N = 276) | “GS 6”-Surgical Gleason = | 0.65 (0.58 to 0.72). | <0.0001 | 4.2 (1.9 to 9.3) | 12.6 (3.5 to 47.2) |
| | 3 + 3 and localized ≤T3a vs. | | | | |
| | “Non-GS 6”- surgical | | | | |
| | Gleason ≥3 + 4 or nonlocalized | | | | |
| | (T3b, N, or M) | | | | |

CI denotes confidence interval; NCCN denotes National Comprehensive Cancer Network; OR denotes odds ratio.

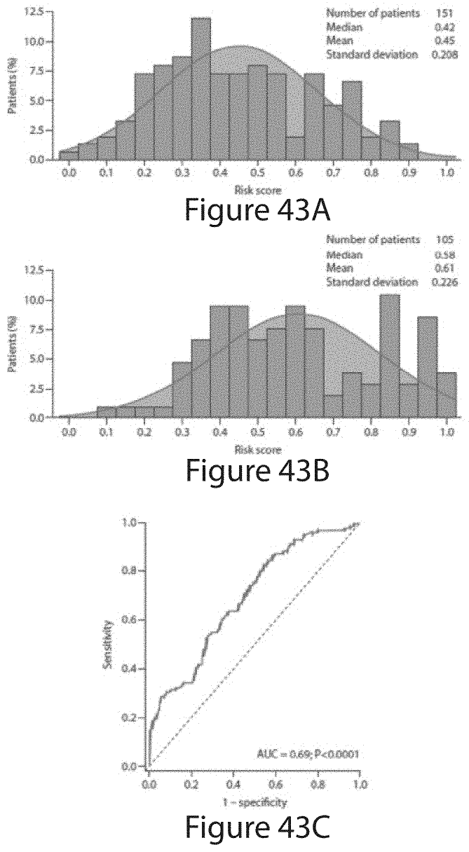

We had sufficient annotation to classify 256 cases according to NCCN and D'Amico criteria. The performance of the biomarker signature assay on this cohort for favorable pathology is shown in FIG. 43 and was similar to the full cohort (AUC, 0.69 [95% CI, 0.63 to 0.76]; P<0.0001; OR for risk score, 26.2 per unit change).

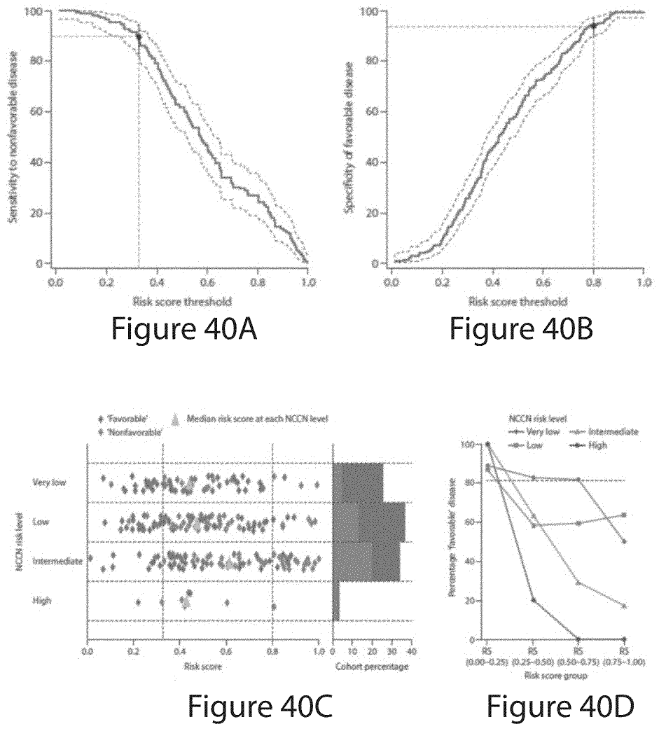

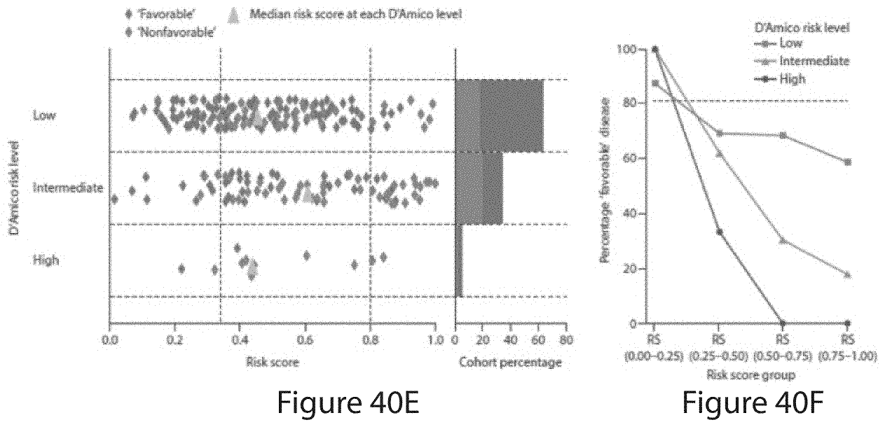

FIG. 40 shows the sensitivity and specificity associated with the risk score as a prognostic aid for favorable/nonfavorable disease, and the distributions of the risk score in the NCCN and D'Amico categories. FIG. 40A shows an example of a favorable category identified in this study population on the basis of the molecular signature. A threshold of 0.33 for the favorable category results in a sensitivity (P[risk score>0.33|nonfavorable pathology]) of 90% (95% CI, 82% to 94%), which limits the false-negative rate among patients with nonfavorable pathology to 10% (95% CI, 6% to 18%). Similarly, in FIG. 40B, a nonfavorable category may be identified in this study population with specificity (P[risk score<0.801favorable pathology]) of 95% (95% CI, 90% to 98%), which limits the false-positive rate among patients with favorable pathology to 5% (95% CI, 2% to 10%).

We assessed the predictive value of the risk score and compared it with those of the NCCN and D'Amico risk categories (Table 22). The PPV for identifying favorable disease at a risk score of ≤0.33 was 83.6% (specificity, 90%). Conversely, at a risk score of >0.80, 23.1% of patients had favorable disease (i.e. 76.9% had nonfavorable disease). Based on the study population, this translates to 39% of patients with risk scores ≤0.33 or >0.8, of which 81% are correctly identified.

TABLE 22
Clinical Validation Study. Comparison of Predictive Value of the Biomarker
Assay for Favorable Pathology with NCCN and D'Amico Risk Categories

| Biomarker | Number of Patients According to | | | |
|---|---|---|---|---|
| Assay | Biomarker Assay Scores | | | |
| Score Range | Total | Favorable | Nonfavorable | % PPV (95% CI) |
| ≤0.33 | 61 | 51 | 10 | 83.6% (71.9% to |
| | | | | 91.8%) |
| 0.33 to 0.80 | 156 | 91 | 65 | 58.3% (50.2% to |
| | | | | 66.2%) |
| >0.80 | 39 | 9 | 30 | 23.1% (11.1% to |
| | | | | 39.3%) |
| All | 256 | 151 | 105 | 59% (52.7% to |
| | | | | 65.1%) |

| | | Number of Patients According to | | | |
|---|---|---|---|---|---|
| | Biomarker | Biomarker Assay Scores | | | |
| | Assay | and SOC Categories | | | |
| SOC: NCCN | Score Range | Total | Favorable | Nonfavorable | % PPV (95% CI) |
| Very low by | — | 66 | 53 | 13 | 80.3% (68.7% to |
| NCCN | | | | | 89.1%) |
| Very low | ≤0.33 | 20 | 19 | 1 | 95% (75.1% to |
| | | | | | 99.9%) |
| Very low | 0.33 to 0.80 | 42 | 33 | 9 | 78.6% (63.2% to |
| | | | | | 89.7%) |
| Very low | >0.80 | 4 | 1 | 3 | 25% (0.6% to |
| | | | | | 80.6%) |
| Low by | — | 94 | 60 | 34 | 63.8% (53.3% to |
| NCCN | | | | | 73.5%) |
| Low | ≤0.33 | 27 | 22 | 5 | 81.5% (61.9% to |
| | | | | | 93.7%) |
| Low | 0.33 to 0.80 | 59 | 34 | 25 | 57.6% (44.1% to |
| | | | | | 70.4%) |
| Low | >0.80 | 8 | 4 | 4 | 50% (15.7% to |
| | | | | | 84.3%) |
| Intermediate | — | 88 | 36 | 52 | 40.9% (30.5% to |
| by NCCN | | | | | 51.9%) |
| Intermediate | ≤0.33 | 12 | 9 | 3 | 75% (42.8% to |
| | | | | | 94.5%) |
| Intermediate | 0.33 to 0.80 | 50 | 23 | 27 | 46% (31.8% to |
| | | | | | 60.7%) |
| Intermediate | >0.80 | 26 | 4 | 22 | 15.40% (4.40% to |
| | | | | | 34.9%) |
| High by | — | 8 | 2 | 6 | 25% (3.2% to |
| NCCN | | | | | 65.1%) |
| High | ≤0.33 | 2 | 1 | 1 | 50% (1.3% to |
| | | | | | 98.7%) |
| High | 0.33 to 0.80 | 5 | 1 | 4 | 20% (0.5% to |
| | | | | | 71.6%) |
| High | >0.80 | 1 | 0 | 1 | 0% (0% to |
| | | | | | 97.5%) |

| | Biomarker | | | | |
|---|---|---|---|---|---|
| SOC: | Assay | | | | |
| D'Amico | Scores Range | Total | Favorable | Nonfavorable | % PPV (95% CI) |
| Low by | — | 160 | 113 | 47 | 70.6% (62.9% to |
| D'Amico | | | | | 77.6%) |
| Low | ≤0.33 | 47 | 41 | 6 | 87.2% (74.3% to |
| | | | | | 95.2%) |
| Low | 0.33 to 0.80 | 101 | 67 | 34 | 66.3% (56.2% to |
| | | | | | 75.4%) |
| Low | >0.80 | 12 | 5 | 7 | 41.7% (15.2% to |
| | | | | | 72.3%) |
| Intermediate | — | 85 | 35 | 50 | 41.2% (30.6% to |
| by D'Amico | | | | | 52.4%) |
| Intermediate | ≤0.33 | 12 | 9 | 3 | 75% (42.8% to |
| | | | | | 94.5%) |
| Intermediate | 0.33 to 0.80 | 48 | 22 | 26 | 45.8% (31.4% to |
| | | | | | 60.8%) |
| Intermediate | >0.80 | 25 | 4 | 21 | 16% (4.5% to |
| | | | | | 36.1%) |
| High by | — | 11 | 3 | 8 | 27.3% (6% to |
| D'Amico | | | | | 61%) |
| High | ≤0.33 | 2 | 1 | 1 | 50% (1.3% to |
| | | | | | 98.7%) |
| High | 0.33 to 0.80 | 7 | 2 | 5 | 28.6% (3.7% to |
| | | | | | 71%) |
| High | >0.80 | 2 | 0 | 2 | 0% (0% to |
| | | | | | 84.2%) |

CI denotes confidence interval; NCCN denotes National Comprehensive Cancer Network; PPV denotes positive predictive value; SOC denotes standard of care.

We further examined the distribution of patients with favorable disease according to our risk score within each NCCN category (FIG. 40C and Table 22). Using a risk score of ≤0.33, the PPV for favorable disease was 75% for NCCN intermediate risk, 81.5% for NCCN low risk, and 95% for NCCN very low risk (FIG. 40C and Table 22). This contrasts with the PPVs obtained for the NCCN risk categories alone, which were 40.9% for intermediate risk, 63.8% for low risk, and 80.3% for very low risk (Table 22). Accordingly, the risk score provided additional information for individual patients relative to NCCN risk categories. As shown in FIG. 40D, an increased risk score correlated with a decreased frequency of favorable cases within each NCCN category. Similar results were obtained when comparing with the D'Amico categories (FIG. 40E and FIG. 40F).

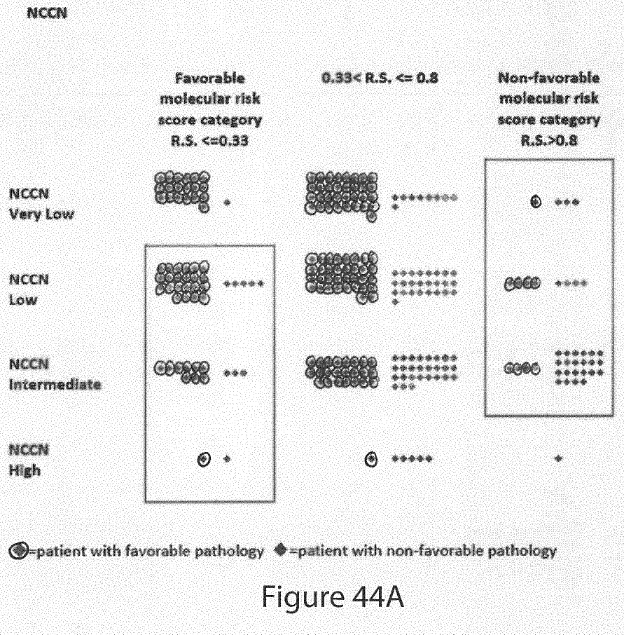

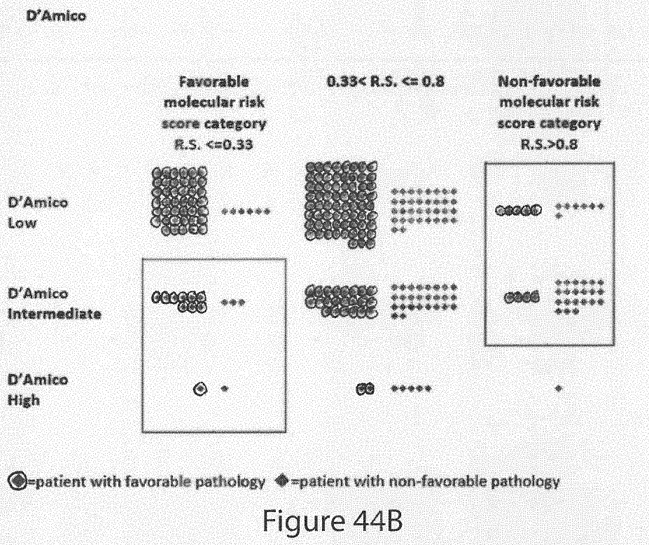

To confirm the benefit of the risk score in the context of SOC, we performed an NRI analysis for the favorable and nonfavorable categories relative to NCCN and D'Amico. Using the underlying data shown in Table 22, we found an NRI for NCCN of 0.34 (P<0.00001; 95% CI, 0.20 to 0.48) and for D'Amico of 0.24 (P=0.0001; 95% CI, 0.12 to 0.35; see FIG. 44). This demonstrates that the test provides additional discriminatory capability to that provided by the SOC classification systems alone.

### DISCUSSION

The results of two clinical studies are reported here: a development study and a blinded validation study, performed on prostate cancer biopsy samples with matched prostatectomy specimens. These studies demonstrate the accuracy and validity of a novel, proteomic multi-biomarker assay that can be used at the time of biopsy to predict the presence of high-risk features in the prostate and the potential for extraprostatic extension and metastases. In our first model-building study, an optimal eight-biomarker signature was determined from 12 candidate biomarkers previously shown to predict tumor aggressiveness and lethality. The study defined the eight-biomarker signature and resulting individualized risk score based on logistic regression analysis for prediction of “Non-GS 6” prostate pathology (surgical Gleason score ≥3+4 or non-localized >T3a, N, M).

The second, blinded clinical validation study met its two co-primary clinical endpoints of predicting prostate pathology independently of clinical and pathological parameters, as follows: “GS 6” pathology, defined as surgical Gleason 3+3=6 and ≤T3a, and ‘favorable’ pathology, defined as organ-confined prostate pathology (surgical Gleason 3+3 or 3+4; ≤T2). Further, our risk score adds differential and complementary personalized information relative to SOC risk stratification.

Recent studies indicate that long-term survival for patients with organ-confined Gleason 3+4 disease is significantly better than for patients with non-organ-confined disease or for tumor with dominant Gleason pattern 4 or higher,^{19,32,33} and that deferred therapy for the former group does not significantly change long-term outcome.³⁵⁻³⁷ Currently, most risk stratification systems do not discriminate between Gleason 7 biopsies, and typically patients considered candidates for active surveillance belong to ‘very-low-risk’ or ‘low-risk’ groups that only contain biopsy Gleason score ≤6.^{3,9} However, around 25% of Gleason grade 3+4 biopsies are ‘downgraded’ and a similar percentage of Gleason grade 3+3 biopsies are ‘upgraded’ when comparing with the surgical Gleason, primarily owing to biopsy sampling error and pathologist discordance.^{16 20} Based on this, the need for a molecular evidence-based test is high for Gleason grade 3+3 and 3+4 biopsies,²¹ and we have developed our test for this indication. The favorable endpoint was developed to discriminate between favorable cases (surgical Gleason 3+3 or 3+4, organ-confined [≤T2] tumors) from nonfavorable cases (extraprostatic extension [T3a], seminal vesicle invasion [T3b], lymph node or distant metastases, or dominant Gleason 4 pattern or higher).

Our study shows that, at a test risk score ≤0.33, the predictive values for identifying patients with favorable pathology in the very-low- and low-risk NCCN and low-risk D'Amico groups are 95%, 81.5%, and 87.2%, respectively, values higher than those achieved by these risk groups alone. Moreover, the test is also able to identify patients with nonfavorable pathology, arguably unsuitable for active surveillance, with high confidence, having a predictive value of 76.9% at risk score >0.8 across all risk groups for both risk stratification systems. The significance of the test-based patient stratification for the individual patient is illustrated by the fact that increased test risk scores correlate with decreased observed frequency of favorable cases across all risk stratification groups. A measure of the additional information provided by the risk score relative to SOC was provided by the NRI analysis. We found an NRI for NCCN of 0.34 (P<0.00001; 95% CI, 0.20 to 0.48) and for D'Amico of 0.24 (P=0.0001; 95% CI, 0.12 to 0.35). Among patients with favorable and nonfavorable pathology, 78% and 76% respectively were correctly adjusted to lower and higher risk than was obvious from NCCN risk group itself (FIG. 44).

In embodiments, our risk score is generated based on quantitative measurements of eight biomarkers in intact tissue using a multiplex proteomics imaging platform (Supplementary Appendix). This approach has several potential advantages compared with gene expression-based tests, where tissue is homogenized before analysis. Firstly, it renders the test robust to variations in the ratios of benign tissue relative to tumor tissue because it does not interfere with the marker measurements from intact cancer cells. Furthermore, the test allows integration of molecular and morphologic information and requires only few cancer cells.

The eight biomarkers in our model comprise a subset of 12 biomarker candidates identified as predictive of both aggressiveness and lethal outcome despite tissue sampling error. This indicates that the pathology endpoint used in the present study is also relevant for long-term outcome, as has been reported.^{29,32,33}

In conclusion, our results demonstrate the utility of this clinical biomarker biopsy test for personalized prognostication of prostate cancer and its impact on therapeutic choice. The ability to provide differential information for the individual patient relative to SOC, where prognostic capabilities are currently limited, makes it a useful aid in clinical decision-making.

### REFERENCES

1. Siegel R, Ma J, Zou Z, Jemal A. Cancer statistics, 2014. CA Cancer J Clin 2014; 64:9-29.
2. Welch H G, Albertsen P C. Prostate cancer diagnosis and treatment after the introduction of prostate-specific antigen screening: 1986-2005. J Natl Cancer Inst 2009; 101:1325-9.
3. Epstein J I. An update of the Gleason grading system. J Urol 2010; 183:433-40.
4. Barocas D A, Cowan J E, Smith J A, Jr., Carroll P R, Ca P I. What percentage of patients with newly diagnosed carcinoma of the prostate are candidates for surveillance? An analysis of the CaPSURE database. The Journal of urology 2008; 180:1330-4; discussion 4-5.
5. Carter H B, Partin A W, Walsh P C, et al. Gleason score 6 adenocarcinoma: should it be labeled as cancer? J Clin Oncol 2012; 30:4294-6.
6. Howrey B T, Kuo Y F, Lin Y L, Goodwin J S. The impact of PSA screening on prostate cancer mortality and overdiagnosis of prostate cancer in the United States. J Gerontol A Biol Sci Med Sci 2013; 68:56-61.
7. Loeb S, Bjurlin M A, Nicholson J, et al. Overdiagnosis and overtreatment of prostate cancer. Eur Urol 2014; pii: S0302-2838(13)01490-5. doi: 10.1016/j.eururo.2013.12.062 [Epub ahead of print].
8. Sandhu G S, Andriole G L. Overdiagnosis of prostate cancer. J Natl Cancer Inst Monogr 2012; 2012:146-51.
9. NCCN. NCCN clinical practice guidelines in oncology: prostate cancer. Version 3.2012. Available at: http:/www.nccn.org/professionals/physician_gls/f_guidelines.asp#prostate_detection. Accessed 18 Feb. 2014.
10. NCCN. Prostate cancer early detection. Version 2.2012. Available at: http://www.nccn.org/professionals/physician_gls/f_guidelines.asp#prostate_detection. Accessed 18 Feb. 2014.
11. D'Amico A V, Whittington R, Malkowicz S B, et al. Biochemical outcome after radical prostatectomy, external beam radiation therapy, or interstitial radiation therapy for clinically localized prostate cancer. JAMA 1998; 280:969-74.
12. Bangma C H, Roobol M J. Defining and predicting indolent and low risk prostate cancer. Crit Rev Oncol Hematol 2012; 83:235-41.
13. Brimo F, Montironi R, Egevad L, et al. Contemporary grading for prostate cancer: implications for patient care. Eur Urol 2013; 63:892-901.
14. Truong M, Slezak J A, Lin C P, et al. Development and multi-institutional validation of an upgrading risk tool for Gleason 6 prostate cancer. Cancer 2013; 119:3992-4002.
15. Pinthus J H, Witkos M, Fleshner N E, et al. Prostate cancers scored as Gleason 6 on prostate biopsy are frequently Gleason 7 tumors at radical prostatectomy: implication on outcome. J Urol 2006; 176:979-84; discussion 84.
16. Epstein J I, Feng Z, Trock B J, Pierorazio P M. Upgrading and downgrading of prostate cancer from biopsy to radical prostatectomy: incidence and predictive factors using the modified Gleason grading system and factoring in tertiary grades. Eur Urol 2012; 61:1019-24.
17. Porten S P, Whitson J M, Cowan J E, et al. Changes in prostate cancer grade on serial biopsy in men undergoing active surveillance. J Clin Oncol 2011; 29:2795-800.
18. Goodman M, Ward K C, Osunkoya A O, et al. Frequency and determinants of disagreement and error in gleason scores: a population-based study of prostate cancer. Prostate 2012; 72:1389-98.
19. Stark J R, Perner S, Stampfer M J, et al. Gleason score and lethal prostate cancer: does 3+4=4+3? J Clin Oncol 2009; 27:3459-64.
20. Eggener S E, Scardino P T, Walsh P C, et al. Predicting 15-year prostate cancer specific mortality after radical prostatectomy. J Urol 2011; 185:869-75.
21. Ross H M, Kryvenko O N, Cowan J E, Simko J P, Wheeler™, Epstein J I. Do adenocarcinomas of the prostate with Gleason score (GS)<1=6 have the potential to metastasize to lymph nodes? Am J Surg Pathol 2012; 36:1346-52.
22. Cooperberg M, Simko J, Falzarano S, et al. Development and validation of the biopsy-based genomic prostate score (GPS) as a predictor of high grade or extracapsular prostate cancer to improve patient selection for active surveillance. J Urol 2013; 189 (Suppplement 4S):Abstract 2131 p e873.
23. Cooperberg M R, Simko J P, Cowan J E, et al. Validation of a cell-cycle progression gene panel to improve risk stratification in a contemporary prostatectomy cohort. J Clin Oncol 2013; 31:1428-34.
24. Cuzick J, Berney D M, Fisher G, et al. Prognostic value of a cell cycle progression signature for prostate cancer death in a conservatively managed needle biopsy cohort. British journal of cancer 2012; 106:1095-9.
25. Cuzick J, Swanson G P, Fisher G, et al. Prognostic value of an RNA expression signature derived from cell cycle proliferation genes in patients with prostate cancer: a retrospective study. The lancet oncology 2011; 12:245-55.
26. Camp R L, Chung G G, Rimm D L. Automated subcellular localization and quantification of protein expression in tissue microarrays. Nat Med 2002; 8:1323-7.
27. Donovan M J, Hamann S, Clayton M, et al. Systems pathology approach for the prediction of prostate cancer progression after radical prostatectomy. J Clin Oncol 2008; 26:3923-9.
28. Ding Z, Wu C J, Chu G C, et al. SMAD4-dependent barrier constrains prostate cancer growth and metastatic progression. Nature 2011; 470:269-73.
29. Eggener S E, Scardino P T, Walsh P C, et al. Predicting 15-year prostate cancer specific mortality after radical prostatectomy. The Journal of urology 2011; 185:869-75.
30. Ross H M, Kryvenko O N, Cowan J E, Simko J P, Wheeler™, Epstein J I. Do adenocarcinomas of the prostate with Gleason score (GS)</=6 have the potential to metastasize to lymph nodes? Am J Surg Pathol 2012; 36:1346-52.
31. Lindsey J K, Jones B. Choosing among generalized linear models applied to medical data. Stat Med 1998; 17:59-68.
32. Pierorazio P M, Walsh P C, Partin A W, Epstein J I. Prognostic Gleason grade grouping: data based on the modified Gleason scoring system. BJU Int 2013; 111:753-60.
33. Mullins J K, Feng Z, Trock B J, Epstein J I, Walsh P C, Loeb S. The impact of anatomical radical retropubic prostatectomy on cancer control: the 30-year anniversary. J Urol 2012; 188:2219-24.
34. Pencina M J, D'Agostino R B, Sr., D'Agostino R B, Jr., Vasan R S. Evaluating the added predictive ability of a new marker: from area under the ROC curve to reclassification and beyond. Stat Med 2008; 27:157-72.
35. Graefen M, Walz J, Chun K H, Schlomm T, Haese A, Huland H. Reasonable delay of surgical treatment in men with localized prostate cancer-impact on prognosis? Eur Urol 2005; 47:756-60.
36. Holmstrom B, Holmberg E, Egevad L, et al. Outcome of primary versus deferred radical prostatectomy in the National Prostate Cancer Register of Sweden Follow-Up Study. The Journal of urology 2010; 184:1322-7.
37. Vickers A J, Bianco F J, Jr., Boorjian S, Scardino P T, Eastham J A. Does a delay between diagnosis and radical prostatectomy increase the risk of disease recurrence? Cancer 2006; 106:576-80.
38. Bishoff J T, Freedland S J, Gerber L, et al. Prognostic utility of the CCP score generated from biopsy in men treated with prostatectomy. J Urol 2014; 6:00248-1.

Supplemental Material

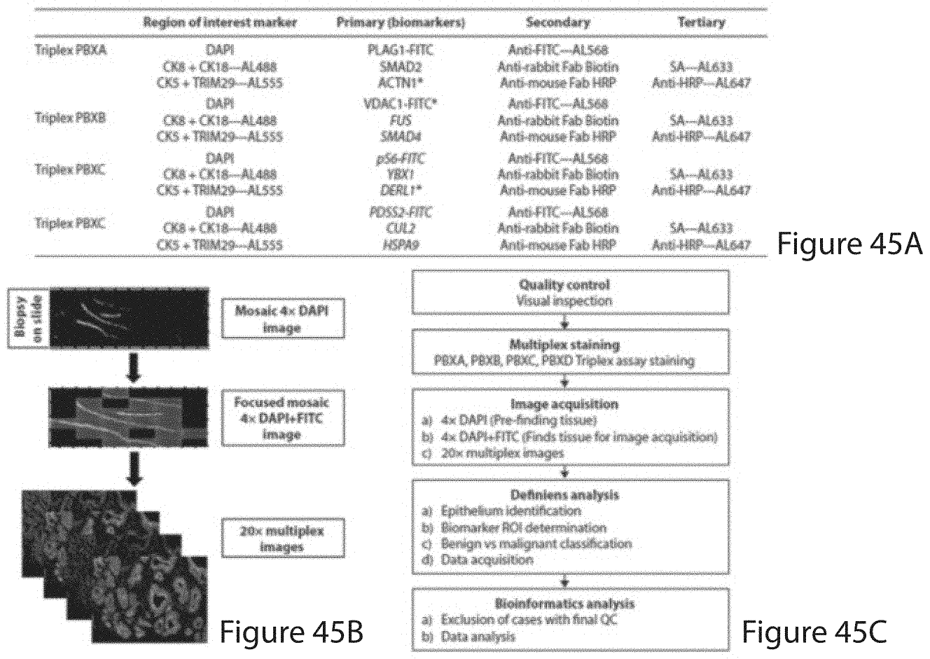

FIG. 41A-E, FIG. 42A-C, FIG. 43A-C, FIG. 44A-B, and FIG. 45A-C provide further information and are described in the description of the drawings above.

Methods for Quantitative Multiplex Proteomics Imaging (QMPI)

Formalin-fixed, paraffin-embedded (FFPE) prostate cancer biopsy tissue slides were analyzed using an quantitative multiplex proteomics imaging (QMPI) platform for intact tissue that integrates morphological object recognition and molecular biomarker measurements from tumor epithelium at the individual slide level. The antibody validation, staining protocols, image acquisition, image analysis, and inter-experimental controls are described below.

Assay Description and Biomarker-Antibody Validation

The assay was executed using four slides, as outlined in the staining protocol depicted in FIG. 45.

Four combinations of three (triplex) biomarkers each were used: A) PLAG1, SMAD2, ACTN1; B) VDAC1, FUS, SMAD4; C) pS6, YBX1, DERL1; D) PDSS2, CUL2, DCC. Each of the primary antibodies used was validated for specificity and it was found that PLAG1 was insufficiently specific; it was thus excluded from the potential signature. Each triplex assay consisted of an initial blocking step followed by five consecutive incubation steps with appropriate washes in between.
1) Incubation with a mixture of anti-biomarker 2 (rabbit monoclonal antibody [MAb]) and anti-biomarker 3 (mouse MAb).
2) Incubation with a mixture of Zenon anti-mouse IgG Fab-horseradish peroxidase (HRP) and Zenon anti-rabbit IgG Fab-biotin.
3) Incubation with anti-biomarker 1 MAb conjugated to FITC.
4) Visualization step with a mixture of anti-FITC MAb-Alexa 568, streptavidin-Alexa 633, anti-HRP-Alexa 647, anti-CK8-Alexa 488, anti-CK18-Alexa 488, anti-CK5-Alexa 555, and anti-Trim29-Alexa 555.
5) A brief incubation with DAPI for nuclear staining.

After final washes, slides were mounted with ProlongGold (Life Technologies), a coverslip was added, and the slides were stored at −20° C. overnight before image acquisition.

Slide Processing and Staining Protocols

Most steps of slide processing and staining were automated to ensure maximal reproducibility. Sections were first deparaffinized in xylene/graded alcohols using StainMate (Thermo Scientific). Antigen retrieval was performed with 0.05% citraconic anhydride solution for 45 min at 95° C. using a Lab Vision PT module (Thermo Scientific). Slides were stained with an Autostainer 360 or 720 (Thermo Scientific) using the assay format described above. Biopsy case samples were stained in batches of 25 slides per Autostainer, with one cell line tissue microarray (TMA) control slide (see below) for each triplex assay format.

Image Acquisition

For each triplex assay, one specific Vectra Intelligent Slide Analysis System (200-slide capacity) was used for quantitative multiplex immunofluorescence image acquisition with optimized DAPI, FITC, TRITC, and Cy5 long-pass filter cubes that allowed maximal spectral resolution and minimum bleed-through between fluorophores. To minimize variation, the light intensity for each system was calibrated before each run with X-Cite Optical Power Measurement System (Lumen Dynamics). Vectra 2.0, Inform 1.3, and Nuance 2.0 softwares (PerkinElmer) were used, respectively, for image acquisition, generation of tissue-finding algorithms, and development of a spectral library.

In the image acquisition process, first, the image of the entire slide was acquired with a mosaic of 4× monochrome DAPI filter images. The initial tissue-finding algorithm included in the image acquisition protocol was then used to locate tissue, which was then subjected to re-acquisition of images, this time with both 4×DAPI and 4×FITC monochrome filters. A final tissue-finding algorithm included in the protocol was then applied to ensure that images of all 20× fields containing a sufficient amount of tissue were acquired (FIG. 45B).

Algorithms included in the image acquisition protocol limited data collection to those 20× fields containing sufficient amounts of tissue. The multispectral acquisition protocol used in the assay had consecutive exposures of DAPI, FITC, TRITC, and Cy5 filters. Upon completion of image acquisition, image cubes were automatically stored on a server for subsequent automatic unmixing into individual channels and processing by Definiens software.

Image Analysis and Inputs for the Risk Score Model

We developed an image-analysis algorithm using Definiens Developer XD (Definiens AG, Munich, Germany) for tumor identification and biomarker quantification. The software was used to delineate malignant and benign epithelial areas of the biopsy tissue, allowing measurement of marker intensity exclusively over malignant areas. For each biopsy sample, several 20× image fields were scanned and saved as multispectral image files using CRi Vectra (PerkinElmer). As many as 140 individual fields were scanned for a given slide in order to acquire images from the entire tissue sample. Eleven different FFPE cell lines in triplicate and two prostatectomy tissue samples in duplicate were used as controls on a separate quality control slide array. For each 1.0-mm quality control cell line or tissue core, two 20× image fields were scanned (i.e. a total of six images for each cell line control and four images for each tissue control). The Vectra multispectral image files were first converted into multilayer TIF format using inForm (PerkinElmer) and a customized spectral library, and then converted to single-layer TIFF files using BioFormats (OME). The single-layer TIFF files were imported into the Definiens workspace using a customized import algorithm so that, for each biopsy sample and each quality control, all of the image field TIFF files were loaded and analyzed as “maps” within a single “scene”.

Autoadaptive thresholding was used to define fluorescence intensity cut-offs for tissue segmentation in each individual tissue sample in our image analysis algorithm. Cell line control cores were automatically distinguished from prostatectomy tissue cores in the Definiens algorithm based on predefined core coordinates on the quality control slides. The biopsy and tissue core samples were segmented using the fluorescent epithelial and basal cell markers, along with DAPI for classification into epithelial cells, basal cells, and stroma, and further compartmentalized into cytoplasm and nuclei. Individual gland regions were classified as malignant or benign based on the relational features between basal cells and adjacent epithelial structures combined with object-related features, such as gland thickness. Epithelial markers are not present in all cell lines, therefore the cell line controls were segmented into tissue versus background using the autofluorescence channel. Fields with artifact staining, insufficient epithelial tissue, or out-of-focus images were removed by a rigorous multi-parameter quality control algorithm.

Epithelial marker, DAPI, ACTN, VDAC, and DERL1 intensities were quantitated in malignant and nonmalignant epithelial regions as quality control measurements. Biomarker values were also measured in the cytoplasm, nucleus, and whole cell of malignant and nonmalignant epithelial regions. The mean biomarker pixel intensity for each subcellular compartment was averaged across each individual map with acceptable quality parameters, and the map-specific values were exported for bioinformatics analysis. A weighted mean was calculated from suitable values to produce a single intensity for each marker on a tissue sample; 20× fields with mean intensity values in the 40th to 90th percentile for the slide or 20× fields encompassing large areas of tumor were considered suitable. This provided the input for the risk score model.

Inter-Experimental Controls: Quality Control Procedures

Cell line controls were used as batch controls. All biopsy case samples received were also subjected to a multistep quality control procedure, serving as the means to include or exclude samples from the clinical studies. Unprocessed slides with sections were examined visually and with a fluorescence microscope for the presence of stains and dyes. Samples with noticeable amounts of fluorescent dyes in biopsy tissue were excluded from further analysis, as they would be during clinical pathology lab practice. Next, one slide from each biopsy case sample was manually stained with ACTN1, CK8/18-Alexa 488, and CK5/Trim29. Stained slides were manually inspected; case samples failed quality control if the tissue was small or fragmented, had little tumor tissue or stained poorly with any of the above three markers.

After multiplex immunofluorescence staining, all 20× images were manually inspected, and those fields containing spurious/non-prostate tissue (e.g. gut tissue) were excluded from further analysis. Once image analysis had separated malignant and benign tissue, cases with inadequate benign or tumor areas were eliminated. Cases with ACTN1, DERL1, or VDAC levels below predetermined minimums were also excluded.

Staining Control Development and Application: Cell-Line Controls

Thirty cell lines were stained with each marker used in the study, from which 11 cell lines were selected to be staining controls on the basis of range, signal intensity, and lowest variability.

Cell lines were grown in prescribed medium to 70% to 80% confluence with uniformity and fixed on plates with formalin. Cells were scraped and spun down, and cell discs were prepared from cell/histogel suspension of cell pellets, which was paraffin-embedded. Using these pellets, TMA blocks were generated for use in reproducibility studies, validation of master mixes, and as control slides during routine sample staining.

One section/slide from the cell line TMA was processed with each batch of biopsy slides. Staining, image acquisition, and data extraction and analysis were performed in exactly the same way as was described earlier for the individual triplex assay format.

Clinical Studies: Statistical Plan

A statistical analysis plan (SAP) was locked, recorded, and communicated with an outside biostatistical expert before clinical study data were available for analysis in the validation study. According to the SAP, all P-values for co-primary outcomes are reported after multiplication by two to reflect a Bonferroni correction. AUC CIs and P-values were estimated using a binomial exact test, while AUC standard error was measured using the method described by DeLong et al. 1988¹ ORs from logistic regression were included in the SAP, as well as comparison with standard of care using exact binomial CIs for positive predictive value, sensitivity and specificity. A statistician otherwise not involved with the assay development performed the statistical analysis.

### REFERENCE

1. DeLong E R, DeLong D M, Clarke-Pearson DL. Comparing the areas under two or more correlated receiver operating characteristic curves: a nonparametric approach. Biometrics 1988; 44:837-45.

LIST OF SEQUENCES

SEQ ID NO: 1-ACTN1 (NP_001093.1)
MDHYDSQQTNDYMQPEEDWDRDLLLDPAWEKQQRKTFTAWCNSHLRKAGTQIENIEEDFRDGLKLMLL
LEVISGERLAKPERGKMRVHKISNVNKALDFIASKGVKLVSIGAEEIVDGNVKMTLGMIWTIILRFAIQDISV
EETSAKEGLLLWCQRKTAPYKNVNIQNFHISWKDGLGFCALIHRHRPELIDYGKLRKDDPLTNLNTAFDVA
EKYLDIPKMLDAEDIVGTARPDEKAIMTYVSSFYHAFSGAQKAETAANRICKVLAVNQENEQLMEDYEKL
ASDLLEWIRRTIPWLENRVPENTMHAMQQKLEDFRDYRRLHKPPKVQEKCQLEINFNTLQTKLRLSNRPAF
MPSEGRMVSDINNAWGCLEQVEKGYEEWLLNEIRRLERLDHLAEKFRQKASIHEAWTDGKEAMLRQKDY
ETATLSEIKALLKKHEAFESDLAAHQDRVEQIAAIAQELNELDYYDSPSVNARCQKICDQWDNLGALTQKR
REALERTEKLLETIDQLYLEYAKRAAPFNNWMEGAMEDLQDTFIVHTIEEIQGLTTAHEQFKATLPDADKE
RLAILGIHNEVSKIVQTYHVNMAGTNPYTTITPQEINGKWDHVRQLVPRRDQALTEEHARQQHNERLRKQF
GAQANVIGPWIQTKMEEIGRISIEMHGTLEDQLSHLRQYEKSIVNYKPKIDQLEGDHQLIQEALIFDNKHTN
YTMEHIRVGWEQLLTTIARTINEVENQILTRDAKGISQEQMNEFRASFNHFDRDHSGTLGPEEFKACLISLG
YDIGNDPQGEAEFARIMSIVDPNRLGVVTFQAFIDFMSRETADTDTADQVMASFKILAGDKNYITMDELRR
ELPPDQAEYCIARMAPYTGPDSVPGALDYMSFSTALYGESDL (SEQ ID NO: 1).
SEQ ID NO: 2-ACTN1 (NM_001102.3)
TCTGCCCCTTCCCCCCGCCCCCGCCCGCCTCGGCTCCCGCAGCGCTAGTGTGTCCGCCTAGTTCAGTGT
GCGTGGAGATTAGGTCCAAGCGCCCGCCCAGAGGCAGGCAGTCCGCGAGCCCAGCCGCCGCTGTCGC
CGCCAGTAGCAGCCTTCGCCAGCAGCGCCGCGGCGGAACCGGGCGCAGGGGAGCGAGCCCGGCCCCG
CCAGCCCAGCCCAGCCCAGCCCTACTCCCTCCCCACGCCAGGGCAGCAGCCGTTGCTCAGAGAGAAGG
TGGAGGAAGAAATCCAGACCCTAGCACGCGCGCACCATCATGGACCATTATGATTCTCAGCAAACCAA
CGATTACATGCAGCCAGAAGAGGACTGGGACCGGGACCTGCTCCTGGACCCGGCCTGGGAGAAGCAG
CAGAGAAAGACATTCACGGCATGGTGTAACTCCCACCTCCGGAAGGCGGGGACACAGATCGAGAACA
TCGAAGAGGACTTCCGGGATGGCCTGAAGCTCATGCTGCTGCTGGAGGTCATCTCAGGTGAACGCTTG
GCCAAGCCAGAGCGAGGCAAGATGAGAGTGCACAAGATCTCCAACGTCAACAAGGCCCTGGATTTCA
TAGCCAGCAAAGGCGTCAAACTGGTGTCCATCGGAGCCGAAGAAATCGTGGATGGGAATGTGAAGAT
GACCCTGGGCATGATCTGGACCATCATCCTGCGCTTTGCCATCCAGGACATCTCCGTGGAAGAGACTT
CAGCCAAGGAAGGGCTGCTCCTGTGGTGTCAGAGAAAGACAGCCCCTTACAAAAATGTCAACATCCA
GAACTTCCACATAAGCTGGAAGGATGGCCTCGGCTTCTGTGCTTTGATCCACCGACACCGGCCCGAGC
TGATTGACTACGGGAAGCTGCGGAAGGATGATCCACTCACAAATCTGAATACGGCTTTTGACGTGGCA
GAGAAGTACCTGGACATCCCCAAGATGCTGGATGCCGAAGACATCGTTGGAACTGCCCGACCGGATG
AGAAAGCCATCATGACTTACGTGTCTAGCTTCTACCACGCCTTCTCTGGAGCCCAGAAGGCGGAGACA
GCAGCCAATCGCATCTGCAAGGTGTTGGCCGTCAACCAGGAGAACGAGCAGCTTATGGAAGACTACG
AGAAGCTGGCCAGTGATCTGTTGGAGTGGATCCGCCGCACAATCCCGTGGCTGGAGAACCGGGTGCCC
GAGAACACCATGCATGCCATGCAACAGAAGCTGGAGGACTTCCGGGACTACCGGCGCCTGCACAAGC
CGCCCAAGGTGCAGGAGAAGTGCCAGCTGGAGATCAACTTCAACACGCTGCAGACCAAGCTGCGGCT
CAGCAACCGGCCTGCCTTCATGCCCTCTGAGGGCAGGATGGTCTCGGACATCAACAATGCCTGGGGCT
GCCTGGAGCAGGTGGAGAAGGGCTATGAGGAGTGGTTGCTGAATGAGATCCGGAGGCTGGAGCGACT
GGACCACCTGGCAGAGAAGTTCCGGCAGAAGGCCTCCATCCACGAGGCCTGGACTGACGGCAAAGAG
GCCATGCTGCGACAGAAGGACTATGAGACCGCCACCCTCTCGGAGATCAAGGCCCTGCTCAAGAAGC
ATGAGGCCTTCGAGAGTGACCTGGCTGCCCACCAGGACCGTGTGGAGCAGATTGCCGCCATCGCACAG
GAGCTCAATGAGCTGGACTATTATGACTCACCCAGTGTCAACGCCCGTTGCCAAAAGATCTGTGACCA
GTGGGACAATCTGGGGGCCCTAACTCAGAAGCGAAGGGAAGCTCTGGAGCGGACCGAGAAACTGCTG
GAGACCATTGACCAGCTGTACTTGGAGTATGCCAAGCGGGCTGCACCCTTCAACAACTGGATGGAGGG
GGCCATGGAGGACCTGCAGGACACCTTCATTGTGCACACCATTGAGGAGATCCAGGGACTGACCACA
GCCCATGAGCAGTTCAAGGCCACCCTCCCTGATGCCGACAAGGAGCGCCTGGCCATCCTGGGCATCCA
CAATGAGGTGTCCAAGATTGTCCAGACCTACCACGTCAATATGGCGGGCACCAACCCCTACACAACCA
TCACGCCTCAGGAGATCAATGGCAAATGGGACCACGTGCGGCAGCTGGTGCCTCGGAGGGACCAAGC
TCTGACGGAGGAGCATGCCCGACAGCAGCACAATGAGAGGCTACGCAAGCAGTTTGGAGCCCAGGCC
AATGTCATCGGGCCCTGGATCCAGACCAAGATGGAGGAGATCGGGAGGATCTCCATTGAGATGCATG
GGACCCTGGAGGACCAGCTCAGCCACCTGCGGCAGTATGAGAAGAGCATCGTCAACTACAAGCCAAA
GATTGATCAGCTGGAGGGCGACCACCAGCTCATCCAGGAGGCGCTCATCTTCGACAACAAGCACACCA
ACTACACCATGGAGCACATCCGTGTGGGCTGGGAGCAGCTGCTCACCACCATCGCCAGGACCATCAAT
GAGGTAGAGAACCAGATCCTGACCCGGGATGCCAAGGGCATCAGCCAGGAGCAGATGAATGAGTTCC
GGGCCTCCTTCAACCACTTTGACCGGGATCACTCCGGCACACTGGGTCCCGAGGAGTTCAAAGCCTGC
CTCATCAGCTTGGGTTATGATATTGGCAACGACCCCCAGGGAGAAGCAGAATTTGCCCGCATCATGAG
CATTGTGGACCCCAACCGCCTGGGGGTAGTGACATTCCAGGCCTTCATTGACTTCATGTCCCGCGAGA
CAGCCGACACAGATACAGCAGACCAAGTCATGGCTTCCTTCAAGATCCTGGCTGGGGACAAGAACTAC
ATTACCATGGACGAGCTGCGCCGCGAGCTGCCACCCGACCAGGCTGAGTACTGCATCGCGCGGATGGC
CCCCTACACCGGCCCCGACTCCGTGCCAGGTGCTCTGGACTACATGTCCTTCTCCACGGCGCTGTACGG
CGAGAGTGACCTCTAATCCACCCCGCCCGGCCGCCCTCGTCTTGTGCGCCGTGCCCTGCCTTGCACCTC
CGCCGTCGCCCATCTCCTGCCTGGGTTCGGTTTCAGCTCCCAGCCTCCACCCGGGTGAGCTGGGGCCCA
CGTGGCATCGATCCTCCCTGCCCGCGAAGTGACAGTTTACAAAATTATTTTCTGCAAAAAAGAAAAAA
AAGTTACGTTAAAAACCAAAAAACTACATATTTTATTATAGAAAAAGTATTTTTTCTCCACCAGACAA
ATGGAAAAAAAGAGGAAAGATTAACTATTTGCACCGAAATGTCTTGTTTTGTTGCGACATAGGAAAAT
AACCAAGCACAAAGTTATATTCCATCCTTTTTACTGATTTTTTTTTCTTCTATCTGTTCCATCTGCTGTAT
TCATTTCTCCAATCTCATGTCCATTTTGGTGTGGGAGTCGGGGTAGGGGGTACTCTTGTCAAAAGGCAC
ATTGGTGCATGTGTGTTTGCTAGCTCACTTGTCCATGAAAATATTTTATGATATTAAAGAAAATCTTTT
GAAATGGCTGTTTTTTAAGGAAGAGAATTTATGTGGCTTCTCATTTTTAAATCCCCTCAGAGGTGTGAC
TAGTCTCTTTATCAGCACACACTTAAAAAATTTTTAATATTGTCTATTAAAAATAGGACAAACTTGGAG
AGTATGGACAACTTTGATATTGCTTGGCACAGATGGTATTAAAAAAACCACACTCCTATGACAAAAAA
AAAAAAAAAAAAA (SEQ ID NO: 2).
SEQ ID NO: 3-CUL2 (NP_001185707.1)
MYRVTWSTFWLRFQHYTCTMSLKPRVVDFDETWNKLLTTIKAVVMLEYVERATWNDRFSDIYALCVAYP
EPLGERLYTETKIFLENHVRHLHKRVLESEEQVLVMYHRYWEEYSKGADYMDCLYRYLNTQFIKKNKLTE
ADLQYGYGGVDMNEPLMEIGELALDMWRKLMVEPLQAILIRMLLREIKNDRGGEDPNQKVIHGVINSFVH
VEQYKKKFPLKFYQEIFESPFLTETGEYYKQEASNLLQESNCSQYMEKVLGRLKDEEIRCRKYLHPSSYTKV
IHECQQRMVADHLQFLHAECHNIIRQEKKNDMANMYVLLRAVSTGLPHMIQELQNHIHDEGLRATSNLTQ
ENMPTLFVESVLEVHGKFVQLINTVLNGDQHFMSALDKALTSVVNYREPKSVCKAPELLAKYCDNLLKKS
AKGMTENEVEDRLTSFITVFKYIDDKDVFQKFYARMLAKRLIHGLSMSMDSEEAMINKLKQACGYEFTSK
LHRMYTDMSVSADLNNKFNNFIKNQDTVIDLGISFQIYVLQAGAWPLTQAPSSTFAIPQELEKSVQMFELFY
SQHFSGRKLTWLHYLCTGEVKMNYLGKPYVAMVTTYQMAVLLAFNNSETVSYKELQDSTQMNEKELTK
TIKSLLDVKMINHDSEKEDIDAESSFSLNMNFSSKRTKFKITTSMQKDTPQEMEQTRSAVDEDRKMYLQAAI
VRIMKARKVLRHNALIQEVISQSRARFNPSISMIKKCIEVLIDKQYIERSQASADEYSYVA (SEQ ID NO: 3)
SEQ ID NO: 4-CUL2 (NM_001198778)
GTCACAGTAGGGAGTACCAGGAGGAGAGGAAGCTTGGGTGCCATGTTGCAGTTGAGCCCAAACTGAA
TGCTGTCTGTAGAAGGAAACAACAAACTTTGTACTTTATGTACAGAGTAACATGGTCAACTTTTTGGCT
TAGATTTCAACACTACACTTGCACAATGTCTTTGAAACCAAGAGTAGTAGATTTTGATGAAACATGGA
ACAAACTTTTGACGACAATAAAAGCCGTGGTCATGTTGGAATACGTCGAAAGAGCAACATGGAATGA
CCGTTTCTCAGATATCTATGCTTTATGTGTGGCCTATCCTGAACCCCTTGGAGAAAGACTTTATACAGA
AACTAAGATTTTTTTGGAAAATCATGTTCGGCATTTGCATAAGAGAGTTTTGGAGTCAGAAGAACAAG
TACTTGTTATGTATCATAGGTACTGGGAAGAATACAGCAAGGGTGCAGACTATATGGACTGCTTATAT
AGGTATCTCAACACCCAGTTTATTAAAAAGAATAAATTAACAGAAGCGGACCTTCAGTATGGCTATGG
TGGTGTAGATATGAATGAACCACTTATGGAAATAGGAGAGCTAGCATTGGATATGTGGAGGAAATTG
ATGGTTGAACCACTTCAGGCCATCCTTATCCGAATGCTGCTCCGAGAAATCAAAAATGATCGTGGTGG
AGAAGACCCAAACCAGAAAGTAATCCATGGGGTTATTAACTCCTTTGTTCATGTTGAACAGTATAAGA
AAAAATTCCCCTTAAAGTTTTATCAGGAAATTTTTGAGTCTCCCTTTCTGACTGAAACAGGAGAGTATT
ACAAACAAGAAGCTTCAAATTTATTACAAGAATCAAACTGCTCACAGTATATGGAAAAGGTTCTAGGT
AGATTAAAAGATGAAGAAATTCGATGTCGAAAATACCTACATCCAAGTTCATATACTAAGGTGATTCA
TGAATGTCAACAACGAATGGTAGCAGACCACTTACAGTTTTTACATGCAGAATGTCATAATATAATTC
GACAAGAGAAAAAAAATGACATGGCAAATATGTACGTCTTACTCCGTGCTGTGTCCACTGGTTTACCT
CATATGATTCAGGAGCTGCAAAACCACATCCATGATGAGGGCCTTCGAGCAACCAGCAACCTTACTCA
GGAAAACATGCCAACACTATTTGTGGAGTCAGTTTTGGAAGTGCATGGTAAATTTGTTCAGCTTATCA
ACACTGTTTTGAATGGTGATCAGCATTTTATGAGTGCGTTGGATAAGGCCCTTACGTCAGTTGTAAATT
ACAGAGAACCTAAGTCTGTTTGCAAAGCACCTGAACTGCTTGCTAAGTACTGTGACAACTTACTGAAG
AAGTCAGCGAAAGGGATGACAGAGAATGAAGTGGAAGACAGGCTCACGAGCTTCATCACAGTGTTCA
AATACATTGATGACAAGGACGTCTTTCAAAAGTTCTACGCAAGAATGCTGGCAAAACGTTTAATTCAT
GGGTTATCCATGTCTATGGACTCTGAAGAAGCCATGATCAACAAATTAAAGCAAGCCTGTGGTTATGA
GTTTACCAGCAAGCTACATCGGATGTATACAGATATGAGTGTCAGCGCTGATCTCAACAATAAGTTCA
ACAATTTTATCAAAAACCAAGACACAGTAATAGATTTGGGAATTAGTTTTCAAATATATGTTCTACAG
GCTGGTGCGTGGCCTCTTACTCAGGCTCCTTCATCTACGTTTGCAATTCCCCAGGAATTAGAAAAAAGT
GTACAGATGTTTGAATTATTTTATAGCCAACATTTCAGTGGAAGGAAACTTACATGGTTACATTATCTG
TGTACAGGTGAAGTTAAAATGAACTATTTGGGCAAACCATATGTAGCCATGGTTACAACATACCAAAT
GGCAGTTCTTCTTGCCTTTAACAACAGTGAAACTGTCAGTTATAAAGAGCTTCAGGACAGCACTCAGA
TGAATGAAAAGGAACTGACAAAAACAATCAAATCATTACTTGATGTGAAAATGATTAACCATGATTCA
GAAAAGGAAGATATTGATGCAGAATCTTCGTTTTCATTAAATATGAACTTTAGCAGTAAAAGAACAAA
ATTTAAAATTACTACATCAATGCAGAAAGACACACCACAAGAAATGGAGCAGACTAGAAGTGCAGTT
GATGAGGACCGGAAAATGTATCTCCAAGCTGCTATAGTTCGTATCATGAAAGCACGAAAAGTGCTTCG
GCACAATGCCCTTATTCAAGAGGTGATTAGCCAGTCAAGAGCTAGGTTTAATCCCAGTATCAGCATGA
TTAAGAAGTGTATTGAAGTTCTGATAGACAAACAATACATAGAACGCAGCCAGGCGTCGGCAGATGA
ATACAGCTACGTCGCGTGATGTCGCTCTCCTCCAGCGTGGTGTGAGAAGATCATTGCCATCACCATTTG
GTGTGTTCCTGTGGGAAAAAGCAGGACTGTGCCTCCATAATTTGGTCATTTGGCAGCCCCTGTTTTCTG
CTGTTTACAACATCACCAGTGCCACGTCATGAGCGTCAAAGAAAATGCCTAGAGATATTTCAAGCTCA
TGTCATTATGACATTTCTTAAAACTTTATTAAAAGAATGAGTGAAGTATTGCTGAAAAGTGGAAATTC
GGTTGGGTACCATGCTTTTTCTCCCCTTCACGTTTGCAGTTGATGTGTCTTTTTTTTTTTTTTTAATGTAT
CTTAAAGGACATAAAATTTAAAAACTTAAATATTGTAATATGACAGATAACCTAATAATTGTATCTAC
ATTAAAATGACAAACATGATACTGCTGCTTGTCAAATAAAAAAAAAATAAAGAAATAGAATGCCTTTT
TTATGTGGATGGAGTATCAGGTTGACCACAAAATATATTGACTCAAAGCAGCTAATGCATCTTTAGTT
GCGTTTTTATCTGAATGGTTTAATTCACTTGTACTCCTATTTAAATCCTACATGAAAAATGTCTAGATTA
TTGTTCTTGACTGCATAGGACTGCATTCAGCATAAAGAATGCTTTATTTTTATGGATTAGATATATTGG
ATCTAAACATTTTGAATCTTGAAGATGTAATTCCATCAGCAGTTTCTGGTGGTGTGCTACTCCACAGAC
ATCGCAGAGTGTGAGCAGGATGCTTGGTGACCTCAAGTCTGGCACAGAGAGAGCTTTTCATTCAAAAG
TTGTCTTTCTTCGGTTGCATAATCCATTAATTCTAGCATAGACTAGTACCCTAGCTCTGTGGCCTTCCCT
GAGTCTTAGGAAATCTATGATACCAACATATTCCTTCTATATGCCTCCCCTACCTGTTACCCTTACAAC
CCTCCTCCAACAGTTTAGATACTAGAGTCACTCTCATCAATCACAGATGTGCTTAGCAATGCATAACCT
AAATACTTTTTTAAAAAAGAAAATTGTACATTGTACTGGGTGCCACATATATAAATCCCATTATTTTGT
TTATTTTATATATATATATATATATAATATATATATATATATATCTCAACAGCAGTGTTAAGAGTACTG
CGATCTATTATCATATTTATTGTCTATCCACACCATCACCACCACCACCACACCCCTCCTCCCTCAACAT
ACAATTTTTCTTTATTTTAAAAAAAATAAGAGACGGGGTTTCGCCATGTTTCCCAGGCTAGTCTGGAAC
TTCTGGCCTCAAGCAATCCTATCTCTGTCTCCCAAAGTGCTGGGATTACAAGCATGAGCCACTGCATCC
ATCCAACACAAAATTTTTAAAATCGGAATATTTTAAAGCAAATCACACAAATTATTTCACTTATAATAC
TTCAGTAAGGCCTTTAAAAAATCCACAGTGATATTATTACTCCTAACAAAAACAATAATTACTTAGTAT
CATCTAATATGTGGTTCATATTTAAATTTGTTGTTTTGAGATGGGTCTTACAATTGGTTTATTCAATTGC
ATTTTTTCTAACTCGTGTCTCAAGTGTTTTAAAAATCTACTGAACTTATAATGACTTATATAATGTATTT
CTCATTTTACCTTTCTTCCAAAAGAGGAAATAATGGCAAACCATATAATATTGTACATTCACTGTCAAA
AAGCAAACCCTTGTTTTGATAACTTGTTGATTGATAAAAGTTTTCCAAATTGAAAAAAAAAAAAAA
(SEQ ID NO: 4)
SEQ ID NO: 5-DCC (NP_005206.2)
MENSLRCVWVPKLAFVLFGASLFSAHLQVTGFQIKAFTALRFLSEPSDAVTMRGGNVLLDCSAESDRGVPV
IKWKKDGIHLALGMDERKQQLSNGSLLIQNILHSRHHKPDEGLYQCEASLGDSGSIISRTAKVAVAGPLRFL
SQTESVTAFMGDTVLLKCEVIGEPMPTIHWQKNQQDLTPIPGDSRVVVLPSGALQISRLQPGDIGIYRCSAR
NPASSRTGNEAEVRILSDPGLHRQLYFLQRPSNVVAIEGKDAVLECCVSGYPPPSFTWLRGEEVIQLRSKKY
SLLGGSNLLISNVTDDDSGMYTCVVTYKNENISASAELTVLVPPWFLNHPSNLYAYESMDIEFECTVSGKPV
PTVNWMKNGDVVIPSDYFQIVGGSNLRILGVVKSDEGFYQCVAENEAGNAQTSAQLIVPKPAIPSSSVLPSA
PRDVVPVLVSSRFVRLSWRPPAEAKGNIQTFTVFFSREGDNRERALNTTQPGSLQLTVGNLKPEAMYTFRV
VAYNEWGPGESSQPIKVATQPELQVPGPVENLQAVSTSPTSILITWEPPAYANGPVQGYRLFCTEVSTGKEQ
NIEVDGLSYKLEGLKKFTEYSLRFLAYNRYGPGVSTDDITVVTLSDVPSAPPQNVSLEVVNSRSIKVSWLPP
PSGTQNGFITGYKIRHRKTTRRGEMETLEPNNLWYLFTGLEKGSQYSFQVSAMTVNGTGPPSNWYTAETPE
NDLDESQVPDQPSSLHVRPQTNCIIMSWTPPLNPNIVVRGYIIGYGVGSPYAETVRVDSKQRYYSIERLESSS
HYVISLKAFNNAGEGVPLYESATTRSITDPTDPVDYYPLLDDFPTSVPDLSTPMLPPVGVQAVALTHDAVR
VSWADNSVPKNQKTSEVRLYTVRWRTSFSASAKYKSEDTTSLSYTATGLKPNTMYEFSVMVTKNRRSST
WSMTAHATTYEAAPTSAPKDLTVITREGKPRAVIVSWQPPLEANGKITAYILFYTLDKNIPIDDWIMETISGD
RLTHQIMDLNLDTMYYFRIQARNSKGVGPLSDPILFRTLKVEHPDKMANDQGRHGDGGYWPVDTNLIDRS
TLNEPPIGQMHPPHGSVTPQKNSNLLVIIVVTVGVITVLVVVIVAVICTRRSSAQQRKKRATHSAGKRKGSQ
KDLRPPDLWIHHEEMEMKNIEKPSGTDPAGRDSPIQSCQDLTPVSHSQSETQLGSKSTSHSGQDTEEAGSSM
STLERSLAARRAPRAKLMIPMDAQSNNPAVVSAIPVPTLESAQYPGILPSPTCGYPHPQFTLRPVPFPTLSVD
RGFGAGRSQSVSEGPTTQQPPMLPPSQPEHSSSEEAPSRTIPTACVRPTHPLRSFANPLLPPPMSAIEPKVPYT
PLLSQPGPTLPKTHVKTASLGLAGKARSPLLPVSVPTAPEVSEESHKPTEDSANVYEQDDLSEQMASLEGL
MKQLNAITGSAF (SEQ ID NO: 5)
SEQ ID NO: 6-DCC (NM_005215)
GTAGTACGGTTCCAACTCCCAGCTCGCACACCGCTGGCGGACACCCCAGTAACAAGTGAGAGCGCTCC
ACCCCGCAGTCCCCCCCGCCTCTCCTCCCTGGGTCCCCTCGGCTCTCGGAAGAAAAACCAACAGCATCT
CCAGCTCTCGCGCGGAATTGTCTCTTCAACTTTACCCAACCGACGACAAGGAACCAGCCTCAACCTTTT
AATGCACAGCCCGGCCACAGGATTGCCTTCCATCTCCTCTTGGTCCCTCCTGGATGTGGTTTATTGATG
ACTTGCGAGCCCCTCAGAGAGCTGTCTTCCCTCCTCTGGCTCCCTCCGTTTCCTTGAGTTAGTTTTCTAA
GGTTTTACCGGGGCTCGGGATCTCTTGGACCGAATGGAACTTTTTGCTGCCTGCTTTTGCTGCTGATTCT
GTCAGTGGACAAGGAAAAAGGCTTCGAAGGCAGCAGAGGCGCAGGGGAGGTGGAGAAAGAGGTGGA
GGAAGAGGACGAGGAGGAGGAGGAAGCCGAAGGGGCTCGGCGCGTGTGTGTGCATGTGTGCATGCGT
GTGTGAGTGCATGTGTGTGAGTGCTGCCGCTGCCCGCGACCCCTGGCCCCGAAGGTGTTGGCTGAAAT
ATGGAGAATAGTCTTAGATGTGTTTGGGTACCCAAGCTGGCTTTTGTACTCTTCGGAGCTTCCTTGTTC
AGCGCGCATCTTCAAGTAACCGGTTTTCAAATTAAAGCTTTCACAGCACTGCGCTTCCTCTCAGAACCT
TCTGATGCCGTCACAATGCGGGGAGGAAATGTCCTCCTCGACTGCTCCGCGGAGTCCGACCGAGGAGT
TCCAGTGATCAAGTGGAAGAAAGATGGCATTCATCTGGCCTTGGGAATGGATGAAAGGAAGCAGCAA
CTTTCAAATGGGTCTCTGCTGATACAAAACATACTTCATTCCAGACACCACAAGCCAGATGAGGGACT
TTACCAATGTGAGGCATCTTTAGGAGATTCTGGCTCAATTATTAGTCGGACAGCAAAAGTTGCAGTAG
CAGGACCACTGAGGTTCCTTTCACAGACAGAATCTGTCACAGCCTTCATGGGAGACACAGTGCTACTC
AAGTGTGAAGTCATTGGGGAGCCCATGCCAACAATCCACTGGCAGAAGAACCAACAAGACCTGACTC
CAATCCCAGGTGACTCCCGAGTGGTGGTCTTGCCCTCTGGAGCATTGCAGATCAGCCGACTCCAACCG
GGGGACATTGGAATTTACCGATGCTCAGCTCGAAATCCAGCCAGCTCAAGAACAGGAAATGAAGCAG
AAGTCAGAATTTTATCAGATCCAGGACTGCATAGACAGCTGTATTTTCTGCAAAGACCATCCAATGTA
GTAGCCATTGAAGGAAAAGATGCTGTCCTGGAATGTTGTGTTTCTGGCTATCCTCCACCAAGTTTTACC
TGGTTACGAGGCGAGGAAGTCATCCAACTCAGGTCTAAAAAGTATTCTTTATTGGGTGGAAGCAACTT
GCTTATCTCCAATGTGACAGATGATGACAGTGGAATGTATACCTGTGTTGTCACATATAAAAATGAGA
ATATTAGTGCCTCTGCAGAGCTCACAGTCTTGGTTCCGCCATGGTTTTTAAATCATCCTTCCAACCTGT
ATGCCTATGAAAGCATGGATATTGAGTTTGAATGTACAGTCTCTGGAAAGCCTGTGCCCACTGTGAAT
TGGATGAAGAATGGAGATGTGGTCATTCCTAGTGATTATTTTCAGATAGTGGGAGGAAGCAACTTACG
GATACTTGGGGTGGTGAAGTCAGATGAAGGCTTTTATCAATGTGTGGCTGAAAATGAGGCTGGAAATG
CCCAGACCAGTGCACAGCTCATTGTCCCTAAGCCTGCTATCCCAAGCTCCAGTGTCCTCCCTTCGGCTC
CCAGAGATGTGGTCCCTGTCTTGGTTTCCAGCCGATTTGTCCGTCTCAGCTGGCGCCCACCTGCAGAAG
CGAAAGGGAACATTCAAACTTTCACGGTCTTTTTCTCCAGAGAAGGTGACAACAGGGAACGAGCATTG
AATACAACACAGCCTGGGTCCCTTCAGCTCACTGTGGGAAACCTGAAGCCAGAAGCCATGTACACCTT
TCGAGTTGTGGCTTACAATGAATGGGGACCGGGAGAGAGTTCTCAACCCATCAAGGTGGCCACACAGC
CTGAGTTGCAAGTTCCAGGGCCAGTAGAAAACCTGCAAGCTGTATCTACCTCACCTACCTCAATTCTTA
TTACCTGGGAACCCCCTGCCTATGCAAACGGTCCAGTCCAAGGTTACAGATTGTTCTGCACTGAGGTGT
CCACAGGAAAAGAACAGAATATAGAGGTTGATGGACTATCTTATAAACTGGAAGGCCTGAAAAAATT
CACCGAATATAGTCTTCGATTCTTAGCTTATAATCGCTATGGTCCGGGCGTCTCTACTGATGATATAAC
AGTGGTTACACTTTCTGACGTGCCAAGTGCCCCGCCTCAGAACGTCTCCCTGGAAGTGGTCAATTCAA
GAAGTATCAAAGTTAGCTGGCTGCCTCCTCCATCAGGAACACAAAATGGATTTATTACCGGCTATAAA
ATTCGACACAGAAAGACGACCCGCAGGGGTGAGATGGAAACACTGGAGCCAAACAACCTCTGGTACC
TATTCACAGGACTGGAGAAAGGAAGTCAGTACAGTTTCCAGGTGTCAGCCATGACAGTCAATGGTACT
GGACCACCTTCCAACTGGTATACTGCAGAGACTCCAGAGAATGATCTAGATGAATCTCAAGTTCCTGA
TCAACCAAGCTCTCTTCATGTGAGGCCCCAGACTAACTGCATCATCATGAGTTGGACTCCTCCCTTGAA
CCCAAACATCGTGGTGCGAGGTTATATTATCGGTTATGGCGTTGGGAGCCCTTACGCTGAGACAGTGC
GTGTGGACAGCAAGCAGCGATATTATTCCATTGAGAGGTTAGAGTCAAGTTCCCATTATGTAATCTCC
CTAAAAGCTTTTAACAATGCCGGAGAAGGAGTTCCTCTTTATGAAAGTGCCACCACCAGGTCTATAAC
CGATCCCACTGACCCAGTTGATTATTATCCTTTGCTTGATGATTTCCCCACCTCGGTCCCAGATCTCTCC
ACCCCCATGCTCCCACCAGTAGGTGTACAGGCTGTGGCTCTTACCCATGATGCTGTGAGGGTCAGCTG
GGCAGACAACTCTGTCCCTAAGAACCAAAAGACGTCTGAGGTGCGACTTTACACCGTCCGGTGGAGAA
CCAGCTTTTCTGCAAGTGCAAAATACAAGTCAGAAGACACAACATCTCTAAGTTACACAGCAACAGGC
CTCAAACCAAACACAATGTATGAATTCTCGGTCATGGTAACAAAAAACAGAAGGTCCAGTACTTGGAG
CATGACTGCACATGCCACCACGTATGAAGCAGCCCCCACCTCTGCTCCCAAGGACTTGACAGTCATTA
CTAGGGAAGGGAAGCCTCGTGCCGTCATTGTGAGTTGGCAGCCTCCCTTGGAAGCCAATGGGAAAATT
ACTGCTTACATCTTATTTTATACCTTGGACAAGAACATCCCAATTGATGACTGGATTATGGAAACAATC
AGTGGTGATAGGCTTACTCATCAAATCATGGATCTCAACCTTGATACTATGTATTACTTTCGAATTCAA
GCACGAAATTCAAAAGGAGTGGGGCCACTCTCTGATCCTATCCTCTTCAGGACTCTGAAAGTGGAACA
CCCTGACAAAATGGCTAATGACCAAGGTCGTCATGGAGATGGAGGTTATTGGCCAGTTGATACTAATT
TGATTGATAGAAGCACCCTAAATGAGCCGCCAATTGGACAAATGCACCCCCCGCATGGCAGTGTCACT
CCTCAGAAGAACAGCAACCTGCTTGTGATCATTGTGGTCACCGTTGGTGTCATCACAGTGCTGGTAGT
GGTCATCGTGGCTGTGATTTGCACCCGACGCTCTTCAGCCCAGCAGAGAAAGAAACGGGCCACCCACA
GTGCTGGCAAAAGGAAGGGCAGCCAGAAGGACCTCCGACCCCCTGATCTTTGGATCCATCATGAAGA
AATGGAGATGAAAAATATTGAAAAGCCATCTGGCACTGACCCTGCAGGAAGGGACTCTCCCATCCAA
AGTTGCCAAGACCTCACACCAGTCAGCCACAGCCAGTCAGAAACCCAACTGGGAAGCAAAAGCACCT
CTCATTCAGGTCAAGACACTGAGGAAGCAGGGAGCTCTATGTCCACTCTGGAGAGGTCGCTGGCTGCA
CGCCGAGCCCCCCGGGCCAAGCTCATGATTCCCATGGATGCCCAGTCCAACAATCCTGCTGTCGTGAG
CGCCATCCCGGTGCCAACGCTAGAAAGTGCCCAGTACCCAGGAATCCTCCCGTCTCCCACCTGTGGAT
ATCCCCACCCGCAGTTCACTCTCCGGCCTGTGCCATTCCCAACACTCTCAGTGGACCGAGGTTTCGGAG
CAGGAAGAAGTCAGTCAGTGAGTGAAGGACCAACTACCCAACAACCACCTATGCTGCCCCCATCTCAG
CCTGAGCATTCTAGCAGCGAGGAGGCACCAAGCAGAACCATCCCCACAGCTTGTGTTCGACCAACTCA
CCCACTCCGCAGCTTTGCTAATCCTTTGCTACCTCCACCAATGAGTGCAATAGAACCGAAAGTCCCTTA
CACACCACTTTTGTCTCAGCCAGGGCCCACTCTTCCTAAGACCCATGTGAAAACAGCCTCCCTTGGGTT
GGCTGGAAAAGCAAGATCCCCTTTGCTTCCTGTGTCTGTGCCAACAGCCCCTGAAGTGTCTGAGGAGA
GCCACAAACCAACAGAGGATTCAGCCAATGTGTATGAACAGGATGATCTGAGTGAACAAATGGCAAG
TTTGGAAGGACTCATGAAGCAGCTTAATGCCATCACAGGCTCAGCCTTTTAACATGTATTTCTGAATGG
ATGAGGTGAATTTTCCGGGAACTTTGCAGCATACCAATTACCCATAAACAGCACACCTGTGTCCAAGA
ACTCTAACCAGTGTACAGGTCACCCATCAGGACCACTCAGTTAAGGAAGATCCTGAAGCAGTTCAGAA
GGAATAAGCATTCCTTCTTTCACAGGCATCAGGAATTGTCAAATGATGATTATGAGTTCCCTAAACAA
AAGCAAAGATGCATTTTCACTGCAATGTCAAAGTTTAAGCTGCTAGAATAGTCATGGGCCTTTGTCACT
GCAGTGACCACACTGTCATAACTAATACCTATGTTTTCCTTTGTCAAGGCCTGTTGTTTAATGTGTAGG
TCTAGTCTTACAAAATGCAAGTGCATTATTTAAGCCTGTACCATGCCATGGCAAACCAGTGCAAGCTC
ACTATTTTGTTTTCAACTTAAACATACAAAGCACCCATGGGAATCTCTCATGCCATAGCACCAAAGGAT
TGGATGTTTTCCTTACAGCACAAAAAGTAAATAGTAAACAAACAAAAGGCAGAGAATGCTTATGTTTG
TAACTCAGTCATTCATCTTGCACAAGTGGTGGATATTAGTGAGTGGCTAAAAATTCACCTATTTTGGCA
AGTATTTGTAAATCCACCCTTGGTTAATATGTATGTCTGGAGTCCAGGAATATAAAAATCTGCAACTAG
TGGCATTCTGCCAGCAGCAGTACATTTCTGGAAAGAGGATATAATATGCAATCTTCTCAGACACATGG
TAATTATATGCTTAAGCTTGTAATAGGACAGTTTTCAATTTGGGTGGCTTTTGTGCCATACCACACTGT
GATACAATTTCAAAGCTTCACTAAGGCCATCTTCCTTAGGAGTTTGGCCAGAAGAATGCCCCCACCCCT
TCACCCCATCCCTCCCTGAGTTCTCCTTGGCAACTAGCGTTGGGTGAAATGGCCAGCTCCACATGTCAT
ATGGTGCACTGGCCAATGTCGCCTGTCTTCTAATCCCGTAGAAATGGCAGACTCCCTGAGAGCAGGAA
GAGAAGGAAAATAAAAGGTAGCTTCTAACAGTACCTTCTCTTAAAGAATGCCAACTCTGCCTACAGGG
TCAGTGTTGGCAAGCATTGGCCACCAGACCCTTTTGTTAAGGGAAACTTTTACACTACACCTGTGTCAG
AGTCAGGGGGAAGCAGAGGGGCAGGTGCCACCTGACACTTCCGACATGTAAATCCAGCAGATACTTTT
CAAAGCAGCATCTTAAACTGTGGACTACAGTTTTAAACTTCTATTGCCATGTTTATCTACAGCTTGGAA
CTAGCTAAAATTAAGAACATTTTGTATGCAGCATTTTAGTTTCTGAATTTTCAGCTGCATTTGGAGTTA
ATCCCTGTTTATGCAGCTGAATCGCCAAAAGGGAGCTAGTTTGCATATTTATCAGTTAGGTGACTTGAA
AACCCAATGAGAGAGTTTCAGCTGAATTATTCCTTTCAGCTCTGCCTTTGATTTCAAGCTTGAGTAGGT
CATAATTTTAAAAGAGCATGGAAGGGATAGGATCTTTACAACCTAATAGCTCCTTTTATTAGGTGGGT
AATTATATATGAATCCCTGAATAAAATATTTTGAGCAAAATGGCACTGTAACAGAAGTAATAATTCAG
TTTATTTTTTTACAGTTTTATGTCGGGAAGGAAATCTGATGTCAAAGAGAGGGCTGTTCAAATGGTTCA
TTAGAAAGTCCGGTCCATTTGCGAATTTGTTCCTTCAACAAGAGTGCTCATTCAAGTTACTCAGATTTT
CTGGAAGTCTTTTCTGAAGAGCTATGTGATGTTGTTCTATGGGACAGACTACTCTTATTTAACATCTGG
GCACTTAGGTAGACAACCTTCTACTGACCTGGAATAAAGTGTTTCCTAACATAATATTGAATTATTCAG
AAATAATCCATTACTTCAAAAAAGAAAATATTCATTGGGCTAGCCCAACCTTCTCTAGGCCCTAAGAA
TTATTACCTCCCCTTTCTAATTCTAGCAAACATGGAACATTCTCCTTAGGCACTTGACACCCACGAGGG
TAATCCTGAGTGCTCAGTTTGGAATAGGTTGCAAATCTCAGATTTTAGGGATTGAGTCACACCTTCAAT
CTATAGAATGAAGTTGACCAATTAAAAAAAAAAAAAAAACCTATCATTTTCACAAATTTCTAGATCCT
TCTAGTCAAAAATAATTATTTAGGAAATAAAATTTTTAAAAATCCATTTAAATACATGTTATTTGTCTT
CAGTGGAAGTTATATTTCTGCTGCATGCTTTTGAAACTTTCTTCATTAAATAGAATGGTTTGTCTTAGTA
ACTGGCAATGCCAGTATTAGCACCATGCATTTAATCTATAATACAATCAATTTAAACATCCTCAAAAA
ACTCTAGTATCATTTACCTGGTAGTATTAATATACAATGATGTCACCACAACTTTTGTATAACTCTGTTC
CCTTTACCCTCAAATGATTCATATATGTATATAATTGCCTGCCCAAGTTTTCAGGTAACTTATTAATTTC
CCAGTCTCCTGATCTCTTGACAAGAAGAAACCTGTGAATACTGCAAACTAGCCTCTGACTTCCTCCTAC
TGAGTCTAGTTCATGGTATCCAGGACTCTTTATGCTCATAACTCTCTCTGATTCCCATTGGGTGATACCT
GACAGCCAACCAGCCGCTCTGCCACCAGAACTCATTTCTCCCTGAAAAAGAAGAAAATCATATTTGGC
AGAGCATTCTCTGGTCTGCCCTGTAATGTGCTTAAATGTCAGGCAACATCCTCTTTTTTTTAAAAAAAA
TGGTATTTTTCTTTAAATTTCACCCTAATAAGAAAGCTATTTTCTCTCCTCTGCAGAAATTTCTGCATTT
GTGAAACTTATAAAAATTTAGATAGTTCAAATGTATAAAGAATATTTGGATGATGCTCTAGCCAAAAG
TTAAATATTTCGTAGTGAATCATAGCCAATAAGAAACCAGTCATACTTGCCTCTTTGAATAACAGAGA
TATAAGCTTCTAGAATATTTAAATAATGAAGTTTTACATTTGGGTATTATAAAATGCATACTCAATTGA
ATGAGCTGAAAAAAATACCAAGCCAGTGATATAAGTGGAGATTTATTAAGGATTCCTGTTGAGTATAT
TCTTAGTTTCCTCAAAATAGGGATTATTCAAAATTAGGTGTATGTTCAATCTCCTGCTTTGGTTCCAGCT
ACACAAGGAGAGCCATCCTGTGCTAGTGTGATGTTTCAGACAACATTCTGAAACTAAAATGTTTGGCA
CTTATTGGCTTTTCCAATAAAGAATCTCTTAAGTACAGGTATTTCTGGAAGCTGTTGGTGTCTGTGCTT
GAAGATGATTGCTGATACTTATCCACCCTTTGGGTACTTCTGTTGACTTTGTTTAAATAATCATCTTATG
GTTGTCCCCAAATGTAATATGGTATCTCAGATATAGCAGCTGGACTGTAATTACAACAAAAGGTTACC
TCTAAAGATAACATCTTACCATTTTAGATAAAATTGTGTCCCAGAATTCTTATGGTTTCGGAATGTACA
TTTCTAGTCAATGAAAGAAAGAAAAATGGAAAAATTGTCTAGTTTCAGGCATGTTTAAAGAAAACAAA
GTCATCTGAACTTTAAAATAGATGCAGAGCAGGGTTACTTTCCCTTTCACTCAGTTCCCTTCATGCAAC
CACAGGCAGTCCTGCAGGCCAGAGGTTACTATCCTAACCTGCTCATAACCATATACTATACAGAGCCC
ACAACTTTCTGGAGATGCAGAAGCAGCCATACACTCAAGTCTCTGTTTTTGTAAATCACATTCAAAGC
AACATTTTACTCATAATTTGCATTTCTCTGGTGACTTTCAGAAATCACTTTAGTATTGTACAGAAAAGC
TTTTTATTTGAGTCTAGTGTTTAAAATTAAATTGGATACTTGGGAAAATCATAGATAGGTGTTTTGTAT
GATATTCCATTCCAATGCAAAATATATGTACCCATGCCTCAATGTATCTTGCTATCTAAATACCTGTTG
CCAAAAAGTATTGATTTGGGAAAAAAAATGCCAATTTCCTGGTCAGTGAGGTTATGTAAAAGACAAAA
TACCACACCCATATCAGCAAATGAATATTACTACTCATCTGGACTCTTCGTTGCCACTATTGCATAACG
TTCACGTGGCAGACTTCCAGTTGCACTCTCTGAAGGACTTTTTTCTCTTACTCTCAATAGAGAGCCTTTG
TACATTGTCATCCTATGATTGTTGTTGGTAGAAGAGCAAGAGCAAAACTCTGCAAGATTTAATAAACA
CAGGGGCATGGGCCAAGGGATCTCACTGTGTGCTGAACATGTATTTTCAGATGCAAGAAAGGAATGAT
GGAGAGGAGGAGAAATGCTGTTTTTTATTATTGTAGGGTAAATCTGACAATTCTGAACTTTGTGAATTG
TCAGCTTGTTTGGGGGAAGGGTGGGCGGGTATGGGGTGTACTTTTTATAAGTAATATTTAATTTATTAT
TTAGAGTGGCTTCTTTTTGGATAATTTATGATAAAAAGGGAGATCTGGTTGGGATCTAGATACGGCTGT
TAAAGCTGCAGTGTTCCATACCTCAGAGGGACCACTTTGGAAATGAATTGTCCATTGCTGAGTATGAA
GAGATGTCCAGTCCAGGCAAAGCCTTCACTGAAGTTCCATCATCGCCACTTCTCCCTTTTTAGGGTCAT
TCAAAGAAGATAACACCAAACCTAAATAATTCTGAAAGCATTTTGCAGATCAGTGCTACTCATTCAAA
GGGCTTTGCAACTCAAACAGATTGTTAGTGTGCTAGTGATAAGTTTATTTGGTAGAAATGGGTATACTA
CAGCTTTAACTAGCCTTAGTGAGAAAAGAAATTTTTTGTTGTTACAAAACACCTTTTTTAACAAAAAGG
TATTTTGAGCCTACAAAAAGTTTCTTTAAACTGTCAGATTCTAGCATTGTTAACCAAATTAGACTAGTG
ATTGCAATATTTAAGTGTAAATCTTGTTCTACAAGAAAGGAAACTTGCTTACAGTTTAAAACAATGACT
GTTTCTACACATGATCTTGTATACTACTACACAAGGAAAAGGGGGTTTTGTAAACACTGTAGAACAGT
CTCATATTCATTTTTTTATAGAAATGTTATTCCAATGGTGCATTTTTTGTTTAATAAATAAAGTTTTGAT
ACAAAGTTC (SEQ ID NO: 6)
SEQ ID NO: 7-DERL1 (NP_077271.1)
MSDIGDWFRSIPAITRYWFAATVAVPLVGKLGLISPAYLFLWPEAFLYRFQIWRPITATFYFPVGPGTGFLYL
VNLYFLYQYSTRLETGAFDGRPADYLFMLLFNWICIVITGLAMDMQLLMIPLIMSVLYVWAQLNRDMIVSF
WFGTRFKACYLPWVILGFNYIIGGSVINELIGNLVGHLYFFLMFRYPMDLGGRNFLSTPQFLYRWLPSRRGG
VSGFGVPPASMRRAADQNGGGGRHNWGQGFRLGDQ (SEQ ID NO: 7)
SEQ ID NO: 8-DERL1 (NM_024295)
ACCTGGCTCCGCCCCCCAGGACGCCGAGCCTCGGCCGGGCGGTAAAATCGGCGCTTACCCTTTAAGCG
GCGGGACTTCTGGTCACGTCGTCCGCGGTCGCCGGAAGGGGAAGTTTCGCCTCAGAAGGCTGCCTCGC
TGGTCCGAATTCGGTGGCGCCACGTCCGCCCGTCTCCGCCTTCTGCATCGCGGCTTCGGCGGCTTCCAC
CTAGACACCTAACAGTCGCGGAGCCGGCCGCGTCGTGAGGGGGTCGGCACGGGGAGTCGGGCGGTCT
TGTGCATCTTGGCTACCTGTGGGTCGAAGATGTCGGACATCGGAGACTGGTTCAGGAGCATCCCGGCG
ATCACGCGCTATTGGTTCGCCGCCACCGTCGCCGTGCCCTTGGTCGGCAAACTCGGCCTCATCAGCCCG
GCCTACCTCTTCCTCTGGCCCGAAGCCTTCCTTTATCGCTTTCAGATTTGGAGGCCAATCACTGCCACCT
TTTATTTCCCTGTGGGTCCAGGAACTGGATTTCTTTATTTGGTCAATTTATATTTCTTATATCAGTATTCT
ACGCGACTTGAAACAGGAGCTTTTGATGGGAGGCCAGCAGACTATTTATTCATGCTCCTCTTTAACTGG
ATTTGCATCGTGATTACTGGCTTAGCAATGGATATGCAGTTGCTGATGATTCCTCTGATCATGTCAGTA
CTTTATGTCTGGGCCCAGCTGAACAGAGACATGATTGTATCATTTTGGTTTGGAACACGATTTAAGGCC
TGCTATTTACCCTGGGTTATCCTTGGATTCAACTATATCATCGGAGGCTCGGTAATCAATGAGCTTATT
GGAAATCTGGTTGGACATCTTTATTTTTTCCTAATGTTCAGATACCCAATGGACTTGGGAGGAAGAAAT
TTTCTATCCACACCTCAGTTTTTGTACCGCTGGCTGCCCAGTAGGAGAGGAGGAGTATCAGGATTTGGT
GTGCCCCCTGCTAGCATGAGGCGAGCTGCTGATCAGAATGGCGGAGGCGGGAGACACAACTGGGGCC
AGGGCTTTCGACTTGGAGACCAGTGAAGGGGCGGCCTCGGGCAGCCGCTCCTCTCAAGCCACATTTCC
TCCCAGTGCTGGGTGCACTTAACAACTGCGTTCTGGCTAACACTGTTGGACCTGACCCACACTGAATGT
AGTCTTTCAGTACGAGACAAAGTTTCTTAAATCCCGAAGAAAAATATAAGTGTTCCACAAGTTTCACG
ATTCTCATTCAAGTCCTTACTGCTGTGAAGAACAAATACCAACTGTGCAAATTGCAAAACTGACTACA
TTTTTTGGTGTCTTCTCTTCTCCCCTTTCCGTCTGAATAATGGGTTTTAGCGGGTCCTAGTCTGCTGGCA
TTGAGCTGGGGCTGGGTCACCAAACCCTTCCCAAAAGGACCCTTATCTCTTTCTTGCACACATGCCTCT
CTCCCACTTTTCCCAACCCCCACATTTGCAACTAGAAGAGGTTGCCCATAAAATTGCTCTGCCCTTGAC
AGGTTCTGTTATTTATTGACTTTTGCCAAGGCTTGGTCACAACAATCATATTCACGTAATTTTCCCCCTT
TGGTGGCAGAACTGTAGCAATAGGGGGAGAAGACAAGCAGCGGATGAAGCGTTTTCTCAGCTTTTGG
AATTGCTTCGACCTGACATCCGTTGTAACCGTTTGCCACTTCTTCAGATATTTTTATAAAAAAGTACCA
CTGAGTCAGTGAGGGCCACAGATTGGTATTAATGAGATACGAGGGTTGTTGCTGGGTGTTTGTTTCCTG
AGCTAAGTGATCAAGACTGTAGTGGAGTTGCAGCTAACATGGGTTAGGTTTAAACCATGGGGGATGCA
ACCCCTTTGCGTTTCATATGTAGGCCTACTGGCTTTGTGTAGCTGGAGTAGTTGGGTTGCTTTGTGTTAG
GAGGATCCAGATCATGTTGGCTACAGGGAGATGCTCTCTTTGAGAGGCTCCTGGGCATTGATTCCATTT
CAATCTCATTCTGGATATGTGTTCATTGAGTAAAGGAGGAGAGACCCTCATACGCTATTTAAATGTCAC
TTTTTTGCCTATCCCCCGTTTTTTGGTCATGTTTCAATTAATTGTGAGGAAGGCGCAGCTCCTCTCTGCA
CGTAGATCATTTTTTAAAGCTAATGTAAGCACATCTAAGGGAATAACATGATTTAAGGTTGAAATGGC
TTTAGAATCATTTGGGTTTGAGGGTGTGTTATTTTGAGTCATGAATGTACAAGCTCTGTGAATCAGACC
AGCTTAAATACCCACACCTTTTTTTCGTAGGTGGGCTTTTCCTATCAGAGCTTGGCTCATAACCAAATA
AAGTTTTTTGAAGGCCATGGCTTTTCACACAGTTATTTTATTTTATGACGTTATCTGAAAGCAGACTGTT
AGGAGCAGTATTGAGTGGCTGTCACACTTTGAGGCAACTAAAAAGGCTTCAAACGTTTTGATCAGTTT
CTTTTCAGGAAACATTGTGCTCTAACAGTATGACTATTCTTTCCCCCACTCTTAAACAGTGTGATGTGT
GTTATCCTAGGAAATGAGAGTTGGCAAACAACTTCTCATTTTGAATAGAGTTTGTGTGTACCTCTCCAT
ATTTAATTTATATGATAAAATAGGTGGGGAGAGTCTGAACCTTAACTGTCATGTTTTGTTGTTCATCTG
TGGCCACAATAAAGTTTACTTGTAAAATTTTAGAGGCCATTACTCCAATTATGTTGCACGTACACTCAT
TGTACAGGCGTGGAGACTCATTGTATGTATAAGAATATTCTGACAGTGAGTGACCCGGAGTCTCTGGT
GTACCCTCTTACCAGTCAGCTGCCTGCGAGCAGTCATTTTTTCCTAAAGGTTTACAAGTATTTAGAACT
CTTCAGTTCAGGGCAAAATGTTCATGAAGTTATTCCTCTTAAACATGGTTAGGAAGCTGATGACGTTAT
TGATTTTGTCTGGATTATGTTTCTGGAATAATTTTACCAAAACAAGCTATTTGAGTTTTGACTTGACAA
GGCAAAACATGACAGTGGATTCTCTTTACAAATGGAAAAAAAAAATCCTTATTTTGTATAAAGGACTT
CCCTTTTTGTAAACTAATCCTTTTTATTGGTAAAAATTGTAAATTAAAATGTGCAACTTGAAGGTTGTC
TGTGTTAAGTTTCCATGTCCCTGCTCTGCTGTCTCTTAGATATCACATAATTTGTGTAACCAATTATCTC
TTGAAGAGCATTTAGGAAGTACCCAGTATTTTTTGCTGGATTAATTCCTGGATGCAGAATTCCTGGGTT
TTCATTTTAATGAAGGAGGATGCTTGCTAACTTTGAAAAA (SEQ ID NO: 8)
SEQ ID NO: 9-FUS (NP_004951.1)
MASNDYTQQATQSYGAYPTQPGQGYSQQSSQPYGQQSYSGYSQSTDTSGYGQSSYSSYGQSQNTGYGTQS
TPQGYGSTGGYGSSQSSQSSYGQQSSYPGYGQQPAPSSTSGSYGSSSQSSSYGQPQSGSYSQQPSYGGQQQS
YGQQQSYNPPQGYGQQNQYNSSSGGGGGGGGGGNYGQDQSSMSSGGGSGGGYGNQDQSGGGGSGGYG
QQDRGGRGRGGSGGGGGGGGGGYNRSSGGYEPRGRGGGRGGRGGMGGSDRGGFNKFGGPRDQGSRHD
SEQDNSDNNTIFVQGLGENVTIESVADYFKQIGIIKTNKKTGQPMINLYTDRETGKLKGEATVSFDDPPSAK
AAIDWFDGKEFSGNPIKVSFATRRADFNRGGGNGRGGRGRGGPMGRGGYGGGGSGGGGRGGFPSGGGGG
GGQQRAGDWKCPNPTCENMNFSWRNECNQCKAPKPDGPGGGPGGSHMGGNYGDDRRGGRGGYDRGGY
RGRGGDRGGFRGGRGGGDRGGFGPGKMDSRGEHRQDRRERPY (SEQ ID NO: 9)
SEQ ID NO: 10-FUS (NM_004960)
ACTTAAGCTTCGACGCAGGAGGCGGGGCTGCTCAGTCCTCCAGGCGTCGGTACTCAGCGGTGTTGGAA
CTTCGTTGCTTGCTTGCCTGTGCGCGCGTGCGCGGACATGGCCTCAAACGATTATACCCAACAAGCAA
CCCAAAGCTATGGGGCCTACCCCACCCAGCCCGGGCAGGGCTATTCCCAGCAGAGCAGTCAGCCCTAC
GGACAGCAGAGTTACAGTGGTTATAGCCAGTCCACGGACACTTCAGGCTATGGCCAGAGCAGCTATTC
TTCTTATGGCCAGAGCCAGAACACAGGCTATGGAACTCAGTCAACTCCCCAGGGATATGGCTCGACTG
GCGGCTATGGCAGTAGCCAGAGCTCCCAATCGTCTTACGGGCAGCAGTCCTCCTACCCTGGCTATGGC
CAGCAGCCAGCTCCCAGCAGCACCTCGGGAAGTTACGGTAGCAGTTCTCAGAGCAGCAGCTATGGGC
AGCCCCAGAGTGGGAGCTACAGCCAGCAGCCTAGCTATGGTGGACAGCAGCAAAGCTATGGACAGCA
GCAAAGCTATAATCCCCCTCAGGGCTATGGACAGCAGAACCAGTACAACAGCAGCAGTGGTGGTGGA
GGTGGAGGTGGAGGTGGAGGTAACTATGGCCAAGATCAATCCTCCATGAGTAGTGGTGGTGGCAGTG
GTGGCGGTTATGGCAATCAAGACCAGAGTGGTGGAGGTGGCAGCGGTGGCTATGGACAGCAGGACCG
TGGAGGCCGCGGCAGGGGTGGCAGTGGTGGCGGCGGCGGCGGCGGCGGTGGTGGTTACAACCGCAGC
AGTGGTGGCTATGAACCCAGAGGTCGTGGAGGTGGCCGTGGAGGCAGAGGTGGCATGGGCGGAAGTG
ACCGTGGTGGCTTCAATAAATTTGGTGGCCCTCGGGACCAAGGATCACGTCATGACTCCGAACAGGAT
AATTCAGACAACAACACCATCTTTGTGCAAGGCCTGGGTGAGAATGTTACAATTGAGTCTGTGGCTGA
TTACTTCAAGCAGATTGGTATTATTAAGACAAACAAGAAAACGGGACAGCCCATGATTAATTTGTACA
CAGACAGGGAAACTGGCAAGCTGAAGGGAGAGGCAACGGTCTCTTTTGATGACCCACCTTCAGCTAA
AGCAGCTATTGACTGGTTTGATGGTAAAGAATTCTCCGGAAATCCTATCAAGGTCTCATTTGCTACTCG
CCGGGCAGACTTTAATCGGGGTGGTGGCAATGGTCGTGGAGGCCGAGGGCGAGGAGGACCCATGGGC
CGTGGAGGCTATGGAGGTGGTGGCAGTGGTGGTGGTGGCCGAGGAGGATTTCCCAGTGGAGGTGGTG
GCGGTGGAGGACAGCAGCGAGCTGGTGACTGGAAGTGTCCTAATCCCACCTGTGAGAATATGAACTTC
TCTTGGAGGAATGAATGCAACCAGTGTAAGGCCCCTAAACCAGATGGCCCAGGAGGGGGACCAGGTG
GCTCTCACATGGGGGGTAACTACGGGGATGATCGTCGTGGTGGCAGAGGAGGCTATGATCGAGGCGG
CTACCGGGGCCGCGGCGGGGACCGTGGAGGCTTCCGAGGGGGCCGGGGTGGTGGGGACAGAGGTGGC
TTTGGCCCTGGCAAGATGGATTCCAGGGGTGAGCACAGACAGGATCGCAGGGAGAGGCCGTATTAAT
TAGCCTGGCTCCCCAGGTTCTGGAACAGCTTTTTGTCCTGTACCCAGTGTTACCCTCGTTATTTTGTAAC
CTTCCAATTCCTGATCACCCAAGGGTTTTTTTGTGTCGGACTATGTAATTGTAACTATACCTCTGGTTCC
CATTAAAAGTGACCATTTTAGTTAAATTTTGTTCCTCTTCCCCCTTTTCACTTTCCTGGAAGATCGATGT
CCCGATCAGGAAGGTAGAGAGTTTTCCTGTTCAGATTACCCTGCCCAGCAGGAACTGGAATACAGTGT
TCGGGGAGAAGGCCAAATGATATCCTTGAGAGCAGAGATTAAACTTTTCTGTCATGGGGAAAGTTGGT
GTATAAATGAGAAATGAAGAACATGGGATGTCATGAGTGTTGGCCTAAATTTGCCCAGCTATGGGGAA
TTTTTCCTTTACCACATTTATTTGCATACTGGTCTTAGTTTATTTGCAGCAGTTTATCCCTTTTTAAGAAC
TCTTTGATCTTTTGGCCCTTTTAATGGTGAGGCTCAAACAAACTACATTTAAATGGGGCAGTATTCAGA
TTTGACCATGGTGGAGAGCGCTTAGCCACTCTGGGTCTTTCACAGGAAGGAGAGTAACTGAGTGCTGC
AGGAGTTTGTGGAGTGGAGTCAGGATCTAGGAGGTGAGTGACTCCCTTCCTAGCTGCCCTGGTGAACA
GCGCTTGGGTAGATACCTGCTATAAGGAGACTGGTCTGGCTGGGTTACTTTCACATCCTGCCTGTACTC
AGAGGGCTTGAGGTCATTGACATTATGAGATTTTAGGCTTGATCCCTTTTTGATTGGAGGGTGGAAGG
CCCTCCTAAGGGAATGATAAGTGATAAGAGGGGGAAGGGGTTGCAGCCAATGAGTTAAAACCTTAGA
GCAGTGCTCCTCAGCCTCTTACCATGTGGTTGTAAACTTGCACGTACCTGCCAACCAGTTATTTAGCAT
GCTTTTTATTTTAGTTACACAGAGCGTAACATTAACCCAAGAGCAGAAAGGTTTTATTTACAGGGTTTT
CGAACTTGGTTTGTAAGACAGCTGCCATCACAAGCATAGCTTACAAATGTGCTGGGGACCCCTAATTG
GGAAGTGCTTTCCTCTCAAATTTTTATTTTTTATTTTTAGAGACAGAGTCTTGCTCTGTCATCCAGGCTG
GAGTGCAGTGGCGTGATCTCGGCTCACTGTAGCCTCTGCCTCCTGAGTTTAAGCGATTCTCCTGCCTCA
GGAGAATCCCAGCTTCTGAGTAGCTGAGACTACAGGCGTGGGCCACCATGCCCACCTAGTTTTTGCAT
TTTTAGTGGAGGTGTGGTTTCACTGTGTTGGCCAGGCTGGTCTCTTAACTCCTGACCTCAGGTGATCCA
CCTGCCTTGGCCTCCCAAAGTGCTGGAATTACAGGCATGAGCCGCTGCATCTGGCCATCCTCTCAAATT
TTCAAGTGTTCCACAAGTATGTTCTCTACTGAAGAGTTGCTGCATCCTTGAATCTTGGGTGATTTGAGG
CACAGAAACTATGACTTTATTTTTTGAGATGGAGTTTTGCTCTTGTTGCCCAGGCTGGAGTGCAATGGC
ACGTTTTTCGGCTTACCGCAACTGCCGCCTCCTGGGTTCAAGCGATAGCTGGGATTACAGGCATGCGCC
ACCATGCCCAGCTAATTTATTTGTATTTTTAGTAGAGACGGGGTTTCTCCATGTTGGTCAGGCTGGTCT
CGAACTCCCGACCTCAGGTGATGTGCACACCTCAGCCTCCCAATAAACCATGACTTTTAAGAGGAATA
GCAGGTTTACTTCCCCTGCCAGCATTGGGGTGCTCTCTAAGCAACAGTAGGCGGAGAGTGGTCTGGCG
TATTAAAAACAAAGGATCGTCAAGTGGGCCTTCCCAGGCATTGCTTTGACTTAGTACATGTAGAGGAT
GTGGCAGTTCTCTCCGTCCCTGCCACTGCTGGTTTCTTTGTTAAATGTTTAGTTGAAATGGCCTGATACG
ATATTTGAGTAGTTCACTGTTGGTGCTTTGCCTAGCAGGATTCTAATCTTGCTTTGGTTGTGGTCCCCTG
ATGCCCTCCTGTTAGGAGTGGAGGAGGTCGAAGCTCCTTGTAAGATATGATTACTGGGACCATTAGTG
TCAAGTTCCTGTGTCCTTCAAATGGCATATGTGATTGGCCTTGACCTTAAAAGGAAATAGGGTCCCAG
GTGACTGTTTAGTGGGTAGGTCCAGTTTGGGGGGATCTTCCAGGAGAATGGATAGAGACACCTAGCAG
CAGAGAGAACATTGGTGCCTCTCAAGCCAACCTCCCACCTCAGCCTCCCAAGTAGCTGGGACTACAGG
TGCTTCCTCGCTACCACACCTGGGTAATTTTTTTTTTAATTACTTTTTTTTTTTTAAGAAACAGGGTTTCA
CTGGGTTGCCCAGGCTGGTCTTGAACTGGCCTCAAGTGACCTGCCTGCCTCAGCCTCCCAAAGTGCTGG
GATCACAGGTGTGACCCACTGCGTTTGGCCAGAATACTCTATTCTTACTGAATGATTGAAATCTGTCTT
GAAGCATTAGGTGTCCCATTTTTGTGAGTTGGAATTGGGACAGGCTAAGTAGGAAGTGAGGAGGGTGG
GGAGAGCTGTGCTGTAGGTCTGTTTGTCCCTTCCTTGATGTAGCCTTCAGTTAGCCCTTTCAGCTTTTTT
CCCCATCTTGTGCCGGGCCTTCCTGGGTTTCAGTACTTGGATGTAGGGCTGCAGTTATGTCAGTGGTGG
GTAGATTGACCAGGAATTAAGGTCTAGGGTCCAGCCCATGTGAGACTTGACTCACTGATCTACCTTTA
GGCATGTCTTCCTTCCAGTCTCATCCTTTTTAAATTTTTTTTTTTTTTTTGAGACGGTCTCACTCTCACCC
AGGCTGGATTGCAGTGGTGTGATCTCGACCAACTGCAACCTCTGCCTCCCACCCGCAAGCTATCTGCCC
ACCTCAGCCTCTGGAGTAGCTGGGACGGGACTACAGGCACCTGCCACCATGACTGGCTAATTTTTTTTT
GTATTTTTTGTGGAGATGGGGTCTTGCCATGTTGCTCAGGCTGGTCTGGTCTCAAAACTGCTCTGGGCT
CAAGTGATTGTCCCACTTTGGCCTCCCAGAGTGCTGGGATTAAGGTGTGAGATACTGTGTCCGGCTATG
AAAATTTTATTTTTAATTAACTTGTATATATTTATGGGGTACAATGTCCTATTTCTGTACATGTACACAT
TGTGGAATCAAATCAGGCTAATATATCCATCACTTCATATCATTAGCATGAATGAGAACATACAAAGC
CACTCTTAGAAAATTTTGAAATTTATGTTATTTCAGCCCTTTTATGCTGGAGGTTGCAAATGTTTTGTGA
ATAATCAGACCAAAAATAAAAACAAAAAATGATTGACTTCAGTCATTCAGTAAGAA
(SEQ ID NO: 10)
SEQ ID NO: 11-PDSS2 (NP_065114.3)
MNFRQLLLHLPRYLGASGSPRRLWWSPSLDTISSVGSWRGRSSKSPAHWNQVVSEAEKIVGYPTSFMSLRC
LLSDELSNIAMQVRKLVGTQHPLLTTARGLVHDSWNSLQLRGLVVLLISKAAGPSSVNTSCQNYDMVSGIY
SCQRSLAEITELIHIALLVHRGIVNLNELQSSDGPLKDMQFGNKIAILSGDFLLANACNGLALLQNTKVVELL
ASALMDLVQGVYHENSTSKESYITDDIGISTWKEQTFLSHGALLAKSCQAAMELAKHDAEVQNMAFQYG
KHMAMSHKINSDVQPFIKEKTSDSMTFNLNSAPVVLHQEFLGRDLWIKQIGEAQEKGRLDYAKLRERIKAG
KGVTSAIDLCRYHGNKALEALESFPPSEARSALENIVFAVTRFS (SEQ ID NO: 11)
SEQ ID NO: 12-PDSS2 (NM_020381)
GGCCGCATTCCATGCCTCCAATATGGCGTCCTCCACATAGGCAGTGGCTGTGGTTTCTACCCCGGGTGG
CCGGGGGCAGTGCTGAGCTGGGACTGTTGTTTGCCCAGCCTGGGCTGCAGAAAGCAGCAGTTAAAGTT
CGTTTCTGGTCACTGCTCCAGGAAGCCACCTTACTCTGAGGGTCAAGAATTGCCGCTTCCTTTTAGTTA
CTGTAAGTTCCTCCTCTGCCCCTGGTTTGTTTCCCGCGGCACTTCTGGATACCCCCAGGTCCCAGACCCT
TCCAGACTCAAACCATGAACTTTCGGCAGCTGCTGTTGCACTTGCCACGTTATCTTGGAGCCTCGGGTT
CCCCGCGTCGCCTGTGGTGGTCCCCGTCCCTCGACACCATCTCCTCGGTGGGCTCTTGGCGTGGTCGGT
CCTCCAAGTCCCCGGCCCACTGGAATCAGGTAGTGTCAGAGGCGGAGAAGATCGTGGGGTACCCCAC
GTCCTTCATGAGCCTTCGCTGCCTGCTGAGCGACGAGCTCAGCAACATCGCTATGCAGGTGCGGAAGC
TGGTGGGCACTCAGCACCCTCTGCTTACCACAGCCAGGGGGCTTGTACATGACAGCTGGAATAGCCTC
CAGTTGAGGGGCTTGGTGGTGCTCCTTATCTCTAAAGCAGCTGGGCCCAGCAGCGTGAACACTTCATG
TCAGAACTATGACATGGTCAGTGGGATCTACTCATGTCAAAGAAGTTTGGCAGAGATCACGGAGCTAA
TTCATATTGCTCTCCTTGTACATCGTGGGATAGTAAATTTAAATGAGTTGCAATCATCTGATGGTCCAC
TGAAAGACATGCAATTTGGAAATAAAATTGCTATCCTGAGTGGAGACTTTCTTCTAGCAAATGCCTGC
AATGGACTAGCTCTGCTACAGAACACCAAGGTTGTGGAACTTTTAGCAAGTGCTCTTATGGACTTGGT
ACAAGGAGTATATCATGAAAATTCTACTTCAAAGGAAAGTTATATCACAGATGATATTGGAATATCGA
CTTGGAAGGAGCAGACTTTTCTCTCCCATGGTGCCTTACTAGCAAAGAGCTGCCAAGCTGCAATGGAA
TTAGCAAAGCATGATGCTGAGGTTCAGAATATGGCATTTCAGTATGGGAAGCACATGGCCATGAGTCA
TAAGATAAATTCTGATGTCCAGCCTTTTATTAAAGAAAAGACCAGTGACTCCATGACTTTTAATCTAAA
CTCAGCTCCTGTAGTCTTACATCAGGAATTTCTTGGAAGAGATTTGTGGATTAAACAGATCGGAGAGG
CTCAAGAAAAAGGAAGATTGGACTATGCTAAGTTGCGAGAAAGAATCAAAGCTGGCAAAGGTGTGAC
TTCAGCTATTGACCTGTGTCGTTACCATGGAAACAAGGCACTGGAGGCCCTGGAGAGCTTTCCTCCCTC
GGAGGCCAGATCTGCTTTAGAAAACATTGTGTTTGCTGTGACCAGATTTTCATGACATCAAATTAAAA
AGACACTATTGTTAGTTAGCTGAAAATCCTAGGGAATGAGGTTGATTGGGAGCGCTTTCACGATGCGT
TAATGACTTTTAAAACATATGCATTTTTCCTTCCTTTTATCACATTGCTAAATGAGTTCTGCTTTCTTTTT
GGAACTGCTACAAACAAAATTAGAAGAAAAAAAGGTCAAGCAGTTTTCACTTGTCACGCCAGAAGCA
CACTTGAGGCTGCAGTCGCAGAAATAATTAATGAGATTCGCTCCTGTGACCTCAGCAAATGGACAGGA
AATAAGTCCTTATTGATTGGACCGAGCCAGGGATGGCGCCAGGGCGGTGGCCTGTGGTTTTTCCTGCT
AGAGAGGACAAAGCAAGTTGGAAGCTGCAGGTGTCAAGAGAAATGCTCTCAATACCAACCAGGGAGG
ATTGTCTAATCAAAAACTAGTGACCAATTTGTCATAATGGAGAGTAGTTCAATGGATTGAGAAAAATA
TGTTTTATTTGTTGGCTTGTAATTATGTCTCTGGATTATTATTATTTTTTTTTTAGATGTAGTTTTGCTCT
TGTTGCCCAGGCTGGAGTGCAATGGTGCAGTTTTGACTCACTGCAACCTCCGCCTCCTGGGTTCAAGTG
ATTCTCCTGCCTCAGCCTCCCGAGTAGCTGGAATTACAGGCACCTGCCACCACGCCTGGCTAATTTTGT
ATTTTTAGTAGAGATGGGGTTTCACTATGTTGGTCAGGCTAGTCTCGAACTCCTGACCTCAGGTGATCC
GCCTGCCTCGGCCTCCCAAAGTGCTGGGATTAGAGGCTTGAGCCACTGCACCTGGCCTCATGTCTCTGG
ATTTATAATGCAGTATGAATATACTTTGTGCTTTATGGTTTTTATAATGTCTTTTTGGAGAAATTGCCGA
AAAGTTGCCAAATACTTGAAGTAGGAGATTAAAATGTTATCAAATGTTAAATTGGTTATATTAGGAAT
AGTCTGTTTTTCTTTCCTGAAGATCAGTTTTTTTATTCAAACACATTTCAAAGAACCAAATTTTTTTTTT
CTTTAAGGAAAAAGGAGCTTTTTTTCAAGTGAAATGTATTCATTTGTAATACTTTGGTTTAAGGCATAC
TTTAATTTTTACGAGTTTCAGAAACAGAATTTTTGTACTAGGGAATTCATTGGTGAGAGTGTTCTTTTA
ACCTCAGAATGTCAAATTTTGGTCTTGAACCACAGACATCCAATTACAGAAAGAATATAAGCAATCTC
ACAGGCCTGCAATCGGACACTGTCTCTGTGTGGTTCATAGGAGATGATTTTTGAGGTTTGCACTCATGC
AATTTGAGAACACCGTTGACAAGAAGGCTGAGTTTACATAAATGATCTAGATTGAAACTCAGCTACCT
TTCTTCCTCATGTGGTGTAATTACAGCCCTATCTGGAGACAGCGAATACAGCAAACAGATTTTATTACC
TAGTTCGCTCAAACACTACATGAAGTTATTTTAGTTAAAGCCCTCCCCCAAAAGTTATAAAACCATTTT
ATCAGGGCCCAACATGTGGCATGCAATGAAGAGAAAATGTAAAGCTACAGAGGTTAATGTATTGTATT
ATAAAATATTTTAAGTGTACTCAAAATATCATAATTGTACAGTTTATGCCACCATAATTTGAGGCCTAT
AGATTTAGCTTAAGAGAACACTGTTCTGTTTGAAATGCTTTCTGTCACTGAAATTGGCTTAATTAGTAA
CCATGGATAAGATGCTTTAGATCAGACTAGGTTTTAATCATTAACTTCCACAAAGAAGTCATACTTTGC
GTTAGGTGTGCTGGTTGGATGTGCAGGAACTTCAGCAAGCAGTAGGTTTTACTAAGCAGATGGTCGGG
CACTGCAGGGCACCAGGCAGGATCCTAGGGCGCCTCTTATTCTGCGTTAGCATCTGGTTTGCTGTATGA
CCTTGCACAAGTCACTTCCTTCTGAGCCTCAATTTTCTCATCTGTACAATGAGATTCAAAAGTTGACCT
GAAAGTCAAGTGTGAAAAAAAAAAAGAGATTAAACAAGATAATTATGAAATTCTTAAAAAAAAAAAA
AAAA (SEQ ID NO: 12)
SEQ ID NO: 13-PLAG1 (NP_001108106.1)
MATVIPGDLSEVRDTQKVPSGKRKRGETKPRKNFPCQLCDKAFNSVEKLKVHSYSHTGERPYKCIQQDCT
KAFVSKYKLQRHMATHSPEKTHKCNYCEKMFHRKDHLKNHLHTHDPNKETFKCEECGKNYNTKLGFKR
HLALHAATSGDLTCKVCLQTFESTGVLLEHLKSHAGKSSGGVKEKKHQCEHCDRRFYTRKDVRRHMVVH
TGRKDFLCQYCAQRFGRKDHLTRHMKKSHNQELLKVKTEPVDFLDPFTCNVSVPIKDELLPVMSLPSSELL
SKPFTNTLQLNLYNTPFQSMQSSGSAHQMITTLPLGMTCPIDMDTVHPSHHLSFKYPFSSTSYAISIPEKEQPL
KGEIESYLMELQGGVPSSSQDSQASSSSKLGLDPQIGSLDDGAGDLSLSKSSISISDPLNTPALDFSQLFNFIPL
NGPPYNPLSVGSLGMSYSQEEAHSSVSQLPPQTQDLQDPANTIGLGSLHSLSAAFTSSLSTSTTLPRFHQAFQ
(SEQ ID NO: 13)
SEQ ID NO: 14-PLAG1 (NM_001114634)
AGCTGCAAGTTGGGCTGCAGGGGCAGCGCATACACTACAATGGCTGCTGGAAAGAGGCGTAAGGAAA
CAATTTCCAGGCCCGCCGCGTCCAGCCCGAAATATGAGAAAAAAATTATTAGAAATTCCGCGGGCGGT
GTAGAGGCGGCGGACGGGCCGGAGGGAGGATGTTAAAGCCCCGCGATTGGCCAAAATGGGAAGGATT
GGATTCCACTCTCTTCCACGAAGAGTCAATGGGACTGGCTAAGATCAAAGTCTGAGGCTTTTTCCATCA
GTAATCAGTCCCTTTTTGCTTTCTTTTACGACCACATGAAACTTGAGAAGCCACCTAAAGCTATATCAT
TTAGTGGAGTTGGGCAGTTCCCAAGTGTCCAACAAGAAGGCCTGGTTTAGGCTGCGATGGCCACTGTC
ATTCCTGGTGATTTGTCAGAAGTAAGAGATACCCAGAAAGTCCCTTCAGGGAAACGTAAGCGTGGTGA
AACCAAACCAAGAAAAAACTTTCCTTGCCAACTGTGTGACAAGGCCTTTAACAGTGTTGAGAAATTAA
AGGTTCACTCCTACTCTCACACAGGAGAGAGGCCCTACAAGTGCATACAACAAGACTGCACCAAGGCC
TTTGTTTCTAAGTACAAATTACAAAGGCACATGGCTACTCATTCTCCTGAGAAAACCCACAAGTGTAAT
TATTGTGAGAAAATGTTTCACCGGAAAGATCATCTGAAGAATCACCTCCATACACACGACCCTAACAA
AGAGACGTTTAAGTGCGAAGAATGTGGCAAGAACTACAATACCAAGCTTGGATTTAAACGTCACTTGG
CCTTGCATGCCGCAACAAGTGGTGACCTCACCTGTAAGGTATGTTTGCAAACTTTTGAAAGCACGGGA
GTGCTTCTGGAGCACCTTAAATCTCATGCAGGCAAGTCGTCTGGTGGGGTTAAAGAAAAAAAGCACCA
GTGCGAACATTGTGATCGCCGGTTCTACACCCGAAAGGATGTCCGGAGACACATGGTGGTGCACACTG
GAAGAAAGGACTTCCTCTGTCAGTATTGTGCACAGAGATTTGGGCGAAAGGATCACCTGACTCGACAT
ATGAAGAAGAGTCACAATCAAGAGCTTCTGAAGGTCAAAACAGAACCAGTGGATTTCCTTGACCCATT
TACCTGCAATGTGTCTGTGCCTATAAAAGACGAGCTCCTTCCGGTGATGTCCTTACCTTCCAGTGAACT
GTTATCAAAGCCATTCACAAACACTTTGCAGTTAAACCTCTACAACACTCCATTTCAGTCCATGCAGAG
CTCGGGATCTGCCCACCAAATGATCACAACTTTACCTTTGGGAATGACATGCCCAATAGATATGGACA
CTGTTCATCCCTCTCACCACCTTTCTTTCAAATATCCGTTCAGTTCTACCTCATATGCAATTTCTATTCCT
GAAAAAGAACAGCCATTAAAGGGGGAAATTGAGAGTTACCTGATGGAGTTACAAGGTGGCGTGCCCT
CTTCATCCCAAGATTCTCAAGCATCGTCATCATCTAAGCTAGGGTTGGATCCTCAGATTGGGTCCCTAG
ATGATGGTGCAGGAGACCTCTCCCTATCCAAAAGCTCTATCTCCATCAGTGACCCCCTAAACACACCA
GCATTGGATTTTTCTCAGTTGTTTAATTTCATACCTTTAAATGGTCCTCCCTATAATCCTCTATCAGTGG
GGAGCCTTGGAATGAGCTATTCCCAGGAAGAAGCACATTCTTCTGTTTCCCAGCTCCCCCCACAAACA
CAGGATCTTCAGGATCCTGCAAACACTATAGGGCTTGGGTCTCTGCACTCACTGTCAGCAGCTTTCACC
AGCAGTTTAAGCACAAGTACCACCCTCCCACGTTTCCATCAAGCTTTTCAGTAGGATTCTGGGACATGG
ATTCATTACAGAAATGTATGTGTAGCTGTGCCCTAGATGACCATTTTTATTTTAGTGCCTACTTTAAAA
CAGTATAAAAATTTCTGCTTTTGTATAATACAAATTTTCATTAAGCCAGTATAAAATAGAAACTAGCTT
TTAAACTGAGCTTTGGAACCATTTGTGTTCAGTTAAGTTTACCTGGGTATTTTGTCCTGATTCACTGCCA
ATTGTCACATTTTAAGACTTTTTTTTTTCCATATAGGAAAGCCATTATTAGTAGTAAACTTTTACAAATC
CCATTTTCAAATTACTTTTAGATCTTAAAATTTTCATTTTTGTCTAATAACAGTGGCTCTACCTTTTGAC
ATCTGGCTCATTAAAAAATTTAGCAATAGAATGTAAATTGTATAAAAAGTTTGTGAATAACTCAAGGG
TTTAAATTTTCTTACTAGCTTCTAAATGGATTAATAATCAAGTGCTTCAAATGAATTAAGAGTCCAGTT
TCGGAAGATAATAAATGTTTGTTAGATACACCATAATTTCAGATCAGTATATTCTGAAGACTCCCTGTT
GTCTGGCTAAAATATTTGCCATCTTTATTATGAGCCTTTAAGGAAAACAAACCCTAAACACAAAGCAT
CAGTATTTATAGCAAAAAGAGACTCTGTTAGGTGACATGGCATTTCGTGTCACTTAATAGTTGGCCCTA
AATTAGTACACAGGATATTTTGTCGTGTTTCATCCTTCTTAACATGCTATCTTTTCATTTAATAATAGTA
ATAGTGTATGGCATTGGGGTCTTCAGAGTCGATATATAGGTAGATCTCTTTAGTCTTTTCCACCTTTCA
CATCCAAGGGGTGGGTCAAGTGCAGCCAGCAATTTATTTTCATTGTTGGCCCACGGTTAGTCCATAATC
TAGAGCCATTGTGGAACTGCAGCCATGAGGTGTGTTTATCCCACAGTGGATTGACTCAGCCTCTGTGG
GTGACAGACTTCTAAGCAGGAAGATAGACGTGAAGCACATGGTTACATTTGGGAACTTGTGTAGGGAT
CATGGCCCCTGTAGCCAGGGTTAAAAACTGGACTTTTTAGAAGTAAAGTAAAAGCATATCGCTTATAT
CATTTCTTGCTGAATTTGATATGTTTTTCTTTCCCTTAAGAATCAAAAGCAGAAAACAAAAACAACAGT
CCTACTCCGATGTTATCTTTCTGATTCAATGTGAATCCATCTTTCCTTGCAATATTTTGGATGGAGAATT
TGAAGTTAAATGCATTAGAAAACTACCTGATGAACTACCACAAAGTTTTAAGTGACTAGAAATATATA
CAGTAAAATCCCACTTTCATGCATCTCTGGGAAATGATAGGAGTATTGCAAATAAGTTGAGTTTGTAG
AGGGTAACAAAGTAAAGTAAAACAAACCTATCTTGGTTAACATGAAAATAACAATTGAGAATATATT
ATATTCACTGAATAATTATAGGCTTTTCCTCACATTAGACAACCAACATAATCTTCTTAAAGGTCTAAT
TAATATATTTTTCTAAGGGTCAGTTGGGACATTAACCTAAGAAACATATCTATTAAGCACTTGTTAACA
CCTTATTTTAGGACCCTTTCCGTTGGGGATGGGGGCAAGGGTGGGAGGTTTTTAGAAGAGTATATATCT
CTTTAAAAAAAAACAGAAAGAAAAATATTTCTGAGCACTCATTAGCCCTATATGGAAACTTCTTTCCTT
TTTGTAGGGCCAGTTATCACTGCAGATTGCAATGTTTACCAAGAATTTCTAAAAATGAGTGCAGATTAC
TGAATATAATACATTATTTAAAATATTTGGGAGTAGTATAATTTGTGGAGAAATGTAAATTGTAATAAT
GTAAATGGGGGCTTCAATATATATATATAATACACACACACACACACATGCACACATACCGCACTTCA
TAGAATCAAAGTTGCTCTCTGAAGGAGCTTTGGCTCCTGATATTTTATCATGCTCCTATATTTTTTTAAT
CCTTGGAGCAGTAGTTTTTATACTTATGTATTTAAATTTTATTATGAAAAATTACATTTATTAAAAAAG
TGTGTTCCAAAGGCATTAAAATTATATATGTTAATAAGGAAGTACATTTTTAAATTTTTCAAACTGCTC
CTAGCTTTTGATTAGGAGAATATTTTTTCTGAAAGTAGGCTTTTCGCTCTGCTTCATTACTGCTTCCTTT
AGTTTCTATGAAACAGATTGCTTACCTAAATCTTTAGTTGAATGATTAGTGTTCAATATTGCTTTAATC
ACCATATAAAAGGAAAAAAATTGGTGACAGAGCACAAATAGAAAACCTATTTTTAAATAGAAATCAC
AAATAGCAAGTGTGGAAGCACTACTTTATTCTGTTTAAAATGTACTTAAGAAGTCATCAAATTAGTGA
ACTGAGACATTGGCCTTAGTAGGCTGTATTCACTGCTAATTTAAAAAAGGGAGTACCAGGATTTATTA
AGTAAAGCATTTTGGAAATGGGGAATAGCGCCATATATGTATGTATGTGTATGTGTGTGTGTGTGTGT
GTATATATACACACACACACACATACTTAAATCTTGCCCTGCATGAAATTCAAATACATGGAGGCACA
TCTTCAGGGCACCAGTGTTAAAATTTTGGAGTCTTAATTTTCATGTGTACACCTCTTTGCCTGTTCCCAC
CCCCAGACTTGAAATAACACTTCAGAGTAAGAGGGAATTCAGCTAATTTGTTTTTAAAATTGACTGTA
GTGGTCACTAAACCCTTTTTGAGAGAATTTCTATTAAAGATGAGGCAGACTCGCTTATTTGAATTGCAC
AATGTTCTAACAAGGATGTAACACAGAATTGGCTTTTTTTTCCCTAGAAAAAGATTGTTTGTTTCTATG
TCAACTAGATATGATTAAAAATAAGTATTGCCAATGCTGTTTTCATTCTCTAGTGGCCAGAATCATTAT
CCTTGAAATTTCTGGTAGTGCCTTAGCTTGGTTAAAAAAAAAAAAAAAAAAAAAAAAAAGGGATTAA
CATTAAATAAAAGTAGTTTAGAATTTGGGCCTCAGACAAGATATTGAACCTCATTCAGTTTCACTTCCA
CATGTATGTACAAGTTAGGTCACCAAACACGGAAGTGTGAGTGTGGAAGGATCTTGGCACTGTAAGCA
ATGCTATCCATTGATGTATACAAGTACCTTTATAGTTATCGATCACTGTTAAAACTTTCATTTTAAAATC
CTATTACCAAGTTCAGTTTTTTAAAACTTCAATTGTCCTGGCTGATTATGCATCACTCTGTGTGCAACTT
TTTTATTTCATTTAGTGTTTCTTTCAAGCTGTGTATTTTTGCCTATTTGTTGCTTGTGCTTTATTTTTCTTA
GTCATTTGTGGAATATAGTGATATATTGTGTTAATTTGGACAGTAGCGGTTTTTAAAAACCATATACTG
ACTGAAACATGAGCCAGAGCCGATTGCTTTATTAAGCTAATAATGAATGTTAAAGAGTACATATTTTC
AGGATCGTTCATCTAGTGAGCAATACACATATTATAGGCCAATATTTTTTTAAAAAATAGAGCTTGGTC
AACCTCTATACTACACATATTACAAGATATAGCACTTTCAAAATGAATCTAAACCTTTACAGAAACTTT
CTTATAGGTTATGCCTTTTATTTTAAGACTTATTATAATTCAAGTGCCATTAGATGATATATATGTAGGC
CTTTGATATATAATGCTTTGTGTACAAAAATGGTAGATGGTATTTTAAACAGGTACATTTTTACAGTGT
TTTCTTATCAATTTGCTATATTGCACAGAATCAGTGTGTGTCTTTTCATAAGGTTTTACAATGGTTTATT
TTTTTACAAGGTTTACGTGTCTCAAAGCACACTGTCTTCCCAGTACGTAAGTTAAAAAATACCAGTTCA
CCCAAGTTGCTTCTAGCCTACTGAGATCCATGTGACATTGGAGGAGATCTTTTAAATGTTTAGTATTCG
TCATTAGCAATGGCTGGCTGTTAGTTCTGGTAAATGTGTGCCTAAGTTGAATTTGTCTTGTTTTTCTCAC
ACTGTGTCAGCAGCCATGTCTACAACACAGATAAGTCTGTTGTGATCACATAGATCTACATAAGTTGT
GCAGTTTTGTGCTAAAAACCCATAGGGAGCTCCTTTGGGATCATAGAAAAGAAGATCATGCAACCAGC
ATTGGTGAAGGCACACTCAGATTGCACTTAGGGCCTTTCTATGATGTTGTCAACCCTCTGAGGATGGA
AGGCAGTGTCTTTTGATGTTATCTAGCCTAGAAATGACACAGAACTATTGCTAATGTATAAAACACTTC
ATTATATAAGCTTCAGTGGTACAGATGAACCAGAATGAATGTTTATCTTCTCAGAAACACTCCTTCAAT
ATTATATTGGATCATGCTGCTAATGTAACTTGGGCTACAACTCTTCATGGTGCTACAAACTTCTCTGTC
TCATTCAGTCGTATTTTTTTATCCATAGAAAAAGGACTACATTAGGTGTAAAAGTGTACAATATATTTT
TATACTGTGACTTAATTTGTCATTAACAAACTTTTACACCACCACAATGTATTCATGTGCACTTGCAAA
AGGAGATCTCGGACATGCAAATGTTACCAGAACAAACCCAGCTTTTGTCCACAAGGTGACTGTAACTC
AGAATGGAAAGTGGGCTTTATAATAGGGTGTGGAGTGAAGAACATGCTGTATGTTACTAACAGCCCTT
TGAATTTAACAAAAACTGGGAATCCATTAGGAAACGGATTGCATCATACCTGAACATAAGCTGGACTG
CTGAAATTGTATTTTTAGCTAATGAAAAAGTGTTTGGACTAGTACTCTAAAAATGTTCTAATGATAAAG
TTTTGAGTCAAAATAGAAAAGAAAAAAATCTGCATTCCAGGCCGAATTTTGTATATTTTTATTGCATTT
AAAATTGCTATTCTGTAATATTGGGAAATCAAGTGGCTTATCATGTATATCGTGTACTTAAAATGTATT
CACAAACTACTGTTGTATTTGTATAAAATATAGACAAAGATCATATTTTTTGTGTGTGTATAAGCTCTG
TAAAATAGCAATCACATTATGAAGCTGCAGTGATACTACATTTTAAACATTCACATCCAAAGAAGCAG
ACTATTTATTGTCCATATACCAGATTTAAAATATTAATTTGCTGCTAATTAAATAATAGTACTGCAGCT
TCTTGTGGCCTACAGTGTTATGTTTGCTGTAAGAATAAGATATGTGAATTCCACAAAATATATGAATAA
AATTATAGAATGGCTTTA (SEQ ID NO: 14)
SEQ ID NO: 15-RpS6 (NP_001001.2)
MKLNISFPATGCQKLIEVDDERKLRTFYEKRMATEVAADALGEEWKGYVVRISGGNDKQGFPMKQGVLT
HGRVRLLLSKGHSCYRPRRTGERKRKSVRGCIVDANLSVLNLVIVKKGEKDIPGLTDTTVPRRLGPKRASRI
RKLFNLSKEDDVRQYVVRKPLNKEGKKPRTKAPKIQRLVTPRVLQHKRRRIALKKQRTKKNKEEAAEYAK
LLAKRMKEAKEKRQEQIAKRRRLSSLRASTSKSESSQK (SEQ ID NO: 15)
SEQ ID NO: 16-RpS6 (NM_001010)
CCTCTTTTCCGTGGCGCCTCGGAGGCGTTCAGCTGCTTCAAGATGAAGCTGAACATCTCCTTCCCAGCC
ACTGGCTGCCAGAAACTCATTGAAGTGGACGATGAACGCAAACTTCGTACTTTCTATGAGAAGCGTAT
GGCCACAGAAGTTGCTGCTGACGCTCTGGGTGAAGAATGGAAGGGTTATGTGGTCCGAATCAGTGGTG
GGAACGACAAACAAGGTTTCCCCATGAAGCAGGGTGTCTTGACCCATGGCCGTGTCCGCCTGCTACTG
AGTAAGGGGCATTCCTGTTACAGACCAAGGAGAACTGGAGAAAGAAAGAGAAAATCAGTTCGTGGTT
GCATTGTGGATGCAAATCTGAGCGTTCTCAACTTGGTTATTGTAAAAAAAGGAGAGAAGGATATTCCT
GGACTGACTGATACTACAGTGCCTCGCCGCCTGGGCCCCAAAAGAGCTAGCAGAATCCGCAAACTTTT
CAATCTCTCTAAAGAAGATGATGTCCGCCAGTATGTTGTAAGAAAGCCCTTAAATAAAGAAGGTAAGA
AACCTAGGACCAAAGCACCCAAGATTCAGCGTCTTGTTACTCCACGTGTCCTGCAGCACAAACGGCGG
CGTATTGCTCTGAAGAAGCAGCGTACCAAGAAAAATAAAGAAGAGGCTGCAGAATATGCTAAACTTT
TGGCCAAGAGAATGAAGGAGGCTAAGGAGAAGCGCCAGGAACAAATTGCGAAGAGACGCAGACTTT
CCTCTCTGCGAGCTTCTACTTCTAAGTCTGAATCCAGTCAGAAATAAGATTTTTTGAGTAACAAATAAA
TAAGATCAGACTCTG (SEQ ID NO: 16)
SEQ ID NO: 17-SMAD2 (NP_001003652.1)
MSSILPFTPPVVKRLLGWKKSAGGSGGAGGGEQNGQEEKWCEKAVKSLVKKLKKTGRLDELEKAITTQNC
NTKCVTIPSTCSEIWGLSTPNTIDQWDTTGLYSFSEQTRSLDGRLQVSHRKGLPHVIYCRLWRWPDLHSHHE
LKAIENCEYAFNLKKDEVCVNPYHYQRVETPVLPPVLVPRHTEILTELPPLDDYTHSIPENTNFPAGIEPQSN
YIPETPPPGYISEDGETSDQQLNQSMDTGSPAELSPTTLSPVNHSLDLQPVTYSEPAFWCSIAYYELNQRVGE
TFHASQPSLTVDGFTDPSNSERFCLGLLSNVNRNATVEMTRRHIGRGVRLYYIGGEVFAECLSDSAIFVQSP
NCNQRYGWHPATVCKIPPGCNLKIFNNQEFAALLAQSVNQGFEAVYQLTRMCTIRMSFVKGWGAEYRRQ
TVTSTPCWIELHLNGPLQWLDKVLTQMGSPSVRCSSMS (SEQ ID NO: 17)
SEQ ID NO: 18-SMAD2 (NM_001003652.3)
CGGCCGGGAGGCGGGGCGGGCCGTAGGCAAAGGGAGGTGGGGAGGCGGTGGCCGGCGACTCCCCGC
GCCCCGCTCGCCCCCCGGCCCTTCCCGCGGTGCTCGGCCTCGTTCCTTTCCTCCTCCGCTCCCTCCGTCT
TCCATACCCGCCCCGCGCGGCTTTCGGCCGGCGTGCCTCGCGCCCTAACGGGCGGCTGGAGGCGCCAA
TCAGCGGGCGGCAGGGTGCCAGCCCCGGGGCTGCGCCGGCGAATCGGCGGGGCCCGCGGCCCAGGGT
GGCAGGCGGGTCTACCCGCGCGGCCGCGGCGGCGGAGAAGCAGCTCGCCAGCCAGCAGCCCGCCAGC
CGCCGGGAGGTTCGATACAAGAGGCTGTTTTCCTAGCGTGGCTTGCTGCCTTTGGTAAGAACATGTCGT
CCATCTTGCCATTCACGCCGCCAGTTGTGAAGAGACTGCTGGGATGGAAGAAGTCAGCTGGTGGGTCT
GGAGGAGCAGGCGGAGGAGAGCAGAATGGGCAGGAAGAAAAGTGGTGTGAGAAAGCAGTGAAAAGT
CTGGTGAAGAAGCTAAAGAAAACAGGACGATTAGATGAGCTTGAGAAAGCCATCACCACTCAAAACT
GTAATACTAAATGTGTTACCATACCAAGCACTTGCTCTGAAATTTGGGGACTGAGTACACCAAATACG
ATAGATCAGTGGGATACAACAGGCCTTTACAGCTTCTCTGAACAAACCAGGTCTCTTGATGGTCGTCTC
CAGGTATCCCATCGAAAAGGATTGCCACATGTTATATATTGCCGATTATGGCGCTGGCCTGATCTTCAC
AGTCATCATGAACTCAAGGCAATTGAAAACTGCGAATATGCTTTTAATCTTAAAAAGGATGAAGTATG
TGTAAACCCTTACCACTATCAGAGAGTTGAGACACCAGTTTTGCCTCCAGTATTAGTGCCCCGACACAC
CGAGATCCTAACAGAACTTCCGCCTCTGGATGACTATACTCACTCCATTCCAGAAAACACTAACTTCCC
AGCAGGAATTGAGCCACAGAGTAATTATATTCCAGAAACGCCACCTCCTGGATATATCAGTGAAGATG
GAGAAACAAGTGACCAACAGTTGAATCAAAGTATGGACACAGGCTCTCCAGCAGAACTATCTCCTACT
ACTCTTTCCCCTGTTAATCATAGCTTGGATTTACAGCCAGTTACTTACTCAGAACCTGCATTTTGGTGTT
CGATAGCATATTATGAATTAAATCAGAGGGTTGGAGAAACCTTCCATGCATCACAGCCCTCACTCACT
GTAGATGGCTTTACAGACCCATCAAATTCAGAGAGGTTCTGCTTAGGTTTACTCTCCAATGTTAACCGA
AATGCCACGGTAGAAATGACAAGAAGGCATATAGGAAGAGGAGTGCGCTTATACTACATAGGTGGGG
AAGTTTTTGCTGAGTGCCTAAGTGATAGTGCAATCTTTGTGCAGAGCCCCAATTGTAATCAGAGATATG
GCTGGCACCCTGCAACAGTGTGTAAAATTCCACCAGGCTGTAATCTGAAGATCTTCAACAACCAGGAA
TTTGCTGCTCTTCTGGCTCAGTCTGTTAATCAGGGTTTTGAAGCCGTCTATCAGCTAACTAGAATGTGC
ACCATAAGAATGAGTTTTGTGAAAGGGTGGGGAGCAGAATACCGAAGGCAGACGGTAACAAGTACTC
CTTGCTGGATTGAACTTCATCTGAATGGACCTCTACAGTGGTTGGACAAAGTATTAACTCAGATGGGA
TCCCCTTCAGTGCGTTGCTCAAGCATGTCATAAAGCTTCACCAATCAAGTCCCATGAAAAGACTTAATG
TAACAACTCTTCTGTCATAGCATTGTGTGTGGTCCCTATGGACTGTTTACTATCCAAAAGTTCAAGAGA
GAAAACAGCACTTGAGGTCTCATCAATTAAAGCACCTTGTGGAATCTGTTTCCTATATTTGAATATTAG
ATGGGAAAATTAGTGTCTAGAAATACTCTCCCATTAAAGAGGAAGAGAAGATTTTAAAGACTTAATGA
TGTCTTATTGGGCATAAAACTGAGTGTCCCAAAGGTTTATTAATAACAGTAGTAGTTATGTGTACAGGT
AATGTATCATGATCCAGTATCACAGTATTGTGCTGTTTATATACATTTTTAGTTTGCATAGATGAGGTG
TGTGTGTGCGCTGCTTCTTGATCTAGGCAAACCTTTATAAAGTTGCAGTACCTAATCTGTTATTCCCACT
TCTCTGTTATTTTTGTGTGTCTTTTTTAATATATAATATATATCAAGATTTTCAAATTATTTAGAAGCAG
ATTTTCCTGTAGAAAAACTAATTTTTCTGCCTTTTACCAAAAATAAACTCTTGGGGGAAGAAAAGTGG
ATTAACTTTTGAAATCCTTGACCTTAATGTGTTCAGTGGGGCTTAAACAGTCATTCTTTTTGTGGTTTTT
TGTTTTTTTTTGTTTTTTTTTTTAACTGCTAAATCTTATTATAAGGAAACCATACTGAAAACCTTTCCAA
GCCTCTTTTTTCCATTCCCATTTTTGTCCTCATAATCAAAACAGCATAACATGACATCATCACCAGTAAT
AGTTGCATTGATACTGCTGGCACCAGTTAATTCTGGGATACAGTAAGAATTCATATGGAGAAAGTCCC
TTTGTCTTATGCCCAAATTTCAACAGGAATAATTGGCTTGTATAATCTAGCAGTCTGTTGATTTATCCTT
CCACCTCATAAAAAATGCATAGGTGGCAGTATAATTATTTTCAGGGATATGCTAGAATTACTTCCACAT
ATTTATCCCTTTTTAAAAAAGCTAATCTATAAATACCGTTTTTCCAAAGGTATTTTACAATATTTCAACA
GCAGACCTTCTGCTCTTCGAGTAGTTTGATTTGGTTTAGTAACCAGATTGCATTATGAAATGGGCCTTT
TGTAAATGTAATTGTTTCTGCAAAATACCTAGAAAAGTGATGCTGAGGTAGGATCAGCAGATATGGGC
CATCTGTTTTTAAAGTATGTTGTATTCAGTTTATAAATTGATTGTTATTCTACACATAATTATGAATTCA
GAATTTTAAAAATTGGGGGAAAAGCCATTTATTTAGCAAGTTTTTTAGCTTATAAGTTACCTGCAGTCT
GAGCTGTTCTTAACTGATCCTGGTTTTGTGATTGACAATATTTCATGCTCTGTAGTGAGAGGAGATTTC
CGAAACTCTGTTGCTAGTTCATTCTGCAGCAAATAATTATTATGTCTGATGTTGACTCATTGCAGTTTA
AACATTTCTTCTTGTTTGCATCTTAGTAGAAATGGAAAATAACCACTCCTGGTCGTCTTTTCATAAATTT
TCATATTTTTGAAGCTGTCTTTGGTACTTGTTCTTTGAAATCATATCCACCTGTCTCTATAGGTATCATT
TTCAATACTTTCAACATTTGGTGGTTTTCTATTGGGTACTCCCCATTTTCCTATATTTGTGTGTATATGT
ATGTGTTCATGTAAATTTGGTATAGTAATTTTTTATTCATTCAACAAATATTTATTGTTCACCTGTTTGT
ACCAGGAACTTTTCTTAGTCTTTGGGTAAAGGTGAACAAGACAACTACAGTTCCTGCCTTTGCTGAGAC
AGCAGTTACACTAACCCTTAATTATCTTACTTGTCTATGAAGGAGATAAACAGGGTACTGTACTGGAG
AATAACAGATGGGATGCTTCAGGTAGGACATCAAGGAAAGCCTCTAAGGAAAGGATGCATGAGCTAA
CACCTGACATTAAAGAAGCAAGCCAAGTGAGGAGCCAGGGGAGATAAGCATTCCTGGCAAAGAGAAT
AGCATCAAATGCAAAAAGGTTCACACTAAAGGAAACTCCTGATTAGGTATTAATGCTTTATACAGAAA
CCTCTATACAAATCCAAACTTGAAGATCAGAATGGTTCTACAGTTCATAACATTTTGAAGGTGGCCTTA
TTTTGTGATAGTCTGCTTCATGTGATTCTCACTAACATATCTCCTTCCTCAACCTTTGCTGTAAAAATTT
CATTTGCACCACATCAGTACTACTTAATTTAACAAGCTTTTGTTGTGTAAGCTCTCACTGTTTTAGTGCC
CTGCTGCTTGCTTCCAGACTTTGTGCTGTCCAGTAATTATGTCTTCCACTACCCATCTTGTGAGCAGAGT
AAATGTCCTAGGTAATACCACTATCAGGCCTGTAGGAGATACTCAGTGGAGCCTCTGCCCTTCTTTTTC
TTACTTGAGAACTTGTAATGGTGTTAGGGAACAGTTGTAGGGGCAGAAAACAACTCTGAAAGTGGTAG
AAGGTCCTGATCTTGGTGGTTACTCTTGCATTACTGTGTTAGGTCAAGCAGTGCCTACTATGCTGTTTC
AGTAGTGGAGCGCATCTCTACAGTTCTGATGCGATTTTTCTGTACAGTATGAAATTGGGACTCAACTCT
TTGAAAACACCTATTGAGCAGTTATACCTGTTGAGCAGTTTACTTCCTGGTTGTAATTACATTTGTGTG
AATGTGTTTGATGCTTTTTAACGAGATGATGTTTTTTGTATTTTATCTACTGTGGCCTGATTTTTTTTTTG
TTTTCTGCCCCTCCCCCCATTTATAGGTGTGGTTTTCATTTTTCTAAGTGATAGAATCCCCTCTTTGTTG
AATTTTTGTCTTTATTTAAATTAGCAACATTACTTAGGATTTATTCTTCACAATACTGTTAATTTTCTAG
GAATGATGACCTGAGAACCGAATGGCCATGCTTTCTATCACATTTCTAAGATGAGTAATATTTTTTCCA
GTAGGTTCCACAGAGACACCTTGGGGGCTGGCTTAGGGGAGGCTGTTGGAGTTCTCACTGACTTAGTG
GCATATTTATTCTGTACTGAAGAACTGCATGGGGTTTCTTTTGGAAAGAGTTTCATTGCTTTAAAAAGA
AGCTCAGAAAGTCTTTATAACCACTGGTCAACGATTAGAAAAATATAACTGGATTTAGGCCTACCTTC
TGGAATACCGCTGATTGTGCTCTTTTTATCCTACTTTAAAGAAGCTTTCATGATTAGATTTGAGCTATAT
CAGTTATACCGATTATACCTTATAATACACATTCAGTTAGTAAACATTTATTGATGCCTGTTGTTTGCCC
AGCCACTGTGATGGATATTGAATAATAAAAAGATGACTAGGACGGGGCCCTGACCCTTGAGCTGTGCT
TGGTCTTGTAGAGGTTGTGTTTTTTTTCCTCAGGACCTGTCACTTTGGCAGAAGGAAATCTGCCTAATTT
TTCTTGAAAGCTAAATTTTCTTTGTAAGTTTTTACAAATTGTTTAATACCTAGTTGTATTTTTTACCTTA
AGCCACATTGAGTTTTGCTTGATTTGTCTGTCTTTTAAACACTGTCAAATGCTTTCCCTTTTGTTAAAAT
TATTTTAATTTCACTTTTTTTGTGCCCTTGTCAATTTAAGACTAAGACTTTGAAGGTAAAACAAACAAA
CAAACATCAGTCTTAGTCTCTTGCTAGTTGAAATCAAATAAAAGAAAATATATACCCAGTTGGTTTCTC
TACCTCTTAAAAGCTTCCCATATATACCTTTAAGATCCTTCTCTTTTTTCTTTAACTACTAAATAGGTTC
AGCATTTATTCAGTGTTAGATACCCTCTTCGTCTGAGGGTGGCGTAGGTTTATGTTGGGATATAAAGTA
ACACAAGACAATCTTCACTGTACATAAAATATGTCTTCATGTACAGTCTTTACTTTAAAAGCTGAACAT
TCCAATTTGCGCCTTCCCTCCCAAGCCCCTGCCCACCAAGTATCTCTTTAGATATCTAGTCTGTGGACA
TGAACAATGAATACTTTTTTCTTACTCTGATCGAAGGCATTGATACTTAGACATATCAAACATTTCTTC
CTTTCATATGCTTTACTTTGCTAAATCTATTATATTCATTGCCTGAATTTTATTCTTCCTTTCTACCTGAC
AACACACATCCAGGTGGTACTTGCTGGTTATCCTCTTTCTTGTTAGCCTTGTTTTTTGTTTTTTTTTTTTT
TTTTTGAGAGGGAGTCTCGCTCTGTTGCCCAACCTGGAGTGCAGTGGTGCGATCTTGGTTCACTGCAAG
CTCCGCCTCCCGGGTTCACGCCATGCTTCTGCCTCAGCCTCCCAAGTAGCTGGGACTACAGGCGCCCAC
CACCACACTCGGCTAATTTTTTGTATTTTTAGTAGAGACGGGGTTTCACCGTGTTGGCCAGGATGGTCT
CGATCTCCTGACCTCGTGATCTGTCCACCTCGGCTTCCCAAAGTGCTGGGATTACAGGCATGAGCCACC
GCGCCCAGCCTAGCCATATTTTTATCTGCATATATCAGAATGTTTCTCTCCTTTGAACTTATTAACAAA
AAAGGAACATGCTTTTCATACCTAGAGTCCTAATTTCTTCATCATGAAGGTTGCTATTCAAATTGATCA
ATCATTTTAATTTTACAAATGGCTCAAAAATTCTGTTCAGTAAATGTCTTTGTGACTGGCAAATGGCAT
AAATTATGTTTAAGATTATGAACTTTTCTGACAGTTGCAGCCAATGTTTTCCCTACGATACCAGATTTC
CATCTTGGGGCATATTGGATTGTTGTATTTAAGACAGTCAGAATAATGATAGTGTGTGGTCTCCAGAG
GTAGTCAGAATCCTGCTATTGAGTTCTTTTTATATCTTCCTTTTCAATTTTTTATTACCATTTTGTTTGTT
TAGACTACACTTTGTAGGGATTGAGGGGCAAATTATCTCTTGGAGTGGAATTCCTGTGTTTTGAGCCTT
ACAACCAGGAAATATGAGCTATACTAGATAGCCTCATGATAGCATTTACGATAAGAACTTATCTCGTG
TGTTCATGTAATTTTTTGAGTAGGAACTGTTTTATCTTGAATATTGTAGCTAACTATATATAGCAGAAC
TGCCTCAGTCTTTTTAAGAAGGAAATAAATAATATATGTGTATGAATTTATATATACATATACACTCAT
AGACAAACTTAACAGTTGGGGTCATTCTAACAGTTAAAACAATTGTTCCATTGTTTAAATCTCAGATCC
TGGTAAAATGTTCTTAATTTGTCTGTGTACATTTTCCTTTCATGGACAGACCATTGGAGTACATTAATTT
TCTTAATCTGCCATTTGGCAGTTCATTTAATATACCATTTTTTGGCAACTTGGTAACTAAGAATCACAG
CCAAAATTTGTTAACATCAAAGAAAGCTCTGCCATATACCCCGTTACTAAATTATTATACATCCAGCAG
ATTCTGGGATGTACTAACTTAGGGTTAACTTTGTTGTTGTTGATAATACTAGATTGCTCCCTCTTTAATT
CTTCTTCTGGTGCAAGGTTGCTGCTTAAGTTACCCTGGGAAATACTACTACAAGGTCAAATTTTCTAGT
ATCTTACAGCCTGATTGAAGGTGATTCAGATCTTTGCTCAATATAAATGGATTTTCCAAGATTCTCTGG
GCCATCCTTGACCCACAGGTGATCTCGCTGGAGTATATTAACTTAACTTCAGTGCCAGTTGGTTTGGTG
CCATGAGATCCATAATGAATCCAGAACTTCACCATTGCTTAGATATAAGAGTCCCTTGGAAGAATAAT
GCCACTGATGATGGGGGTCAGAAGGTGTATTAACTCAACATAGAGGGCTTTTAGATTTTTCTTCAAAA
AAATTTCGAGAAAAGTATTCTTTTACCCTCCAAACAGTTAACAGCTCTTAGTTTCTCCAAATATGCTCT
TTGATTTACTTATTTTTAATTAAAGATGGTAATTTATTGAACAATGAAATCCGTAATATATTGATTTAA
GGACAAAAGTGAAGTTTTAGAATTATAAAAGTACTTAAATATTATATATTTTCCATTTCATAATTGTTT
TCCTTTCTCTGTGGCTTTAAAGTTTTTGACTATTTTACAATGTTAATCACTAGGTAACTTGCCATATTTC
TGGTTCTATATTAAGTTCTATCCTTTATAATGCTGTTATTATAAAGCTGGTTTTTAGCATTTGTCTGTAG
CAATAGAAATTTTACTAAGTCTCTGTTCTCCCAGTAAGTTTTTTCTTTTCTCAGTAAGTCCCTAAGAAAA
CATTTGTTTGCCACTCTTACTATTCCCAATCTTGGATTGTTCGAGCTGAAAAAAAATTTGATGAGAAAC
AGGAGGATCCTTTTCTGGTGAATATAGGTTCCTGCTTTAAGAATGTGGAAATCCATTGCTTTATATAAC
TAATATACACACAGATTAATTAAAATTGTGAGAAATAATTCACACATGACAAGTAGGTAACATGCATG
AGTTTTGAATTTTTTTAAAAACCCAACTGTTTGACAAAATATAGAACCCAAATTGGTACTTTCTTAGAC
CAGTGTAACCTCACACCTCAGTTTTGCTTTTCCAACCCTGACTTGAAAGGCATATTTGTATCTTTTTATT
AGTGATAGTGAAGCTGTGACACTAACCTTTTATACAAAAGAGTAAAGAAAGAAAAACTACAGCGATT
AAGATGAGAACAGTTCTGCAGTTGTTGAACTAGATCACAGCATTGTAGGCAGAATAAAAAATGTTCAT
ATCTGAGAATATTCCTTTCGCCATCTTTTCCCAAGGCCAGACCTCCTGGTGGAGCACAGTTAAAAGTAA
CATTCTGGGCCTTTGTAATCGGAGGGCTGTGTCTCCAGCTGGCAGCCTTTGTTTTAATATATAATGCAG
GACTGTGGAAAACAGTTGGCATAGAATATTTTCACCTAAAAAAGAAAGAAAAGACATACAAAACTGG
ATTAATTGCAAAAAGAGAATACAGTAAAATACCATATAACTGGACAAAGCTAGAAGAACCTTTAGAA
GATTTGTCTGAAAACAGATTTCAAGAGTGAGCTTTTATACACTGCTCACTAATTTGCTTGATTACTACC
AACTCTTCTTAAAGTTAACACGTTTAAGGTATTTCTGGACTTCCTAGCCTTTTAGCAAGCTTAGAGGAA
CTAGCCATTAGCTAGTGATGTAAAAATATTTTGGGGACTGATGCCCTTAAAGGTTATGCCCTTGAAAGT
TCTTACCTTTTCTCTAGTGATATTAAGGAACGAGTGGGTAGTGTTCTCAGGGTGACCAGCTGCCCTAAA
GTGCCTGGGATTGAGGGTTTCCCTGGATGCGGGACTTTCCCTGGATACAAAACTTTTAGCAGAGTTTTG
TATATATGTGGATTTTTCTGATAAGTAGCACATCAGAGGCCTTAACCACTGCCCAAAAGCGATTCTCCA
TTGAGAGTACATATCTTGAACTTAAGAAATTCATTTGCTCTGATTTTTAATCTTGTAAAGTTTTTGCTAA
ACTCAAAACAAGTCCCAGGCACACCAGAAGGAGCTGACCACCTTAGGTGTTCTTGTGATTTATCCTTA
CTTCCCTATGTTGTCATAGTTGCTTCTAAACTCAGCTGCACTATGGCTGTCAACATTTCTGATACTTATT
GGGATATGTGCCATCCAGTCATTTAGTACTTTGAATGGAACATGAGATTTATAACACAGGTAATAGCT
GAAGGTACCAGTATGGTGGTGAGACTCACACTTAGTGATCCAGCTAAGGTAACTGATGTTATAATGGA
ACAGAGAAGAGGCCAACTAGATAGCTAAGTTCTTCTGAACCTATGTGTATATGTAAGTACAAATCATG
CGTCCTTATGGGGTTAAACTTAATCTGAAATTTACATTTTTCATAGTAAAAGGAAACCAATTGTTGCAG
ATTTCTTTTCTTGTGAGGAAATACATGGCCTTTGATGCTCTGGCGTCTACTGCATTTCCCAGTCTGTTCT
GCTCGAGAAGCCAGAATGTGTTGTTAACATTTTTCCGTGAATGTTGTGTTAAAATGATTAAATGCATCA
GCCAATGGCAAGTGAAGGAATTGGGTGTCCTGATGCAGACTGAGCAGTTTCTCTCAATTGTAGCCTCA
TACTCATAAGGTGCTTACCAGCTAGAACATTGAGCACGTGAGGTGAGATTTTTTTTCTCTGATGGCATT
AACTTTGTAATGCAATATGATGGATGCAGACCCTGTTCTTGTTTCCCTCTGGAAGTCCTTAGTGGCTGC
ATCCTTGGTGCACTGTGATGGAGATATTAAATGTGTTCTTTGTGAGCTTTCGTTCTATGATTGTCAAAA
GTACGATGTGGTTCCTTTTTTATTTTTATTAAACAATGAGCTGAGGCTTTATTACAGCTGGTTTTCAAGT
TAAAATTGTTGAATACTGATGTCTTTCTCCCACCTACACCAAATATTTTAGTCTATTTAAAGTACAAAA
AAAGTTCTGCTTAAGAAAACATTGCTTACATGTCCTGTGATTTCTGGTCAATTTTTATATATATTTGTGT
GCATCATCTGTATGTGCTTTCACTTTTTACCTTGTTTGCTCTTACCTGTGTTAACAGCCCTGTCACCGTT
GAAAGGTGGACAGTTTTCCTAGCATTAAAAGAAAGCCATTTGAGTTGTTTACCATGTTAAAAAAAAAA
AAAAAA (SEQ ID NO: 18)
SEQ ID NO: 19-SMAD4 (NP_005350.1)
MDNMSITNTPTSNDACLSIVHSLMCHRQGGESETFAKRAIESLVKKLKEKKDELDSLITAITTNGAHPSKCV
TIQRTLDGRLQVAGRKGFPHVIYARLWRWPDLHKNELKHVKYCQYAFDLKCDSVCVNPYHYERVVSPGI
DLSGLTLQSNAPSSMMVKDEYVHDFEGQPSLSTEGHSIQTIQHPPSNRASTETYSTPALLAPSESNATSTANF
PNIPVASTSQPASILGGSHSEGLLQIASGPQPGQQQNGFTGQPATYHHNSTTTWTGSRTAPYTPNLPHHQNG
HLQHHPPMPPHPGHYWPVHNELAFQPPISNHPAPEYWCSIAYFEMDVQVGETFKVPSSCPIVTVDGYVDPS
GGDRFCLGQLSNVHRTEAIERARLHIGKGVQLECKGEGDVWVRCLSDHAVFVQSYYLDREAGRAPGDAV
HKIYPSAYIKVFDLRQCHRQMQQQAATAQAAAAAQAAAVAGNIPGPGSVGGIAPAISLSAAAGIGVDDLR
RLCILRMSFVKGWGPDYPRQSIKETPCWIEIHLHRALQLLDEVLHTMPIADPQPLD (SEQ ID NO: 19)
SEQ ID NO: 20-SMAD4 (NM_005359)
ATGCTCAGTGGCTTCTCGACAAGTTGGCAGCAACAACACGGCCCTGGTCGTCGTCGCCGCTGCGGTAA
CGGAGCGGTTTGGGTGGCGGAGCCTGCGTTCGCGCCTTCCCGCTCTCCTCGGGAGGCCCTTCCTGCTCT
CCCCTAGGCTCCGCGGCCGCCCAGGGGGTGGGAGCGGGTGAGGGGAGCCAGGCGCCCAGCGAGAGAG
GCCCCCCGCCGCAGGGCGGCCCGGGAGCTCGAGGCGGTCCGGCCCGCGCGGGCAGCGGCGCGGCGCT
GAGGAGGGGCGGCCTGGCCGGGACGCCTCGGGGCGGGGGCCGAGGAGCTCTCCGGGCCGCCGGGGA
AAGCTACGGGCCCGGTGCGTCCGCGGACCAGCAGCGCGGGAGAGCGGACTCCCCTCGCCACCGCCCG
AGCCCAGGTTATCCTGAATACATGTCTAACAATTTTCCTTGCAACGTTAGCTGTTGTTTTTCACTGTTTC
CAAAGGATCAAAATTGCTTCAGAAATTGGAGACATATTTGATTTAAAAGGAAAAACTTGAACAAATG
GACAATATGTCTATTACGAATACACCAACAAGTAATGATGCCTGTCTGAGCATTGTGCATAGTTTGAT
GTGCCATAGACAAGGTGGAGAGAGTGAAACATTTGCAAAAAGAGCAATTGAAAGTTTGGTAAAGAAG
CTGAAGGAGAAAAAAGATGAATTGGATTCTTTAATAACAGCTATAACTACAAATGGAGCTCATCCTAG
TAAATGTGTTACCATACAGAGAACATTGGATGGGAGGCTTCAGGTGGCTGGTCGGAAAGGATTTCCTC
ATGTGATCTATGCCCGTCTCTGGAGGTGGCCTGATCTTCACAAAAATGAACTAAAACATGTTAAATATT
GTCAGTATGCGTTTGACTTAAAATGTGATAGTGTCTGTGTGAATCCATATCACTACGAACGAGTTGTAT
CACCTGGAATTGATCTCTCAGGATTAACACTGCAGAGTAATGCTCCATCAAGTATGATGGTGAAGGAT
GAATATGTGCATGACTTTGAGGGACAGCCATCGTTGTCCACTGAAGGACATTCAATTCAAACCATCCA
GCATCCACCAAGTAATCGTGCATCGACAGAGACATACAGCACCCCAGCTCTGTTAGCCCCATCTGAGT
CTAATGCTACCAGCACTGCCAACTTTCCCAACATTCCTGTGGCTTCCACAAGTCAGCCTGCCAGTATAC
TGGGGGGCAGCCATAGTGAAGGACTGTTGCAGATAGCATCAGGGCCTCAGCCAGGACAGCAGCAGAA
TGGATTTACTGGTCAGCCAGCTACTTACCATCATAACAGCACTACCACCTGGACTGGAAGTAGGACTG
CACCATACACACCTAATTTGCCTCACCACCAAAACGGCCATCTTCAGCACCACCCGCCTATGCCGCCCC
ATCCCGGACATTACTGGCCTGTTCACAATGAGCTTGCATTCCAGCCTCCCATTTCCAATCATCCTGCTC
CTGAGTATTGGTGTTCCATTGCTTACTTTGAAATGGATGTTCAGGTAGGAGAGACATTTAAGGTTCCTT
CAAGCTGCCCTATTGTTACTGTTGATGGATACGTGGACCCTTCTGGAGGAGATCGCTTTTGTTTGGGTC
AACTCTCCAATGTCCACAGGACAGAAGCCATTGAGAGAGCAAGGTTGCACATAGGCAAAGGTGTGCA
GTTGGAATGTAAAGGTGAAGGTGATGTTTGGGTCAGGTGCCTTAGTGACCACGCGGTCTTTGTACAGA
GTTACTACTTAGACAGAGAAGCTGGGCGTGCACCTGGAGATGCTGTTCATAAGATCTACCCAAGTGCA
TATATAAAGGTCTTTGATTTGCGTCAGTGTCATCGACAGATGCAGCAGCAGGCGGCTACTGCACAAGC
TGCAGCAGCTGCCCAGGCAGCAGCCGTGGCAGGAAACATCCCTGGCCCAGGATCAGTAGGTGGAATA
GCTCCAGCTATCAGTCTGTCAGCTGCTGCTGGAATTGGTGTTGATGACCTTCGTCGCTTATGCATACTC
AGGATGAGTTTTGTGAAAGGCTGGGGACCGGATTACCCAAGACAGAGCATCAAAGAAACACCTTGCT
GGATTGAAATTCACTTACACCGGGCCCTCCAGCTCCTAGACGAAGTACTTCATACCATGCCGATTGCA
GACCCACAACCTTTAGACTGAGGTCTTTTACCGTTGGGGCCCTTAACCTTATCAGGATGGTGGACTACA
AAATACAATCCTGTTTATAATCTGAAGATATATTTCACTTTTGTTCTGCTTTATCTTTTCATAAAGGGTT
GAAAATGTGTTTGCTGCCTTGCTCCTAGCAGACAGAAACTGGATTAAAACAATTTTTTTTTTCCTCTTC
AGAACTTGTCAGGCATGGCTCAGAGCTTGAAGATTAGGAGAAACACATTCTTATTAATTCTTCACCTGT
TATGTATGAAGGAATCATTCCAGTGCTAGAAAATTTAGCCCTTTAAAACGTCTTAGAGCCTTTTATCTG
CAGAACATCGATATGTATATCATTCTACAGAATAATCCAGTATTGCTGATTTTAAAGGCAGAGAAGTT
CTCAAAGTTAATTCACCTATGTTATTTTGTGTACAAGTTGTTATTGTTGAACATACTTCAAAAATAATG
TGCCATGTGGGTGAGTTAATTTTACCAAGAGTAACTTTACTCTGTGTTTAAAAAGTAAGTTAATAATGT
ATTGTAATCTTTCATCCAAAATATTTTTTGCAAGTTATATTAGTGAAGATGGTTTCAATTCAGATTGTCT
TGCAACTTCAGTTTTATTTTTGCCAAGGCAAAAAACTCTTAATCTGTGTGTATATTGAGAATCCCTTAA
AATTACCAGACAAAAAAATTTAAAATTACGTTTGTTATTCCTAGTGGATGACTGTTGATGAAGTATACT
TTTCCCCTGTTAAACAGTAGTTGTATTCTTCTGTATTTCTAGGCACAAGGTTGGTTGCTAAGAAGCCTA
TAAGAGGAATTTCTTTTCCTTCATTCATAGGGAAAGGTTTTGTATTTTTTAAAACACTAAAAGCAGCGT
CACTCTACCTAATGTCTCACTGTTCTGCAAAGGTGGCAATGCTTAAACTAAATAATGAATAAACTGAA
TATTTTGGAAACTGCTAAATTCTATGTTAAATACTGTGCAGAATAATGGAAACATTACAGTTCATAATA
GGTAGTTTGGATATTTTTGTACTTGATTTGATGTGACTTTTTTTGGTATAATGTTTAAATCATGTATGTT
ATGATATTGTTTAAAATTCAGTTTTTGTATCTTGGGGCAAGACTGCAAACTTTTTTATATCTTTTGGTTA
TTCTAAGCCCTTTGCCATCAATGATCATATCAATTGGCAGTGACTTTGTATAGAGAATTTAAGTAGAAA
AGTTGCAGATGTATTGACTGTACCACAGACACAATATGTATGCTTTTTACCTAGCTGGTAGCATAAATA
AAACTGAATCTCAACATACAAAGTTGAATTCTAGGTTTGATTTTTAAGATTTTTTTTTTCTTTTGCACTT
TTGAGTCCAATCTCAGTGATGAGGTACCTTCTACTAAATGACAGGCAACAGCCAGTTCTATTGGGCAG
CTTTGTTTTTTTCCCTCACACTCTACCGGGACTTCCCCATGGACATTGTGTATCATGTGTAGAGTTGGTT
TTTTTTTTTTTTAATTTTTATTTTACTATAGCAGAAATAGACCTGATTATCTACAAGATGATAAATAGAT
TGTCTACAGGATAAATAGTATGAAATAAAATCAAGGATTATCTTTCAGATGTGTTTACTTTTGCCTGGA
GAACTTTTAGCTATAGAAACACTTGTGTGATGATAGTCCTCCTTATATCACCTGGAATGAACACAGCTT
CTACTGCCTTGCTCAGAAGGTCTTTTAAATAGACCATCCTAGAAACCACTGAGTTTGCTTATTTCTGTG
ATTTAAACATAGATCTTGATCCAAGCTACATGACTTTTGTCTTTAAATAACTTATCTACCACCTCATTTG
TACTCTTGATTACTTACAAATTCTTTCAGTAAACACCTAATTTTCTTCTGTAAAAGTTTGGTGATTTAAG
TTTTATTGGCAGTTTTATAAAAAGACATCTTCTCTAGAAATTGCTAACTTTAGGTCCATTTTACTGTGAA
TGAGGAATAGGAGTGAGTTTTAGAATAACAGATTTTTAAAAATCCAGATGATTTGATTAAAACCTTAA
TCATACATTGACATAATTCATTGCTTCTTTTTTTTGAGATATGGAGTCTTGCTGTGTTGCCCAGGCAGGA
GTGCAGTGGTATGATCTCAGCTCACTGCAACCTCTGCCTCCCGGGTTCAACTGATTCTCCTGCCTCAGC
CTCCCTGGTAGCTAGGATTACAGGTGCCCGCCACCATGCCTGGCTAACTTTTGTAGTTTTAGTAGAGAC
GGGGTTTTGCCTGTTGGCCAGGCTGGTCTTGAACTCCTGACCTCAAGTGATCCATCCACCTTGGCCTCC
CAAAGTGCTGGGATTACGGGCGTGAGCCACTGTCCCTGGCCTCATTGTTCCCTTTTCTACTTTAAGGAA
AGTTTTCATGTTTAATCATCTGGGGAAAGTATGTGAAAAATATTTGTTAAGAAGTATCTCTTTGGAGCC
AAGCCACCTGTCTTGGTTTCTTTCTACTAAGAGCCATAAAGTATAGAAATACTTCTAGTTGTTAAGTGC
TTATATTTGTACCTAGATTTAGTCACACGCTTTTGAGAAAACATCTAGTATGTTATGATCAGCTATTCCT
GAGAGCTTGGTTGTTAATCTATATTTCTATTTCTTAGTGGTAGTCATCTTTGATGAATAAGACTAAAGA
TTCTCACAGGTTTAAAATTTTATGTCTACTTTAAGGGTAAAATTATGAGGTTATGGTTCTGGGTGGGTT
TTCTCTAGCTAATTCATATCTCAAAGAGTCTCAAAATGTTGAATTTCAGTGCAAGCTGAATGAGAGATG
AGCCATGTACACCCACCGTAAGACCTCATTCCATGTTTGTCCAGTGCCTTTCAGTGCATTATCAAAGGG
AATCCTTCATGGTGTTGCCTTTATTTTCCGGGGAGTAGATCGTGGGATATAGTCTATCTCATTTTTAATA
GTTTACCGCCCCTGGTATACAAAGATAATGACAATAAATCACTGCCATATAACCTTGCTTTTTCCAGAA
ACATGGCTGTTTTGTATTGCTGTAACCACTAAATAGGTTGCCTATACCATTCCTCCTGTGAACAGTGCA
GATTTACAGGTTGCATGGTCTGGCTTAAGGAGAGCCATACTTGAGACATGTGAGTAAACTGAACTCAT
ATTAGCTGTGCTGCATTTCAGACTTAAAATCCATTTTTGTGGGGCAGGGTGTGGTGTGTAAAGGGGGG
TGTTTGTAATACAAGTTGAAGGCAAAATAAAATGTCCTGTCTCCCAGATGATATACATCTTATTATTTT
TAAAGTTTATTGCTAATTGTAGGAAGGTGAGTTGCAGGTATCTTTGACTATGGTCATCTGGGGAAGGA
AAATTTTACATTTTACTATTAATGCTCCTTAAGTGTCTATGGAGGTTAAAGAATAAAATGGTAAATGTT
TCTGTGCCTGGTTTGATGGTAACTGGTTAATAGTTACTCACCATTTTATGCAGAGTCACATTAGTTCAC
ACCCTTTCTGAGAGCCTTTTGGGAGAAGCAGTTTTATTCTCTGAGTGGAACAGAGTTCTTTTTGTTGAT
AATTTCTAGTTTGCTCCCTTCGTTATTGCCAACTTTACTGGCATTTTATTTAATGATAGCAGATTGGGAA
AATGGCAAATTTAGGTTACGGAGGTAAATGAGTATATGAAAGCAATTACCTCTAAAGCCAGTTAACAA
TTATTTTGTAGGTGGGGTACACTCAGCTTAAAGTAATGCATTTTTTTTTCCCGTAAAGGCAGAATCCAT
CTTGTTGCAGATAGCTATCTAAATAATCTCATATCCTCTTTTGCAAAGACTACAGAGAATAGGCTATGA
CAATCTTGTTCAAGCCTTTCCATTTTTTTCCCTGATAACTAAGTAATTTCTTTGAACATACCAAGAAGTA
TGTAAAAAGTCCATGGCCTTATTCATCCACAAAGTGGCATCCTAGGCCCAGCCTTATCCCTAGCAGTTG
TCCCAGTGCTGCTAGGTTGCTTATCTTGTTTATCTGGAATCACTGTGGAGTGAAATTTTCCACATCATCC
AGAATTGCCTTATTTAAGAAGTAAAACGTTTTAATTTTTAGCCTTTTTTTGGTGGAGTTATTTAATATGT
ATATCAGAGGATATACTAGATGGTAACATTTCTTTCTGTGCTTGGCTATCTTTGTGGACTTCAGGGGCT
TCTAAAACAGACAGGACTGTGTTGCCTTTACTAAATGGTCTGAGACAGCTATGGTTTTGAATTTTTAGT
TTTTTTTTTTTAACCCACTTCCCCTCCTGGTCTCTTCCCTCTCTGATAATTACCATTCATATGTGAGTGTT
AGTGTGCCTCCTTTTAGCATTTTCTTCTTCTCTTTCTGATTCTTCATTTCTGACTGCCTAGGCAAGGAAA
CCAGATAACCAAACTTACTAGAACGTTCTTTAAAACACAAGTACAAACTCTGGGACAGGACCCAAGAC
ACTTTCCTGTGAAGTGCTGAAAAAGACCTCATTGTATTGGCATTTGATATCAGTTTGATGTAGCTTAGA
GTGCTTCCTGATTCTTGCTGAGTTTCAGGTAGTTGAGATAGAGAGAAGTGAGTCATATTCATATTTTCC
CCCTTAGAATAATATTTTGAAAGGTTTCATTGCTTCCACTTGAATGCTGCTCTTACAAAAACTGGGGTT
ACAAGGGTTACTAAATTAGCATCAGTAGCCAGAGGCAATACCGTTGTCTGGAGGACACCAGCAAACA
ACACACAACAAAGCAAAACAAACCTTGGGAAACTAAGGCCATTTGTTTTGTTTTGGTGTCCCCTTTGA
AGCCCTGCCTTCTGGCCTTACTCCTGTACAGATATTTTTGACCTATAGGTGCCTTTATGAGAATTGAGG
GTCTGACATCCTGCCCCAAGGAGTAGCTAAAGTAATTGCTAGTGTTTTCAGGGATTTTAACATCAGACT
GGAATGAATGAATGAAACTTTTTGTCCTTTTTTTTTCTGTTTTTTTTTTTCTAATGTAGTAAGGACTAAG
GAAAACCTTTGGTGAAGACAATCATTTCTCTCTGTTGATGTGGATACTTTTCACACCGTTTATTTAAAT
GCTTTCTCAATAGGTCCAGAGCCAGTGTTCTTGTTCAACCTGAAAGTAATGGCTCTGGGTTGGGCCAGA
CAGTTGCACTCTCTAGTTTGCCCTCTGCCACAAATTTGATGTGTGACCTTTGGGCAAGTCATTTATCTTC
TCTGGGCCTTAGTTGCCTCATCTGTAAAATGAGGGAGTTGGAGTAGATTAATTATTCCAGCTCTGAAAT
TCTAAGTGACCTTGGCTACCTTGCAGCAGTTTTGGATTTCTTCCTTATCTTTGTTCTGCTGTTTGAGGGG
GCTTTTTACTTATTTCCATGTTATTCAAAGGAGACTAGGCTTGATATTTTATTACTGTTCTTTTATGGAC
AAAAGGTTACATAGTATGCCCTTAAGACTTAATTTTAACCAAAGGCCTAGCACCACCTTAGGGGCTGC
AATAAACACTTAACGCGCGTGCGCACGCGCGCGCGCACACACACACACACACACACACACACACACA
GGTCAGAGTTTAAGGCTTTCGAGTCATGACATTCTAGCTTTTGAATTGCGTGCACACACACACGCACGC
ACACACTCTGGTCAGAGTTTATTAAGGCTTTCGAGTCATGACATTATAGCTTTTGAGTTGGTGTGTGTG
ACACCACCCTCCTAAGTGGTGTGTGCTTGTAATTTTTTTTTTCAGTGAAAATGGATTGAAAACCTGTTG
TTAATGCTTAGTGATATTATGCTCAAAACAAGGAAATTCCCTTGAACCGTGTCAATTAAACTGGTTTAT
ATGACTCAAGAAAACAATACCAGTAGATGATTATTAACTTTATTCTTGGCTCTTTTTAGGTCCATTTTG
ATTAAGTGACTTTTGGCTGGATCATTCAGAGCTCTCTTCTAGCCTACCCTTGGATGAGTACAATTAATG
AAATTCATATTTTCAAGGACCTGGGAGCCTTCCTTGGGGCTGGGTTGAGGGTGGGGGGTTGGGGAGTC
CTGGTAGAGGCCAGCTTTGTGGTAGCTGGAGAGGAAGGGATGAAACCAGCTGCTGTTGCAAAGGCTG
CTTGTCATTGATAGAAGGACTCACGGGCTTGGATTGATTAAGACTAAACATGGAGTTGGCAAACTTTC
TTCAAGTATTGAGTTCTGTTCAATGCATTGGACATGTGATTTAAGGGAAAAGTGTGAATGCTTATAGAT
GATGAAAACCTGGTGGGCTGCAGAGCCCAGTTTAGAAGAAGTGAGTTGGGGGTTGGGGACAGATTTG
GTGGTGGTATTTCCCAACTGTTTCCTCCCCTAAATTCAGAGGAATGCAGCTATGCCAGAAGCCAGAGA
AGAGCCACTCGTAGCTTCTGCTTTGGGGACAACTGGTCAGTTGAAAGTCCCAGGAGTTCCTTTGTGGCT
TTCTGTATACTTTTGCCTGGTTAAAGTCTGTGGCTAAAAAATAGTCGAACCTTTCTTGAGAACTCTGTA
ACAAAGTATGTTTTTGATTAAAAGAGAAAGCCAACTAAAAAAAAAAAAAAAAAAAA (SEQ ID NO: 20)
SEQ ID NO: 21-VDAC1 (NP_003365.1)
MAVPPTYADLGKSARDVFTKGYGFGLIKLDLKTKSENGLEFTSSGSANTETTKVTGSLETKYRWTEYGLTF
TEKWNTDNTLGTEITVEDQLARGLKLTFDSSFSPNTGKKNAKIKTGYKREHINLGCDMDFDIAGPSIRGALV
LGYEGWLAGYQMNFETAKSRVTQSNFAVGYKTDEFQLHTNVNDGTEFGGSIYQKVNKKLETAVNLAWT
AGNSNTRFGIAAKYQIDPDACFSAKVNNSSLIGLGYTQTLKPGIKLTLSALLDGKNVNAGGHKLGLGLEFQ
A (SEQ ID NO: 21)
SEQ ID NO: 22-VDAC1 (NM_003374.2)
ATTAGCGCAGGGACCTCCGGGCCACAGCTCAGAGAATCGGAAGGCCTCCTCCCCCTTCCCGAGCGCTG
CCACTGGGGCCGAGGTTTCCAGCAAGAACCCGCGTGTCCCTGCGCACGCACACACGGTGCACACGTCA
GTCCGGCGCCTCCCCGTGCCCCGACTCACGCAGGTCCTCCCGCGCGCCCGCAACACGCCCGCAGGCTC
CTGTGTCTGCTGCCGGGGCAGCGGGGCCCGGAAGGCAGAAGATGGCTGTGCCACCCACGTATGCCGAT
CTTGGCAAATCTGCCAGGGATGTCTTCACCAAGGGCTATGGATTTGGCTTAATAAAGCTTGATTTGAA
AACAAAATCTGAGAATGGATTGGAATTTACAAGCTCAGGCTCAGCCAACACTGAGACCACCAAAGTG
ACGGGCAGTCTGGAAACCAAGTACAGATGGACTGAGTACGGCCTGACGTTTACAGAGAAATGGAATA
CCGACAATACACTAGGCACCGAGATTACTGTGGAAGATCAGCTTGCACGTGGACTGAAGCTGACCTTC
GATTCATCCTTCTCACCTAACACTGGGAAAAAAAATGCTAAAATCAAGACAGGGTACAAGCGGGAGC
ACATTAACCTGGGCTGCGACATGGATTTCGACATTGCTGGGCCTTCCATCCGGGGTGCTCTGGTGCTAG
GTTACGAGGGCTGGCTGGCCGGCTACCAGATGAATTTTGAGACTGCAAAATCCCGAGTGACCCAGAGC
AACTTTGCAGTTGGCTACAAGACTGATGAATTCCAGCTTCACACTAATGTGAATGACGGGACAGAGTT
TGGCGGCTCCATTTACCAGAAAGTGAACAAGAAGTTGGAGACCGCTGTCAATCTTGCCTGGACAGCAG
GAAACAGTAACACGCGCTTCGGAATAGCAGCCAAGTATCAGATTGACCCTGACGCCTGCTTCTCGGCT
AAAGTGAACAACTCCAGCCTGATAGGTTTAGGATACACTCAGACTCTAAAGCCAGGTATTAAACTGAC
ACTGTCAGCTCTTCTGGATGGCAAGAACGTCAATGCTGGTGGCCACAAGCTTGGTCTAGGACTGGAAT
TTCAAGCATAAATGAATACTGTACAATTGTTTAATTTTAAACTATTTTGCAGCATAGCTACCTTCAGAA
TTTAGTGTATCTTTTAATGTTGTATGTCTGGGATGCAAGTATTGCTAAATATGTTAGCCCTCCAGGTTA
AAGTTGATTCAGCTTTAAGATGTTACCCTTCCAGAGGTACAGAAGAAACCTATTTCCAAAAAAGGTCC
TTTCAGTGGTAGACTCGGGGAGAACTTGGTGGCCCCTTTGAGATGCCAGGTTTCTTTTTTATCTAGAAA
TGGCTGCAAGTGGAAGCGGATAATATGTAGGCACTTTGTAAATTCATATTGAGTAAATGAATGAAATT
GTGATTTCCTGAGAATCGAACCTTGGTTCCCTAACCCTAATTGATGAGAGGCTCGCTGCTTGATGGTGT
GTACAAACTCACCTGAATGGGACTTTTTTAGACAGATCTTCATGACCTGTTCCCACCCCAGTTCATCAT
CATCTCTTTTACACCAAAAGGTCTGCAGGGTGTGGTAACTGTTTCTTTTGTGCCATTTTGGGGTGGAGA
AGGTGGATGTGATGAAGCCAATAATTCAGGACTTATTCCTTCTTGTGTTGTGTTTTTTTTTGGCCCTTGC
ACCAGAGTATGAAATAGCTTCCAGGAGCTCCAGCTATAAGCTTGGAAGTGTCTGTGTGATTGTAATCA
CATGGTGACAACACTCAGAATCTAAATTGGACTTCTGTTGTATTCTCACCACTCAATTTGTTTTTTAGC
AGTTTAATGGGTACATTTTAGAGTCTTCCATTTTGTTGGAATTAGATCCTCCCCTTCAAATGCTGTAATT
AACAACACTTAAAAAACTTGAATAAAATATTGAAACCTCATCCTTCTTCTGTTGTCTTTATTAATAAAA
TATAAATAAAC (SEQ ID NO: 22)
SEQ ID NO: 23-Ybx1 (NP_004550.2)
MSSEAETQQPPAAPPAAPALSAADTKPGTTGSGAGSGGPGGLTSAAPAGGDKKVIATKVLGTVKWFNVRN
GYGFINRNDTKEDVFVHQTAIKKNNPRKYLRSVGDGETVEFDVVEGEKGAEAANVTGPGGVPVQGSKYA
ADRNHYRRYPRRRGPPRNYQQNYQNSESGEKNEGSESAPEGQAQQRRPYRRRRFPPYYMRRPYGRRPQYS
NPPVQGEVMEGADNQGAGEQGRPVRQNMYRGYRPRFRRGPPRQRQPREDGNEEDKENQGDETQGQQPP
QRRYRRNFNYRRRRPENPKPQDGKETKAADPPAENSSAPEAEQGGAE (SEQ ID NO: 23)
SEQ ID NO: 24-Ybx1 (NM_004559)
GGGCTTATCCCGCCTGTCCCGCCATTCTCGCTAGTTCGATCGGTAGCGGGAGCGGAGAGCGGACCCCA
GAGAGCCCTGAGCAGCCCCACCGCCGCCGCCGGCCTAGTTACCATCACACCCCGGGAGGAGCCGCAG
CTGCCGCAGCCGGCCCCAGTCACCATCACCGCAACCATGAGCAGCGAGGCCGAGACCCAGCAGCCGC
CCGCCGCCCCCCCCGCCGCCCCCGCCCTCAGCGCCGCCGACACCAAGCCCGGCACTACGGGCAGCGGC
GCAGGGAGCGGTGGCCCGGGCGGCCTCACATCGGCGGCGCCTGCCGGCGGGGACAAGAAGGTCATCG
CAACGAAGGTTTTGGGAACAGTAAAATGGTTCAATGTAAGGAACGGATATGGTTTCATCAACAGGAAT
GACACCAAGGAAGATGTATTTGTACACCAGACTGCCATAAAGAAGAATAACCCCAGGAAGTACCTTC
GCAGTGTAGGAGATGGAGAGACTGTGGAGTTTGATGTTGTTGAAGGAGAAAAGGGTGCGGAGGCAGC
AAATGTTACAGGTCCTGGTGGTGTTCCAGTTCAAGGCAGTAAATATGCAGCAGACCGTAACCATTATA
GACGCTATCCACGTCGTAGGGGTCCTCCACGCAATTACCAGCAAAATTACCAGAATAGTGAGAGTGGG
GAAAAGAACGAGGGATCGGAGAGTGCTCCCGAAGGCCAGGCCCAACAACGCCGGCCCTACCGCAGGC
GAAGGTTCCCACCTTACTACATGCGGAGACCCTATGGGCGTCGACCACAGTATTCCAACCCTCCTGTG
CAGGGAGAAGTGATGGAGGGTGCTGACAACCAGGGTGCAGGAGAACAAGGTAGACCAGTGAGGCAG
AATATGTATCGGGGATATAGACCACGATTCCGCAGGGGCCCTCCTCGCCAAAGACAGCCTAGAGAGG
ACGGCAATGAAGAAGATAAAGAAAATCAAGGAGATGAGACCCAAGGTCAGCAGCCACCTCAACGTCG
GTACCGCCGCAACTTCAATTACCGACGCAGACGCCCAGAAAACCCTAAACCACAAGATGGCAAAGAG
ACAAAAGCAGCCGATCCACCAGCTGAGAATTCGTCCGCTCCCGAGGCTGAGCAGGGCGGGGCTGAGT
AAATGCCGGCTTACCATCTCTACCATCATCCGGTTTAGTCATCCAACAAGAAGAAATATGAAATTCCA
GCAATAAGAAATGAACAAAAGATTGGAGCTGAAGACCTAAAGTGCTTGCTTTTTGCCCGTTGACCAGA
TAAATAGAACTATCTGCATTATCTATGCAGCATGGGGTTTTTATTATTTTTACCTAAAGACGTCTCTTTT
TGGTAATAACAAACGTGTTTTTTAAAAAAGCCTGGTTTTTCTCAATACGCCTTTAAAGGTTTTTAAATT
GTTTCATATCTGGTCAAGTTGAGATTTTTAAGAACTTCATTTTTAATTTGTAATAAAAGTTTACAACTTG
ATTTTTTCAAAAAAGTCAACAAACTGCAAGCACCTGTTAATAAAGGTCTTAAATAATAAAAAAAAAAA
AAAA (SEQ ID NO: 24)
SEQ ID NO: 25-HSPA9 (NP_004125.3)
misasraaaa rlvgaaasrg ptaarhqdsw nglsheafrl vsrrdyasea ikgavvgidl
gttnscvavm egkqakvlen aegarttpsv vaftadgerl vgmpakrqav tnpnntfyat
krligrrydd pevqkdiknv pfkivrasng dawveahgkl yspsqigafv lmkmketaen
ylghtaknav itvpayfnds qrqatkdagq isglnvlrvi neptaaalay gldksedkvi
avydlgggtf disileiqkg vfevkstngd tflggedfdq allrhivkef kretgvdltk
dnmalqrvre aaekakcels ssvqtdinlp yltmdssgpk hlnmkltraq fegivtdlir
rtiapcqkam qdaevsksdi gevilvggmt rmpkvqqtvq dlfgrapska vnpdeavaig
aaiqggvlag dvtdvllldv tplslgietl ggvftklinr nttiptkksq vfstaadgqt
qveikvcqge remagdnkll gqftligipp aprgvpqiev tfdidangiv hvsakdkgtg
reqqiviqss gglskddien mvknaekyae edrrkkerve avnmaegiih dtetkmeefk
dqlpadecnk lkeeiskmre llarkdsetg enirqaassl qqaslklfem aykkmasere
gsgssgtgeq kedqkeekq
SEQ ID NO: 26--HSPA9 (NM_004134.6)
ttcctcccct ggactctttc tgagctcaga gccgccgcag ccgggacagg agggcaggct
ttctccaacc atcatgctgc ggagcatatt acctgtacgc cctggctccg ggagcggcag
tcgagtatcc tctggtcagg cggcgcgggc ggcgcctcag cggaagagcg ggcctctggg
ccgcagtgac caacccccgc ccctcacccc acgtggttgg aggtttccag aagcgctgcc
gccaccgcat cgcgcagctc tttgccgtcg gagcgcttgt ttgctgcctc gtactcctcc
atttatccgc catgataagt gccagccgag ctgcagcagc ccgtctcgtg ggcgccgcag
cctcccgggg ccctacggcc gcccgccacc aggatagctg gaatggcctt agtcatgagg
cttttagact tgtttcaagg cgggattatg catcagaagc aatcaaggga gcagttgttg
gtattgattt gggtactacc aactcctgcg tggcagttat ggaaggtaaa caagcaaagg
tgctggagaa tgccgaaggt gccagaacca ccccttcagt tgtggccttt acagcagatg
gtgagcgact tgttggaatg ccggccaagc gacaggctgt caccaaccca aacaatacat
tttatgctac caagcgtctc attggccggc gatatgatga tcctgaagta cagaaagaca
ttaaaaatgt tccctttaaa attgtccgtg cctccaatgg tgatgcctgg gttgaggctc
atgggaaatt gtattctccg agtcagattg gagcatttgt gttgatgaag atgaaagaga
ctgcagaaaa ttacttgggg cacacagcaa aaaatgctgt gatcacagtc ccagcttatt
tcaatgactc gcagagacag gccactaaag atgctggcca gatatctgga ctgaatgtgc
ttcgggtgat taatgagccc acagctgctg ctcttgccta tggtctagac aaatcagaag
acaaagtcat tgctgtatat gatttaggtg gtggaacttt tgatatttct atcctggaaa
ttcagaaagg agtatttgag gtgaaatcca caaatgggga taccttctta ggtggggaag
actttgacca ggccttgcta cggcacattg tgaaggagtt caagagagag acaggggttg
atttgactaa agacaacatg gcacttcaga gggtacggga agctgctgaa aaggctaaat
gtgaactctc ctcatctgtg cagactgaca tcaatttgcc ctatcttaca atggattctt
ctggacccaa gcatttgaat atgaagttga cccgtgctca atttgaaggg attgtcactg
atctaatcag aaggactatc gctccatgcc aaaaagctat gcaagatgca gaagtcagca
agagtgacat aggagaagtg attcttgtgg gtggcatgac taggatgccc aaggttcagc
agactgtaca ggatcttttt ggcagagccc caagtaaagc tgtcaatcct gatgaggctg
tggccattgg agctgccatt cagggaggtg tgttggccgg cgatgtcacg gatgtgctgc
tccttgatgt cactcccctg tctctgggta ttgaaactct aggaggtgtc tttaccaaac
ttattaatag gaataccact attccaacca agaagagcca ggtattctct actgccgctg
atggtcaaac gcaagtggaa attaaagtgt gtcagggtga aagagagatg gctggagaca
acaaactcct tggacagttt actttgattg gaattccacc agcccctcgt ggagttcctc
agattgaagt tacatttgac attgatgcca atgggatagt acatgtttct gctaaagata
aaggcacagg acgtgagcag cagattgtaa tccagtcttc tggtggatta agcaaagatg
atattgaaaa tatggttaaa aatgcagaga aatatgctga agaagaccgg cgaaagaagg
aacgagttga agcagttaat atggctgaag gaatcattca cgacacagaa accaagatgg
aagaattcaa ggaccaatta cctgctgatg agtgcaacaa gctgaaagaa gagatttcca
aaatgaggga gctcctggct agaaaagaca gcgaaacagg agaaaatatt agacaggcag
catcctctct tcagcaggca tcactgaagc tgttcgaaat ggcatacaaa aagatggcat
ctgagcgaga aggctctgga agttctggca ctggggaaca aaaggaagat caaaaggagg
aaaaacagta ataatagcag aaattttgaa gccagaagga caacatatga agcttaggag
tgaagagact tcctgagcag aaatgggcga acttcagtct ttttactgtg tttttgcagt
attctatata taatttcctt aatttgtaaa tttagtgacc attagctagt gatcatttaa
tggacagtga ttctaacagt ataaagttca caatattcta tgtccctagc ctgtcatttt
tcagctgcat gtaaaaggag gtaggatgaa ttgatcatta taaagattta actattttat
gctgaagtga ccatattttc aaggggtgaa accatctcgc acacagcaat gaaggtagtc
atccatagac ttgaaatgag accacatatg gggatgagat ccttctagtt agcctagtac
tgctgtactg gcctgtatgt acatggggtc cttcaactga ggccttgcaa gtcaagctgg
ctgtgccatg tttgtagatg gggcagagga atctagaaca atgggaaact tagctattta
tattaggtac agctattaaa acaaggtagg aatgaggcta gacctttaac ttccctaagg
catacttttc tagctacctt ctgccctgtg tctggcacct acatccttga tgattgttct
cttacccatt ctggaatttt ttttttttta aataaataca gaaagcatct tgatctcttg
tttgtgaggg gtgatgccct gagatttagc ttcaagaata tgccatggct catgcttccc
atatttccca aagagggaaa tacaggattt gctaacactg gttaaaaatg caaattcaag
atttggaagg gctgttataa tgaaataatg agcagtatca gcatgtgcaa atcttgtttg
aaggatttta ttttctcccc ttagaccttt ggtacattta gaatcttgaa agtttctaga
tctctaacat gaaagtttct agatctctaa catgaaagtt tttagatctc taacatgaaa
accaaggtgg ctattttcag gttgctttca gctccaagta gaaataacca gaattggctt
acattaaaga aactgcatct agaaataagt cctaagatac tatttctatg gctcaaaaat
aaaaggaacc cagatttctt tcccta

1. A method of evaluating a cancer sample from a patient, comprising:
identifying, the level, e.g., the amount of, or the level of expression for, 1, 2, 3, 4, 5, 6, 7, or 8 tumor markers of FUS, SMAD4, DERL1, YBX1, pS6, PDSS2, CUL2, and HSPA9 (the tumor marker set), or of a DNA or mRNA for said tumor marker,
thereby evaluating said tumor sample.

2-4. (canceled)

5. A reaction mixture comprising:
a cancer sample; and
a detection reagent for each of 4, 5, 6, 7, or 8 tumor markers of FUS, SMAD4, DERL1, YBX1, pS6, PDSS2, CUL2, and HSPA9 (the tumor marker set), or of a DNA or mRNA for said tumor marker.

6. The reaction mixture of claim 5, wherein said cancer sample comprises a plurality of portions.

7. The reaction mixture of claim 5, wherein a first portion of said cancer sample comprises a detection reagent for a first, but not all of said markers, and a second portion of said cancer sample comprises a detection reagent for a detection marker for one of said markers but does not comprise a detection reagent for said first marker.

8. A method of evaluating a sample from a patient, comprising:
(a) identifying, in a region of interest (ROI), from said sample, a level of a first region phenotype marker, e.g., a first tumor marker,
thereby evaluating said sample.

9-34. (canceled)

35. An automated method of evaluating a tumor sample from a patient, comprising:
(a) identifying, in a ROI, e.g., a cancerous ROI, a level of, e.g., the amount of, a first region-phenotype marker, e.g., a first tumor marker, e.g., wherein said first tumor marker is selected from FUS, SMAD4, DERL1, YBX1, pS6, PDSS2, CUL2, and HSPA9 (the tumor marker set), or of a DNA or mRNA for said first tumor marker,
thereby evaluating said tumor sample.

36-128. (canceled)

129. The method of claim 35, comprising:
(i.a) acquiring, directly or indirectly, a signal for a total epithelium specific marker, e.g., CK8;
(i.b) acquiring, directly or indirectly, a signal for a second total epithelium specific marker, e.g., CK18;
(ii.a) acquiring, directly or indirectly, a signal for a basal epithelium specific marker, e.g., CK5;
(ii.b) acquiring, directly or indirectly, a signal for a second basal epithelium specific marker, e.g., TRIM29;
(iii) acquiring, directly or indirectly, a signal for a nuclear marker;
(iv) acquiring, directly or indirectly, a signal for a first tumor marker;
(v) acquiring, directly or indirectly, a signal for a second tumor marker; or
(vi) acquiring, directly or indirectly, a signal for a third tumor marker.

130-158. (canceled)

159. The method of claim 35, comprising acquiring an image of the area of the sample to be analyzed, e.g., as a DAPI filter image.

160-219. (canceled)

220. A kit comprising a detection reagent for 1, 2, 3, 4, 5, 6, 7, or all of the tumor markers FUS, SMAD4, DERL1, YBX1, pS6, PDSS2, CUL2, or HSPA9.

221. The kit of claim 220, further comprising a detection reagent for a total epithelial marker and a basal epithelial marker.

222. A cancer sample having disposed thereon:
a detection reagent for a total epithelial marker;
a detection reagent for a basal epithelial marker;
a detection reagent for a tumor marker selected from FUS, SMAD4, DERL1, YBX1, pS6, PDSS2, CUL2, or HSPA9 (the tumor marker set).

223. The prostate tumor sample of claim 222, wherein said sample comprises a plurality of portions.

224. The cancer sample of claim 222, having further disposed thereon, a detection reagent for a second tumor marker selected from a FUS, SMAD4, DERL1, YBX1, pS6, PDSS2, CUL2, or HSPA9.

225. A computer-implemented method of evaluating a prostate tumor sample, from a patient, comprising:
(i) identifying a ROI of said tumor sample that corresponds to tumor epithelium (a cancerous ROI);
(ii) identifying, the level of each of the following tumor markers, FUS, SMAD4, DERL1, YBX1, pS6, PDSS2, CUL2, and HSPA9 (the tumor marker set), in a cancerous ROI, wherein identifying a level of tumor marker comprises acquiring a signal related to, e.g., proportional to, the binding of an antibody for said tumor marker;
(iii) providing a value for the level of each of the tumor markers in a cancerous ROI; and
(iv) responsive to said values, evaluating said tumor sample, comprising, e.g., assigning a risk score to said patient by algorithmically combining said levels, thereby evaluating a prostate tumor sample.

226-229. (canceled)

230. The cancer sample of claim 222, which comprises a prostate tumor sample.

231. The cancer sample of claim 222, wherein the detection reagent comprises an antibody.

232. The cancer sample of claim 222, which further comprises a noncancerous region.

233. A reaction mixture comprising:
a cancer sample; and
a detection reagent for each of 1, 2, 3, 4, or 5 tumor markers of FUS, YBX1, pS6, PDSS2, and HSPA9, or of a DNA or mRNA for said tumor marker.

234. A method of identifying a compound capable of reducing the risk of cancer progression, or delaying or slowing the cancer progression, comprising: (a) providing a cell expressing a prognosis determinant selected from the group consisting of ACTN1, CUL2, DCC, DERL1, FUS, PDSS2, PLAG1, RpS6, SMAD2, SMAD4, VDAC1, and YBX1;
(b) contacting the cell with a candidate compound; and
(c) determining whether the candidate compound alters the expression or activity of the selected prognosis determinant; whereby the alteration observed in the presence of the compound indicates that the compound is capable of reducing the risk of cancer progression, or delaying or slowing the cancer progression.

235. A kit comprising a detection reagent for 2 or more of CK8, CK18, CK5, and TRIM29.


SEQUENCE LISTING (61 sequences)

SEQ ID NO: 1 - protein, 892 aa, Homo sapiens
MDHYDSQQTNDYMQPEEDWDRDLLLDPAWEKQQRKTFTAWCNSHLRKAGTQIENIEEDFRDGLKLMLLLEVISGERLAKPERGKMRVHKISNVNKALDFIASKGVKLVSIGAEEIVDGNVKMTLGMIWTIILRFAIQDISVEETSAKEGLLLWCQRKTAPYKNVNIQNFHISWKDGLGFCALIHRHRPELIDYGKLRKDDPLTNLNTAFDVAEKYLDIPKMLDAEDIVGTARPDEKAIMTYVSSFYHAFSGAQKAETAANRICKVLAVNQENEQLMEDYEKLASDLLEWIRRTIPWLENRVPENTMHAMQQKLEDFRDYRRLHKPPKVQEKCQLEINFNTLQTKLRLSNRPAFMPSEGRMVSDINNAWGCLEQVEKGYEEWLLNEIRRLERLDHLAEKFRQKASIHEAWTDGKEAMLRQKDYETATLSEIKALLKKHEAFESDLAAHQDRVEQIAAIAQELNELDYYDSPSVNARCQKICDQWDNLGALTQKRREALERTEKLLETIDQLYLEYAKRAAPFNNWMEGAMEDLQDTFIVHTIEEIQGLTTAHEQFKATLPDADKERLAILGIHNEVSKIVQTYHVNMAGTNPYTTITPQEINGKWDHVRQLVPRRDQALTEEHARQQHNERLRKQFGAQANVIGPWIQTKMEEIGRISIEMHGTLEDQLSHLRQYEKSIVNYKPKIDQLEGDHQLIQEALIFDNKHTNYTMEHIRVGWEQLLTTIARTINEVENQILTRDAKGISQEQMNEFRASFNHFDRDHSGTLGPEEFKACLISLGYDIGNDPQGEAEFARIMSIVDPNRLGVVTFQAFIDFMSRETADTDTADQVMASFKILAGDKNYITMDELRRELPPDQAEYCIARMAPYTGPDSVPGALDYMSFSTALYGESDL

SEQ ID NO: 2 - DNA, 3743 bp, Homo sapiens
tctgccccttccccccgcccccgcccgcctcggctcccgcagcgctagtgtgtccgcctagttcagtgtgcgtggagattaggtccaagcgcccgcccagaggcaggcagtccgcgagcccagccgccgctgtcgccgccagtagcagccttcgccagcagcgccgcggcggaaccgggcgcaggggagcgagcccggccccgccagcccagcccagcccagccctactccctccccacgccagggcagcagccgttgctcagagagaaggtggaggaagaaatccagaccctagcacgcgcgcaccatcatggaccattatgattctcagcaaaccaacgattacatgcagccagaagaggactgggaccgggacctgctcctggacccggcctgggagaagcagcagagaaagacattcacggcatggtgtaactcccacctccggaaggcggggacacagatcgagaacatcgaagaggacttccgggatggcctgaagctcatgctgctgctggaggtcatctcaggtgaacgcttggccaagccagagcgaggcaagatgagagtgcacaagatctccaacgtcaacaaggccctggatttcatagccagcaaaggcgtcaaactggtgtccatcggagccgaagaaatcgtggatgggaatgtgaagatgaccctgggcatgatctggaccatcatcctgcgctttgccatccaggacatctccgtggaagagacttcagccaaggaagggctgctcctgtggtgtcagagaaagacagccccttacaaaaatgtcaacatccagaacttccacataagctggaaggatggcctcggcttctgtgctttgatccaccgacaccggcccgagctgattgactacgggaagctgcggaaggatgatccactcacaaatctgaatacggcttttgacgtggcagagaagtacctggacatccccaagatgctggatgccgaagacatcgttggaactgcccgaccggatgagaaagccatcatgacttacgtgtctagcttctaccacgccttctctggagcccagaaggcggagacagcagccaatcgcatctgcaaggtgttggccgtcaaccaggagaacgagcagcttatggaagactacgagaagctggccagtgatctgttggagtggatccgccgcacaatcccgtggctggagaaccgggtgcccgagaacaccatgcatgccatgcaacagaagctggaggacttccgggactaccggcgcctgcacaagccgcccaaggtgcaggagaagtgccagctggagatcaacttcaacacgctgcagaccaagctgcggctcagcaaccggcctgccttcatgccctctgagggcaggatggtctcggacatcaacaatgcctggggctgcctggagcaggtggagaagggctatgaggagtggttgctgaatgagatccggaggctggagcgactggaccacctggcagagaagttccggcagaaggcctccatccacgaggcctggactgacggcaaagaggccatgctgcgacagaaggactatgagaccgccaccctctcggagatcaaggccctgctcaagaagcatgaggccttcgagagtgacctggctgcccaccaggaccgtgtggagcagattgccgccatcgcacaggagctcaatgagctggactattatgactcacccagtgtcaacgcccgttgccaaaagatctgtgaccagtgggacaatctgggggccctaactcagaagcgaagggaagctctggagcggaccgagaaactgctggagaccattgaccagctgtacttggagtatgccaagcgggctgcacccttcaacaactggatggagggggccatggaggacctgcaggacaccttcattgtgcacaccattgaggagatccagggactgaccacagcccatgagcagttcaaggccaccctccctgatgccgacaaggagcgcctggccatcctgggcatccacaatgaggtgtccaagattgtccagacctaccacgtcaatatggcgggcaccaacccctacacaaccatcacgcctcaggagatcaatggcaaatgggaccacgtgcggcagctggtgcctcggagggaccaagctctgacggaggagcatgcccgacagcagcacaatgagaggctacgcaagcagtttggagcccaggccaatgtcatcgggccctggatccagaccaagatggaggagatcgggaggatctccattgagatgcatgggaccctggaggaccagctcagccacctgcggcagtatgagaagagcatcgtcaactacaagccaaagattgatcagctggagggcgaccaccagctcatccaggaggcgctcatcttcgacaacaagcacaccaactacaccatggagcacatccgtgtgggctgggagcagctgctcaccaccatcgccaggaccatcaatgaggtagagaaccagatcctgacccgggatgccaagggcatcagccaggagcagatgaatgagttccgggcctccttcaaccactttgaccgggatcactccggcacactgggtcccgaggagttcaaagcctgcctcatcagcttgggttatgatattggcaacgacccccagggagaagcagaatttgcccgcatcatgagcattgtggaccccaaccgcctgggggtagtgacattccaggccttcattgacttcatgtcccgcgagacagccgacacagatacagcagaccaagtcatggcttccttcaagatcctggctggggacaagaactacattaccatggacgagctgcgccgcgagctgccacccgaccaggctgagtactgcatcgcgcggatggccccctacaccggccccgactccgtgccaggtgctctggactacatgtccttctccacggcgctgtacggcgagagtgacctctaatccaccccgcccggccgccctcgtcttgtgcgccgtgccctgccttgcacctccgccgtcgcccatctcctgcctgggttcggtttcagctcccagcctccacccgggtgagctggggcccacgtggcatcgatcctccctgcccgcgaagtgacagtttacaaaattattttctgcaaaaaagaaaaaaaagttacgttaaaaaccaaaaaactacatattttattatagaaaaagtattttttctccaccagacaaatggaaaaaaagaggaaagattaactatttgcaccgaaatgtcttgttttgttgcgacataggaaaataaccaagcacaaagttatattccatcctttttactgatttttttttcttctatctgttccatctgctgtattcatttctccaatctcatgtccattttggtgtgggagtcggggtagggggtactcttgtcaaaaggcacattggtgcatgtgtgtttgctagctcacttgtccatgaaaatattttatgatattaaagaaaatcttttgaaatggctgttttttaaggaagagaatttatgtggcttctcatttttaaatcccctcagaggtgtgactagtctctttatcagcacacacttaaaaaatttttaatattgtctattaaaaataggacaaacttggagagtatggacaactttgatattgcttggcacagatggtattaaaaaaaccacactcctatgacaaaaaaaaaaaaaaaaaaa

SEQ ID NO: 3 - protein, 764 aa, Homo sapiens
MYRVTWSTFWLRFQHYTCTMSLKPRVVDFDETWNKLLTTIKAVVMLEYVERATWNDRFSDIYALCVAYPEPLGERLYTETKIFLENHVRHLHKRVLESEEQVLVMYHRYWEEYSKGADYMDCLYRYLNTQFIKKNKLTEADLQYGYGGVDMNEPLMEIGELALDMWRKLMVEPLQAILIRMLLREIKNDRGGEDPNQKVIHGVINSFVHVEQYKKKFPLKFYQEIFESPFLTETGEYYKQEASNLLQESNCSQYMEKVLGRLKDEEIRCRKYLHPSSYTKVIHECQQRMVADHLQFLHAECHNIIRQEKKNDMANMYVLLRAVSTGLPHMIQELQNHIHDEGLRATSNLTQENMPTLFVESVLEVHGKFVQLINTVLNGDQHFMSALDKALTSVVNYREPKSVCKAPELLAKYCDNLLKKSAKGMTENEVEDRLTSFITVFKYIDDKDVFQKFYARMLAKRLIHGLSMSMDSEEAMINKLKQACGYEFTSKLHRMYTDMSVSADLNNKFNNFIKNQDTVIDLGISFQIYVLQAGAWPLTQAPSSTFAIPQELEKSVQMFELFYSQHFSGRKLTWLHYLCTGEVKMNYLGKPYVAMVTTYQMAVLLAFNNSETVSYKELQDSTQMNEKELTKTIKSLLDVKMINHDSEKEDIDAESSFSLNMNFSSKRTKFKITTSMQKDTPQEMEQTRSAVDEDRKMYLQAAIVRIMKARKVLRHNALIQEVISQSRARFNPSISMIKKCIEVLIDKQYIERSQASADEYSYVA

SEQ ID NO: 4 - DNA, 4172 bp, Homo sapiens
gtcacagtagggagtaccaggaggagaggaagcttgggtgccatgttgcagttgagcccaaactgaatgctgtctgtagaaggaaacaacaaactttgtactttatgtacagagtaacatggtcaactttttggcttagatttcaacactacacttgcacaatgtctttgaaaccaagagtagtagattttgatgaaacatggaacaaacttttgacgacaataaaagccgtggtcatgttggaatacgtcgaaagagcaacatggaatgaccgtttctcagatatctatgctttatgtgtggcctatcctgaaccccttggagaaagactttatacagaaactaagatttttttggaaaatcatgttcggcatttgcataagagagttttggagtcagaagaacaagtacttgttatgtatcataggtactgggaagaatacagcaagggtgcagactatatggactgcttatataggtatctcaacacccagtttattaaaaagaataaattaacagaagcggaccttcagtatggctatggtggtgtagatatgaatgaaccacttatggaaataggagagctagcattggatatgtggaggaaattgatggttgaaccacttcaggccatccttatccgaatgctgctccgagaaatcaaaaatgatcgtggtggagaagacccaaaccagaaagtaatccatggggttattaactcctttgttcatgttgaacagtataagaaaaaattccccttaaagttttatcaggaaatttttgagtctccctttctgactgaaacaggagagtattacaaacaagaagcttcaaatttattacaagaatcaaactgctcacagtatatggaaaaggttctaggtagattaaaagatgaagaaattcgatgtcgaaaatacctacatccaagttcatatactaaggtgattcatgaatgtcaacaacgaatggtagcagaccacttacagtttttacatgcagaatgtcataatataattcgacaagagaaaaaaaatgacatggcaaatatgtacgtcttactccgtgctgtgtccactggtttacctcatatgattcaggagctgcaaaaccacatccatgatgagggccttcgagcaaccagcaaccttactcaggaaaacatgccaacactatttgtggagtcagttttggaagtgcatggtaaatttgttcagcttatcaacactgttttgaatggtgatcagcattttatgagtgcgttggataaggcccttacgtcagttgtaaattacagagaacctaagtctgtttgcaaagcacctgaactgcttgctaagtactgtgacaacttactgaagaagtcagcgaaagggatgacagagaatgaagtggaagacaggctcacgagcttcatcacagtgttcaaatacattgatgacaaggacgtctttcaaaagttctacgcaagaatgctggcaaaacgtttaattcatgggttatccatgtctatggactctgaagaagccatgatcaacaaattaaagcaagcctgtggttatgagtttaccagcaagctacatcggatgtatacagatatgagtgtcagcgctgatctcaacaataagttcaacaattttatcaaaaaccaagacacagtaatagatttgggaattagttttcaaatatatgttctacaggctggtgcgtggcctcttactcaggctccttcatctacgtttgcaattccccaggaattagaaaaaagtgtacagatgtttgaattattttatagccaacatttcagtggaaggaaacttacatggttacattatctgtgtacaggtgaagttaaaatgaactatttgggcaaaccatatgtagccatggttacaacataccaaatggcagttcttcttgcctttaacaacagtgaaactgtcagttataaagagcttcaggacagcactcagatgaatgaaaaggaactgacaaaaacaatcaaatcattacttgatgtgaaaatgattaaccatgattcagaaaaggaagatattgatgcagaatcttcgttttcattaaatatgaactttagcagtaaaagaacaaaatttaaaattactacatcaatgcagaaagacacaccacaagaaatggagcagactagaagtgcagttgatgaggaccggaaaatgtatctccaagctgctatagttcgtatcatgaaagcacgaaaagtgcttcggcacaatgcccttattcaagaggtgattagccagtcaagagctaggtttaatcccagtatcagcatgattaagaagtgtattgaagttctgatagacaaacaatacatagaacgcagccaggcgtcggcagatgaatacagctacgtcgcgtgatgtcgctctcctccagcgtggtgtgagaagatcattgccatcaccatttggtgtgttcctgtgggaaaaagcaggactgtgcctccataatttggtcatttggcagcccctgttttctgctgtttacaacatcaccagtgccacgtcatgagcgtcaaagaaaatgcctagagatatttcaagctcatgtcattatgacatttcttaaaactttattaaaagaatgagtgaagtattgctgaaaagtggaaattcggttgggtaccatgctttttctccccttcacgtttgcagttgatgtgtctttttttttttttttaatgtatcttaaaggacataaaatttaaaaacttaaatattgtaatatgacagataacctaataattgtatctacattaaaatgacaaacatgatactgctgcttgtcaaataaaaaaaaaataaagaaatagaatgccttttttatgtggatggagtatcaggttgaccacaaaatatattgactcaaagcagctaatgcatctttagttgcgtttttatctgaatggtttaattcacttgtactcctatttaaatcctacatgaaaaatgtctagattattgttcttgactgcataggactgcattcagcataaagaatgctttatttttatggattagatatattggatctaaacattttgaatcttgaagatgtaattccatcagcagtttctggtggtgtgctactccacagacatcgcagagtgtgagcaggatgcttggtgacctcaagtctggcacagagagagcttttcattcaaaagttgtctttcttcggttgcataatccattaattctagcatagactagtaccctagctctgtggccttccctgagtcttaggaaatctatgataccaacatattccttctatatgcctcccctacctgttacccttacaaccctcctccaacagtttagatactagagtcactctcatcaatcacagatgtgcttagcaatgcataacctaaatacttttttaaaaaagaaaattgtacattgtactgggtgccacatatataaatcccattattttgtttattttatatatatatatatatataatatatatatatatatatctcaacagcagtgttaagagtactgcgatctattatcatatttattgtctatccacaccatcaccaccaccaccacacccctcctccctcaacatacaatttttctttattttaaaaaaaataagagacggggtttcgccatgtttcccaggctagtctggaacttctggcctcaagcaatcctatctctgtctcccaaagtgctgggattacaagcatgagccactgcatccatccaacacaaaatttttaaaatcggaatattttaaagcaaatcacacaaattatttcacttataatacttcagtaaggcctttaaaaaatccacagtgatattattactcctaacaaaaacaataattacttagtatcatctaatatgtggttcatatttaaatttgttgttttgagatgggtcttacaattggtttattcaattgcattttttctaactcgtgtctcaagtgttttaaaaatctactgaacttataatgacttatataatgtatttctcattttacctttcttccaaaagaggaaataatggcaaaccatataatattgtacattcactgtcaaaaagcaaacccttgttttgataacttgttgattgataaaagttttccaaattgaaaaaaaaaaaaaa

SEQ ID NO: 5 - protein, 1447 aa, Homo sapiens
MENSLRCVWVPKLAFVLFGASLFSAHLQVTGFQIKAFTALRFLSEPSDAVTMRGGNVLLDCSAESDRGVPVIKWKKDGIHLALGMDERKQQLSNGSLLIQNILHSRHHKPDEGLYQCEASLGDSGSIISRTAKVAVAGPLRFLSQTESVTAFMGDTVLLKCEVIGEPMPTIHWQKNQQDLTPIPGDSRVVVLPSGALQISRLQPGDIGIYRCSARNPASSRTGNEAEVRILSDPGLHRQLYFLQRPSNVVAIEGKDAVLECCVSGYPPPSFTWLRGEEVIQLRSKKYSLLGGSNLLISNVTDDDSGMYTCVVTYKNENISASAELTVLVPPWFLNHPSNLYAYESMDIEFECTVSGKPVPTVNWMKNGDVVIPSDYFQIVGGSNLRILGVVKSDEGFYQCVAENEAGNAQTSAQLIVPKPAIPSSSVLPSAPRDVVPVLVSSRFVRLSWRPPAEAKGNIQTFTVFFSREGDNRERALNTTQPGSLQLTVGNLKPEAMYTFRVVAYNEWGPGESSQPIKVATQPELQVPGPVENLQAVSTSPTSILITWEPPAYANGPVQGYRLFCTEVSTGKEQNIEVDGLSYKLEGLKKFTEYSLRFLAYNRYGPGVSTDDITVVTLSDVPSAPPQNVSLEVVNSRSIKVSWLPPPSGTQNGFITGYKIRHRKTTRRGEMETLEPNNLWYLFTGLEKGSQYSFQVSAMTVNGTGPPSNWYTAETPENDLDESQVPDQPSSLHVRPQTNCIIMSWTPPLNPNIVVRGYIIGYGVGSPYAETVRVDSKQRYYSIERLESSSHYVISLKAFNNAGEGVPLYESATTRSITDPTDPVDYYPLLDDFPTSVPDLSTPMLPPVGVQAVALTHDAVRVSWADNSVPKNQKTSEVRLYTVRWRTSFSASAKYKSEDTTSLSYTATGLKPNTMYEFSVMVTKNRRSSTWSMTAHATTYEAAPTSAPKDLTVITREGKPRAVIVSWQPPLEANGKITAYILFYTLDKNIPIDDWIMETISGDRLTHQIMDLNLDTMYYFRIQARNSKGVGPLSDPILFRTLKVEHPDKMANDQGRHGDGGYWPVDTNLIDRSTLNEPPIGQMHPPHGSVTPQKNSNLLVIIVVTVGVITVLVVVIVAVICTRRSSAQQRKKRATHSAGKRKGSQKDLRPPDLWIHHEEMEMKNIEKPSGTDPAGRDSPIQSCQDLTPVSHSQSETQLGSKSTSHSGQDTEEAGSSMSTLERSLAARRAPRAKLMIPMDAQSNNPAVVSAIPVPTLESAQYPGILPSPTCGYPHPQFTLRPVPFPTLSVDRGFGAGRSQSVSEGPTTQQPPMLPPSQPEHSSSEEAPSRTIPTACVRPTHPLRSFANPLLPPPMSAIEPKVPYTPLLSQPGPTLPKTHVKTASLGLAGKARSPLLPVSVPTAPEVSEESHKPTEDSANVYEQDDLSEQMASLEGLMKQLNAITGSAF

SEQ ID NO: 6 - DNA, 10210 bp, Homo sapiens
gtagtacggttccaactcccagctcgcacaccgctggcggacaccccagtaacaagtgagagcgctccaccccgcagtcccccccgcctctcctccctgggtcccctcggctctcggaagaaaaaccaacagcatctccagctctcgcgcggaattgtctcttcaactttacccaaccgacgacaaggaaccagcctcaaccttttaatgcacagcccggccacaggattgccttccatctcctcttggtccctcctggatgtggtttattgatgacttgcgagcccctcagagagctgtcttccctcctctggctccctccgtttccttgagttagttttctaaggttttaccggggctcgggatctcttggaccgaatggaactttttgctgcctgcttttgctgctgattctgtcagtggacaaggaaaaaggcttcgaaggcagcagaggcgcaggggaggtggagaaagaggtggaggaagaggacgaggaggaggaggaagccgaaggggctcggcgcgtgtgtgtgcatgtgtgcatgcgtgtgtgagtgcatgtgtgtgagtgctgccgctgcccgcgacccctggccccgaaggtgttggctgaaatatggagaatagtcttagatgtgtttgggtacccaagctggcttttgtactcttcggagcttccttgttcagcgcgcatcttcaagtaaccggttttcaaattaaagctttcacagcactgcgcttcctctcagaaccttctgatgccgtcacaatgcggggaggaaatgtcctcctcgactgctccgcggagtccgaccgaggagttccagtgatcaagtggaagaaagatggcattcatctggccttgggaatggatgaaaggaagcagcaactttcaaatgggtctctgctgatacaaaacatacttcattccagacaccacaagccagatgagggactttaccaatgtgaggcatctttaggagattctggctcaattattagtcggacagcaaaagttgcagtagcaggaccactgaggttcctttcacagacagaatctgtcacagccttcatgggagacacagtgctactcaagtgtgaagtcattggggagcccatgccaacaatccactggcagaagaaccaacaagacctgactccaatcccaggtgactcccgagtggtggtcttgccctctggagcattgcagatcagccgactccaaccgggggacattggaatttaccgatgctcagctcgaaatccagccagctcaagaacaggaaatgaagcagaagtcagaattttatcagatccaggactgcatagacagctgtattttctgcaaagaccatccaatgtagtagccattgaaggaaaagatgctgtcctggaatgttgtgtttctggctatcctccaccaagttttacctggttacgaggcgaggaagtcatccaactcaggtctaaaaagtattctttattgggtggaagcaacttgcttatctccaatgtgacagatgatgacagtggaatgtatacctgtgttgtcacatataaaaatgagaatattagtgcctctgcagagctcacagtcttggttccgccatggtttttaaatcatccttccaacctgtatgcctatgaaagcatggatattgagtttgaatgtacagtctctggaaagcctgtgcccactgtgaattggatgaagaatggagatgtggtcattcctagtgattattttcagatagtgggaggaagcaacttacggatacttggggtggtgaagtcagatgaaggcttttatcaatgtgtggctgaaaatgaggctggaaatgcccagaccagtgcacagctcattgtccctaagcctgctatcccaagctccagtgtcctcccttcggctcccagagatgtggtccctgtcttggtttccagccgatttgtccgtctcagctggcgcccacctgcagaagcgaaagggaacattcaaactttcacggtctttttctccagagaaggtgacaacagggaacgagcattgaatacaacacagcctgggtcccttcagctcactgtgggaaacctgaagccagaagccatgtacacctttcgagttgtggcttacaatgaatggggaccgggagagagttctcaacccatcaaggtggccacacagcctgagttgcaagttccagggccagtagaaaacctgcaagctgtatctacctcacctacctcaattcttattacctgggaaccccctgcctatgcaaacggtccagtccaaggttacagattgttctgcactgaggtgtccacaggaaaagaacagaatatagaggttgatggactatcttataaactggaaggcctgaaaaaattcaccgaatatagtcttcgattcttagcttataatcgctatggtccgggcgtctctactgatgatataacagtggttacactttctgacgtgccaagtgccccgcctcagaacgtctccctggaagtggtcaattcaagaagtatcaaagttagctggctgcctcctccatcaggaacacaaaatggatttattaccggctataaaattcgacacagaaagacgacccgcaggggtgagatggaaacactggagccaaacaacctctggtacctattcacaggactggagaaaggaagtcagtacagtttccaggtgtcagccatgacagtcaatggtactggaccaccttccaactggtatactgcagagactccagagaatgatctagatgaatctcaagttcctgatcaaccaagctctcttcatgtgaggccccagactaactgcatcatcatgagttggactcctcccttgaacccaaacatcgtggtgcgaggttatattatcggttatggcgttgggagcccttacgctgagacagtgcgtgtggacagcaagcagcgatattattccattgagaggttagagtcaagttcccattatgtaatctccctaaaagcttttaacaatgccggagaaggagttcctctttatgaaagtgccaccaccaggtctataaccgatcccactgacccagttgattattatcctttgcttgatgatttccccacctcggtcccagatctctccacccccatgctcccaccagtaggtgtacaggctgtggctcttacccatgatgctgtgagggtcagctgggcagacaactctgtccctaagaaccaaaagacgtctgaggtgcgactttacaccgtccggtggagaaccagcttttctgcaagtgcaaaatacaagtcagaagacacaacatctctaagttacacagcaacaggcctcaaaccaaacacaatgtatgaattctcggtcatggtaacaaaaaacagaaggtccagtacttggagcatgactgcacatgccaccacgtatgaagcagcccccacctctgctcccaaggacttgacagtcattactagggaagggaagcctcgtgccgtcattgtgagttggcagcctcccttggaagccaatgggaaaattactgcttacatcttattttataccttggacaagaacatcccaattgatgactggattatggaaacaatcagtggtgataggcttactcatcaaatcatggatctcaaccttgatactatgtattactttcgaattcaagcacgaaattcaaaaggagtggggccactctctgatcctatcctcttcaggactctgaaagtggaacaccctgacaaaatggctaatgaccaaggtcgtcatggagatggaggttattggccagttgatactaatttgattgatagaagcaccctaaatgagccgccaattggacaaatgcaccccccgcatggcagtgtcactcctcagaagaacagcaacctgcttgtgatcattgtggtcaccgttggtgtcatcacagtgctggtagtggtcatcgtggctgtgatttgcacccgacgctcttcagcccagcagagaaagaaacgggccacccacagtgctggcaaaaggaagggcagccagaaggacctccgaccccctgatctttggatccatcatgaagaaatggagatgaaaaatattgaaaagccatctggcactgaccctgcaggaagggactctcccatccaaagttgccaagacctcacaccagtcagccacagccagtcagaaacccaactgggaagcaaaagcacctctcattcaggtcaagacactgaggaagcagggagctctatgtccactctggagaggtcgctggctgcacgccgagccccccgggccaagctcatgattcccatggatgcccagtccaacaatcctgctgtcgtgagcgccatcccggtgccaacgctagaaagtgcccagtacccaggaatcctcccgtctcccacctgtggatatccccacccgcagttcactctccggcctgtgccattcccaacactctcagtggaccgaggtttcggagcaggaagaagtcagtcagtgagtgaaggaccaactacccaacaaccacctatgctgcccccatctcagcctgagcattctagcagcgaggaggcaccaagcagaaccatccccacagcttgtgttcgaccaactcacccactccgcagctttgctaatcctttgctacctccaccaatgagtgcaatagaaccgaaagtcccttacacaccacttttgtctcagccagggcccactcttcctaagacccatgtgaaaacagcctcccttgggttggctggaaaagcaagatcccctttgcttcctgtgtctgtgccaacagcccctgaagtgtctgaggagagccacaaaccaacagaggattcagccaatgtgtatgaacaggatgatctgagtgaacaaatggcaagtttggaaggactcatgaagcagcttaatgccatcacaggctcagccttttaacatgtatttctgaatggatgaggtgaattttccgggaactttgcagcataccaattacccataaacagcacacctgtgtccaagaactctaaccagtgtacaggtcacccatcaggaccactcagttaaggaagatcctgaagcagttcagaaggaataagcattccttctttcacaggcatcaggaattgtcaaatgatgattatgagttccctaaacaaaagcaaagatgcattttcactgcaatgtcaaagtttaagctgctagaatagtcatgggcctttgtcactgcagtgaccacactgtcataactaatacctatgttttcctttgtcaaggcctgttgtttaatgtgtaggtctagtcttacaaaatgcaagtgcattatttaagcctgtaccatgccatggcaaaccagtgcaagctcactattttgttttcaacttaaacatacaaagcacccatgggaatctctcatgccatagcaccaaaggattggatgttttccttacagcacaaaaagtaaatagtaaacaaacaaaaggcagagaatgcttatgtttgtaactcagtcattcatcttgcacaagtggtggatattagtgagtggctaaaaattcacctattttggcaagtatttgtaaatccacccttggttaatatgtatgtctggagtccaggaatataaaaatctgcaactagtggcattctgccagcagcagtacatttctggaaagaggatataatatgcaatcttctcagacacatggtaattatatgcttaagcttgtaataggacagttttcaatttgggtggcttttgtgccataccacactgtgatacaatttcaaagcttcactaaggccatcttccttaggagtttggccagaagaatgcccccaccccttcaccccatccctccctgagttctccttggcaactagcgttgggtgaaatggccagctccacatgtcatatggtgcactggccaatgtcgcctgtcttctaatcccgtagaaatggcagactccctgagagcaggaagagaaggaaaataaaaggtagcttctaacagtaccttctcttaaagaatgccaactctgcctacagggtcagtgttggcaagcattggccaccagacccttttgttaagggaaacttttacactacacctgtgtcagagtcagggggaagcagaggggcaggtgccacctgacacttccgacatgtaaatccagcagatacttttcaaagcagcatcttaaactgtggactacagttttaaacttctattgccatgtttatctacagcttggaactagctaaaattaagaacattttgtatgcagcattttagtttctgaattttcagctgcatttggagttaatccctgtttatgcagctgaatcgccaaaagggagctagtttgcatatttatcagttaggtgacttgaaaacccaatgagagagtttcagctgaattattcctttcagctctgcctttgatttcaagcttgagtaggtcataattttaaaagagcatggaagggataggatctttacaacctaatagctccttttattaggtgggtaattatatatgaatccctgaataaaatattttgagcaaaatggcactgtaacagaagtaataattcagtttatttttttacagttttatgtcgggaaggaaatctgatgtcaaagagagggctgttcaaatggttcattagaaagtccggtccatttgcgaatttgttccttcaacaagagtgctcattcaagttactcagattttctggaagtcttttctgaagagctatgtgatgttgttctatgggacagactactcttatttaacatctgggcacttaggtagacaaccttctactgacctggaataaagtgtttcctaacataatattgaattattcagaaataatccattacttcaaaaaagaaaatattcattgggctagcccaaccttctctaggccctaagaattattacctcccctttctaattctagcaaacatggaacattctccttaggcacttgacacccacgagggtaatcctgagtgctcagtttggaataggttgcaaatctcagattttagggattgagtcacaccttcaatctatagaatgaagttgaccaattaaaaaaaaaaaaaaaacctatcattttcacaaatttctagatccttctagtcaaaaataattatttaggaaataaaatttttaaaaatccatttaaatacatgttatttgtcttcagtggaagttatatttctgctgcatgcttttgaaactttcttcattaaatagaatggtttgtcttagtaactggcaatgccagtattagcaccatgcatttaatctataatacaatcaatttaaacatcctcaaaaaactctagtatcatttacctggtagtattaatatacaatgatgtcaccacaacttttgtataactctgttccctttaccctcaaatgattcatatatgtatataattgcctgcccaagttttcaggtaacttattaatttcccagtctcctgatctcttgacaagaagaaacctgtgaatactgcaaactagcctctgacttcctcctactgagtctagttcatggtatccaggactctttatgctcataactctctctgattcccattgggtgatacctgacagccaaccagccgctctgccaccagaactcatttctccctgaaaaagaagaaaatcatatttggcagagcattctctggtctgccctgtaatgtgcttaaatgtcaggcaacatcctcttttttttaaaaaaaatggtatttttctttaaatttcaccctaataagaaagctattttctctcctctgcagaaatttctgcatttgtgaaacttataaaaatttagatagttcaaatgtataaagaatatttggatgatgctctagccaaaagttaaatatttcgtagtgaatcatagccaataagaaaccagtcatacttgcctctttgaataacagagatataagcttctagaatatttaaataatgaagttttacatttgggtattataaaatgcatactcaattgaatgagctgaaaaaaataccaagccagtgatataagtggagatttattaaggattcctgttgagtatattcttagtttcctcaaaatagggattattcaaaattaggtgtatgttcaatctcctgctttggttccagctacacaaggagagccatcctgtgctagtgtgatgtttcagacaacattctgaaactaaaatgtttggcacttattggcttttccaataaagaatctcttaagtacaggtatttctggaagctgttggtgtctgtgcttgaagatgattgctgatacttatccaccctttgggtacttctgttgactttgtttaaataatcatcttatggttgtccccaaatgtaatatggtatctcagatatagcagctggactgtaattacaacaaaaggttacctctaaagataacatcttaccattttagataaaattgtgtcccagaattcttatggtttcggaatgtacatttctagtcaatgaaagaaagaaaaatggaaaaattgtctagtttcaggcatgtttaaagaaaacaaagtcatctgaactttaaaatagatgcagagcagggttactttccctttcactcagttcccttcatgcaaccacaggcagtcctgcaggccagaggttactatcctaacctgctcataaccatatactatacagagcccacaactttctggagatgcagaagcagccatacactcaagtctctgtttttgtaaatcacattcaaagcaacattttactcataatttgcatttctctggtgactttcagaaatcactttagtattgtacagaaaagctttttatttgagtctagtgtttaaaattaaattggatacttgggaaaatcatagataggtgttttgtatgatattccattccaatgcaaaatatatgtacccatgcctcaatgtatcttgctatctaaatacctgttgccaaaaagtattgatttgggaaaaaaaatgccaatttcctggtcagtgaggttatgtaaaagacaaaataccacacccatatcagcaaatgaatattactactcatctggactcttcgttgccactattgcataacgttcacgtggcagacttccagttgcactctctgaaggacttttttctcttactctcaatagagagcctttgtacattgtcatcctatgattgttgttggtagaagagcaagagcaaaactctgcaagatttaataaacacaggggcatgggccaagggatctcactgtgtgctgaacatgtattttcagatgcaagaaaggaatgatggagaggaggagaaatgctgttttttattattgtagggtaaatctgacaattctgaactttgtgaattgtcagcttgtttgggggaagggtgggcgggtatggggtgtactttttataagtaatatttaatttattatttagagtggcttctttttggataatttatgataaaaagggagatctggttgggatctagatacggctgttaaagctgcagtgttccatacctcagagggaccactttggaaatgaattgtccattgctgagtatgaagagatgtccagtccaggcaaagccttcactgaagttccatcatcgccacttctccctttttagggtcattcaaagaagataacaccaaacctaaataattctgaaagcattttgcagatcagtgctactcattcaaagggctttgcaactcaaacagattgttagtgtgctagtgataagtttatttggtagaaatgggtatactacagctttaactagccttagtgagaaaagaaattttttgttgttacaaaacaccttttttaacaaaaaggtattttgagcctacaaaaagtttctttaaactgtcagattctagcattgttaaccaaattagactagtgattgcaatatttaagtgtaaatcttgttctacaagaaaggaaacttgcttacagtttaaaacaatgactgtttctacacatgatcttgtatactactacacaaggaaaagggggttttgtaaacactgtagaacagtctcatattcatttttttatagaaatgttattccaatggtgcattttttgtttaataaataaagttttgatacaaagttc

SEQ ID NO: 7 - protein, 251 aa, Homo sapiens
MSDIGDWFRSIPAITRYWFAATVAVPLVGKLGLISPAYLFLWPEAFLYRFQIWRPITATFYFPVGPGTGFLYLVNLYFLYQYSTRLETGAFDGRPADYLFMLLFNWICIVITGLAMDMQLLMIPLIMSVLYVWAQLNRDMIVSFWFGTRFKACYLPWVILGFNYIIGGSVINELIGNLVGHLYFFLMFRYPMDLGGRNFLSTPQFLYRWLPSRRGGVSGFGVPPASMRRAADQNGGGGRHNWGQGFRLGDQ

SEQ ID NO: 8 - DNA, 3344 bp, Homo sapiens
acctggctccgccccccaggacgccgagcctcggccgggcggtaaaatcggcgcttaccctttaagcggcgggacttctggtcacgtcgtccgcggtcgccggaaggggaagtttcgcctcagaaggctgcctcgctggtccgaattcggtggcgccacgtccgcccgtctccgccttctgcatcgcggcttcggcggcttccacctagacacctaacagtcgcggagccggccgcgtcgtgagggggtcggcacggggagtcgggcggtcttgtgcatcttggctacctgtgggtcgaagatgtcggacatcggagactggttcaggagcatcccggcgatcacgcgctattggttcgccgccaccgtcgccgtgcccttggtcggcaaactcggcctcatcagcccggcctacctcttcctctggcccgaagccttcctttatcgctttcagatttggaggccaatcactgccaccttttatttccctgtgggtccaggaactggatttctttatttggtcaatttatatttcttatatcagtattctacgcgacttgaaacaggagcttttgatgggaggccagcagactatttattcatgctcctctttaactggatttgcatcgtgattactggcttagcaatggatatgcagttgctgatgattcctctgatcatgtcagtactttatgtctgggcccagctgaacagagacatgattgtatcattttggtttggaacacgatttaaggcctgctatttaccctgggttatccttggattcaactatatcatcggaggctcggtaatcaatgagcttattggaaatctggttggacatctttattttttcctaatgttcagatacccaatggacttgggaggaagaaattttctatccacacctcagtttttgtaccgctggctgcccagtaggagaggaggagtatcaggatttggtgtgccccctgctagcatgaggcgagctgctgatcagaatggcggaggcgggagacacaactggggccagggctttcgacttggagaccagtgaaggggcggcctcgggcagccgctcctctcaagccacatttcctcccagtgctgggtgcacttaacaactgcgttctggctaacactgttggacctgacccacactgaatgtagtctttcagtacgagacaaagtttcttaaatcccgaagaaaaatataagtgttccacaagtttcacgattctcattcaagtccttactgctgtgaagaacaaataccaactgtgcaaattgcaaaactgactacattttttggtgtcttctcttctcccctttccgtctgaataatgggttttagcgggtcctagtctgctggcattgagctggggctgggtcaccaaacccttcccaaaaggacccttatctctttcttgcacacatgcctctctcccacttttcccaacccccacatttgcaactagaagaggttgcccataaaattgctctgcccttgacaggttctgttatttattgacttttgccaaggcttggtcacaacaatcatattcacgtaattttccccctttggtggcagaactgtagcaatagggggagaagacaagcagcggatgaagcgttttctcagcttttggaattgcttcgacctgacatccgttgtaaccgtttgccacttcttcagatatttttataaaaaagtaccactgagtcagtgagggccacagattggtattaatgagatacgagggttgttgctgggtgtttgtttcctgagctaagtgatcaagactgtagtggagttgcagctaacatgggttaggtttaaaccatgggggatgcaacccctttgcgtttcatatgtaggcctactggctttgtgtagctggagtagttgggttgctttgtgttaggaggatccagatcatgttggctacagggagatgctctctttgagaggctcctgggcattgattccatttcaatctcattctggatatgtgttcattgagtaaaggaggagagaccctcatacgctatttaaatgtcacttttttgcctatcccccgttttttggtcatgtttcaattaattgtgaggaaggcgcagctcctctctgcacgtagatcattttttaaagctaatgtaagcacatctaagggaataacatgatttaaggttgaaatggctttagaatcatttgggtttgagggtgtgttattttgagtcatgaatgtacaagctctgtgaatcagaccagcttaaatacccacacctttttttcgtaggtgggcttttcctatcagagcttggctcataaccaaataaagttttttgaaggccatggcttttcacacagttattttattttatgacgttatctgaaagcagactgttaggagcagtattgagtggctgtcacactttgaggcaactaaaaaggcttcaaacgttttgatcagtttcttttcaggaaacattgtgctctaacagtatgactattctttcccccactcttaaacagtgtgatgtgtgttatcctaggaaatgagagttggcaaacaacttctcattttgaatagagtttgtgtgtacctctccatatttaatttatatgataaaataggtggggagagtctgaaccttaactgtcatgttttgttgttcatctgtggccacaataaagtttacttgtaaaattttagaggccattactccaattatgttgcacgtacactcattgtacaggcgtggagactcattgtatgtataagaatattctgacagtgagtgacccggagtctctggtgtaccctcttaccagtcagctgcctgcgagcagtcattttttcctaaaggtttacaagtatttagaactcttcagttcagggcaaaatgttcatgaagttattcctcttaaacatggttaggaagctgatgacgttattgattttgtctggattatgtttctggaataattttaccaaaacaagctatttgagttttgacttgacaaggcaaaacatgacagtggattctctttacaaatggaaaaaaaaaatccttattttgtataaaggacttccctttttgtaaactaatcctttttattggtaaaaattgtaaattaaaatgtgcaacttgaaggttgtctgtgttaagtttccatgtccctgctctgctgtctcttagatatcacataatttgtgtaaccaattatctcttgaagagcatttaggaagtacccagtattttttgctggattaattcctggatgcagaattcctgggttttcattttaatgaaggaggatgcttgctaactttgaaaaa

SEQ ID NO: 9 - protein, 526 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 10 - DNA, 5119 bp, Homo sapiens
acttaagcttcgacgcaggaggcggggctgctcagtcctccaggcgtcggtactcagcggtgttggaacttcgttgcttgcttgcctgtgcgcgcgtgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatgggcggaagtgaccgtggtggcttcaataaatttggtggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaattagcctggctccccaggttctggaacagctttttgtcctgtacccagtgttaccctcgttattttgtaaccttccaattcctgatcacccaagggtttttttgtgtcggactatgtaattgtaactatacctctggttcccattaaaagtgaccattttagttaaattttgttcctcttcccccttttcactttcctggaagatcgatgtcccgatcaggaaggtagagagttttcctgttcagattaccctgcccagcaggaactggaatacagtgttcggggagaaggccaaatgatatccttgagagcagagattaaacttttctgtcatggggaaagttggtgtataaatgagaaatgaagaacatgggatgtcatgagtgttggcctaaatttgcccagctatggggaatttttcctttaccacatttatttgcatactggtcttagtttatttgcagcagtttatccctttttaagaactctttgatcttttggcccttttaatggtgaggctcaaacaaactacatttaaatggggcagtattcagatttgaccatggtggagagcgcttagccactctgggtctttcacaggaaggagagtaactgagtgctgcaggagtttgtggagtggagtcaggatctaggaggtgagtgactcccttcctagctgccctggtgaacagcgcttgggtagatacctgctataaggagactggtctggctgggttactttcacatcctgcctgtactcagagggcttgaggtcattgacattatgagattttaggcttgatccctttttgattggagggtggaaggccctcctaagggaatgataagtgataagagggggaaggggttgcagccaatgagttaaaaccttagagcagtgctcctcagcctcttaccatgtggttgtaaacttgcacgtacctgccaaccagttatttagcatgctttttattttagttacacagagcgtaacattaacccaagagcagaaaggttttatttacagggttttcgaacttggtttgtaagacagctgccatcacaagcatagcttacaaatgtgctggggacccctaattgggaagtgctttcctctcaaatttttattttttatttttagagacagagtcttgctctgtcatccaggctggagtgcagtggcgtgatctcggctcactgtagcctctgcctcctgagtttaagcgattctcctgcctcaggagaatcccagcttctgagtagctgagactacaggcgtgggccaccatgcccacctagtttttgcatttttagtggaggtgtggtttcactgtgttggccaggctggtctcttaactcctgacctcaggtgatccacctgccttggcctcccaaagtgctggaattacaggcatgagccgctgcatctggccatcctctcaaattttcaagtgttccacaagtatgttctctactgaagagttgctgcatccttgaatcttgggtgatttgaggcacagaaactatgactttattttttgagatggagttttgctcttgttgcccaggctggagtgcaatggcacgtttttcggcttaccgcaactgccgcctcctgggttcaagcgatagctgggattacaggcatgcgccaccatgcccagctaatttatttgtatttttagtagagacggggtttctccatgttggtcaggctggtctcgaactcccgacctcaggtgatgtgcacacctcagcctcccaataaaccatgacttttaagaggaatagcaggtttacttcccctgccagcattggggtgctctctaagcaacagtaggcggagagtggtctggcgtattaaaaacaaaggatcgtcaagtgggccttcccaggcattgctttgacttagtacatgtagaggatgtggcagttctctccgtccctgccactgctggtttctttgttaaatgtttagttgaaatggcctgatacgatatttgagtagttcactgttggtgctttgcctagcaggattctaatcttgctttggttgtggtcccctgatgccctcctgttaggagtggaggaggtcgaagctccttgtaagatatgattactgggaccattagtgtcaagttcctgtgtccttcaaatggcatatgtgattggccttgaccttaaaaggaaatagggtcccaggtgactgtttagtgggtaggtccagtttggggggatcttccaggagaatggatagagacacctagcagcagagagaacattggtgcctctcaagccaacctcccacctcagcctcccaagtagctgggactacaggtgcttcctcgctaccacacctgggtaattttttttttaattacttttttttttttaagaaacagggtttcactgggttgcccaggctggtcttgaactggcctcaagtgacctgcctgcctcagcctcccaaagtgctgggatcacaggtgtgacccactgcgtttggccagaatactctattcttactgaatgattgaaatctgtcttgaagcattaggtgtcccatttttgtgagttggaattgggacaggctaagtaggaagtgaggagggtggggagagctgtgctgtaggtctgtttgtcccttccttgatgtagccttcagttagccctttcagcttttttccccatcttgtgccgggccttcctgggtttcagtacttggatgtagggctgcagttatgtcagtggtgggtagattgaccaggaattaaggtctagggtccagcccatgtgagacttgactcactgatctacctttaggcatgtcttccttccagtctcatcctttttaaattttttttttttttttgagacggtctcactctcacccaggctggattgcagtggtgtgatctcgaccaactgcaacctctgcctcccacccgcaagctatctgcccacctcagcctctggagtagctgggacgggactacaggcacctgccaccatgactggctaatttttttttgtattttttgtggagatggggtcttgccatgttgctcaggctggtctggtctcaaaactgctctgggctcaagtgattgtcccactttggcctcccagagtgctgggattaaggtgtgagatactgtgtccggctatgaaaattttatttttaattaacttgtatatatttatggggtacaatgtcctatttctgtacatgtacacattgtggaatcaaatcaggctaatatatccatcacttcatatcattagcatgaatgagaacatacaaagccactcttagaaaattttgaaatttatgttatttcagcccttttatgctggaggttgcaaatgttttgtgaataatcagaccaaaaataaaaacaaaaaatgattgacttcagtcattcagtaagaa

SEQ ID NO: 11 - protein, 399 aa, Homo sapiens
MNFRQLLLHLPRYLGASGSPRRLWWSPSLDTISSVGSWRGRSSKSPAHWNQVVSEAEKIVGYPTSFMSLRCLLSDELSNIAMQVRKLVGTQHPLLTTARGLVHDSWNSLQLRGLVVLLISKAAGPSSVNTSCQNYDMVSGIYSCQRSLAEITELIHIALLVHRGIVNLNELQSSDGPLKDMQFGNKIAILSGDFLLANACNGLALLQNTKVVELLASALMDLVQGVYHENSTSKESYITDDIGISTWKEQTFLSHGALLAKSCQAAMELAKHDAEVQNMAFQYGKHMAMSHKINSDVQPFIKEKTSDSMTFNLNSAPVVLHQEFLGRDLWIKQIGEAQEKGRLDYAKLRERIKAGKGVTSAIDLCRYHGNKALEALESFPPSEARSALENIVFAVTRFS

SEQ ID NO: 12 - DNA, 3568 bp, Homo sapiens
ggccgcattccatgcctccaatatggcgtcctccacataggcagtggctgtggtttctaccccgggtggccgggggcagtgctgagctgggactgttgtttgcccagcctgggctgcagaaagcagcagttaaagttcgtttctggtcactgctccaggaagccaccttactctgagggtcaagaattgccgcttccttttagttactgtaagttcctcctctgcccctggtttgtttcccgcggcacttctggatacccccaggtcccagacccttccagactcaaaccatgaactttcggcagctgctgttgcacttgccacgttatcttggagcctcgggttccccgcgtcgcctgtggtggtccccgtccctcgacaccatctcctcggtgggctcttggcgtggtcggtcctccaagtccccggcccactggaatcaggtagtgtcagaggcggagaagatcgtggggtaccccacgtccttcatgagccttcgctgcctgctgagcgacgagctcagcaacatcgctatgcaggtgcggaagctggtgggcactcagcaccctctgcttaccacagccagggggcttgtacatgacagctggaatagcctccagttgaggggcttggtggtgctccttatctctaaagcagctgggcccagcagcgtgaacacttcatgtcagaactatgacatggtcagtgggatctactcatgtcaaagaagtttggcagagatcacggagctaattcatattgctctccttgtacatcgtgggatagtaaatttaaatgagttgcaatcatctgatggtccactgaaagacatgcaatttggaaataaaattgctatcctgagtggagactttcttctagcaaatgcctgcaatggactagctctgctacagaacaccaaggttgtggaacttttagcaagtgctcttatggacttggtacaaggagtatatcatgaaaattctacttcaaaggaaagttatatcacagatgatattggaatatcgacttggaaggagcagacttttctctcccatggtgccttactagcaaagagctgccaagctgcaatggaattagcaaagcatgatgctgaggttcagaatatggcatttcagtatgggaagcacatggccatgagtcataagataaattctgatgtccagccttttattaaagaaaagaccagtgactccatgacttttaatctaaactcagctcctgtagtcttacatcaggaatttcttggaagagatttgtggattaaacagatcggagaggctcaagaaaaaggaagattggactatgctaagttgcgagaaagaatcaaagctggcaaaggtgtgacttcagctattgacctgtgtcgttaccatggaaacaaggcactggaggccctggagagctttcctccctcggaggccagatctgctttagaaaacattgtgtttgctgtgaccagattttcatgacatcaaattaaaaagacactattgttagttagctgaaaatcctagggaatgaggttgattgggagcgctttcacgatgcgttaatgacttttaaaacatatgcatttttccttccttttatcacattgctaaatgagttctgctttctttttggaactgctacaaacaaaattagaagaaaaaaaggtcaagcagttttcacttgtcacgccagaagcacacttgaggctgcagtcgcagaaataattaatgagattcgctcctgtgacctcagcaaatggacaggaaataagtccttattgattggaccgagccagggatggcgccagggcggtggcctgtggtttttcctgctagagaggacaaagcaagttggaagctgcaggtgtcaagagaaatgctctcaataccaaccagggaggattgtctaatcaaaaactagtgaccaatttgtcataatggagagtagttcaatggattgagaaaaatatgttttatttgttggcttgtaattatgtctctggattattattattttttttttagatgtagttttgctcttgttgcccaggctggagtgcaatggtgcagttttgactcactgcaacctccgcctcctgggttcaagtgattctcctgcctcagcctcccgagtagctggaattacaggcacctgccaccacgcctggctaattttgtatttttagtagagatggggtttcactatgttggtcaggctagtctcgaactcctgacctcaggtgatccgcctgcctcggcctcccaaagtgctgggattagaggcttgagccactgcacctggcctcatgtctctggatttataatgcagtatgaatatactttgtgctttatggtttttataatgtctttttggagaaattgccgaaaagttgccaaatacttgaagtaggagattaaaatgttatcaaatgttaaattggttatattaggaatagtctgtttttctttcctgaagatcagtttttttattcaaacacatttcaaagaaccaaattttttttttctttaaggaaaaaggagctttttttcaagtgaaatgtattcatttgtaatactttggtttaaggcatactttaatttttacgagtttcagaaacagaatttttgtactagggaattcattggtgagagtgttcttttaacctcagaatgtcaaattttggtcttgaaccacagacatccaattacagaaagaatataagcaatctcacaggcctgcaatcggacactgtctctgtgtggttcataggagatgatttttgaggtttgcactcatgcaatttgagaacaccgttgacaagaaggctgagtttacataaatgatctagattgaaactcagctacctttcttcctcatgtggtgtaattacagccctatctggagacagcgaatacagcaaacagattttattacctagttcgctcaaacactacatgaagttattttagttaaagccctcccccaaaagttataaaaccattttatcagggcccaacatgtggcatgcaatgaagagaaaatgtaaagctacagaggttaatgtattgtattataaaatattttaagtgtactcaaaatatcataattgtacagtttatgccaccataatttgaggcctatagatttagcttaagagaacactgttctgtttgaaatgctttctgtcactgaaattggcttaattagtaaccatggataagatgctttagatcagactaggttttaatcattaacttccacaaagaagtcatactttgcgttaggtgtgctggttggatgtgcaggaacttcagcaagcagtaggttttactaagcagatggtcgggcactgcagggcaccaggcaggatcctagggcgcctcttattctgcgttagcatctggtttgctgtatgaccttgcacaagtcacttccttctgagcctcaattttctcatctgtacaatgagattcaaaagttgacctgaaagtcaagtgtgaaaaaaaaaaagagattaaacaagataattatgaaattcttaaaaaaaaaaaaaaaa

SEQ ID NO: 13 - protein, 500 aa, Homo sapiens
MATVIPGDLSEVRDTQKVPSGKRKRGETKPRKNFPCQLCDKAFNSVEKLKVHSYSHTGERPYKCIQQDCTKAFVSKYKLQRHMATHSPEKTHKCNYCEKMFHRKDHLKNHLHTHDPNKETFKCEECGKNYNTKLGFKRHLALHAATSGDLTCKVCLQTFESTGVLLEHLKSHAGKSSGGVKEKKHQCEHCDRRFYTRKDVRRHMVVHTGRKDFLCQYCAQRFGRKDHLTRHMKKSHNQELLKVKTEPVDFLDPFTCNVSVPIKDELLPVMSLPSSELLSKPFTNTLQLNLYNTPFQSMQSSGSAHQMITTLPLGMTCPIDMDTVHPSHHLSFKYPFSSTSYAISIPEKEQPLKGEIESYLMELQGGVPSSSQDSQASSSSKLGLDPQIGSLDDGAGDLSLSKSSISISDPLNTPALDFSQLFNFIPLNGPPYNPLSVGSLGMSYSQEEAHSSVSQLPPQTQDLQDPANTIGLGSLHSLSAAFTSSLSTSTTLPRFHQAFQ

SEQ ID NO: 14 - DNA, 7233 bp, Homo sapiens
agctgcaagttgggctgcaggggcagcgcatacactacaatggctgctggaaagaggcgtaaggaaacaatttccaggcccgccgcgtccagcccgaaatatgagaaaaaaattattagaaattccgcgggcggtgtagaggcggcggacgggccggagggaggatgttaaagccccgcgattggccaaaatgggaaggattggattccactctcttccacgaagagtcaatgggactggctaagatcaaagtctgaggctttttccatcagtaatcagtccctttttgctttcttttacgaccacatgaaacttgagaagccacctaaagctatatcatttagtggagttgggcagttcccaagtgtccaacaagaaggcctggtttaggctgcgatggccactgtcattcctggtgatttgtcagaagtaagagatacccagaaagtcccttcagggaaacgtaagcgtggtgaaaccaaaccaagaaaaaactttccttgccaactgtgtgacaaggcctttaacagtgttgagaaattaaaggttcactcctactctcacacaggagagaggccctacaagtgcatacaacaagactgcaccaaggcctttgtttctaagtacaaattacaaaggcacatggctactcattctcctgagaaaacccacaagtgtaattattgtgagaaaatgtttcaccggaaagatcatctgaagaatcacctccatacacacgaccctaacaaagagacgtttaagtgcgaagaatgtggcaagaactacaataccaagcttggatttaaacgtcacttggccttgcatgccgcaacaagtggtgacctcacctgtaaggtatgtttgcaaacttttgaaagcacgggagtgcttctggagcaccttaaatctcatgcaggcaagtcgtctggtggggttaaagaaaaaaagcaccagtgcgaacattgtgatcgccggttctacacccgaaaggatgtccggagacacatggtggtgcacactggaagaaaggacttcctctgtcagtattgtgcacagagatttgggcgaaaggatcacctgactcgacatatgaagaagagtcacaatcaagagcttctgaaggtcaaaacagaaccagtggatttccttgacccatttacctgcaatgtgtctgtgcctataaaagacgagctccttccggtgatgtccttaccttccagtgaactgttatcaaagccattcacaaacactttgcagttaaacctctacaacactccatttcagtccatgcagagctcgggatctgcccaccaaatgatcacaactttacctttgggaatgacatgcccaatagatatggacactgttcatccctctcaccacctttctttcaaatatccgttcagttctacctcatatgcaatttctattcctgaaaaagaacagccattaaagggggaaattgagagttacctgatggagttacaaggtggcgtgccctcttcatcccaagattctcaagcatcgtcatcatctaagctagggttggatcctcagattgggtccctagatgatggtgcaggagacctctccctatccaaaagctctatctccatcagtgaccccctaaacacaccagcattggatttttctcagttgtttaatttcatacctttaaatggtcctccctataatcctctatcagtggggagccttggaatgagctattcccaggaagaagcacattcttctgtttcccagctccccccacaaacacaggatcttcaggatcctgcaaacactatagggcttgggtctctgcactcactgtcagcagctttcaccagcagtttaagcacaagtaccaccctcccacgtttccatcaagcttttcagtaggattctgggacatggattcattacagaaatgtatgtgtagctgtgccctagatgaccatttttattttagtgcctactttaaaacagtataaaaatttctgcttttgtataatacaaattttcattaagccagtataaaatagaaactagcttttaaactgagctttggaaccatttgtgttcagttaagtttacctgggtattttgtcctgattcactgccaattgtcacattttaagacttttttttttccatataggaaagccattattagtagtaaacttttacaaatcccattttcaaattacttttagatcttaaaattttcatttttgtctaataacagtggctctaccttttgacatctggctcattaaaaaatttagcaatagaatgtaaattgtataaaaagtttgtgaataactcaagggtttaaattttcttactagcttctaaatggattaataatcaagtgcttcaaatgaattaagagtccagtttcggaagataataaatgtttgttagatacaccataatttcagatcagtatattctgaagactccctgttgtctggctaaaatatttgccatctttattatgagcctttaaggaaaacaaaccctaaacacaaagcatcagtatttatagcaaaaagagactctgttaggtgacatggcatttcgtgtcacttaatagttggccctaaattagtacacaggatattttgtcgtgtttcatccttcttaacatgctatcttttcatttaataatagtaatagtgtatggcattggggtcttcagagtcgatatataggtagatctctttagtcttttccacctttcacatccaaggggtgggtcaagtgcagccagcaatttattttcattgttggcccacggttagtccataatctagagccattgtggaactgcagccatgaggtgtgtttatcccacagtggattgactcagcctctgtgggtgacagacttctaagcaggaagatagacgtgaagcacatggttacatttgggaacttgtgtagggatcatggcccctgtagccagggttaaaaactggactttttagaagtaaagtaaaagcatatcgcttatatcatttcttgctgaatttgatatgtttttctttcccttaagaatcaaaagcagaaaacaaaaacaacagtcctactccgatgttatctttctgattcaatgtgaatccatctttccttgcaatattttggatggagaatttgaagttaaatgcattagaaaactacctgatgaactaccacaaagttttaagtgactagaaatatatacagtaaaatcccactttcatgcatctctgggaaatgataggagtattgcaaataagttgagtttgtagagggtaacaaagtaaagtaaaacaaacctatcttggttaacatgaaaataacaattgagaatatattatattcactgaataattataggcttttcctcacattagacaaccaacataatcttcttaaaggtctaattaatatatttttctaagggtcagttgggacattaacctaagaaacatatctattaagcacttgttaacaccttattttaggaccctttccgttggggatgggggcaagggtgggaggtttttagaagagtatatatctctttaaaaaaaaacagaaagaaaaatatttctgagcactcattagccctatatggaaacttctttcctttttgtagggccagttatcactgcagattgcaatgtttaccaagaatttctaaaaatgagtgcagattactgaatataatacattatttaaaatatttgggagtagtataatttgtggagaaatgtaaattgtaataatgtaaatgggggcttcaatatatatatataatacacacacacacacacatgcacacataccgcacttcatagaatcaaagttgctctctgaaggagctttggctcctgatattttatcatgctcctatatttttttaatccttggagcagtagtttttatacttatgtatttaaattttattatgaaaaattacatttattaaaaaagtgtgttccaaaggcattaaaattatatatgttaataaggaagtacatttttaaatttttcaaactgctcctagcttttgattaggagaatattttttctgaaagtaggcttttcgctctgcttcattactgcttcctttagtttctatgaaacagattgcttacctaaatctttagttgaatgattagtgttcaatattgctttaatcaccatataaaaggaaaaaaattggtgacagagcacaaatagaaaacctatttttaaatagaaatcacaaatagcaagtgtggaagcactactttattctgtttaaaatgtacttaagaagtcatcaaattagtgaactgagacattggccttagtaggctgtattcactgctaatttaaaaaagggagtaccaggatttattaagtaaagcattttggaaatggggaatagcgccatatatgtatgtatgtgtatgtgtgtgtgtgtgtgtgtatatatacacacacacacacatacttaaatcttgccctgcatgaaattcaaatacatggaggcacatcttcagggcaccagtgttaaaattttggagtcttaattttcatgtgtacacctctttgcctgttcccacccccagacttgaaataacacttcagagtaagagggaattcagctaatttgtttttaaaattgactgtagtggtcactaaaccctttttgagagaatttctattaaagatgaggcagactcgcttatttgaattgcacaatgttctaacaaggatgtaacacagaattggcttttttttccctagaaaaagattgtttgtttctatgtcaactagatatgattaaaaataagtattgccaatgctgttttcattctctagtggccagaatcattatccttgaaatttctggtagtgccttagcttggttaaaaaaaaaaaaaaaaaaaaaaaaaagggattaacattaaataaaagtagtttagaatttgggcctcagacaagatattgaacctcattcagtttcacttccacatgtatgtacaagttaggtcaccaaacacggaagtgtgagtgtggaaggatcttggcactgtaagcaatgctatccattgatgtatacaagtacctttatagttatcgatcactgttaaaactttcattttaaaatcctattaccaagttcagttttttaaaacttcaattgtcctggctgattatgcatcactctgtgtgcaacttttttatttcatttagtgtttctttcaagctgtgtatttttgcctatttgttgcttgtgctttatttttcttagtcatttgtggaatatagtgatatattgtgttaatttggacagtagcggtttttaaaaaccatatactgactgaaacatgagccagagccgattgctttattaagctaataatgaatgttaaagagtacatattttcaggatcgttcatctagtgagcaatacacatattataggccaatatttttttaaaaaatagagcttggtcaacctctatactacacatattacaagatatagcactttcaaaatgaatctaaacctttacagaaactttcttataggttatgccttttattttaagacttattataattcaagtgccattagatgatatatatgtaggcctttgatatataatgctttgtgtacaaaaatggtagatggtattttaaacaggtacatttttacagtgttttcttatcaatttgctatattgcacagaatcagtgtgtgtcttttcataaggttttacaatggtttatttttttacaaggtttacgtgtctcaaagcacactgtcttcccagtacgtaagttaaaaaataccagttcacccaagttgcttctagcctactgagatccatgtgacattggaggagatcttttaaatgtttagtattcgtcattagcaatggctggctgttagttctggtaaatgtgtgcctaagttgaatttgtcttgtttttctcacactgtgtcagcagccatgtctacaacacagataagtctgttgtgatcacatagatctacataagttgtgcagttttgtgctaaaaacccatagggagctcctttgggatcatagaaaagaagatcatgcaaccagcattggtgaaggcacactcagattgcacttagggcctttctatgatgttgtcaaccctctgaggatggaaggcagtgtcttttgatgttatctagcctagaaatgacacagaactattgctaatgtataaaacacttcattatataagcttcagtggtacagatgaaccagaatgaatgtttatcttctcagaaacactccttcaatattatattggatcatgctgctaatgtaacttgggctacaactcttcatggtgctacaaacttctctgtctcattcagtcgtatttttttatccatagaaaaaggactacattaggtgtaaaagtgtacaatatatttttatactgtgacttaatttgtcattaacaaacttttacaccaccacaatgtattcatgtgcacttgcaaaaggagatctcggacatgcaaatgttaccagaacaaacccagcttttgtccacaaggtgactgtaactcagaatggaaagtgggctttataatagggtgtggagtgaagaacatgctgtatgttactaacagccctttgaatttaacaaaaactgggaatccattaggaaacggattgcatcatacctgaacataagctggactgctgaaattgtatttttagctaatgaaaaagtgtttggactagtactctaaaaatgttctaatgataaagttttgagtcaaaatagaaaagaaaaaaatctgcattccaggccgaattttgtatatttttattgcatttaaaattgctattctgtaatattgggaaatcaagtggcttatcatgtatatcgtgtacttaaaatgtattcacaaactactgttgtatttgtataaaatatagacaaagatcatattttttgtgtgtgtataagctctgtaaaatagcaatcacattatgaagctgcagtgatactacattttaaacattcacatccaaagaagcagactatttattgtccatataccagatttaaaatattaatttgctgctaattaaataatagtactgcagcttcttgtggcctacagtgttatgtttgctgtaagaataagatatgtgaattccacaaaatatatgaataaaattatagaatggcttta

SEQ ID NO: 15 - protein, 249 aa, Homo sapiens
MKLNISFPATGCQKLIEVDDERKLRTFYEKRMATEVAADALGEEWKGYVVRISGGNDKQGFPMKQGVLTHGRVRLLLSKGHSCYRPRRTGERKRKSVRGCIVDANLSVLNLVIVKKGEKDIPGLTDTTVPRRLGPKRASRIRKLFNLSKEDDVRQYVVRKPLNKEGKKPRTKAPKIQRLVTPRVLQHKRRRIALKKQRTKKNKEEAAEYAKLLAKRMKEAKEKRQEQIAKRRRLSSLRASTSKSESSQK

SEQ ID NO: 16 - DNA, 829 bp, Homo sapiens
cctcttttccgtggcgcctcggaggcgttcagctgcttcaagatgaagctgaacatctccttcccagccactggctgccagaaactcattgaagtggacgatgaacgcaaacttcgtactttctatgagaagcgtatggccacagaagttgctgctgacgctctgggtgaagaatggaagggttatgtggtccgaatcagtggtgggaacgacaaacaaggtttccccatgaagcagggtgtcttgacccatggccgtgtccgcctgctactgagtaaggggcattcctgttacagaccaaggagaactggagaaagaaagagaaaatcagttcgtggttgcattgtggatgcaaatctgagcgttctcaacttggttattgtaaaaaaaggagagaaggatattcctggactgactgatactacagtgcctcgccgcctgggccccaaaagagctagcagaatccgcaaacttttcaatctctctaaagaagatgatgtccgccagtatgttgtaagaaagcccttaaataaagaaggtaagaaacctaggaccaaagcacccaagattcagcgtcttgttactccacgtgtcctgcagcacaaacggcggcgtattgctctgaagaagcagcgtaccaagaaaaataaagaagaggctgcagaatatgctaaacttttggccaagagaatgaaggaggctaaggagaagcgccaggaacaaattgcgaagagacgcagactttcctctctgcgagcttctacttctaagtctgaatccagtcagaaataagattttttgagtaacaaataaataagatcagactctg

SEQ ID NO: 17 - protein, 467 aa, Homo sapiens
MSSILPFTPPVVKRLLGWKKSAGGSGGAGGGEQNGQEEKWCEKAVKSLVKKLKKTGRLDELEKAITTQNCNTKCVTIPSTCSEIWGLSTPNTIDQWDTTGLYSFSEQTRSLDGRLQVSHRKGLPHVIYCRLWRWPDLHSHHELKAIENCEYAFNLKKDEVCVNPYHYQRVETPVLPPVLVPRHTEILTELPPLDDYTHSIPENTNFPAGIEPQSNYIPETPPPGYISEDGETSDQQLNQSMDTGSPAELSPTTLSPVNHSLDLQPVTYSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSVRCSSMS

SEQ ID NO: 18 - DNA, 10551 bp, Homo sapiens
cggccgggaggcggggcgggccgtaggcaaagggaggtggggaggcggtggccggcgactccccgcgccccgctcgccccccggcccttcccgcggtgctcggcctcgttcctttcctcctccgctccctccgtcttccatacccgccccgcgcggctttcggccggcgtgcctcgcgccctaacgggcggctggaggcgccaatcagcgggcggcagggtgccagccccggggctgcgccggcgaatcggcggggcccgcggcccagggtggcaggcgggtctacccgcgcggccgcggcggcggagaagcagctcgccagccagcagcccgccagccgccgggaggttcgatacaagaggctgttttcctagcgtggcttgctgcctttggtaagaacatgtcgtccatcttgccattcacgccgccagttgtgaagagactgctgggatggaagaagtcagctggtgggtctggaggagcaggcggaggagagcagaatgggcaggaagaaaagtggtgtgagaaagcagtgaaaagtctggtgaagaagctaaagaaaacaggacgattagatgagcttgagaaagccatcaccactcaaaactgtaatactaaatgtgttaccataccaagcacttgctctgaaatttggggactgagtacaccaaatacgatagatcagtgggatacaacaggcctttacagcttctctgaacaaaccaggtctcttgatggtcgtctccaggtatcccatcgaaaaggattgccacatgttatatattgccgattatggcgctggcctgatcttcacagtcatcatgaactcaaggcaattgaaaactgcgaatatgcttttaatcttaaaaaggatgaagtatgtgtaaacccttaccactatcagagagttgagacaccagttttgcctccagtattagtgccccgacacaccgagatcctaacagaacttccgcctctggatgactatactcactccattccagaaaacactaacttcccagcaggaattgagccacagagtaattatattccagaaacgccacctcctggatatatcagtgaagatggagaaacaagtgaccaacagttgaatcaaagtatggacacaggctctccagcagaactatctcctactactctttcccctgttaatcatagcttggatttacagccagttacttactcagaacctgcattttggtgttcgatagcatattatgaattaaatcagagggttggagaaaccttccatgcatcacagccctcactcactgtagatggctttacagacccatcaaattcagagaggttctgcttaggtttactctccaatgttaaccgaaatgccacggtagaaatgacaagaaggcatataggaagaggagtgcgcttatactacataggtggggaagtttttgctgagtgcctaagtgatagtgcaatctttgtgcagagccccaattgtaatcagagatatggctggcaccctgcaacagtgtgtaaaattccaccaggctgtaatctgaagatcttcaacaaccaggaatttgctgctcttctggctcagtctgttaatcagggttttgaagccgtctatcagctaactagaatgtgcaccataagaatgagttttgtgaaagggtggggagcagaataccgaaggcagacggtaacaagtactccttgctggattgaacttcatctgaatggacctctacagtggttggacaaagtattaactcagatgggatccccttcagtgcgttgctcaagcatgtcataaagcttcaccaatcaagtcccatgaaaagacttaatgtaacaactcttctgtcatagcattgtgtgtggtccctatggactgtttactatccaaaagttcaagagagaaaacagcacttgaggtctcatcaattaaagcaccttgtggaatctgtttcctatatttgaatattagatgggaaaattagtgtctagaaatactctcccattaaagaggaagagaagattttaaagacttaatgatgtcttattgggcataaaactgagtgtcccaaaggtttattaataacagtagtagttatgtgtacaggtaatgtatcatgatccagtatcacagtattgtgctgtttatatacatttttagtttgcatagatgaggtgtgtgtgtgcgctgcttcttgatctaggcaaacctttataaagttgcagtacctaatctgttattcccacttctctgttatttttgtgtgtcttttttaatatataatatatatcaagattttcaaattatttagaagcagattttcctgtagaaaaactaatttttctgccttttaccaaaaataaactcttgggggaagaaaagtggattaacttttgaaatccttgaccttaatgtgttcagtggggcttaaacagtcattctttttgtggttttttgtttttttttgtttttttttttaactgctaaatcttattataaggaaaccatactgaaaacctttccaagcctcttttttccattcccatttttgtcctcataatcaaaacagcataacatgacatcatcaccagtaatagttgcattgatactgctggcaccagttaattctgggatacagtaagaattcatatggagaaagtccctttgtcttatgcccaaatttcaacaggaataattggcttgtataatctagcagtctgttgatttatccttccacctcataaaaaatgcataggtggcagtataattattttcagggatatgctagaattacttccacatatttatccctttttaaaaaagctaatctataaataccgtttttccaaaggtattttacaatatttcaacagcagaccttctgctcttcgagtagtttgatttggtttagtaaccagattgcattatgaaatgggccttttgtaaatgtaattgtttctgcaaaatacctagaaaagtgatgctgaggtaggatcagcagatatgggccatctgtttttaaagtatgttgtattcagtttataaattgattgttattctacacataattatgaattcagaattttaaaaattgggggaaaagccatttatttagcaagttttttagcttataagttacctgcagtctgagctgttcttaactgatcctggttttgtgattgacaatatttcatgctctgtagtgagaggagatttccgaaactctgttgctagttcattctgcagcaaataattattatgtctgatgttgactcattgcagtttaaacatttcttcttgtttgcatcttagtagaaatggaaaataaccactcctggtcgtcttttcataaattttcatatttttgaagctgtctttggtacttgttctttgaaatcatatccacctgtctctataggtatcattttcaatactttcaacatttggtggttttctattgggtactccccattttcctatatttgtgtgtatatgtatgtgttcatgtaaatttggtatagtaattttttattcattcaacaaatatttattgttcacctgtttgtaccaggaacttttcttagtctttgggtaaaggtgaacaagacaactacagttcctgcctttgctgagacagcagttacactaacccttaattatcttacttgtctatgaaggagataaacagggtactgtactggagaataacagatgggatgcttcaggtaggacatcaaggaaagcctctaaggaaaggatgcatgagctaacacctgacattaaagaagcaagccaagtgaggagccaggggagataagcattcctggcaaagagaatagcatcaaatgcaaaaaggttcacactaaaggaaactcctgattaggtattaatgctttatacagaaacctctatacaaatccaaacttgaagatcagaatggttctacagttcataacattttgaaggtggccttattttgtgatagtctgcttcatgtgattctcactaacatatctccttcctcaacctttgctgtaaaaatttcatttgcaccacatcagtactacttaatttaacaagcttttgttgtgtaagctctcactgttttagtgccctgctgcttgcttccagactttgtgctgtccagtaattatgtcttccactacccatcttgtgagcagagtaaatgtcctaggtaataccactatcaggcctgtaggagatactcagtggagcctctgcccttctttttcttacttgagaacttgtaatggtgttagggaacagttgtaggggcagaaaacaactctgaaagtggtagaaggtcctgatcttggtggttactcttgcattactgtgttaggtcaagcagtgcctactatgctgtttcagtagtggagcgcatctctacagttctgatgcgatttttctgtacagtatgaaattgggactcaactctttgaaaacacctattgagcagttatacctgttgagcagtttacttcctggttgtaattacatttgtgtgaatgtgtttgatgctttttaacgagatgatgttttttgtattttatctactgtggcctgattttttttttgttttctgcccctccccccatttataggtgtggttttcatttttctaagtgatagaatcccctctttgttgaatttttgtctttatttaaattagcaacattacttaggatttattcttcacaatactgttaattttctaggaatgatgacctgagaaccgaatggccatgctttctatcacatttctaagatgagtaatattttttccagtaggttccacagagacaccttgggggctggcttaggggaggctgttggagttctcactgacttagtggcatatttattctgtactgaagaactgcatggggtttcttttggaaagagtttcattgctttaaaaagaagctcagaaagtctttataaccactggtcaacgattagaaaaatataactggatttaggcctaccttctggaataccgctgattgtgctctttttatcctactttaaagaagctttcatgattagatttgagctatatcagttataccgattataccttataatacacattcagttagtaaacatttattgatgcctgttgtttgcccagccactgtgatggatattgaataataaaaagatgactaggacggggccctgacccttgagctgtgcttggtcttgtagaggttgtgttttttttcctcaggacctgtcactttggcagaaggaaatctgcctaatttttcttgaaagctaaattttctttgtaagtttttacaaattgtttaatacctagttgtattttttaccttaagccacattgagttttgcttgatttgtctgtcttttaaacactgtcaaatgctttcccttttgttaaaattattttaatttcactttttttgtgcccttgtcaatttaagactaagactttgaaggtaaaacaaacaaacaaacatcagtcttagtctcttgctagttgaaatcaaataaaagaaaatatatacccagttggtttctctacctcttaaaagcttcccatatatacctttaagatccttctcttttttctttaactactaaataggttcagcatttattcagtgttagataccctcttcgtctgagggtggcgtaggtttatgttgggatataaagtaacacaagacaatcttcactgtacataaaatatgtcttcatgtacagtctttactttaaaagctgaacattccaatttgcgccttccctcccaagcccctgcccaccaagtatctctttagatatctagtctgtggacatgaacaatgaatacttttttcttactctgatcgaaggcattgatacttagacatatcaaacatttcttcctttcatatgctttactttgctaaatctattatattcattgcctgaattttattcttcctttctacctgacaacacacatccaggtggtacttgctggttatcctctttcttgttagccttgttttttgttttttttttttttttttgagagggagtctcgctctgttgcccaacctggagtgcagtggtgcgatcttggttcactgcaagctccgcctcccgggttcacgccatgcttctgcctcagcctcccaagtagctgggactacaggcgcccaccaccacactcggctaattttttgtatttttagtagagacggggtttcaccgtgttggccaggatggtctcgatctcctgacctcgtgatctgtccacctcggcttcccaaagtgctgggattacaggcatgagccaccgcgcccagcctagccatatttttatctgcatatatcagaatgtttctctcctttgaacttattaacaaaaaaggaacatgcttttcatacctagagtcctaatttcttcatcatgaaggttgctattcaaattgatcaatcattttaattttacaaatggctcaaaaattctgttcagtaaatgtctttgtgactggcaaatggcataaattatgtttaagattatgaacttttctgacagttgcagccaatgttttccctacgataccagatttccatcttggggcatattggattgttgtatttaagacagtcagaataatgatagtgtgtggtctccagaggtagtcagaatcctgctattgagttctttttatatcttccttttcaattttttattaccattttgtttgtttagactacactttgtagggattgaggggcaaattatctcttggagtggaattcctgtgttttgagccttacaaccaggaaatatgagctatactagatagcctcatgatagcatttacgataagaacttatctcgtgtgttcatgtaattttttgagtaggaactgttttatcttgaatattgtagctaactatatatagcagaactgcctcagtctttttaagaaggaaataaataatatatgtgtatgaatttatatatacatatacactcatagacaaacttaacagttggggtcattctaacagttaaaacaattgttccattgtttaaatctcagatcctggtaaaatgttcttaatttgtctgtgtacattttcctttcatggacagaccattggagtacattaattttcttaatctgccatttggcagttcatttaatataccattttttggcaacttggtaactaagaatcacagccaaaatttgttaacatcaaagaaagctctgccatataccccgttactaaattattatacatccagcagattctgggatgtactaacttagggttaactttgttgttgttgataatactagattgctccctctttaattcttcttctggtgcaaggttgctgcttaagttaccctgggaaatactactacaaggtcaaattttctagtatcttacagcctgattgaaggtgattcagatctttgctcaatataaatggattttccaagattctctgggccatccttgacccacaggtgatctcgctggagtatattaacttaacttcagtgccagttggtttggtgccatgagatccataatgaatccagaacttcaccattgcttagatataagagtcccttggaagaataatgccactgatgatgggggtcagaaggtgtattaactcaacatagagggcttttagatttttcttcaaaaaaatttcgagaaaagtattcttttaccctccaaacagttaacagctcttagtttctccaaatatgctctttgatttacttatttttaattaaagatggtaatttattgaacaatgaaatccgtaatatattgatttaaggacaaaagtgaagttttagaattataaaagtacttaaatattatatattttccatttcataattgttttcctttctctgtggctttaaagtttttgactattttacaatgttaatcactaggtaacttgccatatttctggttctatattaagttctatcctttataatgctgttattataaagctggtttttagcatttgtctgtagcaatagaaattttactaagtctctgttctcccagtaagttttttcttttctcagtaagtccctaagaaaacatttgtttgccactcttactattcccaatcttggattgttcgagctgaaaaaaaatttgatgagaaacaggaggatccttttctggtgaatataggttcctgctttaagaatgtggaaatccattgctttatataactaatatacacacagattaattaaaattgtgagaaataattcacacatgacaagtaggtaacatgcatgagttttgaatttttttaaaaacccaactgtttgacaaaatatagaacccaaattggtactttcttagaccagtgtaacctcacacctcagttttgcttttccaaccctgacttgaaaggcatatttgtatctttttattagtgatagtgaagctgtgacactaaccttttatacaaaagagtaaagaaagaaaaactacagcgattaagatgagaacagttctgcagttgttgaactagatcacagcattgtaggcagaataaaaaatgttcatatctgagaatattcctttcgccatcttttcccaaggccagacctcctggtggagcacagttaaaagtaacattctgggcctttgtaatcggagggctgtgtctccagctggcagcctttgttttaatatataatgcaggactgtggaaaacagttggcatagaatattttcacctaaaaaagaaagaaaagacatacaaaactggattaattgcaaaaagagaatacagtaaaataccatataactggacaaagctagaagaacctttagaagatttgtctgaaaacagatttcaagagtgagcttttatacactgctcactaatttgcttgattactaccaactcttcttaaagttaacacgtttaaggtatttctggacttcctagccttttagcaagcttagaggaactagccattagctagtgatgtaaaaatattttggggactgatgcccttaaaggttatgcccttgaaagttcttaccttttctctagtgatattaaggaacgagtgggtagtgttctcagggtgaccagctgccctaaagtgcctgggattgagggtttccctggatgcgggactttccctggatacaaaacttttagcagagttttgtatatatgtggatttttctgataagtagcacatcagaggccttaaccactgcccaaaagcgattctccattgagagtacatatcttgaacttaagaaattcatttgctctgatttttaatcttgtaaagtttttgctaaactcaaaacaagtcccaggcacaccagaaggagctgaccaccttaggtgttcttgtgatttatccttacttccctatgttgtcatagttgcttctaaactcagctgcactatggctgtcaacatttctgatacttattgggatatgtgccatccagtcatttagtactttgaatggaacatgagatttataacacaggtaatagctgaaggtaccagtatggtggtgagactcacacttagtgatccagctaaggtaactgatgttataatggaacagagaagaggccaactagatagctaagttcttctgaacctatgtgtatatgtaagtacaaatcatgcgtccttatggggttaaacttaatctgaaatttacatttttcatagtaaaaggaaaccaattgttgcagatttcttttcttgtgaggaaatacatggcctttgatgctctggcgtctactgcatttcccagtctgttctgctcgagaagccagaatgtgttgttaacatttttccgtgaatgttgtgttaaaatgattaaatgcatcagccaatggcaagtgaaggaattgggtgtcctgatgcagactgagcagtttctctcaattgtagcctcatactcataaggtgcttaccagctagaacattgagcacgtgaggtgagattttttttctctgatggcattaactttgtaatgcaatatgatggatgcagaccctgttcttgtttccctctggaagtccttagtggctgcatccttggtgcactgtgatggagatattaaatgtgttctttgtgagctttcgttctatgattgtcaaaagtacgatgtggttccttttttatttttattaaacaatgagctgaggctttattacagctggttttcaagttaaaattgttgaatactgatgtctttctcccacctacaccaaatattttagtctatttaaagtacaaaaaaagttctgcttaagaaaacattgcttacatgtcctgtgatttctggtcaatttttatatatatttgtgtgcatcatctgtatgtgctttcactttttaccttgtttgctcttacctgtgttaacagccctgtcaccgttgaaaggtggacagttttcctagcattaaaagaaagccatttgagttgtttaccatgttaaaaaaaaaaaaaaaa

SEQ ID NO: 19 - protein, 552 aa, Homo sapiens
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 20 - DNA, 8789 bp, Homo sapiens
atgctcagtggcttctcgacaagttggcagcaacaacacggccctggtcgtcgtcgccgctgcggtaacggagcggtttgggtggcggagcctgcgttcgcgccttcccgctctcctcgggaggcccttcctgctctcccctaggctccgcggccgcccagggggtgggagcgggtgaggggagccaggcgcccagcgagagaggccccccgccgcagggcggcccgggagctcgaggcggtccggcccgcgcgggcagcggcgcggcgctgaggaggggcggcctggccgggacgcctcggggcgggggccgaggagctctccgggccgccggggaaagctacgggcccggtgcgtccgcggaccagcagcgcgggagagcggactcccctcgccaccgcccgagcccaggttatcctgaatacatgtctaacaattttccttgcaacgttagctgttgtttttcactgtttccaaaggatcaaaattgcttcagaaattggagacatatttgatttaaaaggaaaaacttgaacaaatggacaatatgtctattacgaatacaccaacaagtaatgatgcctgtctgagcattgtgcatagtttgatgtgccatagacaaggtggagagagtgaaacatttgcaaaaagagcaattgaaagtttggtaaagaagctgaaggagaaaaaagatgaattggattctttaataacagctataactacaaatggagctcatcctagtaaatgtgttaccatacagagaacattggatgggaggcttcaggtggctggtcggaaaggatttcctcatgtgatctatgcccgtctctggaggtggcctgatcttcacaaaaatgaactaaaacatgttaaatattgtcagtatgcgtttgacttaaaatgtgatagtgtctgtgtgaatccatatcactacgaacgagttgtatcacctggaattgatctctcaggattaacactgcagagtaatgctccatcaagtatgatggtgaaggatgaatatgtgcatgactttgagggacagccatcgttgtccactgaaggacattcaattcaaaccatccagcatccaccaagtaatcgtgcatcgacagagacatacagcaccccagctctgttagccccatctgagtctaatgctaccagcactgccaactttcccaacattcctgtggcttccacaagtcagcctgccagtatactggggggcagccatagtgaaggactgttgcagatagcatcagggcctcagccaggacagcagcagaatggatttactggtcagccagctacttaccatcataacagcactaccacctggactggaagtaggactgcaccatacacacctaatttgcctcaccaccaaaacggccatcttcagcaccacccgcctatgccgccccatcccggacattactggcctgttcacaatgagcttgcattccagcctcccatttccaatcatcctgctcctgagtattggtgttccattgcttactttgaaatggatgttcaggtaggagagacatttaaggttccttcaagctgccctattgttactgttgatggatacgtggacccttctggaggagatcgcttttgtttgggtcaactctccaatgtccacaggacagaagccattgagagagcaaggttgcacataggcaaaggtgtgcagttggaatgtaaaggtgaaggtgatgtttgggtcaggtgccttagtgaccacgcggtctttgtacagagttactacttagacagagaagctgggcgtgcacctggagatgctgttcataagatctacccaagtgcatatataaaggtctttgatttgcgtcagtgtcatcgacagatgcagcagcaggcggctactgcacaagctgcagcagctgcccaggcagcagccgtggcaggaaacatccctggcccaggatcagtaggtggaatagctccagctatcagtctgtcagctgctgctggaattggtgttgatgaccttcgtcgcttatgcatactcaggatgagttttgtgaaaggctggggaccggattacccaagacagagcatcaaagaaacaccttgctggattgaaattcacttacaccgggccctccagctcctagacgaagtacttcataccatgccgattgcagacccacaacctttagactgaggtcttttaccgttggggcccttaaccttatcaggatggtggactacaaaatacaatcctgtttataatctgaagatatatttcacttttgttctgctttatcttttcataaagggttgaaaatgtgtttgctgccttgctcctagcagacagaaactggattaaaacaattttttttttcctcttcagaacttgtcaggcatggctcagagcttgaagattaggagaaacacattcttattaattcttcacctgttatgtatgaaggaatcattccagtgctagaaaatttagccctttaaaacgtcttagagccttttatctgcagaacatcgatatgtatatcattctacagaataatccagtattgctgattttaaaggcagagaagttctcaaagttaattcacctatgttattttgtgtacaagttgttattgttgaacatacttcaaaaataatgtgccatgtgggtgagttaattttaccaagagtaactttactctgtgtttaaaaagtaagttaataatgtattgtaatctttcatccaaaatattttttgcaagttatattagtgaagatggtttcaattcagattgtcttgcaacttcagttttatttttgccaaggcaaaaaactcttaatctgtgtgtatattgagaatcccttaaaattaccagacaaaaaaatttaaaattacgtttgttattcctagtggatgactgttgatgaagtatacttttcccctgttaaacagtagttgtattcttctgtatttctaggcacaaggttggttgctaagaagcctataagaggaatttcttttccttcattcatagggaaaggttttgtattttttaaaacactaaaagcagcgtcactctacctaatgtctcactgttctgcaaaggtggcaatgcttaaactaaataatgaataaactgaatattttggaaactgctaaattctatgttaaatactgtgcagaataatggaaacattacagttcataataggtagtttggatatttttgtacttgatttgatgtgactttttttggtataatgtttaaatcatgtatgttatgatattgtttaaaattcagtttttgtatcttggggcaagactgcaaacttttttatatcttttggttattctaagccctttgccatcaatgatcatatcaattggcagtgactttgtatagagaatttaagtagaaaagttgcagatgtattgactgtaccacagacacaatatgtatgctttttacctagctggtagcataaataaaactgaatctcaacatacaaagttgaattctaggtttgatttttaagattttttttttcttttgcacttttgagtccaatctcagtgatgaggtaccttctactaaatgacaggcaacagccagttctattgggcagctttgtttttttccctcacactctaccgggacttccccatggacattgtgtatcatgtgtagagttggttttttttttttttaatttttattttactatagcagaaatagacctgattatctacaagatgataaatagattgtctacaggataaatagtatgaaataaaatcaaggattatctttcagatgtgtttacttttgcctggagaacttttagctatagaaacacttgtgtgatgatagtcctccttatatcacctggaatgaacacagcttctactgccttgctcagaaggtcttttaaatagaccatcctagaaaccactgagtttgcttatttctgtgatttaaacatagatcttgatccaagctacatgacttttgtctttaaataacttatctaccacctcatttgtactcttgattacttacaaattctttcagtaaacacctaattttcttctgtaaaagtttggtgatttaagttttattggcagttttataaaaagacatcttctctagaaattgctaactttaggtccattttactgtgaatgaggaataggagtgagttttagaataacagatttttaaaaatccagatgatttgattaaaaccttaatcatacattgacataattcattgcttcttttttttgagatatggagtcttgctgtgttgcccaggcaggagtgcagtggtatgatctcagctcactgcaacctctgcctcccgggttcaactgattctcctgcctcagcctccctggtagctaggattacaggtgcccgccaccatgcctggctaacttttgtagttttagtagagacggggttttgcctgttggccaggctggtcttgaactcctgacctcaagtgatccatccaccttggcctcccaaagtgctgggattacgggcgtgagccactgtccctggcctcattgttcccttttctactttaaggaaagttttcatgtttaatcatctggggaaagtatgtgaaaaatatttgttaagaagtatctctttggagccaagccacctgtcttggtttctttctactaagagccataaagtatagaaatacttctagttgttaagtgcttatatttgtacctagatttagtcacacgcttttgagaaaacatctagtatgttatgatcagctattcctgagagcttggttgttaatctatatttctatttcttagtggtagtcatctttgatgaataagactaaagattctcacaggtttaaaattttatgtctactttaagggtaaaattatgaggttatggttctgggtgggttttctctagctaattcatatctcaaagagtctcaaaatgttgaatttcagtgcaagctgaatgagagatgagccatgtacacccaccgtaagacctcattccatgtttgtccagtgcctttcagtgcattatcaaagggaatccttcatggtgttgcctttattttccggggagtagatcgtgggatatagtctatctcatttttaatagtttaccgcccctggtatacaaagataatgacaataaatcactgccatataaccttgctttttccagaaacatggctgttttgtattgctgtaaccactaaataggttgcctataccattcctcctgtgaacagtgcagatttacaggttgcatggtctggcttaaggagagccatacttgagacatgtgagtaaactgaactcatattagctgtgctgcatttcagacttaaaatccatttttgtggggcagggtgtggtgtgtaaaggggggtgtttgtaatacaagttgaaggcaaaataaaatgtcctgtctcccagatgatatacatcttattatttttaaagtttattgctaattgtaggaaggtgagttgcaggtatctttgactatggtcatctggggaaggaaaattttacattttactattaatgctccttaagtgtctatggaggttaaagaataaaatggtaaatgtttctgtgcctggtttgatggtaactggttaatagttactcaccattttatgcagagtcacattagttcacaccctttctgagagccttttgggagaagcagttttattctctgagtggaacagagttctttttgttgataatttctagtttgctcccttcgttattgccaactttactggcattttatttaatgatagcagattgggaaaatggcaaatttaggttacggaggtaaatgagtatatgaaagcaattacctctaaagccagttaacaattattttgtaggtggggtacactcagcttaaagtaatgcatttttttttcccgtaaaggcagaatccatcttgttgcagatagctatctaaataatctcatatcctcttttgcaaagactacagagaataggctatgacaatcttgttcaagcctttccatttttttccctgataactaagtaatttctttgaacataccaagaagtatgtaaaaagtccatggccttattcatccacaaagtggcatcctaggcccagccttatccctagcagttgtcccagtgctgctaggttgcttatcttgtttatctggaatcactgtggagtgaaattttccacatcatccagaattgccttatttaagaagtaaaacgttttaatttttagcctttttttggtggagttatttaatatgtatatcagaggatatactagatggtaacatttctttctgtgcttggctatctttgtggacttcaggggcttctaaaacagacaggactgtgttgcctttactaaatggtctgagacagctatggttttgaatttttagttttttttttttaacccacttcccctcctggtctcttccctctctgataattaccattcatatgtgagtgttagtgtgcctccttttagcattttcttcttctctttctgattcttcatttctgactgcctaggcaaggaaaccagataaccaaacttactagaacgttctttaaaacacaagtacaaactctgggacaggacccaagacactttcctgtgaagtgctgaaaaagacctcattgtattggcatttgatatcagtttgatgtagcttagagtgcttcctgattcttgctgagtttcaggtagttgagatagagagaagtgagtcatattcatattttcccccttagaataatattttgaaaggtttcattgcttccacttgaatgctgctcttacaaaaactggggttacaagggttactaaattagcatcagtagccagaggcaataccgttgtctggaggacaccagcaaacaacacacaacaaagcaaaacaaaccttgggaaactaaggccatttgttttgttttggtgtcccctttgaagccctgccttctggccttactcctgtacagatatttttgacctataggtgcctttatgagaattgagggtctgacatcctgccccaaggagtagctaaagtaattgctagtgttttcagggattttaacatcagactggaatgaatgaatgaaactttttgtcctttttttttctgtttttttttttctaatgtagtaaggactaaggaaaacctttggtgaagacaatcatttctctctgttgatgtggatacttttcacaccgtttatttaaatgctttctcaataggtccagagccagtgttcttgttcaacctgaaagtaatggctctgggttgggccagacagttgcactctctagtttgccctctgccacaaatttgatgtgtgacctttgggcaagtcatttatcttctctgggccttagttgcctcatctgtaaaatgagggagttggagtagattaattattccagctctgaaattctaagtgaccttggctaccttgcagcagttttggatttcttccttatctttgttctgctgtttgagggggctttttacttatttccatgttattcaaaggagactaggcttgatattttattactgttcttttatggacaaaaggttacatagtatgcccttaagacttaattttaaccaaaggcctagcaccaccttaggggctgcaataaacacttaacgcgcgtgcgcacgcgcgcgcgcacacacacacacacacacacacacacacacaggtcagagtttaaggctttcgagtcatgacattctagcttttgaattgcgtgcacacacacacgcacgcacacactctggtcagagtttattaaggctttcgagtcatgacattatagcttttgagttggtgtgtgtgacaccaccctcctaagtggtgtgtgcttgtaattttttttttcagtgaaaatggattgaaaacctgttgttaatgcttagtgatattatgctcaaaacaaggaaattcccttgaaccgtgtcaattaaactggtttatatgactcaagaaaacaataccagtagatgattattaactttattcttggctctttttaggtccattttgattaagtgacttttggctggatcattcagagctctcttctagcctacccttggatgagtacaattaatgaaattcatattttcaaggacctgggagccttccttggggctgggttgagggtggggggttggggagtcctggtagaggccagctttgtggtagctggagaggaagggatgaaaccagctgctgttgcaaaggctgcttgtcattgatagaaggactcacgggcttggattgattaagactaaacatggagttggcaaactttcttcaagtattgagttctgttcaatgcattggacatgtgatttaagggaaaagtgtgaatgcttatagatgatgaaaacctggtgggctgcagagcccagtttagaagaagtgagttgggggttggggacagatttggtggtggtatttcccaactgtttcctcccctaaattcagaggaatgcagctatgccagaagccagagaagagccactcgtagcttctgctttggggacaactggtcagttgaaagtcccaggagttcctttgtggctttctgtatacttttgcctggttaaagtctgtggctaaaaaatagtcgaacctttcttgagaactctgtaacaaagtatgtttttgattaaaagagaaagccaactaaaaaaaaaaaaaaaaaaaa

SEQ ID NO: 21 - protein, 283 aa, Homo sapiens
MAVPPTYADLGKSARDVFTKGYGFGLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGGHKLGLGLEFQA

SEQ ID NO: 22 - DNA, 1993 bp, Homo sapiens
attagcgcagggacctccgggccacagctcagagaatcggaaggcctcctcccccttcccgagcgctgccactggggccgaggtttccagcaagaacccgcgtgtccctgcgcacgcacacacggtgcacacgtcagtccggcgcctccccgtgccccgactcacgcaggtcctcccgcgcgcccgcaacacgcccgcaggctcctgtgtctgctgccggggcagcggggcccggaaggcagaagatggctgtgccacccacgtatgccgatcttggcaaatctgccagggatgtcttcaccaagggctatggatttggcttaataaagcttgatttgaaaacaaaatctgagaatggattggaatttacaagctcaggctcagccaacactgagaccaccaaagtgacgggcagtctggaaaccaagtacagatggactgagtacggcctgacgtttacagagaaatggaataccgacaatacactaggcaccgagattactgtggaagatcagcttgcacgtggactgaagctgaccttcgattcatccttctcacctaacactgggaaaaaaaatgctaaaatcaagacagggtacaagcgggagcacattaacctgggctgcgacatggatttcgacattgctgggccttccatccggggtgctctggtgctaggttacgagggctggctggccggctaccagatgaattttgagactgcaaaatcccgagtgacccagagcaactttgcagttggctacaagactgatgaattccagcttcacactaatgtgaatgacgggacagagtttggcggctccatttaccagaaagtgaacaagaagttggagaccgctgtcaatcttgcctggacagcaggaaacagtaacacgcgcttcggaatagcagccaagtatcagattgaccctgacgcctgcttctcggctaaagtgaacaactccagcctgataggtttaggatacactcagactctaaagccaggtattaaactgacactgtcagctcttctggatggcaagaacgtcaatgctggtggccacaagcttggtctaggactggaatttcaagcataaatgaatactgtacaattgtttaattttaaactattttgcagcatagctaccttcagaatttagtgtatcttttaatgttgtatgtctgggatgcaagtattgctaaatatgttagccctccaggttaaagttgattcagctttaagatgttacccttccagaggtacagaagaaacctatttccaaaaaaggtcctttcagtggtagactcggggagaacttggtggcccctttgagatgccaggtttcttttttatctagaaatggctgcaagtggaagcggataatatgtaggcactttgtaaattcatattgagtaaatgaatgaaattgtgatttcctgagaatcgaaccttggttccctaaccctaattgatgagaggctcgctgcttgatggtgtgtacaaactcacctgaatgggacttttttagacagatcttcatgacctgttcccaccccagttcatcatcatctcttttacaccaaaaggtctgcagggtgtggtaactgtttcttttgtgccattttggggtggagaaggtggatgtgatgaagccaataattcaggacttattccttcttgtgttgtgtttttttttggcccttgcaccagagtatgaaatagcttccaggagctccagctataagcttggaagtgtctgtgtgattgtaatcacatggtgacaacactcagaatctaaattggacttctgttgtattctcaccactcaatttgttttttagcagtttaatgggtacattttagagtcttccattttgttggaattagatcctccccttcaaatgctgtaattaacaacacttaaaaaacttgaataaaatattgaaacctcatccttcttctgttgtctttattaataaaatataaataaac

SEQ ID NO: 23 - protein, 324 aa, Homo sapiens
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE

SEQ ID NO: 24 - DNA, 1561 bp, Homo sapiens
gggcttatcccgcctgtcccgccattctcgctagttcgatcggtagcgggagcggagagcggaccccagagagccctgagcagccccaccgccgccgccggcctagttaccatcacaccccgggaggagccgcagctgccgcagccggccccagtcaccatcaccgcaaccatgagcagcgaggccgagacccagcagccgcccgccgccccccccgccgcccccgccctcagcgccgccgacaccaagcccggcactacgggcagcggcgcagggagcggtggcccgggcggcctcacatcggcggcgcctgccggcggggacaagaaggtcatcgcaacgaaggttttgggaacagtaaaatggttcaatgtaaggaacggatatggtttcatcaacaggaatgacaccaaggaagatgtatttgtacaccagactgccataaagaagaataaccccaggaagtaccttcgcagtgtaggagatggagagactgtggagtttgatgttgttgaaggagaaaagggtgcggaggcagcaaatgttacaggtcctggtggtgttccagttcaaggcagtaaatatgcagcagaccgtaaccattatagacgctatccacgtcgtaggggtcctccacgcaattaccagcaaaattaccagaatagtgagagtggggaaaagaacgagggatcggagagtgctcccgaaggccaggcccaacaacgccggccctaccgcaggcgaaggttcccaccttactacatgcggagaccctatgggcgtcgaccacagtattccaaccctcctgtgcagggagaagtgatggagggtgctgacaaccagggtgcaggagaacaaggtagaccagtgaggcagaatatgtatcggggatatagaccacgattccgcaggggccctcctcgccaaagacagcctagagaggacggcaatgaagaagataaagaaaatcaaggagatgagacccaaggtcagcagccacctcaacgtcggtaccgccgcaacttcaattaccgacgcagacgcccagaaaaccctaaaccacaagatggcaaagagacaaaagcagccgatccaccagctgagaattcgtccgctcccgaggctgagcagggcggggctgagtaaatgccggcttaccatctctaccatcatccggtttagtcatccaacaagaagaaatatgaaattccagcaataagaaatgaacaaaagattggagctgaagacctaaagtgcttgctttttgcccgttgaccagataaatagaactatctgcattatctatgcagcatggggtttttattatttttacctaaagacgtctctttttggtaataacaaacgtgttttttaaaaaagcctggtttttctcaatacgcctttaaaggtttttaaattgtttcatatctggtcaagttgagatttttaagaacttcatttttaatttgtaataaaagtttacaacttgattttttcaaaaaagtcaacaaactgcaagcacctgttaataaaggtcttaaataataaaaaaaaaaaaaaa

SEQ ID NO: 25 - protein, 679 aa, Homo sapiens
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 26 - DNA, 3506 bp, Homo sapiens
ttcctcccctggactctttctgagctcagagccgccgcagccgggacaggagggcaggctttctccaaccatcatgctgcggagcatattacctgtacgccctggctccgggagcggcagtcgagtatcctctggtcaggcggcgcgggcggcgcctcagcggaagagcgggcctctgggccgcagtgaccaacccccgcccctcaccccacgtggttggaggtttccagaagcgctgccgccaccgcatcgcgcagctctttgccgtcggagcgcttgtttgctgcctcgtactcctccatttatccgccatgataagtgccagccgagctgcagcagcccgtctcgtgggcgccgcagcctcccggggccctacggccgcccgccaccaggatagctggaatggccttagtcatgaggcttttagacttgtttcaaggcgggattatgcatcagaagcaatcaagggagcagttgttggtattgatttgggtactaccaactcctgcgtggcagttatggaaggtaaacaagcaaaggtgctggagaatgccgaaggtgccagaaccaccccttcagttgtggcctttacagcagatggtgagcgacttgttggaatgccggccaagcgacaggctgtcaccaacccaaacaatacattttatgctaccaagcgtctcattggccggcgatatgatgatcctgaagtacagaaagacattaaaaatgttccctttaaaattgtccgtgcctccaatggtgatgcctgggttgaggctcatgggaaattgtattctccgagtcagattggagcatttgtgttgatgaagatgaaagagactgcagaaaattacttggggcacacagcaaaaaatgctgtgatcacagtcccagcttatttcaatgactcgcagagacaggccactaaagatgctggccagatatctggactgaatgtgcttcgggtgattaatgagcccacagctgctgctcttgcctatggtctagacaaatcagaagacaaagtcattgctgtatatgatttaggtggtggaacttttgatatttctatcctggaaattcagaaaggagtatttgaggtgaaatccacaaatggggataccttcttaggtggggaagactttgaccaggccttgctacggcacattgtgaaggagttcaagagagagacaggggttgatttgactaaagacaacatggcacttcagagggtacgggaagctgctgaaaaggctaaatgtgaactctcctcatctgtgcagactgacatcaatttgccctatcttacaatggattcttctggacccaagcatttgaatatgaagttgacccgtgctcaatttgaagggattgtcactgatctaatcagaaggactatcgctccatgccaaaaagctatgcaagatgcagaagtcagcaagagtgacataggagaagtgattcttgtgggtggcatgactaggatgcccaaggttcagcagactgtacaggatctttttggcagagccccaagtaaagctgtcaatcctgatgaggctgtggccattggagctgccattcagggaggtgtgttggccggcgatgtcacggatgtgctgctccttgatgtcactcccctgtctctgggtattgaaactctaggaggtgtctttaccaaacttattaataggaataccactattccaaccaagaagagccaggtattctctactgccgctgatggtcaaacgcaagtggaaattaaagtgtgtcagggtgaaagagagatggctggagacaacaaactccttggacagtttactttgattggaattccaccagcccctcgtggagttcctcagattgaagttacatttgacattgatgccaatgggatagtacatgtttctgctaaagataaaggcacaggacgtgagcagcagattgtaatccagtcttctggtggattaagcaaagatgatattgaaaatatggttaaaaatgcagagaaatatgctgaagaagaccggcgaaagaaggaacgagttgaagcagttaatatggctgaaggaatcattcacgacacagaaaccaagatggaagaattcaaggaccaattacctgctgatgagtgcaacaagctgaaagaagagatttccaaaatgagggagctcctggctagaaaagacagcgaaacaggagaaaatattagacaggcagcatcctctcttcagcaggcatcactgaagctgttcgaaatggcatacaaaaagatggcatctgagcgagaaggctctggaagttctggcactggggaacaaaaggaagatcaaaaggaggaaaaacagtaataatagcagaaattttgaagccagaaggacaacatatgaagcttaggagtgaagagacttcctgagcagaaatgggcgaacttcagtctttttactgtgtttttgcagtattctatatataatttccttaatttgtaaatttagtgaccattagctagtgatcatttaatggacagtgattctaacagtataaagttcacaatattctatgtccctagcctgtcatttttcagctgcatgtaaaaggaggtaggatgaattgatcattataaagatttaactattttatgctgaagtgaccatattttcaaggggtgaaaccatctcgcacacagcaatgaaggtagtcatccatagacttgaaatgagaccacatatggggatgagatccttctagttagcctagtactgctgtactggcctgtatgtacatggggtccttcaactgaggccttgcaagtcaagctggctgtgccatgtttgtagatggggcagaggaatctagaacaatgggaaacttagctatttatattaggtacagctattaaaacaaggtaggaatgaggctagacctttaacttccctaaggcatacttttctagctaccttctgccctgtgtctggcacctacatccttgatgattgttctcttacccattctggaatttttttttttttaaataaatacagaaagcatcttgatctcttgtttgtgaggggtgatgccctgagatttagcttcaagaatatgccatggctcatgcttcccatatttcccaaagagggaaatacaggatttgctaacactggttaaaaatgcaaattcaagatttggaagggctgttataatgaaataatgagcagtatcagcatgtgcaaatcttgtttgaaggattttattttctccccttagacctttggtacatttagaatcttgaaagtttctagatctctaacatgaaagtttctagatctctaacatgaaagtttttagatctctaacatgaaaaccaaggtggctattttcaggttgctttcagctccaagtagaaataaccagaattggcttacattaaagaaactgcatctagaaataagtcctaagatactatttctatggctcaaaaataaaaggaacccagatttctttcccta

SEQ ID NO: 27 - RNA, 19 nt, Artificial Sequence
  Description of Artificial Sequence Synthetic oligonucleotide
gagacagccgacacagaua

SEQ ID NO: 28 - RNA, 19 nt, Artificial Sequence
  Description of Artificial Sequence Synthetic oligonucleotide
ugacuuacgugucuagcuu

SEQ ID NO: 29 - RNA, 19 nt, Artificial Sequence
  Description of Artificial Sequence Synthetic oligonucleotide
gaacugcccgaccggauga

SEQ ID NO: 30 - RNA, 19 nt, Artificial Sequence
  Description of Artificial Sequence Synthetic oligonucleotide
gaauacggcuuuugacgug

SEQ ID NO: 31 - RNA, 19 nt, Artificial Sequence
  Description of Artificial Sequence Synthetic oligonucleotide
ggaagugcaugguaaauuu

SEQ ID NO: 32 - RNA, 19 nt, Artificial Sequence
  Description of Artificial Sequence Synthetic oligonucleotide
cauccaaguucauauacua

SEQ ID NO: 33 - RNA, 19 nt, Artificial Sequence
  Description of Artificial Sequence Synthetic oligonucleotide
gcagaaagacacaccacaa

SEQ ID NO: 34 - RNA, 19 nt, Artificial Sequence
  Description of Artificial Sequence Synthetic oligonucleotide
ugguuuaccucauaugauu

SEQ ID NO: 35 - RNA, 19 nt, Artificial Sequence
  Description of Artificial Sequence Synthetic oligonucleotide
gggccagggcuuucgacuu

SEQ ID NO: 36 - RNA, 19 nt, Artificial Sequence
  Description of Artificial Sequence Synthetic oligonucleotide
caacaaucauauucacguu

SEQ ID NO: 37 - RNA, 19 nt, Artificial Sequence
  Description of Artificial Sequence Synthetic oligonucleotide
gaucaauccuccaugagua

SEQ ID NO: 38 - RNA, 19 nt, Artificial Sequence
  Description of Artificial Sequence Synthetic oligonucleotide
gagcagcuauucuucuuau

SEQ ID NO: 39 - RNA, 19 nt, Artificial Sequence
  Description of Artificial Sequence Synthetic oligonucleotide
ggaagagauuuguggauua

SEQ ID NO: 40 - RNA, 19 nt, Artificial Sequence
  Description of Artificial Sequence Synthetic oligonucleotide
ggccagaucugcuuuagaa

SEQ ID NO: 41 - RNA, 19 nt, Artificial Sequence
  Description of Artificial Sequence Synthetic oligonucleotide
gaauauggcauuucaguau

SEQ ID NO: 42 - RNA, 19 nt, Artificial Sequence
  Description of Artificial Sequence Synthetic oligonucleotide
gaagauuggacuaugcuaa

SEQ ID NO: 43 - RNA, 19 nt, Artificial Sequence
  Description of Artificial Sequence Synthetic oligonucleotide
gaauugagccacagaguaa

SEQ ID NO: 44 - RNA, 19 nt, Artificial Sequence
  Description of Artificial Sequence Synthetic oligonucleotide
gguuuacucuccaauguua

SEQ ID NO: 45 - RNA, 19 nt, Artificial Sequence
  Description of Artificial Sequence Synthetic oligonucleotide
ucauaaagcuucaccaauc

SEQ ID NO: 46 - RNA, 19 nt, Artificial Sequence
  Description of Artificial Sequence Synthetic oligonucleotide
acuagaaugugcaccauaa

SEQ ID NO: 47 - RNA, 19 nt, Artificial Sequence
  Description of Artificial Sequence Synthetic oligonucleotide
uaacacgcgcuucggaaua

SEQ ID NO: 48 - RNA, 19 nt, Artificial Sequence
  Description of Artificial Sequence Synthetic oligonucleotide
gaaaccaaguacagaugga

SEQ ID NO: 49 - RNA, 19 nt, Artificial Sequence
  Description of Artificial Sequence Synthetic oligonucleotide
gaguacggccugacguuua

SEQ ID NO: 50 - RNA, 19 nt, Artificial Sequence
  Description of Artificial Sequence Synthetic oligonucleotide
ccugauagguuuaggauac

SEQ ID NO: 51 - RNA, 19 nt, Artificial Sequence
  Description of Artificial Sequence Synthetic oligonucleotide
cugaguaaaugccggcuua

SEQ ID NO: 52 - RNA, 20 nt, Artificial Sequence
  Description of Artificial Sequence Synthetic oligonucleotide
cgacgcagacgcccagaaaa

SEQ ID NO: 53 - RNA, 19 nt, Artificial Sequence
  Description of Artificial Sequence Synthetic oligonucleotide
guaaggaacggauaugguu

SEQ ID NO: 54 - RNA, 19 nt, Artificial Sequence
  Description of Artificial Sequence Synthetic oligonucleotide
gcggaggcagcaaauguua

SEQ ID NO: 55 - RNA, 19 nt, Artificial Sequence
  Description of Artificial Sequence Synthetic oligonucleotide
ggaagcaacuuacggauac

SEQ ID NO: 56 - RNA, 19 nt, Artificial Sequence
  Description of Artificial Sequence Synthetic oligonucleotide
gauucuggcucaauuauua

SEQ ID NO: 57 - RNA, 19 nt, Artificial Sequence
  Description of Artificial Sequence Synthetic oligonucleotide
gaagucagaugaaggcuuu

SEQ ID NO: 58 - RNA, 19 nt, Artificial Sequence
  Description of Artificial Sequence Synthetic oligonucleotide
gugaacaaaugggaaguuu

SEQ ID NO: 59 - RNA, 19 nt, Artificial Sequence
  Description of Artificial Sequence Synthetic oligonucleotide
ggaauggccuuagucauga

SEQ ID NO: 60 - RNA, 19 nt, Artificial Sequence
  Description of Artificial Sequence Synthetic oligonucleotide
ccaaugggauaguacaugu

SEQ ID NO: 61 - RNA, 19 nt, Artificial Sequence
  Description of Artificial Sequence Synthetic oligonucleotide
ccuauggucuagacaaauc